(19) United States
(12) Patent Application Publication
(10) Pub. No.: US 2023/0357324 A1
(43) Pub. Date: Nov. 9, 2023

(54) IMPROVED DEPENDOPARVOVIRUS PRODUCTION COMPOSITIONS AND METHODS

(71) Applicant: DYNO THERAPEUTICS, INC., Watertown, MA (US)

(72) Inventors: Jeffrey GEROLD, Watertown, MA (US); Eric KELSIC, Watertown, MA (US); Sam SINAI, Watertown, MA (US); Adrian VERES, Watertown, MA (US); Samuel WOLOCK, Watertown, MA (US)

(21) Appl. No.: 18/042,890

(22) PCT Filed: Aug. 26, 2021
(86) PCT No.: PCT/US2021/047700

§ 371 Date: Feb. 24, 2023

Related U.S. Application Data
(60) Provisional application No. 63/070,763, filed on Aug. 26, 2020.

(51) Int. Cl. C07K 14/005

(52) U.S. Cl. CPC C07K 14/005; C12N 2750/14122; C12N 2750/14151; C12N 2750/14143; C12N 2840/50

(57) ABSTRACT

The disclosure is directed in part to improved methods of making dependoparvovirus particles utilizing a nucleic acid comprising an open reading from encoding membrane-associated accessory protein (MAAP) comprising an exogenous start codon.

### CROSS-REFERENCE TO RELATED APPLICATIONS

This application claims priority to U.S. Provisional Application No. 63/070,763, filed Aug. 26, 2020, which is hereby incorporated by reference in its entirety.

### BACKGROUND

Dependoparvoviruses, e.g. adeno-associated dependoparvoviruses, e.g. adeno-associated viruses (AAVs), are of interest as vectors for delivering various payloads to cells, including in human subjects.

### SUMMARY

The present disclosure provides, in part, improved methods of producing a dependoparvovirus, compositions for use in the same, as well as viral particles produced by the same. The disclosure is based, in part, on the discovery that a cell comprising a mutated open reading frame (ORF) encoding Membrane-Associated Accessory Protein (MAAP) when used to produce dependoparvovirus exhibits an improvement in a production characteristic involved in production of a dependoparvovirus particle. Such production characteristics include, e.g., an increase in the amount of dependoparvovirus polypeptide or particle produced intracellularly, an increase in the amount of correctly folded dependoparvovirus polypeptide, an increase in the amount of dependoparvovirus particle secreted from the cell, or an overall increase in the amount of dependoparvovirus particle produced. In an embodiment, the improvement is relative to what is seen with an otherwise similar cell comprising an ORF encoding MAAP not comprising the mutation, e.g., the improvement is relative to a unit of time or resource expended or relative to an otherwise similar cell comprising an ORF encoding MAAP not comprising the mutation. Without wishing to be bound by theory, the presence of an exogenous start codon in the ORF encoding MAAP is thought to improve one or more production characteristics associated with production of dependoparvovirus in a cell.

In one aspect, the disclosure is directed, in part, to a nucleic acid comprising a sequence encoding an ORF for a functional dependoparvovirus B MAAP which ORF comprises an exogenous start codon. In some embodiments, the dependoparvovirus B is an adeno-associated dependoparvovirus (AAV). In some embodiments, the AAV is AAV5.

In another aspect, the disclosure is directed, in part, to a dependoparvovirus particle comprising a nucleic acid described herein (e.g., a nucleic acid comprising a sequence encoding an ORF for a functional dependoparvovirus B MAAP which ORF comprises an exogenous start codon). In some embodiments, the dependoparvovirus particle is of a different clade or strain than the ORF encoding the dependoparvovirus B MAAP. In some embodiments, the dependoparvovirus particle is of the same clade or strain as the ORF encoding the dependoparvovirus B MAAP.

In another aspect, the disclosure is directed, in part, to a vector, e.g., a plasmid, comprising a nucleic acid described herein (e.g., a nucleic acid comprising a sequence encoding an ORF for a functional dependoparvovirus B MAAP which ORF comprises an exogenous start codon).

In another aspect, the disclosure is directed, in part, to a cell, cell-free system, or other translation system comprising a nucleic acid described herein (e.g., a nucleic acid comprising a sequence encoding an ORF for a functional dependoparvovirus B MAAP which ORF comprises an exogenous start codon). In some embodiments, the cell, cell-free system, or other translation system comprises a vector described herein. In some embodiments, the cell, cell-free system, or other translation system comprises a dependoparvovirus particle described herein.

In another aspect, the disclosure is directed, in part, to a dependoparvovirus B MAAP polypeptide (e.g., a mutant polypeptide), an amino acid of which (e.g., the first amino acid) corresponds to an exogenous start codon. In some embodiments, the dependoparvovirus B MAAP polypeptide (e.g., a mutant polypeptide) is encoded by a nucleic acid described herein. In some embodiments, disclosure is directed to a purified or isolated preparation of a dependoparvovirus B MAAP polypeptide described herein.

In another aspect, the disclosure is directed, in part, to a cell, cell-free system, or other translation system comprising a dependoparvovirus B MAAP polypeptide (e.g., a mutant polypeptide), an amino acid of which (e.g., the first amino acid) corresponds to an exogenous start codon. In some embodiments, the cell, cell-free system, or other translation system comprises a vector described herein. In some embodiments, the cell, cell-free system, or other translation system comprises a dependoparvovirus particle described herein.

In another aspect, the disclosure is directed, in part, to a nucleic acid comprising a sequence encoding a VP1 polypeptide, wherein the VP1 encoding sequence comprises a change or mutation corresponding to or arising from the presence of sequence encoding an exogenous start codon in the MAAP polypeptide encoding sequence. Without wishing to be bound by theory, in the dependoparvovirus genome the sequence encoding VP1 overlaps with the sequence encoding MAAP. In some embodiments, the sequences encoding MAAP and VP1 are in different reading frames. In some embodiments, a mutation that creates an exogenous start codon in an ORF encoding a MAAP polypeptide alters the amino acid sequence of the VP1 polypeptide.

In another aspect, the disclosure is directed, in part, to a dependoparvovirus particle comprising a nucleic acid described herein (e.g., a nucleic acid comprising a sequence encoding a VP1 polypeptide, wherein the VP1 encoding sequence comprises a change or mutation corresponding to or arising from the presence of sequence encoding an exogenous start codon in the MAAP polypeptide encoding sequence).

In another aspect, the disclosure is directed, in part, to a VP1 polypeptide described herein (e.g., wherein the VP1 encoding sequence comprises a change or mutation corresponding to or arising from the presence of sequence encoding an exogenous start codon in the MAAP polypeptide encoding sequence, e.g., wherein the exogenous start codon in an ORF encoding a MAAP polypeptide alters the amino acid sequence of the VP1 polypeptide).

In another aspect, the disclosure is directed, in part, to a vector comprising a nucleic acid described herein, e.g., a nucleic acid comprising a sequence encoding a VP1 polypeptide, wherein the VP1 encoding sequence comprises a change or mutation corresponding to or arising from the presence of sequence encoding an exogenous start codon in the MAAP polypeptide encoding sequence.

In another aspect, the disclosure is directed, in part, to a cell, cell-free system, or other translation system comprising a nucleic acid or vector described herein, e.g., comprising a sequence encoding a VP1 polypeptide, wherein the VP1 encoding sequence comprises a change or mutation corresponding to or arising from the presence of sequence encoding an exogenous start codon in the MAAP polypeptide encoding sequence. In some embodiments, the cell, cell-free system, or other translation system comprises a dependoparvovirus particle described herein, e.g., wherein the particle comprises a nucleic acid comprising a sequence encoding a VP1 polypeptide, wherein the VP1 encoding sequence comprises a change or mutation corresponding to or arising from the presence of sequence encoding an exogenous start codon in the MAAP polypeptide encoding sequence.

In another aspect, the disclosure is directed, in part, to a cell, cell-free system, or other translation system comprising a VP1 polypeptide described herein, wherein the VP1 encoding sequence comprises a change or mutation corresponding to or arising from the presence of sequence encoding an exogenous start codon in the MAAP polypeptide encoding sequence. In some embodiments, the cell, cell-free system, or other translation system comprises a dependoparvovirus particle described herein, e.g., wherein the particle comprises a nucleic acid comprising a sequence encoding a VP1 polypeptide, wherein the VP1 encoding sequence comprises a change or mutation corresponding to or arising from the presence of sequence encoding an exogenous start codon in the MAAP polypeptide encoding sequence.

In another aspect, the disclosure is directed, in part, to a method of delivering a payload to a cell comprising contacting the cell with a dependoparvovirus particle comprising a nucleic acid described herein. In another aspect, the disclosure is directed, in part, to a method of delivering a payload to a cell comprising contacting the cell with a dependoparvovirus particle comprising a VP1 polypeptide described herein.

In another aspect, the disclosure is directed, in part, to a method of making a dependoparvovirus particle, comprising providing a cell, cell-free system, or other translation system, comprising a nucleic acid described herein (e.g., a nucleic acid comprising a sequence encoding an ORF for a functional dependoparvovirus B MAAP which ORF comprises an exogenous start codon); and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In another aspect, the disclosure is directed, in part, to a method of making a dependoparvovirus particle, comprising providing a cell, cell-free system, or other translation system, comprising a polypeptide described herein, a dependoparvovirus B MAAP polypeptide (e.g., a mutant polypeptide), an amino acid of which (e.g., the first amino acid) corresponds to an exogenous start codon; and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In another aspect, the disclosure is directed, in part, to a dependoparvovirus particle made in a cell, cell-free system, or other translation system, wherein the cell, cell-free system, or other translation system comprises a nucleic acid encoding a dependoparvovirus B MAAP ORF comprising an exogenous stop codon or a MAAP polypeptide encoded by the MAAP ORF.

In another aspect, the disclosure is directed, in part, to a method of treating a disease or condition in a subject, comprising administering to the subject a dependoparvovirus particle described herein in an amount effective to treat the disease or condition.

The invention is further described with reference to the following numbered embodiments.

### ENUMERATED EMBODIMENTS

1. A nucleic acid comprising a sequence encoding an ORF for a functional dependoparvovirus B (e.g., an adeno-associated dependoparvovirus (AAV), e.g. AAV5) MAAP polypeptide, which ORF comprises an exogenous start codon.
2. The nucleic acid of embodiment 1, wherein a cell, cell-free system, or other translation system, comprising the nucleic acid packages, secretes, and/or produces dependoparvovirus (e.g., dependoparvovirus A or B, e.g., an adeno-associated dependoparvovirus (AAV), e.g. AAV5 or a serotype other than AAV5) particle at a level of at least 50% or more than that of a cell, cell-free system, or other translation system, comprising an otherwise similar nucleic acid that does not comprise the exogenous start codon.
3a. The nucleic acid of either of embodiments 1 or 2, wherein the sequence comprises a change or mutation at a position between or including nucleotides 14 to 250 of a VP1 encoding sequence (e.g., a sequence encoding AAV5 VP1, e.g., SEQ ID NO: 327) that creates an exogenous start codon at the position.
3b. The nucleic acid of any of embodiments 1-3a, wherein the sequence comprises a change or mutation at any of the positions listed in columns 4 or 5 of Table 1, or at a site one or two nucleotides downstream of said position, that creates an exogenous start codon at the position.
4. The nucleic acid of any of the above embodiments, wherein the change or mutation is relative to a reference sequence.
5. The nucleic acid of embodiment 4, wherein the reference sequence comprises a wildtype sequence, e.g., SEQ ID NO: 331, or a sequence with at least 90 or 95% sequence identity with a wildtype sequence, e.g., SEQ ID NO: 331.
6. The nucleic acid of any of the above embodiments, wherein the exogenous start codon is at a position listed in columns 4 or 5 of Table 1.
7. The nucleic acid of any of the above embodiments, wherein the functional dependoparvovirus B (e.g., AAV5) MAAP polypeptide ORF:
   (a) mediates detectable translation initiation in a cell, e.g., a human cell, cell-free system, or other translation system, or
   (b) if present in a cell, cell-free system, or other translation system, otherwise competent for producing dependoparvovirus particles, allows for the production of dependoparvovirus particles.
8. The nucleic acid of any of the above embodiments, wherein the MAAP polypeptide comprises an amino acid sequence with at least 80% sequence identity to SEQ ID NO: 325.
9. The nucleic acid of any of the above embodiments, wherein the MAAP polypeptide comprises an amino acid sequence with at least 85% sequence identity to SEQ ID NO: 325.
10. The nucleic acid of any of the above embodiments, wherein the MAAP polypeptide comprises an amino acid sequence with at least 90% sequence identity to SEQ ID NO: 325.
11. The nucleic acid of any of the above embodiments, wherein the MAAP polypeptide comprises an amino acid sequence with at least 95% sequence identity to SEQ ID NO: 325.
12. The nucleic acid of any of the above embodiments, wherein the MAAP polypeptide, except for the amino acid specified by the exogenous start codon, differs from the sequence of SEQ ID NO: 325, by no more than 20 amino acid residues.
13. The nucleic acid of any of the above embodiments, wherein the MAAP polypeptide, except for the amino acid specified by the exogenous start codon, differs from the sequence of SEQ ID NO: 325, by no more than 15 amino acid residues.
14. The nucleic acid of any of the above embodiments, wherein the MAAP polypeptide, except for the amino acid specified by the exogenous start codon, differs from the sequence of SEQ ID NO: 325, by no more than 10 amino acid residues.
15. The nucleic acid of any of the above embodiments, wherein the MAAP polypeptide, except for the amino acid specified by the exogenous start codon, differs from the sequence of SEQ ID NO: 325, by no more than 5 amino acid residues.
16. The nucleic acid of any of the above embodiments, wherein the MAAP polypeptide, except for the amino acid specified by the exogenous start codon, differs from the sequence of SEQ ID NO: 325, by no more than 2 amino acid residues.
17. The nucleic acid of any of the above embodiments, wherein the MAAP polypeptide differs from the sequence of SEQ ID NO: 325 in a pattern specified by a CIGAR string listed in column 8 of Table 1.
18. The nucleic acid of any of the preceding embodiments wherein the exogenous start codon is an ATG, CTG, GTG, ACG, TTG, ATT, ATC, ATA, or AGG.
19. The nucleic acid of any of the preceding embodiments wherein the exogenous start codon is an ATG.
20. The nucleic acid of any of embodiments 1-18, wherein the exogenous start codon is an CTG.
21. The nucleic acid of any of the above embodiments, wherein the sequence encoding the exogenous start codon results in an amino acid change in VP1.
22. The nucleic acid of any of embodiments 1-20, wherein the sequence encoding the exogenous start codon does not result in an amino acid change in VP1.
23. The nucleic acid of any of the above embodiments, wherein the MAAP polypeptide comprises one, two, three, four, five, or all of:
   an N-terminal disordered region, optionally capable of binding to a polypeptide;
   a short hydrophobic region comprising a beta-strand, optionally capable of binding to a polypeptide;
   a T/S rich disordered region, optionally enriched in charged amino acids;
   a region devoid of predicted secondary structure, optionally capable of binding to a polypeptide;
   a disordered region, optionally capable of forming an alpha-helix, or
   a C-terminal amphipathic region comprising an alpha-helix, optionally capable of binding a membrane.
24. The nucleic acid of any of the above embodiments, wherein the MAAP polypeptide comprises from most N-terminal to most C-terminal, one, two, three, four, five, or all of:
   an N-terminal disordered region, optionally capable of binding to a polypeptide;
   a short hydrophobic region comprising a beta-strand, optionally capable of binding to a polypeptide;
   a T/S rich disordered region, optionally enriched in charged amino acids;
   a region devoid of predicted secondary structure, optionally capable of binding to a polypeptide;
   a disordered region, optionally capable of forming an alpha-helix, and
   a C-terminal amphipathic region comprising an alpha-helix, optionally capable of binding a membrane.
25. The nucleic acid of any of the above embodiments, wherein the MAAP polypeptide comprises, from most N-terminal to most C-terminal:
   an N-terminal disordered region, optionally capable of binding to a polypeptide;
   a short hydrophobic region comprising a beta-strand, optionally capable of binding to a polypeptide;
   a T/S rich disordered region, optionally enriched in charged amino acids;
   a region devoid of predicted secondary structure, optionally capable of binding to a polypeptide;
   a disordered region, optionally capable of forming an alpha-helix, and
   a C-terminal amphipathic region comprising an alpha-helix, optionally capable of binding a membrane.
26. The nucleic acid of any of the above embodiments, wherein the MAAP polypeptide comprises at least 80, 85, 90, 95, 100, 105, 110, 115, or 116 amino acids (e.g., a full length MAAP polypeptide) and optionally no more than 120, 119, 118, 117, 116, 115, 110, 105, or 100 amino acids.
27. The nucleic acid of any of the above embodiments, wherein the ORF encoding MAAP comprises a nucleic acid sequence with at least 80, 85, 90, 91, 92, 93, 94, 95, 96, 97, 98, 99, or 100% identity to any of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, or 320.
28. The nucleic acid of any of the above embodiments, wherein the ORF encoding MAAP comprises a nucleic acid sequence that differs by no more than 20, 19, 18, 17, 16, 15, 14, 13, 12, 11, 10, 9, 8, 7, 6, 5, 4, 3, 2, or 1 nucleotides from the sequence of any of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, or 320.
29. The nucleic acid of any of the above embodiments, wherein the MAAP polypeptide comprises an amino acid sequence with at least 80, 85, 90, 91, 92, 93, 94, 95, 96, 97, 98, 99, or 100% identity to any of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319.
30. The nucleic acid of any of the above embodiments, wherein the MAAP polypeptide comprises an amino acid sequence that differs by no more than 20, 19, 18, 17, 16, 15, 14, 13, 12, 11, 10, 9, 8, 7, 6, 5, 4, 3, 2, or 1 amino acids from the sequence of any of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319.
31. The nucleic acid of any of embodiments 2-30, wherein the dependoparvovirus particle is a dependoparvovirus A particle.
32. The nucleic acid of any of embodiments 2-30, wherein the dependoparvovirus particle is a dependoparvovirus B particle.
33. The nucleic acid of any of embodiments 2-32, wherein the dependoparvovirus particle is an adeno-associated dependoparvovirus (AAV) particle.
34a. The nucleic acid of embodiment 33, wherein the AAV particle is an AAV5 particle.
34b. The nucleic acid of embodiment 33, wherein the AAV particle is a particle of a serotype other than AAV5.
35. The nucleic acid of any of the above embodiments, wherein the MAAP polypeptide is an AAV5 MAAP polypeptide.
36. The nucleic acid of any of the above embodiments, further comprising a sequence encoding a dependoparvovirus, e.g., dependoparvovirus B, e.g., an AAV5, VP1 polypeptide.
37. The nucleic acid of embodiment 36, wherein the VP1 polypeptide comprises an amino acid sequence with at least 80% sequence identity to SEQ ID NO: 321.
38. The nucleic acid of either of embodiments 36 or 37, wherein the VP1 polypeptide comprises an amino acid sequence with at least 85% sequence identity to SEQ ID NO: 321.
39. The nucleic acid of any of embodiments 36-38, wherein the VP1 polypeptide comprises an amino acid sequence with at least 90% sequence identity to SEQ ID NO: 321.
40. The nucleic acid of any of embodiments 36-39, wherein the VP1 polypeptide comprises an amino acid sequence with at least 95% sequence identity to SEQ ID NO: 321.
41. The nucleic acid of any of embodiments 36-40, wherein the VP1 polypeptide, except for the amino acid specified by the exogenous start codon, differs from the sequence of SEQ ID NO: 321, by no more than 20 amino acid residues.
42. The nucleic acid of any of embodiments 36-41, wherein the VP1 polypeptide, except for the amino acid specified by the exogenous start codon, differs from the sequence of SEQ ID NO: 321, by no more than 15 amino acid residues.
43. The nucleic acid of any of embodiments 36-42, wherein the VP1 polypeptide, except for the amino acid specified by the exogenous start codon, differs from the sequence of SEQ ID NO: 321, by no more than 10 amino acid residues.
44. The nucleic acid of any of embodiments 36-43, wherein the VP1 polypeptide, except for the amino acid specified by the exogenous start codon, differs from the sequence of SEQ ID NO: 321, by no more than 5 amino acid residues.
45. The nucleic acid of any of embodiments 36-44, wherein the VP1 polypeptide, except for the amino acid specified by the exogenous start codon, differs from the sequence of SEQ ID NO: 321, by no more than 2 amino acid residues.
46. The nucleic acid of any of embodiments 36-45, wherein the sequence encoding the VP1 polypeptide comprises a nucleic acid sequence with at least 80, 85, 90, 91, 92, 93, 94, 95, 96, 97, 98, 99, or 100% identity to any of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, or 318.
47. The nucleic acid of any of embodiments 36-46, wherein the sequence encoding the VP1 polypeptide comprises a nucleic acid sequence that, except for the amino acid specified by the exogenous start codon, differs by no more than 20, 19, 18, 17, 16, 15, 14, 13, 12, 11, 10, 9, 8, 7, 6, 5, 4, 3, 2, or 1 nucleotides from the sequence of any of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, or 318.
48. The nucleic acid of any of embodiments 36-47, wherein the VP1 polypeptide comprises an amino acid sequence with at least 80, 85, 90, 91, 92, 93, 94, 95, 96, 97, 98, 99, or 100% identity to any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317.
49. The nucleic acid of any of embodiments 36-48, wherein the VP1 polypeptide comprises an amino acid sequence that, except for the amino acid specified by the exogenous start codon, differs by no more than 20, 19, 18, 17, 16, 15, 14, 13, 12, 11, 10, 9, 8, 7, 6, 5, 4, 3, 2, or 1 amino acids from the sequence of any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317.
50. The nucleic acid of any of the above embodiments, further comprising a sequence encoding a dependoparvovirus, e.g., dependoparvovirus B, e.g., an AAV5, VP2 polypeptide.
51. The nucleic acid of embodiment 50, wherein the VP2 polypeptide comprises an amino acid sequence with at least 80% sequence identity to SEQ ID NO: 322.
52. The nucleic acid of either of embodiments 50 or 51, wherein the VP2 polypeptide comprises an amino acid sequence with at least 85% sequence identity to SEQ ID NO: 322.
53. The nucleic acid of any of embodiments 50-52, wherein the VP2 polypeptide comprises an amino acid sequence with at least 90% sequence identity to SEQ ID NO: 322.
54. The nucleic acid of any of embodiments 50-53, wherein the VP2 polypeptide comprises an amino acid sequence with at least 95% sequence identity to SEQ ID NO: 322.
55. The nucleic acid of any of embodiments 50-54, wherein the VP2 polypeptide differs from the sequence of SEQ ID NO: 322 by no more than 20 amino acid residues.
56. The nucleic acid of any of embodiments 50-55, wherein the VP2 polypeptide differs from the sequence of SEQ ID NO: 322 by no more than 15 amino acid residues.
57. The nucleic acid of any of embodiments 50-56, wherein the VP2 polypeptide differs from the sequence of SEQ ID NO: 322 by no more than 10 amino acid residues.
58. The nucleic acid of any of embodiments 50-57, wherein the VP2 polypeptide differs from the sequence of SEQ ID NO: 322 by no more than 5 amino acid residues.
59. The nucleic acid of any of embodiments 50-58, wherein the VP2 polypeptide differs from the sequence of SEQ ID NO: 322 by no more than 2 amino acid residues.
60. The nucleic acid of any of the above embodiments, further comprising a sequence encoding a dependoparvovirus, e.g., dependoparvovirus B, e.g., an AAV5, VP3 polypeptide.
61. The nucleic acid of embodiment 60, wherein the VP3 polypeptide comprises an amino acid sequence with at least 80% sequence identity to SEQ ID NO: 323.
62. The nucleic acid of either of embodiments 60 or 61, wherein the VP3 polypeptide comprises an amino acid sequence with at least 85% sequence identity to SEQ ID NO: 323.
63. The nucleic acid of any of embodiments 60-62, wherein the VP3 polypeptide comprises an amino acid sequence with at least 90% sequence identity to SEQ ID NO: 323.
64. The nucleic acid of any of embodiments 60-63, wherein the VP3 polypeptide comprises an amino acid sequence with at least 95% sequence identity to SEQ ID NO: 323.
65. The nucleic acid of any of embodiments 60-64, wherein the VP3 polypeptide differs from the sequence of SEQ ID NO: 323, by no more than 20 amino acid residues.
66. The nucleic acid of any of embodiments 60-65, wherein the VP3 polypeptide differs from the sequence of SEQ ID NO: 323, by no more than 15 amino acid residues.
67. The nucleic acid of any of embodiments 60-66, wherein the VP3 polypeptide differs from the sequence of SEQ ID NO: 323, by no more than 10 amino acid residues.
68. The nucleic acid of any of embodiments 60-67, wherein the VP3 polypeptide differs from the sequence of SEQ ID NO: 323, by no more than 5 amino acid residues.
69. The nucleic acid of any of embodiments 60-68, wherein the VP3 polypeptide differs from the sequence of SEQ ID NO: 323, by no more than 2 amino acid residues.
70. The nucleic acid of any of the above embodiments, further comprising a sequence encoding a dependoparvovirus, e.g., dependoparvovirus B, e.g., an AAV5 or serotype other than AAV5, Cap polypeptide.
71. The nucleic acid of embodiment 70, wherein the Cap polypeptide comprises an amino acid sequence with at least 80% sequence identity to SEQ ID NO: 321.
72. The nucleic acid of either of embodiments 70 or 71, wherein the Cap polypeptide comprises an amino acid sequence with at least 85% sequence identity to SEQ ID NO: 321.
73. The nucleic acid of any of embodiments 70-72, wherein the Cap polypeptide comprises an amino acid sequence with at least 90% sequence identity to SEQ ID NO: 321.
74. The nucleic acid of any of embodiments 70-73, wherein the Cap polypeptide comprises an amino acid sequence with at least 95% sequence identity to SEQ ID NO: 321.
75. The nucleic acid of any of embodiments 70-74, wherein the Cap polypeptide, except for the amino acid specified by the exogenous start codon, differs from the sequence of SEQ ID NO: 321, by no more than 20 amino acid residues.
76. The nucleic acid of any of embodiments 70-75, wherein the Cap polypeptide, except for the amino acid specified by the exogenous start codon, differs from the sequence of SEQ ID NO: 321, by no more than 15 amino acid residues.
77. The nucleic acid of any of embodiments 70-76, wherein the Cap polypeptide, except for the amino acid specified by the exogenous start codon, differs from the sequence of SEQ ID NO: 321, by no more than 10 amino acid residues.
78. The nucleic acid of any of embodiments 70-77, wherein the Cap polypeptide, except for the amino acid specified by the exogenous start codon, differs from the sequence of SEQ ID NO: 321, by no more than 5 amino acid residues.
79. The nucleic acid of any of embodiments 70-78, wherein the Cap polypeptide, except for the amino acid specified by the exogenous start codon, differs from the sequence of SEQ ID NO: 321, by no more than 2 amino acid residues.
80. The nucleic acid of any of the above embodiments, further comprising a sequence encoding a dependoparvovirus, e.g., dependoparvovirus A or B, Rep polypeptide, e.g, encoding an AAV5 or serotype other than AAV5 Rep polypeptide.
81. The nucleic acid of any of the above embodiments further comprising a sequence encoding an AAV2 Rep gene.
82. The nucleic acid of either of embodiments 80 or 81, wherein the Rep polypeptide comprises an amino acid sequence with at least 80% sequence identity to any of SEQ ID NOs: 333-336.
83. The nucleic acid of any of embodiments 80-82, wherein the Rep polypeptide comprises an amino acid sequence with at least 85% sequence identity to any of SEQ ID NOs: 333-336.
84. The nucleic acid of any of embodiments 80-83, wherein the Rep polypeptide comprises an amino acid sequence with at least 90% sequence identity to any of SEQ ID NOs: 333-336.
85. The nucleic acid of any of embodiments 80-84, wherein the Rep polypeptide comprises an amino acid sequence with at least 95% sequence identity to any of SEQ ID NOs: 333-336.
86. The nucleic acid of any of embodiments 80-85, wherein the Rep polypeptide differs from the sequence of any of SEQ ID NOs: 333-336, by no more than 20 amino acid residues.
87. The nucleic acid of any of embodiments 80-86, wherein the Rep polypeptide differs from the sequence of any of SEQ ID NOs: 333-336, by no more than 15 amino acid residues.
88. The nucleic acid of any of embodiments 80-87, wherein the Rep polypeptide differs from the sequence of any of SEQ ID NOs: 333-336, by no more than 10 amino acid residues.
89. The nucleic acid of any of embodiments 80-88, wherein the Rep polypeptide differs from the sequence of any of SEQ ID NOs: 333-336, by no more than 5 amino acid residues.
90. The nucleic acid of any of embodiments 80-89, wherein the Rep polypeptide differs from the sequence of any of SEQ ID NOs: 333-336, by no more than 2 amino acid residues.
91. The nucleic acid of any of embodiments 36-90, wherein one or more or all of the VP1, VP2, VP3, Cap, or Rep polypeptides is, respectively, an AAV5 VP1, VP2, VP3, Cap, or Rep polypeptide.
92. The nucleic acid of any of embodiments 36-90, wherein one or more of the VP1, VP2, VP3, Cap, or Rep polypeptides is, respectively, not an AAV5 VP1, VP2, VP3, Cap, or Rep polypeptide.
93. The nucleic acid of any of the above embodiments, further comprising an AAV Cap gene that comprises a sequence encoding VP3, VP2, VP1, AAP, Rep, or X gene that does not naturally occur in an AAV5 genome.
94. The nucleic acid of any of embodiments 36-93, wherein one or more (e.g., all) of the VP1, VP2, VP3, or Cap polypeptides is an AAV5 VP1, VP2, VP3, or Cap polypeptide, and the Rep polypeptide is an AAV2 Rep polypeptide.
95. The nucleic acid of any of the above embodiments, wherein the nucleic acid comprises a mutation, e.g, at any of the positions listed in columns 4 or 5 of Table 1, or at a site within two nucleotides of said position, that creates the exogenous start codon.
96. The nucleic acid of embodiment 95, wherein a mutation at the positions listed in columns 4 or 5 of Table 1, or at a site within two nucleotides of said position, results in a silent nucleic acid mutation in VP1.
97. The nucleic acid of embodiment 95, wherein a mutation at the positions listed in columns 4 or 5 of Table 1, or at a site within two nucleotides of said position, results in an amino acid change in VP1.
98. The nucleic acid of embodiment 97, the amino acid change in VP1 is a conservative change.
99. The nucleic acid of any of embodiments 36-95, 97, or 98, wherein the polypeptide sequence encoded by the dependoparvovirus Cap gene, e.g., the VP1 polypeptide sequence, comprises a mutation (e.g., a substitution) corresponding to the exogenous start codon in the MAAP polypeptide ORF.
100. The nucleic acid of any of embodiments 36-94 or 96, wherein the polypeptide sequence encoded by the dependoparvovirus Cap gene, e.g., the VP1 polypeptide sequence, does not comprises a mutation (e.g., a substitution) corresponding to the exogenous start codon in the MAAP polypeptide ORF.
101. The nucleic acid of any of embodiments 36-100, wherein the polypeptide sequence encoded by the dependoparvovirus Cap gene, e.g., the VP1 polypeptide sequence, comprises a mutation corresponding to a difference between any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317, and a wildtype VP1 polypeptide sequence, e.g., SEQ ID NO: 321.
102. The nucleic acid of any of embodiments 36-101, wherein the VP1 polypeptide differs from the sequence of SEQ ID NO: 321 in a pattern specified by a CIGAR string listed in column 7 of Table 1.
103. The nucleic acid of any of embodiments 70-102, wherein the polypeptide produced from the Cap gene is functional.
104. The nucleic acid of any of embodiments 70-102, wherein the polypeptide produced from the Cap gene is capable of assembling into a dependoparvovirus capsid.
105. The nucleic acid of any of embodiments 70-104, wherein the polypeptide produced from the Cap gene is capable of packaging dependoparvovirus DNA into a dependoparvovirus capsid.
106. The nucleic acid of any of embodiments 70-105, wherein a dependoparvovirus capsid assembled from the polypeptide produced from the Cap gene is capable of infecting a target cell.
107. The nucleic acid of any of the above embodiments, wherein a cell, cell-free system, or other translation system, comprising the nucleic acid secretes functional dependoparvovirus particle at a level of at least 60, 70, 80, 90, 100, 110, 120, 130, 140, 150, 160, 170, 180, 190, 200, 250, 300, 350, 400, 450, 500, 600, 700, 800, 900, or 1000% that of a cell, cell-free system, or other translation system, comprising an otherwise similar nucleic acid that does not comprise the exogenous start codon.
108. The nucleic acid of any of the above embodiments, wherein a cell, cell-free system, or other translation system, comprising the nucleic acid secretes more functional dependoparvovirus particle than a cell, cell-free system, or other translation system, comprising an otherwise similar nucleic acid that does not comprise the exogenous start codon.
109. A dependoparvovirus particle comprising the nucleic acid of any of the above embodiments.
110. The dependoparvovirus particle of embodiment 109, wherein the dependoparvovirus particle is a dependoparvovirus A particle.
111. The dependoparvovirus particle of embodiment 109, wherein the dependoparvovirus particle is a dependoparvovirus B particle.
112. The dependoparvovirus particle of any of embodiments 109-111, wherein the dependoparvovirus particle is an adeno-associated dependoparvovirus (AAV) particle.
113. A vector, e.g., a plasmid, comprising the nucleic acid of any of embodiments 1-108.
114. A cell comprising the nucleic acid of any of embodiments 1-108.
115. A cell-free system comprising the nucleic acid of any of embodiments 1-108.
116. A translation system comprising the nucleic acid of any of embodiments 1-108.
117. A cell, comprising the vector of embodiment 113.
118. A cell-free system comprising the vector of embodiment 113.
119. A translation system comprising the vector of embodiment 113.
120. A cell comprising the dependoparvovirus particle of any of embodiments 109-112.
121. A cell-free system comprising the dependoparvovirus particle of any of embodiments 109-112.
122. A translation system comprising the dependoparvovirus particle of any of embodiments 109-112.
123. A dependoparvovirus B (e.g., AAV5) MAAP polypeptide, an amino acid of which corresponds to an exogenous start codon.
124. The dependoparvovirus B (e.g., AAV5) MAAP polypeptide of embodiment 123, wherein a cell, cell-free system, or other translation system, comprising the MAAP polypeptide packages, secretes, and/or produces dependoparvovirus (e.g., dependoparvovirus A or B, e.g., an adeno-associated dependoparvovirus (AAV), e.g. AAV5 or a serotype other than AAV5) particle at a level of at least 50% or more than that of a cell, cell-free system, or other translation system, comprising an otherwise similar MAAP polypeptide that does not comprise the amino acid corresponding to the exogenous start codon.
125. The dependoparvovirus B (e.g., AAV5) MAAP polypeptide of either of embodiments 123 or 124, wherein the amino acid corresponding to the exogenous start codon comprises a methionine.
126. The dependoparvovirus B (e.g., AAV5) MAAP polypeptide of either of embodiments 123 or 124, wherein the amino acid corresponding to the exogenous start codon comprises a leucine.
127. The dependoparvovirus B (e.g., AAV5) MAAP polypeptide of any of embodiments 123-126, wherein the MAAP polypeptide differs from the sequence of SEQ ID NO: 325 in a pattern specified by a CIGAR string listed in column 8 of Table 1.
128. The dependoparvovirus B (e.g., AAV5) MAAP polypeptide of any of embodiments 123-127, wherein the MAAP polypeptide comprises an amino acid sequence with at least 80, 85, 90, 91, 92, 93, 94, 95, 96, 97, 98, 99, or 100% identity to any of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319.
129. The dependoparvovirus B (e.g., AAV5) MAAP polypeptide of any of embodiments 123-128, wherein the MAAP polypeptide comprises an amino acid sequence that differs by no more than 20, 19, 18, 17, 16, 15, 14, 13, 12, 11, 10, 9, 8, 7, 6, 5, 4, 3, 2, or 1 amino acids from the sequence of any of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319.
130. A dependoparvovirus B (e.g., AAV5) MAAP polypeptide encoded by the nucleic acid of any of embodiments 1-108.
131. An isolated or purified preparation of the polypeptide of any of embodiments 123-130.
132. The MAAP polypeptide or isolated or purified preparation of any of embodiments 123-131, wherein the MAAP polypeptide is an AAV5 MAAP polypeptide.
133. A nucleic acid comprising a sequence encoding a VP1 polypeptide, wherein the VP1 encoding sequence comprises a change or mutation corresponding to or arising from the presence of sequence encoding an exogenous start codon in the MAAP polypeptide encoding sequence.
134. The nucleic acid of embodiment 133, wherein the change or mutation is silent with respect to VP1 amino acid sequence.
135. The nucleic acid of embodiment 133, wherein the change or mutation results in a change to the VP1 amino acid sequence.
136. The nucleic acid of embodiment 135, wherein the change to the VP1 amino acid sequence is a conservative change.
137. The nucleic acid of embodiment 135, wherein the change to the VP1 amino acid sequence is a non-conservative change.
138. The nucleic acid of any of embodiments 133-137, wherein the VP1 polypeptide differs from the sequence of SEQ ID NO: 321 in a pattern specified by a CIGAR string listed in column 7 of Table 1.
139. The nucleic acid of any of embodiments 133-138, wherein the sequence encoding the VP1 polypeptide comprises a nucleic acid sequence with at least 80, 85, 90, 91, 92, 93, 94, 95, 96, 97, 98, 99, or 100% identity to any of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, or 318.
140. The nucleic acid of any of embodiments 133-139, wherein the sequence encoding the VP1 polypeptide comprises a nucleic acid sequence that, except for the amino acid specified by the exogenous start codon, differs by no more than 20, 19, 18, 17, 16, 15, 14, 13, 12, 11, 10, 9, 8, 7, 6, 5, 4, 3, 2, or 1 nucleotides from the sequence of any of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, or 318.
141. The nucleic acid of any of embodiments 133-140, wherein the VP1 polypeptide comprises an amino acid sequence with at least 80, 85, 90, 91, 92, 93, 94, 95, 96, 97, 98, 99, or 100% identity to any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317.
142. The nucleic acid of any of embodiments 133-141, wherein the VP1 polypeptide comprises an amino acid sequence that, except for the amino acid specified by the exogenous start codon, differs by no more than 20, 19, 18, 17, 16, 15, 14, 13, 12, 11, 10, 9, 8, 7, 6, 5, 4, 3, 2, or 1 amino acids from the sequence of any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317.
143a. The nucleic acid of any of embodiments 133-142, wherein the MAAP polypeptide is a dependoparvovirus B (e.g., AAV5) MAAP polypeptide.
143b. The nucleic acid of any of embodiments 133-143, wherein the VP1 polypeptide is other than an AAV5 VP1 polypeptide.
144. The nucleic acid of any of embodiments 133-143, wherein the VP1 polypeptide is a dependoparvovirus (e.g., dependoparvovirus B, e.g., AAV5) VP1 polypeptide.
145. A dependoparvovirus (e.g., dependoparvovirus A or B, e.g., AAV5 or a serotype other than AAV5) particle comprising the nucleic acid of any of embodiments 133-144.
146. A VP1 polypeptide encoded by the nucleic acid of any of embodiments 36-108 or 133-144.
147. A dependoparvovirus (e.g., dependoparvovirus A or B, e.g., AAV5 or a serotype other than AAV5) particle comprising the VP1 polypeptide of embodiment 146.
148. An isolated or purified preparation of the polypeptide of embodiment 146.
149. A vector, e.g., a plasmid, comprising the nucleic acid of any of embodiments 133-144.
150. A cell comprising the nucleic acid of any of embodiments 133-144.
151. A cell-free system comprising the nucleic acid of any of embodiments 133-144.
152. A translation system comprising the nucleic acid of any of embodiments 133-144.
153. A cell comprising the vector of embodiment 149.
154. A cell-free system comprising the vector of embodiment 149.
155. A translation system comprising the vector of embodiment 149.
156. A cell comprising the AAV particle of embodiment 145 or 147.
157. A cell-free system comprising the AAV particle of embodiment 145 or 147.
158. A translation system comprising the AAV particle of embodiment 145 or 147.
159. A method of delivering a payload to a cell comprising contacting the cell with a viral particle comprising the nucleic acid of any of embodiments 1-108 or 133-144.
160. A method of delivering a payload to a subject comprising administering to the subject, a viral particle comprising the VP1 polypeptide of embodiment 146.
161. A method of making a dependoparvovirus (e.g., dependoparvovirus A or B, e.g., an adeno-associated dependoparvovirus (AAV), e.g. AAV5 or a serotype other than AAV5) particle, comprising:
   providing a cell, cell-free system, or other translation system, comprising:
      a nucleic acid of any of embodiments 1-108; and
   cultivating the cell, cell-free system, or other translation system, under conditions suitable for the production of the dependoparvovirus particle,
   thereby making the dependoparvovirus particle.
162. The method of embodiment 161, wherein the nucleic acid of 1-108 is disposed in the genome of the dependoparvovirus and is packaged into the dependoparvovirus particle.
163. The method of embodiment 161, wherein the cell, cell-free system, or other translation system comprises a second nucleic acid molecule and said second nucleic acid molecule is packaged in the dependoparvovirus particle.
164. The method of embodiment 163, wherein the second nucleic acid comprises an exogenous sequence.
165. The method of embodiment 163, wherein the exogenous sequence encodes an exogenous polypeptide.
166. The method of either of embodiment 164 or 165, wherein the exogenous sequence encodes a therapeutic product.
167. The method of any of embodiments 163-166, wherein a nucleic acid of any of embodiments 1-108 mediates the production of a dependoparvovirus particle which does not include said nucleic acid of any of embodiments 1-108.
168. A method of making a dependoparvovirus (e.g., dependoparvovirus A or B, e.g., an adeno-associated dependoparvovirus (AAV), e.g. AAV5 or a serotype other than AAV5) particle, comprising:
   providing a cell, cell-free system, or other translation system, comprising:
      a MAAP polypeptide of any of embodiments 123-130 or 132; and
   cultivating the cell, cell-free system, or other translation system, under conditions suitable for the production of the dependoparvovirus particle,
   thereby making the dependoparvovirus particle.
169. A method of making a dependoparvovirus (e.g., dependoparvovirus A or B, e.g., an adeno-associated dependoparvovirus (AAV), e.g. AAV5 or a serotype other than AAV5) particle, comprising:
   providing a cell, cell-free system, or other translation system, comprising:
      a VP1 polypeptide of embodiment 146; and
   cultivating the cell, cell-free system, or other translation system, under conditions suitable for the production of the dependoparvovirus particle,
   thereby making the dependoparvovirus particle.
170. The method of any of embodiments 161-169, wherein the dependoparvovirus particle is a dependoparvovirus A particle.
171. The method of any of embodiments 161-169, wherein the dependoparvovirus particle is a dependoparvovirus B particle.
172. The method of any of embodiments 161-169, wherein the dependoparvovirus particle is an adeno-associated dependoparvovirus (AAV) particle.
173. The method of any of embodiments 161-169, wherein the dependoparvovirus particle is an AAV5 particle.
174. The method of any of embodiments 161-169, wherein the dependoparvovirus particle is a particle other than an AAV5 particle.
175. A dependoparvovirus (e.g., dependoparvovirus A or B, e.g., an adeno-associated dependoparvovirus (AAV), e.g. AAV5 or a serotype other than AAV5) particle made in a cell, cell-free system, or other translation system, the cell, cell-free system, or other translation system, comprising a nucleic acid encoding a dependoparvovirus B (e.g., AAV5) MAAP ORF comprising an exogenous stop codon or a MAAP polypeptide encoded by the MAAP ORF.
176. The dependoparvovirus particle of embodiment 175, wherein the cell, cell-free system, or other translation system, comprising the nucleic acid packages, secretes, and/or produces dependoparvovirus (e.g., dependoparvovirus A or B, e.g., an adeno-associated dependoparvovirus (AAV), e.g. AAV5 or a serotype other than AAV5) particle at a level of at least 50% or more than that of a cell, cell-free system, or other translation system, comprising an otherwise similar nucleic acid that does not comprise the exogenous start codon.
177. The particle of embodiment of either embodiment 175 or 176, wherein the particle is made by the method of any of embodiments 161-174.
178. The particle of any of embodiments 175-177, further comprising a packaged nucleic acid molecule.
179. The particle of any of embodiments 175-178, wherein a nucleic acid of 1-108 is packaged into the dependoparvovirus (e.g., dependoparvovirus A or B, e.g., an adeno-associated dependoparvovirus (AAV), e.g. AAV5 or a serotype other than AAV5) particle.
180. The particle of any of embodiments 175-178, wherein a nucleic acid comprising an exogenous sequence is packaged into the dependoparvovirus (e.g., dependoparvovirus A or B, e.g., an adeno-associated dependoparvovirus (AAV), e.g. AAV5 or a serotype other than AAV5) particle.
181. The particle of any of embodiment 180, wherein a nucleic acid sequence encoding an exogenous polypeptide is packaged into the dependoparvovirus (e.g., dependoparvovirus A or B, e.g., an adeno-associated dependoparvovirus (AAV), e.g. AAV5 or a serotype other than AAV5) particle.
182. The particle of any of embodiment 180-181, wherein the nucleic acid encoding an exogenous polypeptide encodes a therapeutic product.
183. The particle of any of embodiments 180-182, wherein the dependoparvovirus particle is a dependoparvovirus B particle.
184. The particle of any of embodiments 180-182, wherein the dependoparvovirus particle is a dependoparvovirus A particle.
185. The particle of any of embodiments 180-184, wherein the dependoparvovirus particle is an AAV particle.
186. The particle of embodiment 185, wherein the dependoparvovirus particle is an AAV5 particle.
187. The particle of embodiment 185, wherein the dependoparvovirus particle is a particle other than an AAV5 particle.
188. The dependoparvovirus particle of any of embodiments 175-187, wherein the particle comprises:
   a capsid packaged nucleic acid molecule,
   a VP1 polypeptide,
   a VP2 polypeptide, and
   optionally a VP3 polypeptide.
189. The dependoparvovirus particle of embodiment 188, wherein:
   a) the ratio of VP1 to VP2 or VP3 polypeptide is greater than the ratio in a reference particle, wherein the production of the reference particle was mediated by a wild type MAAP polypeptide (e.g., a MAAP polypeptide encoded by an ORF not comprising an exogenous start codon);
   b) the ratio of VP1 to either of VP2 or VP3, is altered in a mutant MAAP polypeptide dependent fashion, e.g., in a fashion mediated by a mutant MAAP polypeptide described herein;
   c) the ratio of VP1 to VP2 is greater than 1.2:1, 1.5:1, or 2:1; or
   d) the ratio of VP1 to VP3 is greater than 1.2:10, 1.5:10, or 2:1.
190. The dependoparvovirus particle of any of embodiments 175-178 or 180-189, wherein the production of the particle was mediated by a MAAP polypeptide encoded by a sequence comprising an exogenous start codon (e.g., a MAAP polypeptide encoded by a nucleic acid of embodiment 1-108), wherein the sequence encoding the MAAP polypeptide comprising the exogenous start codon is not packaged into the particle.
191. The particle of either of embodiments 189 or 190, wherein the ratio of VP1, VP2, and VP3 polypeptide in the capsid is 1:1:X, wherein X is less than 8 and may be 0 (e.g., VP3 may not be present in the capsid).
192. The particle of any of embodiments 188-191, wherein the nucleic acid used to produce VP3 does not comprise a mutation, e.g., a VP3 mutation, that decreases or abrogates the expression of the VP3 polypeptide (e.g., relative to a reference dependoparvovirus (e.g., dependoparvovirus A or B, e.g., an adeno-associated dependoparvovirus (AAV), e.g. AAV5 or a serotype other than AAV5)).
193. The particle of any of embodiments 188-192, wherein the nucleic acid used to produce VP2 does not comprise a mutation, e.g., a VP2 mutation, that decreases or abrogates the expression of the VP2 polypeptide (e.g., relative to a reference dependoparvovirus (e.g., dependoparvovirus A or B, e.g., an adeno-associated dependoparvovirus (AAV), e.g. AAV5 or a serotype other than AAV5)).
194. A method of delivering a payload (e.g., a nucleic acid) to a cell comprising contacting the cell with a particle described herein comprising the payload.
195. The method of embodiment 194, wherein the particle is a particle of embodiments 147 or 180-193 or a particle made by a method of embodiments 161-174.
196. A method of delivering a payload (e.g., a nucleic acid) to a subject comprising administering to the subject a particle described herein comprising the payload.
197. The method of embodiment 196, wherein the particle is a particle of embodiments 147 or 180-193, or a particle made by a method of embodiments 161-174.
198. The method of any of embodiments 194-197, wherein the particle delivers the payload to a preselected target cell, organ, tissue, or region.
199. The method of any of embodiments 194-198, wherein the particle comprises a mutant Cap polypeptide which preferentially targets the payload to a preselected target cell, organ, tissue, or region.
200. A method of treating a disease or condition in a subject, comprising administering to the subject a particle described herein in an amount effective to treat the disease or condition.
201. The method of embodiment 200, wherein the particle is a particle of embodiments 147 or 180-193, or a particle made by a method of embodiments 161-174.
202. The method of either of embodiments 200 or 201, wherein the particle comprises a payload, e.g., a therapeutic product.

### BRIEF DESCRIPTION OF THE DRAWINGS

FIG. 1, Panels A, B, and C shows three graphs of the dependoparvovirus production efficiency of mutant dependoparvovirus variants relative to wildtype AAV5. Each mutant dependoparvovirus variant comprises an exogenous start codon (ATG) in the VP1 frame (+0) (shown in Panel A), the +1 frame (shown in Panel B), or the +2 frame (shown in Panel C). The x-axis shows the position at which the ATG was introduced. Black dots denote exemplary capsid variants, and gray dots represent all other variants. Gray bars indicate the boundaries of VP1, VP2, VP3, and AAP, and the putative boundaries of MAAP.

FIG. 2, Panels A and B, shows two graphs of the dependoparvovirus production efficiency of mutant dependoparvovirus variants relative to wildtype AAV5. Each mutant dependoparvovirus variant comprises an exogenous start codon (ATG in Panel A or CTG in Panel B) in the +1 frame. The x-axis shows the position at which the exogenous start codon was introduced. Black dots denote exemplary capsid variants, and gray dots represent all other variants. Gray bars indicate the boundaries of VP1, VP2, VP3, and AAP, and the putative boundaries of MAAP.

### DETAILED DESCRIPTION

The present disclosure is directed, in part, to the discovery that a cell comprising a mutated open reading frame (ORF) encoding Membrane-Associated Accessory Protein (MAAP) may exhibit an improvement in a production characteristic involved in production of a dependoparvovirus particle. Without wishing to be bound by theory, MAAP is thought to play a role in packaging and/or secretion of dependoparvovirus particles from a host cell. Some dependoparvovirus clades or strains have a genome comprising a MAAP encoding ORF comprising (e.g., at the start of the coding sequence) non-canonical start codons. Other dependoparvovirus clades or strains have a genome comprising a MAAP encoding ORF that does not comprise a canonical or non-canonical start codon (e.g., proximal to the start of the coding sequence). In some embodiments, a MAAP encoding ORF comprising a non-canonical start codon or not comprising a non-canonical or canonical start codon (e.g., proximal to the start of the coding sequence) does not appreciably express (e.g., does not express) in a cell. Without wishing to be bound by theory, it is thought that the presence of an exogenous start codon in the ORF encoding MAAP may increase expression of MAAP, e.g., relative to an otherwise similar ORF not comprising the exogenous start codon. Without wishing to be bound by theory, it is thought that the presence of an exogenous start codon, e.g., that more strongly promotes translation initiation than the codon endogenously present, may increase expression of MAAP, e.g., relative to an otherwise similar ORF not comprising the exogenous start codon. Such an improved ORF encoding MAAP may be useful to improve production of dependoparvovirus particles by cells, cell free systems, or translation systems comprising said ORF.

### Definitions

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values.

Dependoparvovirus capsid: As used herein, the term “dependoparvovirus capsid” refers to an assembled viral capsid comprising dependoparvovirus polypeptides. In some embodiments, a dependoparvovirus capsid is a functional dependoparvovirus capsid, e.g., is fully folded and/or assembled, is competent to infect a target cell, or remains stable (e.g., folded/assembled and/or competent to infect a target cell) for at least a threshold time.

Dependoparvovirus particle: As used herein, the term “dependoparvovirus particle” refers to an assembled viral capsid comprising dependoparvovirus polypeptides and a packaged nucleic acid, e.g., comprising a payload, one or more components of a dependoparvovirus genome (e.g., a whole dependoparvovirus genome), or both. In some embodiments, a dependoparvovirus particle is a functional dependoparvovirus particle, e.g., comprises a desired payload, is fully folded and/or assembled, is competent to infect a target cell, or remains stable (e.g., folded/assembled and/or competent to infect a target cell) for at least a threshold time.

Dependoparvovirus X particle/capsid: As used herein, the term “dependoparvovirus X particle/capsid” refers to a dependoparvovirus particle/capsid comprising at least one polypeptide or polypeptide encoding nucleic acid sequence derived from a naturally occurring dependoparvovirus X species. For example, a dependoparvovirus B particle refers to a dependoparvovirus particle comprising at least one polypeptide or polypeptide encoding nucleic acid sequence derived from a naturally occurring dependoparvovirus B sequence. Derived from, as used in this context, means having at least 80, 85, 90, 91, 92, 93, 94, 95, 96, 97, 98, 99, or 100% identity to the sequence in question. Correspondingly, an AAVX particle/capsid, as used herein, refers to an AAV particle/caspid comprising at least one polypeptide or polypeptide encoding nucleic acid sequence derived from a naturally occurring AAV X serotype. For example, an AAV5 particle refers to an AAV particle comprising at least one polypeptide or polypeptide encoding nucleic acid sequence derived from a naturally occurring AAV5 sequence.

Exogenous: As used herein, the term “exogenous” refers to a feature, sequence, or component present in a circumstance (e.g., in a nucleic acid, polypeptide, or cell) that does not naturally occur in said circumstance. For example, a nucleic acid sequence comprising an ORF encoding a polypeptide may comprise an exogenous start codon. Use of the term exogenous in this fashion means that an ORF encoding a polypeptide comprising the start codon in question at this position does not occur naturally, e.g., is not present in AAV2, AAV5, or AAV9, e.g., is not present in SEQ ID NO: 331. In some embodiments, the exogenous start codon may replace an endogenous start codon. In some embodiments, the exogenous start codon may replace a codon that is not recognized as a start codon by the host cell. A person of skill will readily understand that a sequence (e.g., a start codon) may be exogenous when provided in a first ORF (e.g., that does not naturally comprise a start codon at the site in question) but may not be exogenous in a second ORF (e.g., that does naturally comprise that particular start codon at the site in question).

Functional: As used herein in reference to a dependoparvovirus MAAP polypeptide, the term “functional” refers to a dependoparvovirus MAAP polypeptide that either: increases the packaging and/or secretion of dependoparvovirus particles when present in a host cell (e.g., relative to an otherwise similar host cell lacking the MAAP polypeptide), or provides at least 50, 60, 70, 80, 90, or 100% of the activity (e.g., packaging and/or secretion promoting activity) of a naturally occurring MAAP polypeptide, e.g., when measured in an otherwise similar cell or system. As used herein in reference to an ORF, the term “functional” means that the ORF mediates translation initiation in a cell, e.g., a human cell, cell-free system, or other translation system (e.g., detectable translation initiation). As used herein in reference to a polypeptide component of a dependoparvovirus capsid (e.g., Cap (e.g., VP1, VP2, and/or VP3) or Rep), the term “functional” refers to a polypeptide which provides at least 50, 60, 70, 80, 90, or 100% of the activity of a naturally occurring version of that polypeptide component (e.g., when present in a host cell). For example, a functional VP1 polypeptide may stably fold and assemble into a dependoparvovirus capsid (e.g., that is competent for packaging and/or secretion). As used herein in reference to a dependoparvovirus capsid or particle, “functional” refers to a capsid or particle comprising one or more of the following production characteristics: comprises a desired payload, is fully folded and/or assembled, is competent to infect a target cell, or remains stable (e.g., folded/assembled and/or competent to infect a target cell) for at least a threshold time.

MAAP Polypeptide: As used herein, a “MAAP polypeptide” refers to: a naturally occurring dependoparvovirus membrane associated accessory polypeptide (MAAP); a mutant, artificial, or synthetic MAAP known in the art; or a polypeptide comprising an amino acid sequence with at least 70, 75, 80, 85, 90, 95, 96, 97, 98, or 99% identity to the aforementioned. In some embodiments, a MAAP polypeptide is a functional MAAP polypeptide. In some embodiments, an ORF encoding a MAAP polypeptide comprises an exogenous start codon. In some embodiments, a MAAP polypeptide is a full length MAAP polypeptide (e.g., comprising all the regions and/or domains corresponding to a naturally occurring dependoparvovirus MAAP). In some embodiments, a MAAP polypeptide comprises a truncation or a deletion (e.g., relative to a naturally occurring MAAP). In some embodiments, a MAAP polypeptide comprises one, two, three, four, five, or all of (e.g., from most N-terminal to most C-terminal): an N-terminal disordered region, optionally capable of binding to a polypeptide; a short hydrophobic region comprising a beta-strand, optionally capable of binding to a polypeptide; a T/S rich disordered region, optionally enriched in charged amino acids; a region devoid of predicted secondary structure, optionally capable of binding to a polypeptide; a disordered region, optionally capable of forming an alpha-helix; or a C-terminal amphipathic region comprising an alpha-helix, optionally capable of binding a membrane. In some embodiments, a MAAP polypeptide comprises one or more amino acids in addition to those present in a naturally occurring MAAP polypeptide. In some embodiments, such additional amino acids are at the N-terminal end of the MAAP polypeptide, e.g., as a consequence of the presence of an exogenous start codon upstream of an endogenous or putative start codon in the ORF encoding MAAP. In some embodiments, the amino acid encoded by the exogenous start codon is an additional amino acid.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers or a longer polynucleotide chain comprising many individual nucleic acid monomers. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid has a nucleotide sequence that encodes a functional gene product such as an RNA or protein. In some embodiments, a nucleic acid is partly or wholly single stranded; in some embodiments, a nucleic acid is partly or wholly double stranded.

Production characteristic: As used herein, the term “production characteristic” refers to a characteristic of a dependoparvovirus production process that is alterable by changing the characteristics of a nucleic acid, polypeptide, or dependoparvovirus particle described herein. Production characteristics include, but are not limited to: the amount of a dependoparvovirus polypeptide or particle produced intracellularly, the amount of correctly folded dependoparvovirus polypeptide, the amount of correctly packaged dependoparvovirus capsid or particle, the amount of dependoparvovirus particle secreted from the cell, the overall amount of dependoparvovirus particle produced, or any preceding characteristic relative to a unit of time or resource expended, or any preceding characteristic relative to an otherwise similar cell (e.g., comprising an ORF encoding MAAP not comprising the exogenous start codon). In some embodiments, changes (e.g., improvements) in a production characteristic are host cell or dependoparvovirus clade or strain dependent. For example, a dependoparvovirus production process may comprise providing a host cell comprising a nucleic acid encoding the components of a dependoparvovirus particle. In some embodiments, the dependoparvovirus production process may comprise providing the host cell with a nucleic acid comprising a sequence encoding an ORF encoding a functional dependoparvovirus B MAAP which ORF comprises an exogenous start codon. Providing the nucleic acid comprising a sequence encoding the ORF may improve one or more production characteristics.

Start codon: As used herein, the term “start codon” refers to any codon recognized by a host cell as a site to initiate translation (e.g., a site that mediates detectable translation initiation). Without wishing to be bound by theory, start codons vary in strength, with strong start codons more strongly promoting translation initiation and weak start codons less strongly promoting translation initiation. The canonical start codon is ATG, which encodes the amino acid methionine, but a number of non-canonical start codons are also recognized by host cells.

### Nucleic Acids Comprising ORFs Encoding MAAP Polypeptide

The disclosure is directed, in part, to a nucleic acid comprising a sequence encoding an open reading frame (ORF) for a functional MAAP polypeptide comprising an exogenous start codon. Without wishing to be bound by theory, it is thought that a cell, cell-free system, or translation system (e.g., for producing a dependoparvovirus particle) comprising a nucleic acid encoding an ORF encoding a functional MAAP polypeptide comprising an exogenous start codon may exhibit one or more improved production characteristics.

In some embodiments, the exogenous start codon is a canonical start codon, e.g., ATG. In some embodiments, the exogenous start codon is a non-canonical start codon. In some embodiments, the exogenous start codon is selected from CTG, GTG, ACG, TTG, ATT, ATC, ATA, or AGG, e.g., CTG. Without wishing to be bound by theory, a naturally occurring ORF encoding MAAP may comprise a non-canonical start codon or in some cases lack a detectable start codon in the expected position near the beginning of the MAAP encoding sequence. The disclosure is based, in part, on the discovery that introducing an exogenous start codon to the ORF encoding MAAP may improve one or more production characteristics relating to the production of a dependoparvovirus.

In some embodiments, the exogenous start codon is positioned at the beginning of the MAAP polypeptide encoding sequence (e.g., the exogenous start codon replaces an endogenous first codon of the MAAP polypeptide encoding sequence). In some embodiments, the exogenous start codon is positioned at a point within the MAAP polypeptide encoding sequence. In such embodiments, the exogenous start codon may become the first codon of a truncated MAAP polypeptide (e.g., that is missing amino acids N-terminal of the exogenous start codon). In some embodiments, the exogenous start codon is positioned outside of the MAAP polypeptide encoding sequence (e.g., in sequence N-terminal of the endogenous start codon or the position corresponding to an endogenous start codon). In such embodiments, the exogenous start codon may become the first codon of an expanded MAAP polypeptide (e.g., that includes additional amino acids N-terminal of the endogenous coding sequence). Without wishing to be bound by theory, it is thought that some naturally occurring ORFs encoding MAAP polypeptide comprise a weak, non-canonical start codon or no observable start codon near the beginning of the MAAP polypeptide encoding sequence or at a position where a start codon exists in another species or serotype. As such, additional amino acids N-terminal of those encoded by an endogenous coding sequence can refer to amino acids encoded upstream of a putative start codon but that are included in the MAAP polypeptide of another species' or serotype's MAAP polypeptide because that species' or serotype's MAAP ORF has a start codon further upstream.

By introducing an exogenous start codon, the level of MAAP translation initiation may be increased. Without wishing to be bound by theory, truncation or expansion of the MAAP polypeptide amino acid sequence (e.g., by introducing an exogenous start codon at some distance N- or C-terminal of the endogenous start codon) may be less important to one or more production characteristics associated with producing a dependoparvovirus particle than increasing the level of MAAP polypeptide (albeit truncated or expanded) in the host cell.

Without wishing to be bound by theory, the ORF encoding wildtype AAV2 MAAP polypeptide comprises a CTG start codon. Some other ORFs encoding MAAP polypeptide, e.g., AAV5 MAAP polypeptide, do not appear to comprise a start codon at the position corresponding to the CTG start codon of AAV2 MAAP, instead having one or more candidate start codons downstream. CIGAR strings given in Table 1, which specify positions of difference between mutant MAAP encoding nucleic acid sequences and wildtype MAAP nucleic acid sequences, are given relative to the nucleic acid sequence encoding AAV5 MAAP which begins at the site of the AAV2 MAAP start codon (SEQ ID NO: 331). A person of skill will understand that a position with a given number in the genome of one dependoparvovirus species or serotype may have a different, readily ascertainable number at the corresponding position in the genome of a different dependoparvovirus species or serotype.

In some embodiments, a CTG start codon encodes a leucine amino acid. In other embodiments, a CTG start codon may be decoded by cell, cell-free system, or other translation system as encoding a methionine. Without wishing to be bound by theory, it is thought that cells and translation systems recognizing alternate, non-ATG start codons may produce polypeptides from transcripts comprising non-ATG start codons where the first amino acid is nonetheless methionine. Without wishing to be bound by theory, it is possible that the cell or translation system decodes the non-ATG start codon (e.g., CTG) as methionine, e.g., via an alternative tRNA or promiscuous binding of a Met-tRNA, or that the non-ATG start codon encoded amino acid is edited or substituted for methionine by some other process (see, e.g., Kearse, M, and Wilusz, J. Genes & Dev. 2017. 31: 1717-1731). In some embodiments, an ORF encoding MAAP polypeptide comprises an exogenous start codon comprising CTG, wherein the first amino acid of the MAAP polypeptide is methionine. In some embodiments, an ORF encoding MAAP polypeptide comprises an exogenous start codon comprising CTG, wherein the first amino acid of the MAAP polypeptide is leucine.

In some embodiments, the exogenous start codon is introduced at any of the positions listed in columns 4 or 5 of Table 1 in a nucleic acid comprising an ORF encoding MAAP, or at a corresponding position in a nucleic acid comprising an ORF encoding MAAP from another dependoparvovirus. A person of skill will understand that in some cases a plurality of mutations may introduce an exogenous start codon at a position listed in columns 4 or 5 of Table 1, e.g., a mutation at the nucleotide of said position or in a nearby (e.g., adjacent) nucleotide. In some embodiments, the exogenous start codon is at a position listed in column 4 or 5 of Table 1 in a nucleic acid comprising an ORF encoding MAAP, or at a corresponding position in a nucleic acid comprising an ORF encoding MAAP from another dependoparvovirus.

In some embodiments, a nucleic acid comprising a sequence encoding an ORF for a functional MAAP polypeptide comprises an alteration relative to a reference sequence that creates an exogenous start codon. In some embodiments, the reference sequence is a naturally occurring dependoparvovirus MAAP. In some embodiments, the reference sequence is a mutant, artificial, or synthetic MAAP known in the art. In some embodiments, the reference sequence comprises a wildtype sequence, e.g., SEQ ID NO: 331, or a sequence with at least 90, 91, 92, 93, 94, 95, 96, 97, 98, or 99% sequence identity with a wildtype sequence, e.g., SEQ ID NO: 331. In some embodiments, the alteration comprises substitution, deletion, or insertion of one or more nucleotides, or a combination of a substitution, deletion, or insertion. In some embodiments, a nucleic acid comprising a sequence encoding an ORF for a functional MAAP polypeptide comprises an alteration at any of the positions listed in columns 4 or 5 of Table 1, or a nearby, e.g., adjacent, position, relative to the AAV5 genome or at a corresponding position in another dependoparvovirus genome, that creates an exogenous start codon.

In some embodiments, the sequence encoding an ORF for a functional MAAP polypeptide has at least 80, 85, 90, 91, 92, 93, 94, 95, 96, 97, 98, 99, or 100% identity to a reference sequence. In some embodiments, the sequence encoding an ORF for a functional MAAP polypeptide has at least 80, 85, 90, 91, 92, 93, 94, 95, 96, 97, 98, 99, or 100% identity to a wildtype dependoparvovirus B MAAP gene. In some embodiments, the sequence encoding an ORF for a functional MAAP polypeptide has at least 80, 85, 90, 91, 92, 93, 94, 95, 96, 97, 98, 99, or 100% identity to a wildtype AAV5 MAAP gene, e.g., SEQ ID NO: 331. In some embodiments, the sequence encoding an ORF for a functional MAAP polypeptide is identical to a wildtype dependoparvovirus B (e.g., AAV5) MAAP encoding sequence except for the exogenous start codon. In some embodiments, the ORF for a functional MAAP polypeptide differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotides from the nucleotide sequence of a wildtype ORF encoding a wildtype MAAP polypeptide (e.g., from a nucleotide sequence of SEQ ID NO: 331). In some embodiments, the ORF for a functional MAAP polypeptide differs by 1-30, 5-30, 10-30, 15-30, 20-30, 25-30, 1-25, 5-25, 10-25, 15-25, 20-25, 1-20, 5-20, 10-20, 15-20, 1-15, 5-15, 10-15, 1-10, 5-10, or 1-5 nucleotides from the nucleotide sequence of a wildtype ORF encoding a wildtype MAAP polypeptide (e.g., from a nucleotide sequence of SEQ ID NO: 331).

In some embodiments, the nucleic acid sequence comprising an ORF encoding a MAAP polypeptide is wildtype (e.g., a wildtype dependoparvovirus B, e.g., an AAV5, sequence encoding MAAP polypeptide) at all other positions besides those affected by the exogenous start codon. In some embodiments, the nucleic acid sequence comprising an ORF encoding a MAAP polypeptide is wildtype (e.g., a wildtype dependoparvovirus B, e.g., an AAV5, sequence encoding MAAP polypeptide) at all other positions besides those affected by the exogenous start codon and a position that is altered (relative to a wildtype sequence, e.g., SEQ ID NO: 331) in any of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, or 320.

In some embodiments, the ORF for a functional dependoparvovirus (e.g., dependoparvovirus B, e.g., AAV5) MAAP polypeptide is a functional ORF. In some embodiments, the ORF for a functional dependoparvovirus (e.g., dependoparvovirus B, e.g., AAV5) MAAP polypeptide mediates detectable translation initiation in a cell, e.g., a human cell, cell-free system, or other translation system. In some embodiments, the ORF for a functional dependoparvovirus (e.g., dependoparvovirus B, e.g., AAV5) MAAP polypeptide allows for the production of dependoparvovirus particles when present in a cell, cell-free system, or other translation system, otherwise competent for producing dependoparvovirus particles.

In some embodiments, a nucleic acid comprising a sequence encoding an ORF for a functional MAAP polypeptide comprises an additional alteration relative to the reference sequence (i.e., in addition to an alteration that creates an exogenous start codon). In some embodiments, the additional alteration comprises substitution, deletion, or insertion of one or more nucleotides. In some embodiments, the additional alteration improves one or more production characteristics, e.g., of a dependoparvovirus particle or method of producing the same in a host cell.

Table 1 lists information regarding exemplary variant dependoparvovirus particles comprising nucleic acids comprising an ORF encoding a MAAP polypeptide comprising an exogenous start codon and the production characteristics of said exemplary variants. In addition, Table 1 lists information regarding the position of the exogenous start codon in a given exemplary variant (position numbers are given based on the VP1 encoding sequence of AAV5), changes to the VP1 polypeptide (if any) in the form of edit distance from AAV5 VP1 sequence, CIGAR notation of sequence alterations (relative to wildtype AAV5) for the VP1 polypeptide and MAAP polypeptide sequences of the variant, and SEQ ID NOs corresponding to the nucleic acid and amino acid sequences of the VP1 and MAAP of the variant (see Table 2). Exemplary sequences of nucleic acids encoding an ORF encoding a functional MAAP polypeptide comprising an exogenous start codon, as well as corresponding MAAP polypeptide sequences, are provided in Table 2 below. Exemplary sequences of nucleic acids encoding VP1 polypeptides, as well as corresponding VP1 polypeptide amino acid sequences, are also provided in Table 2 below.

TABLE 1

| | Column 2 | | Column | Column | Column 6 | | |
|---|---|---|---|---|---|---|---|
| | log2 | Column | 4 | 5 | SEQ ID | | |
| | (production | 3 | New | New | NOS | | |
| | efficiency | VP1 AA | MAAP | MAAP | associated | | |
| Column 1 | relative | edit | ATG | CTG | with | Column 7 | Column 8 |
| Variant | to | distance | nucleotide | nucleotide | Variant | VP1 | MAAP |
| No. | AAV5) | to AAV5 | position(s) | position(s) | No. | CIGAR | CIGAR |
| 1 | 9.63 | 1 | 74 | | 1, 2, 3, 4 | 24 = 1X700 = | 1I119 = |
| 2 | 9.07 | 2 | 74 | | 5, 6, 7, 8 | 24 = 1X5 = | 1I4 = 2X113= |
| | | | | | | 1X694= | |
| 3 | 8.68 | 5 | 122 | 143 | 9, 10, 11, | 38 = 1I2X9 = | 14D1X9 = |
| | | | | | 12 | 1X11 = | 1X10 = |
| | | | | | | 1X663= | 2X82= |
| 4 | 8.16 | 1 | 47 | | 13, 14, 15, | 15 = 1X709= | 10I119= |
| | | | | | 16 | | |
| 5 | 7.85 | 1 | 56 | | 17, 18, 19, | 18 = 1X706= | 7I119 = |
| | | | | | 20 | | |
| 6 | 7.74 | 1 | 44 | | 21, 22, 23, | 14 = 1X710= | 11I119 = |
| | | | | | 24 | | |
| 7 | 7.67 | 3 | 116 | 143, 191 | 25, 26, 27, | 37 = 1X1 = 112 | 12D2X26 = 1 |
| | | | | | 28 | 6 = 1X659= | X78= |
| 8 | 7.60 | 3 | 65 | | 29, 30, 31, | 18 = 1X2 = 1X | 412 = 1X116= |
| | | | | | 32 | 5 = 1X697= | |
| 9 | 7.58 | 1 | 248 | | 33, 34, 35, | 82 = 1X642= | 57D1X61= |
| | | | | | 36 | | |
| 10 | 7.46 | 6 | 119 | | 37, 38, 39, | 39 = 2X8 = | 14D2X8 = 1X4 = |
| | | | | | 40 | 1X4 = 1X4 = | 1X4 = 1X5 = |
| | | | | | | 1X5 = 1X659= | 1X78= |
| 11 | 7.43 | 1 | 101 | | 41, 42, 43, | 34 = 1I691 = | 7D2X110= |
| | | | | | 44 | | |
| 12 | 7.38 | 2 | 47 | | 45, 46, 47, | 15 = 1X3 = | 10I119= |
| | | | | | 48 | 1X705= | |
| 13 | 7.33 | 1 | 35 | | 49, 50, 51, | 11 = 1X713= | 14I119= |
| | | | | | 52 | | |
| 14 | 7.29 | 1 | 119 | | 53, 54, 55, | 39 = 1X685= | 14D1X104= |
| | | | | | 56 | | |
| 15 | 7.26 | 2 | 146 | | 57, 58, 59, | 49 = 1X4 = | 23D2X4 = |
| | | | | | 60 | 1X670= | 1X89= |
| 16 | 7.03 | 1 | 248 | | 61, 62, 63, | 82 = 1X642= | 57D1X61= |
| | | | | | 64 | | |
| 17 | 6.96 | 1 | 146 | | 65, 66, 67, | 49 = 1X675= | 23D2X94= |
| | | | | | 68 | | |
| 18 | 6.96 | 2 | 119 | | 69, 70, 71, | 39 = 1X18 = | 14D1X18 = |
| | | | | | 72 | 1X666= | 1X85= |
| 19 | 6.89 | 6 | | 77 | 73, 74, 75, | 1 = 1X19 = | 1X1 = 1X116= |
| | | | | | 76 | 2X1 = 2X1 = | |
| | | | | | | 1X697= | |
| 20 | 6.76 | 1 | 248 | | 77, 78, 79, | 82 = 1X642= | 57D1X61= |
| | | | | | 80 | | |
| 21 | 6.74 | 4 | 38, 104 | | 81, 82, 83, | 12 = 1I8 = | 8D1X110= |
| | | | | | 84 | 1X1 = 1X11 = | |
| | | | | | | 1X690= | |
| 22 | 6.42 | 2 | 47 | | 85, 86, 87, | 15 = 1X17 = | 1018 = 1X110= |
| | | | | | 88 | 1X691= | |
| 23 | 6.41 | 2 | 35 | | 89, 90, 91, | 11 = 1X12 = | 14I119= |
| | | | | | 92 | 1X700= | |
| 24 | 6.08 | 1 | 113 | | 93, 94, 95, | 37 = 1X687= | 12D1X106= |
| | | | | | 96 | | |
| 25 | 6.06 | 4 | 248 | | 97, 98, 99, | 65 = 1X2 = 2X | 57D1X61= |
| | | | | | 100 | 12 = 1X642= | |
| 26 | 5.94 | 3 | 101 | | 101, 102, | 34 = 1X2 = 1X | 8D1X3 = |
| | | | | | 103, 104 | 16 = 1X670= | 1X16 = 1X89= |
| 27 | 5.82 | 4 | 248 | | 105, 106, | 65 = 1X2 = | 57D1X61= |
| | | | | | 107, 108 | 1X2 = 1X10 = | |
| | | | | | | 1X642= | |
| 28 | 5.75 | 3 | 119 | 197 | 109, 110, | 39 = 1X9 = | 14D1X9 = |
| | | | | | 111, 112 | 1X15 = 1X659= | 1X15 = 1X78= |
| 29 | 5.73 | 1 | 236 | | 113, 114, | 78 = 1X646= | 53D1X65= |
| | | | | | 115, 116 | | |
| 30 | 5.62 | 1 | 176 | | 117, 118, | 59 = 1X665= | 33D2X84= |
| | | | | | 119, 120 | | |
| 31 | 5.62 | 1 | 146 | | 121, 122, | 49 = 1X675= | 23D2X94= |
| | | | | | 123, 124 | | |
| 32 | 5.55 | 5 | 101 | | 125, 126, | 12 = 2X8 = | 8D3X108= |
| | | | | | 127, 128 | 1X11 = 2X689= | |
| 33 | 5.24 | 4 | 248 | | 129, 130, | 65 = 1X3 = | 57D1X61= |
| | | | | | 131, 132 | 1X9 = 1X2 = | |
| | | | | | | 1X642= | |
| 34 | 5.15 | 1 | 197 | | 133, 134, | 65 = 1X659= | 40D1X78= |
| | | | | | 135, 136 | | |
| 35 | 5.02 | 4 | 248 | | 137, 138, | 69 = 1X5 = | 57D1X61= |
| | | | | | 139, 140 | 2X5 = 1X642= | |
| 36 | 4.94 | 1 | 101 | | 141, 142, | 34 = 1I691= | 7D2X110= |
| | | | | | 143, 144 | | |
| 37 | 4.93 | 6 | 110 | 137 | 145, 146, | 34 = 2X1 = | 12D1X1 = |
| | | | | | 147, 148 | 1D2 = 1X4 = | 2X4 = 1X3 = |
| | | | | | | 1X3 = 1X675= | 1X94= |
| 38 | 4.93 | 1 | 68 | | 149, 150, | 22 = 1I703= | 4I119= |
| | | | | | 151, 152 | | |
| 39 | 4.76 | 2 | 74 | | 153, 154, | 24 = 1X6 = | 116 = 1X112= |
| | | | | | 155, 156 | 1X693= | |
| 40 | 4.66 | 5 | 248 | | 157, 158, | 75 = 2X1 = | 57D1X61= |
| | | | | | 159, 160 | 2X2 = 1X642= | |
| 41 | 4.57 | 1 | 254 | | 161, 162, | 84 = 1I641= | 58D1X60= |
| | | | | | 163, 164 | | |
| 42 | 4.53 | 1 | 146 | | 165, 166, | 49 = 1X675= | 23D2X94= |
| | | | | | 167, 168 | | |
| 43 | 4.42 | 1 | 74 | | 169, 170, | 24 = 1I701= | 2I119= |
| | | | | | 171, 172 | | |
| 44 | 4.41 | 2 | 101 | | 173, 174, | 34 = 1X4 = | 8D1X5 = |
| | | | | | 175, 176 | 1X685= | 1X104= |
| 45 | 4.17 | 1 | 101 | | 177, 178, | 34 = 1X690= | 8D2X109= |
| | | | | | 179, 180 | | |
| 46 | 4.14 | 3 | 146 | | 181, 182, | 33 = 1X3 = | 23D2X94= |
| | | | | | 183, 184 | 1X11 = 1X675= | |
| 47 | 4.07 | 8 | | 125, 149 | 185, 186, | 36 = 3I1 = | 1X1 = 13D1X8 = |
| | | | | | 187, 188 | 4X8 = 1X675= | 1X94= |
| 48 | 4.05 | 1 | 101 | | 189, 190, | 34 = 1X690= | 8D2X109= |
| | | | | | 191, 192 | | |
| 49 | 3.93 | 2 | 236 | | 193, 194, | 75 = 1X2 = | 53D1X65= |
| | | | | | 195, 196 | 1X646= | |
| 50 | 3.87 | 1 | 65 | | 197, 198, | 21 = 1X703= | 4I119= |
| | | | | | 199, 200 | | |
| 51 | 3.85 | 7 | | 77, 101 | 201, 202, | 12 = 2X7 = | 1X7 = 1X110= |
| | | | | | 203, 204 | 1X2 = 2X7 = | |
| | | | | | | 2X690= | |
| 52 | 3.84 | 1 | 110 | | 205, 206, | 37 = 1X687= | 11D2X106= |
| | | | | | 207, 208 | | |
| 53 | 3.79 | 1 | 14 | | 209, 210, | 5 = 1X719= | 21I119= |
| | | | | | 211, 212 | | |
| 54 | 3.75 | 9 | 116 | | 213, 214, | 33 = 1X1 = | 13D3X8 = |
| | | | | | 215, 216 | 1X1 = 1X1 = | 1X4 = 1X4 = |
| | | | | | | 2X8 = 1X4 = | 1X5 = 1X78= |
| | | | | | | 1X4 = 1X5 = | |
| | | | | | | 1X659= | |
| 55 | 3.71 | 2 | 110 | 137, 185 | 217, 218, | 37 = 1D27 = | 12D1X27 = |
| | | | | | 219, 220 | 1X659= | 1X78= |
| 56 | 3.70 | 1 | 110 | | 221, 222, | 37 = 1X687= | 11D2X106= |
| | | | | | 223, 224 | | |
| 57 | 3.49 | 4 | 116 | 137 | 225, 226, | 36 = 1X2 = | 14D1X9 = |
| | | | | | 227, 228 | 1D9 = 1X4 = | 1X4 = 1X89= |
| | | | | | | 1X670= | |
| 58 | 3.37 | 1 | 110 | | 229, 230, | 37 = 1I688= | 10D2X107= |
| | | | | | 231, 232 | | |
| 59 | 3.22 | 3 | 101 | | 233, 234, | 34 = 1X14 = | 8D2X14 = |
| | | | | | 235, 236 | 1X4 = 1X670= | 1X4 = 1X89= |
| 60 | 3.21 | 5 | 236 | | 237, 238, | 68 = 1X6 = | 53D1X3 = |
| | | | | | 239, 240 | 2X1 = 1X3 = | 1X61 = |
| | | | | | | 1X642= | |
| 61 | 3.18 | 1 | 110 | | 241, 242, | 37 = 1X687= | 11D2X106= |
| | | | | | 243, 244 | | |
| 62 | 3.17 | 1 | | 77 | 245, 246, | 25 = 1X699= | 1X118= |
| | | | | | 247, 248 | | |
| 63 | 3.15 | 1 | 236 | | 249, 250, | 78 = 1X646= | 53D1X65 = |
| | | | | | 251, 252 | | |
| 64 | 3.14 | 3 | 116 | 146 | 253, 254, | 38 = 2I16 = | 11D2X16 = |
| | | | | | 255, 256 | 1X670= | 1X89= |
| 65 | 3.09 | 1 | 35 | | 257, 258, | 11 = 1I714= | 15I119= |
| | | | | | 259, 260 | | |
| 66 | 2.95 | 4 | 116 | | 261, 262, | 33 = 2X2 = | 13D2X104= |
| | | | | | 263, 264 | 1X1 = 1X685= | |
| 67 | 2.84 | 10 | 116 | 101 | 265, 266, | 33 = 3X1 = | 13D3X8 = |
| | | | | | 267, 268 | 1X1 = 2X8 = | 1X4 = 1X4 = |
| | | | | | | 1X4 = 1X4 = | 1X5 = 1X78= |
| | | | | | | 1X5 = 1X659= | |
| 68 | 2.80 | 10 | | 113, 125, | 269, 270, | 33 = 1X2 = | 9D6X16 = |
| | | | | 149, 197 | 271, 272 | 314X16 = | 1X8 = |
| | | | | | | 1X8 = 1X659= | 1X78= |
| 69 | 2.80 | 1 | 101 | | 273, 274, | 34 = 1X690= | 8D2X109= |
| | | | | | 275, 276 | | |
| 70 | 2.78 | 1 | | 74 | 277, 278, | 24 = 1X700= | 1I119= |
| | | | | | 279, 280 | | |
| 71 | 2.72 | 3 | 146 | | 281, 282, | 49 = 1X8 = | 23D2X8 = |
| | | | | | 283, 284 | 1X2 = 1X663= | 1X2 = 1X82= |
| 72 | 2.68 | 7 | 116 | | 285, 286, | 33 = 2X2 = | 13D2X14 = |
| | | | | | 287, 288 | 1X1 = 1X14 = | 1X4 = 1X5 = |
| | | | | | | 1X4 = 1X5 = | 1X78= |
| | | | | | | 1X659= | |
| 73 | 2.65 | 5 | 116, 119 | | 289, 290, | 33 = 2X2 = | 13D2X9 = |
| | | | | | 291, 292 | 1X1 = 1X9 = | 1X94= |
| | | | | | | 1X675= | |
| 74 | 2.47 | 3 | 116, 119 | | 293, 294, | 39 = 1X14 = | 13D2X14 = |
| | | | | | 295, 296 | 1X10 = | 1X10 = 1X78= |
| | | | | | | 1X659= | |
| 75 | 2.45 | 5 | 116, 119 | | 297, 298, | 36 = 1X2 = | 13D2X9 = |
| | | | | | 299, 300 | 1X9 = 1X4 = | 1X4 = 1X10 = |
| | | | | | | 1X10 = 1X659= | 1X78= |
| 76 | 2.45 | 5 | 116 | 146, 194 | 301, 302, | 35 = 212 = | 11D3X15 = |
| | | | | | 303, 304 | 1X16 = 1X10 = | 1X10 = 1X78= |
| | | | | | | 1X659= | |
| 77 | 2.28 | 4 | 101 | | 305, 306, | 34 = 2X1 = | 8D1X1 = |
| | | | | | 307, 308 | 1X11 = | 1X1 = 1X11 = |
| | | | | | | 1X675= | 1X94= |
| 78 | 2.22 | 4 | 236 | | 309, 310, | 68 = 1X9 = | 53D1X11 = |
| | | | | | 311, 312 | 1X3 = 1X7 = | 1X53= |
| | | | | | | 1X634= | |
| 79 | 1.97 | 1 | 101 | | 313, 314, | 34 = 1X690= | 8D2X109= |
| | | | | | 315, 316 | | |
| 80 | 1.78 | 4 | 236 | 197 | 317, 318, | 65 = 1X10 = | 53D1X3 = |
| | | | | | 319, 320 | 1X1 = 1X3 = | 1X61= |
| | | | | | | 1X642= | |

TABLE 2

| SEQ | | | |
|---|---|---|---|
| ID | Variant | Sequence | |
| NO | ID NO | type | Sequence |
| 1 | 1 | VP1_aa | MSFVDHPPDWLEEVGEGLREFLGLDAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 2 | 1 | VP1_nt | ATGTCTTTTGTTGATCACCCTCCAGATTGGTTGGAAGAAGTTGGTGAAGG |
| | | | TCTACGCGAGTTTTTGGGCCTTGATGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 3 | 1 | MAAP_aa | MRAHRNQNPISSIKIKPVVLCCLVITISDPETGSIEESLSTGQTRSREST |
| | | | TSRTTSSLRRETTPTSSTTTRTPSFRRSSPTTHPSGETSERQSFRPRKGF |
| | | | SNLLAWLKRVLRRPLPESG* |
| 4 | 1 | MAAP_nt | ATGCGGGCCCACCGAAACCAAAACCCAATCAGCAGCATCAAGATCAAGCC |
| | | | CGTGGTCTTGTGCTGCCTGGTTATAACTATCTCGGACCCGGAAACGGGCT |
| | | | CGATCGAGGAGAGCCTGTCAACAGGGCAGACGAGGTCGCGCGAGAGCACG |
| | | | ACATCTCGTACAACGAGCAGCTTGAGGCGGGAGACAACCCCTACCTCAAG |
| | | | TACAACCACGCGGACGCCGAGTTTCAGGAGAAGCTCGCCGACGACACATC |
| | | | CTTCGGGGGAAACCTCGGAAAGGCAGTCTTTCAGGCCAAGAAAAGGGTTC |
| | | | TCGAACCTTTTGGCCTGGTTGAAGAGGGTGCTAAGACGGCCCCTACCGGA |
| | | | AAGCGGATAG |
| 5 | 2 | VP1_aa | MSFVDHPPDWLEEVGEGLREFLGLHAGPPKIKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 6 | 2 | VP1_nt | ATGTCTTTTGTTGATCACCCTCCAGATTGGTTGGAAGAAGTTGGTGAAGG |
| | | | CCTTCGCGAGTTTTTGGGCCTTCATGCGGGCCCACCGAAAATTAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 7 | 2 | MAAP_aa | MRAHRKLNPISSIKIKPVVLCCLVITISDPETGSIEESLSTGQTRSREST |
| | | | TSRTTSSLRRETTPTSSTTTRTPSFRRSSPTTHPSGETSERQSFRPRKGF |
| | | | SNLLAWLKRVLRRPLPESG* |
| 8 | 2 | MAAP_nt | ATGCGGGCCCACCGAAAATTAAACCCAATCAGCAGCATCAAGATCAAGCC |
| | | | CGTGGTCTTGTGCTGCCTGGTTATAACTATCTCGGACCCGGAAACGGGCT |
| | | | CGATCGAGGAGAGCCTGTCAACAGGGCAGACGAGGTCGCGCGAGAGCACG |
| | | | ACATCTCGTACAACGAGCAGCTTGAGGCGGGAGACAACCCCTACCTCAAG |
| | | | TACAACCACGCGGACGCCGAGTTTCAGGAGAAGCTCGCCGACGACACATC |
| | | | CTTCGGGGGAAACCTCGGAAAGGCAGTCTTTCAGGCCAAGAAAAGGGTTC |
| | | | TCGAACCTTTTGGCCTGGTTGAAGAGGGTGCTAAGACGGCCCCTACCGGA |
| | | | AAGCGGATAG |
| 9 | 3 | VP1_aa | MSFVDHPPDWLEEVGEGLREFLGLEAGPPKPKPNQQHQSTNARGLVLPGY |
| | | | KYLGPGNGLDRGPPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 10 | 3 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAATCGACAAATGCCCGTGGTCTTGTGCTGCCTGGTTAT |
| | | | AAATATCTCGGACCCGGAAACGGGCTCGATCGAGGACCACCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 11 | 3 | MAAP_aa | MPVVLCCLVINISDPETGSIEDHLSTGQTRSRESTTSRTTSSLRRETTPT |
| | | | SSTTTRTPSFRRSSPTTHPSGETSERQSFRPRKGFSNLLAWLKRVLRRPL |
| | | | PESG* |
| 12 | 3 | MAAP_nt | ATGCCCGTGGTCTTGTGCTGCCTGGTTATAAATATCTCGGACCCGGAAAC |
| | | | GGGCTCGATCGAGGACCACCTGTCAACAGGGCAGACGAGGTCGCGCGAGA |
| | | | GCACGACATCTCGTACAACGAGCAGCTTGAGGCGGGAGACAACCCCTACC |
| | | | TCAAGTACAACCACGCGGACGCCGAGTTTCAGGAGAAGCTCGCCGACGAC |
| | | | ACATCCTTCGGGGGAAACCTCGGAAAGGCAGTCTTTCAGGCCAAGAAAAG |
| | | | GGTTCTCGAACCTTTTGGCCTGGTTGAAGAGGGTGCTAAGACGGCCCCTA |
| | | | CCGGAAAGCGGATAG |
| 13 | 4 | VP1_aa | MSFVDHPPDWLEEVGNGLREFLGLEAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 14 | 4 | VP1_nt | ATGTCTTTTGTTGATCACCCTCCAGATTGGTTGGAAGAAGTTGGTAATGG |
| | | | TCTGCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 15 | 4 | MAAP_aa | MVCASFWALKRAHRNQNPISSIKIKPVVLCCLVITISDPETGSIEESLST |
| | | | GQTRSRESTTSRTTSSLRRETTPTSSTTTRTPSFRRSSPTTHPSGETSER |
| | | | QSFRPRKGFSNLLAWLKRVLRRPLPESG* |
| 16 | 4 | MAAP_nt | ATGGTCTGCGCGAGTTTTTGGGCCTTGAAGCGGGCCCACCGAAACCAAAA |
| | | | CCCAATCAGCAGCATCAAGATCAAGCCCGTGGTCTTGTGCTGCCTGGTTA |
| | | | TAACTATCTCGGACCCGGAAACGGGCTCGATCGAGGAGAGCCTGTCAACA |
| | | | GGGCAGACGAGGTCGCGCGAGAGCACGACATCTCGTACAACGAGCAGCTT |
| | | | GAGGCGGGAGACAACCCCTACCTCAAGTACAACCACGCGGACGCCGAGTT |
| | | | TCAGGAGAAGCTCGCCGACGACACATCCTTCGGGGGAAACCTCGGAAAGG |
| | | | CAGTCTTTCAGGCCAAGAAAAGGGTTCTCGAACCTTTTGGCCTGGTTGAA |
| | | | GAGGGTGCTAAGACGGCCCCTACCGGAAAGCGGATAG |
| 17 | 5 | VP1_aa | MSFVDHPPDWLEEVGEGLDEFLGLEAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 18 | 5 | VP1_nt | ATGTCTTTTGTTGATCACCCTCCAGATTGGTTGGAAGAAGTTGGTGAAGG |
| | | | TCTTGATGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 19 | 5 | MAAP_aa | MSFWALKRAHRNQNPISSIKIKPVVLCCLVITISDPETGSIEESLSTGQT |
| | | | RSRESTTSRTTSSLRRETTPTSSTTTRTPSFRRSSPTTHPSGETSERQSF |
| | | | RPRKGFSNLLAWLKRVLRRPLPESG* |
| 20 | 5 | MAAP_nt | ATGAGTTTTTGGGCCTTGAAGCGGGCCCACCGAAACCAAAACCCAATCAG |
| | | | CAGCATCAAGATCAAGCCCGTGGTCTTGTGCTGCCTGGTTATAACTATCT |
| | | | CGGACCCGGAAACGGGCTCGATCGAGGAGAGCCTGTCAACAGGGCAGACG |
| | | | AGGTCGCGCGAGAGCACGACATCTCGTACAACGAGCAGCTTGAGGCGGGA |
| | | | GACAACCCCTACCTCAAGTACAACCACGCGGACGCCGAGTTTCAGGAGAA |
| | | | GCTCGCCGACGACACATCCTTCGGGGGAAACCTCGGAAAGGCAGTCTTTC |
| | | | AGGCCAAGAAAAGGGTTCTCGAACCTTTTGGCCTGGTTGAAGAGGGTGCT |
| | | | AAGACGGCCCCTACCGGAAAGCGGATAG |
| 21 | 6 | VP1_aa | MSFVDHPPDWLEEVYEGLREFLGLEAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 22 | 6 | VP1_nt | ATGTCTTTTGTTGATCACCCTCCAGATTGGTTGGAAGAAGTTTATGAAGG |
| | | | TCTGCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 23 | 6 | MAAP_aa | MKVCASFWALKRAHRNQNPISSIKIKPVVLCCLVITISDPETGSIEESLS |
| | | | TGQTRSRESTTSRTTSSLRRETTPTSSTTTRTPSFRRSSPTTHPSGETSE |
| | | | RQSFRPRKGFSNLLAWLKRVLRRPLPESG* |
| 24 | 6 | MAAP_nt | ATGAAGGTCTGCGCGAGTTTTTGGGCCTTGAAGCGGGCCCACCGAAACCA |
| | | | AAACCCAATCAGCAGCATCAAGATCAAGCCCGTGGTCTTGTGCTGCCTGG |
| | | | TTATAACTATCTCGGACCCGGAAACGGGCTCGATCGAGGAGAGCCTGTCA |
| | | | ACAGGGCAGACGAGGTCGCGCGAGAGCACGACATCTCGTACAACGAGCAG |
| | | | CTTGAGGCGGGAGACAACCCCTACCTCAAGTACAACCACGCGGACGCCGA |
| | | | GTTTCAGGAGAAGCTCGCCGACGACACATCCTTCGGGGGAAACCTCGGAA |
| | | | AGGCAGTCTTTCAGGCCAAGAAAAGGGTTCTCGAACCTTTTGGCCTGGTT |
| | | | GAAGAGGGTGCTAAGACGGCCCCTACCGGAAAGCGGATAG |
| 25 | 7 | VP1_aa | MSFVDHPPDWLEEVGEGLREFLGLEAGPPKPKPNQQHSDEQARGLVLPGY |
| | | | NYLGPGNGLDRGEPVNEADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 26 | 7 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATTCCGATGAGCAAGCCCGTGGTCTTGTGCTGCCTGGTTAT |
| | | | AACTATCTCGGACCCGGAAACGGGCTCGATCGAGGAGAGCCTGTCAACGA |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 27 | 7 | MAAP_aa | MSKPVVLCCLVITISDPETGSIEESLSTRQTRSRESTTSRTTSSLRRETT |
| | | | PTSSTTTRTPSFRRSSPTTHPSGETSERQSFRPRKGFSNLLAWLKRVLRR |
| | | | PLPESG* |
| 28 | 7 | MAAP_nt | ATGAGCAAGCCCGTGGTCTTGTGCTGCCTGGTTATAACTATCTCGGACCC |
| | | | GGAAACGGGCTCGATCGAGGAGAGCCTGTCAACGAGGCAGACGAGGTCGC |
| | | | GCGAGAGCACGACATCTCGTACAACGAGCAGCTTGAGGCGGGAGACAACC |
| | | | CCTACCTCAAGTACAACCACGCGGACGCCGAGTTTCAGGAGAAGCTCGCC |
| | | | GACGACACATCCTTCGGGGGAAACCTCGGAAAGGCAGTCTTTCAGGCCAA |
| | | | GAAAAGGGTTCTCGAACCTTTTGGCCTGGTTGAAGAGGGTGCTAAGACGG |
| | | | CCCCTACCGGAAAGCGGATAG |
| 29 | 8 | VP1_aa | MSFVDHPPDWLEEVGEGLFEFYGLEAGFPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 30 | 8 | VP1_nt | ATGTCTTTTGTTGATCACCCTCCAGATTGGTTGGAAGAAGTTGGTGAAGG |
| | | | TCTTTTCGAGTTTTATGGCCTTGAAGCGGGCTTTCCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 31 | 8 | MAAP_aa | MALKRAFRNQNPISSIKIKPVVLCCLVITISDPETGSIEESLSTGQTRSR |
| | | | ESTTSRTTSSLRRETTPTSSTTTRTPSFRRSSPTTHPSGETSERQSFRPR |
| | | | KGFSNLLAWLKRVLRRPLPESG* |
| 32 | 8 | MAAP_nt | ATGGCCTTGAAGCGGGCTTTCCGAAACCAAAACCCAATCAGCAGCATCAA |
| | | | GATCAAGCCCGTGGTCTTGTGCTGCCTGGTTATAACTATCTCGGACCCGG |
| | | | AAACGGGCTCGATCGAGGAGAGCCTGTCAACAGGGCAGACGAGGTCGCGC |
| | | | GAGAGCACGACATCTCGTACAACGAGCAGCTTGAGGCGGGAGACAACCCC |
| | | | TACCTCAAGTACAACCACGCGGACGCCGAGTTTCAGGAGAAGCTCGCCGA |
| | | | CGACACATCCTTCGGGGGAAACCTCGGAAAGGCAGTCTTTCAGGCCAAGA |
| | | | AAAGGGTTCTCGAACCTTTTGGCCTGGTTGAAGAGGGTGCTAAGACGGCC |
| | | | CCTACCGGAAAGCGGATAG |
| 33 | 9 | VP1_aa | MSFVDHPPDWLEEVGEGLREFLGLEAGPPKPKPNQQHQDQARGLVLPGYN |
| | | | YLGPGNGLDRGEPVNRADEVAREHDISYNEQLDAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 34 | 9 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 35 | 9 | MAAP_aa | MRETTPTSSTTTRTPSFRRSSPTTHPSGETSERQSFRPRKGFSNLLAWLK |
| | | | RVLRRPLPESG* |
| 36 | 9 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 37 | 10 | VP1_aa | MSFVDHPPDWLEEVGEGLREFLGLEAGPPKPKPNQQHQDNGRGLVLPGYK |
| | | | YLGPFNGLDKGEPVNEADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 38 | 10 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATAATGGACGTGGTCTTGTGCTGCCTGGTTATAAA |
| | | | TATCTCGGACCCTTCAACGGGCTCGATAAGGGAGAGCCTGTCAACGAAGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 39 | 10 | MAAP_aa | MDVVLCCLVINISDPSTGSIRESLSTKQTRSRESTTSRTTSSLRRETTPT |
| | | | SSTTTRTPSFRRSSPTTHPSGETSERQSFRPRKGFSNLLAWLKRVLRRPL |
| | | | PESG* |
| 40 | 10 | MAAP_nt | ATGGACGTGGTCTTGTGCTGCCTGGTTATAAATATCTCGGACCCTTCAAC |
| | | | GGGCTCGATAAGGGAGAGCCTGTCAACGAAGCAGACGAGGTCGCGCGAGA |
| | | | GCACGACATCTCGTACAACGAGCAGCTTGAGGCGGGAGACAACCCCTACC |
| | | | TCAAGTACAACCACGCGGACGCCGAGTTTCAGGAGAAGCTCGCCGACGAC |
| | | | ACATCCTTCGGGGGAAACCTCGGAAAGGCAGTCTTTCAGGCCAAGAAAAG |
| | | | GGTTCTCGAACCTTTTGGCCTGGTTGAAGAGGGTGCTAAGACGGCCCCTA |
| | | | CCGGAAAGCGGATAG |
| 41 | 11 | VP1_aa | MSFVDHPPDWLEEVGEGLREFLGLEAGPPKPKPNEQQHQDQARGLVLPGY |
| | | | NYLGPGNGLDRGEPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLISTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 42 | 11 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGAGCAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 43 | 11 | MAAP_aa | MSSSIKIKPVVLCCLVITISDPETGSIEESLSTGQTRSRESTTSRTTSSL |
| | | | RRETTPTSSTTTRTPSFRRSSPTTHPSGETSERQSFRPRKGFSNLLAWLK |
| | | | RVLRRPLPESG* |
| 44 | 11 | MAAP_nt | ATGAGCAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 45 | 12 | VP1_aa | MSFVDHPPDWLEEVGDGLRLFLGLEAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 46 | 12 | VP1_nt | ATGTCTTTTGTTGATCACCCTCCAGATTGGTTGGAAGAAGTTGGTGATGG |
| | | | TCTACGCCTGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 47 | 12 | MAAP_aa | MVYACFWALKRAHRNQNPISSIKIKPVVLCCLVITISDPETGSIEESLST |
| | | | GQTRSRESTTSRTTSSLRRETTPTSSTTTRTPSFRRSSPTTHPSGETSER |
| | | | QSFRPRKGFSNLLAWLKRVLRRPLPESG* |
| 48 | 12 | MAAP_nt | ATGGTCTACGCCTGTTTTTGGGCCTTGAAGCGGGCCCACCGAAACCAAAA |
| | | | CCCAATCAGCAGCATCAAGATCAAGCCCGTGGTCTTGTGCTGCCTGGTTA |
| | | | TAACTATCTCGGACCCGGAAACGGGCTCGATCGAGGAGAGCCTGTCAACA |
| | | | GGGCAGACGAGGTCGCGCGAGAGCACGACATCTCGTACAACGAGCAGCTT |
| | | | GAGGCGGGAGACAACCCCTACCTCAAGTACAACCACGCGGACGCCGAGTT |
| | | | TCAGGAGAAGCTCGCCGACGACACATCCTTCGGGGGAAACCTCGGAAAGG |
| | | | CAGTCTTTCAGGCCAAGAAAAGGGTTCTCGAACCTTTTGGCCTGGTTGAA |
| | | | GAGGGTGCTAAGACGGCCCCTACCGGAAAGCGGATAG |
| 49 | 13 | VP1_aa | MSFVDHPPDWLNEVGEGLREFLGLEAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 50 | 13 | VP1_nt | ATGTCTTTTGTTGATCACCCTCCAGATTGGTTGAATGAAGTTGGTGAAGG |
| | | | TCTACGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 51 | 13 | MAAP_aa | MKLVKVYASFWALKRAHRNQNPISSIKIKPVVLCCLVITISDPETGSIEE |
| | | | SLSTGQTRSRESTTSRTTSSLRRETTPTSSTTTRIPSFRRSSPTTHPSGE |
| | | | TSERQSFRPRKGFSNLLAWLKRVLRRPLPESG* |
| 52 | 13 | MAAP_nt | ATGAAGTTGGTGAAGGTCTACGCGAGTTTTTGGGCCTTGAAGCGGGCCCA |
| | | | CCGAAACCAAAACCCAATCAGCAGCATCAAGATCAAGCCCGTGGTCTTGT |
| | | | GCTGCCTGGTTATAACTATCTCGGACCCGGAAACGGGCTCGATCGAGGAG |
| | | | AGCCTGTCAACAGGGCAGACGAGGTCGCGCGAGAGCACGACATCTCGTAC |
| | | | AACGAGCAGCTTGAGGCGGGAGACAACCCCTACCTCAAGTACAACCACGC |
| | | | GGACGCCGAGTTTCAGGAGAAGCTCGCCGACGACACATCCTTCGGGGGAA |
| | | | ACCTCGGAAAGGCAGTCTTTCAGGCCAAGAAAAGGGTTCTCGAACCTTTT |
| | | | GGCCTGGTTGAAGAGGGTGCTAAGACGGCCCCTACCGGAAAGCGGATAG |
| 53 | 14 | VP1_aa | MSFVDHPPDWLEEVGEGLREFLGLEAGPPKPKPNQQHQDHARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 54 | 14 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCATGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 55 | 14 | MAAP_aa | MPVVLCCLVITISDPETGSIEESLSTGQTRSRESTTSRTTSSLRRETTPT |
| | | | SSTTTRTPSFRRSSPTTHPSGETSERQSFRPRKGFSNLLAWLKRVLRRPL |
| | | | PESG* |
| 56 | 14 | MAAP_nt | ATGCCCGTGGTCTTGTGCTGCCTGGTTATAACTATCTCGGACCCGGAAAC |
| | | | GGGCTCGATCGAGGAGAGCCTGTCAACAGGGCAGACGAGGTCGCGCGAGA |
| | | | GCACGACATCTCGTACAACGAGCAGCTTGAGGCGGGAGACAACCCCTACC |
| | | | TCAAGTACAACCACGCGGACGCCGAGTTTCAGGAGAAGCTCGCCGACGAC |
| | | | ACATCCTTCGGGGGAAACCTCGGAAAGGCAGTCTTTCAGGCCAAGAAAAG |
| | | | GGTTCTCGAACCTTTTGGCCTGGTTGAAGAGGGTGCTAAGACGGCCCCTA |
| | | | CCGGAAAGCGGATAG |
| 57 | 15 | VP1_aa | MSFVDHPPDWLEEVGEGLREFLGLEAGPPKPKPNQQHQDQARGLVLPGYA |
| | | | YLGPFNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 58 | 15 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATGCC |
| | | | TATCTCGGACCCTTCAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 59 | 15 | MAAP_aa | MPISDPSTGSIEESLSTGQTRSRESTTSRTTSSLRRETTPTSSTTTRTPS |
| | | | FRRSSPTTHPSGETSERQSFRPRKGFSNLLAWLKRVLRRPLPESG* |
| 60 | 15 | MAAP_nt | ATGCCTATCTCGGACCCTTCAACGGGCTCGATCGAGGAGAGCCTGTCAAC |
| | | | AGGGCAGACGAGGTCGCGCGAGAGCACGACATCTCGTACAACGAGCAGCT |
| | | | TGAGGCGGGAGACAACCCCTACCTCAAGTACAACCACGCGGACGCCGAGT |
| | | | TTCAGGAGAAGCTCGCCGACGACACATCCTTCGGGGGAAACCTCGGAAAG |
| | | | GCAGTCTTTCAGGCCAAGAAAAGGGTTCTCGAACCTTTTGGCCTGGTTGA |
| | | | AGAGGGTGCTAAGACGGCCCCTACCGGAAAGCGGATAG |
| 61 | 16 | VP1_aa | MSFVDHPPDWLEEVGEGLREFLGLEAGPPKPKPNQQHQDQARGLVLPGYN |
| | | | YLGPGNGLDRGEPVNRADEVAREHDISYNEQLHAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 62 | 16 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTCATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 63 | 16 | MAAP_aa | MRETTPTSSTTTRTPSFRRSSPTTHPSGETSERQSFRPRKGFSNLLAWLK |
| | | | RVLRRPLPESG* |
| 64 | 16 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 65 | 17 | VP1_aa | MSFVDHPPDWLEEVGEGLREFLGLEAGPPKPKPNQQHQDQARGLVLPGYA |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 66 | 17 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATGCT |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 67 | 17 | MAAP_aa | MLISDPETGSIEESLSTGQTRSRESTTSRTTSSLRRETTPTSSTTTRTPS |
| | | | FRRSSPTTHPSGETSERQSFRPRKGFSNLLAWLKRVLRRPLPESG* |
| 68 | 17 | MAAP_nt | ATGCTTATCTCGGACCCGGAAACGGGCTCGATCGAGGAGAGCCTGTCAAC |
| | | | AGGGCAGACGAGGTCGCGCGAGAGCACGACATCTCGTACAACGAGCAGCT |
| | | | TGAGGCGGGAGACAACCCCTACCTCAAGTACAACCACGCGGACGCCGAGT |
| | | | TTCAGGAGAAGCTCGCCGACGACACATCCTTCGGGGGAAACCTCGGAAAG |
| | | | GCAGTCTTTCAGGCCAAGAAAAGGGTTCTCGAACCTTTTGGCCTGGTTGA |
| | | | AGAGGGTGCTAAGACGGCCCCTACCGGAAAGCGGATAG |
| 69 | 18 | VP1_aa | MSFVDHPPDWLEEVGEGLREFLGLEAGPPKPKPNQQHQDHARGLVLPGYN |
| | | | YLGPGNGLERGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 70 | 18 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCATGCCCGTGGTCTTGTGCTGCCTGGTTATAAC |
| | | | TATCTCGGACCCGGAAACGGGCTCGAGCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 71 | 18 | MAAP_aa | MPVVLCCLVITISDPETGSSEESLSTGQTRSRESTTSRTTSSLRRETTPT |
| | | | SSTTTRTPSFRRSSPTTHPSGETSERQSFRPRKGFSNLLAWLKRVLRRPL |
| | | | PESG* |
| 72 | 18 | MAAP_nt | ATGCCCGTGGTCTTGTGCTGCCTGGTTATAACTATCTCGGACCCGGAAAC |
| | | | GGGCTCGAGCGAGGAGAGCCTGTCAACAGGGCAGACGAGGTCGCGCGAGA |
| | | | GCACGACATCTCGTACAACGAGCAGCTTGAGGCGGGAGACAACCCCTACC |
| | | | TCAAGTACAACCACGCGGACGCCGAGTTTCAGGAGAAGCTCGCCGACGAC |
| | | | ACATCCTTCGGGGGAAACCTCGGAAAGGCAGTCTTTCAGGCCAAGAAAAG |
| | | | GGTTCTCGAACCTTTTGGCCTGGTTGAAGAGGGTGCTAAGACGGCCCCTA |
| | | | CCGGAAAGCGGATAG |
| 73 | 19 | VP1_aa | MAFVDHPPDWLEEVGEGLREFWDLKPGAPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 74 | 19 | VP1_nt | ATGGCGTTTGTTGATCACCCTCCAGATTGGTTGGAAGAAGTTGGTGAAGG |
| | | | TCTACGCGAGTTTTGGGACCTTAAACCTGGCGCTCCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 75 | 19 | MAAP_aa | LALRNQNPISSIKIKPVVLCCLVITISDPETGSIEESLSTGQTRSRESTT |
| | | | SRTTSSLRRETTPTSSTTTRTPSFRRSSPTTHPSGETSERQSFRPRKGFS |
| | | | NLLAWLKRVLRRPLPESG* |
| 76 | 19 | MAAP_nt | CTGGCGCTCCGAAACCAAAACCCAATCAGCAGCATCAAGATCAAGCCCGT |
| | | | GGTCTTGTGCTGCCTGGTTATAACTATCTCGGACCCGGAAACGGGCTCGA |
| | | | TCGAGGAGAGCCTGTCAACAGGGCAGACGAGGTCGCGCGAGAGCACGACA |
| | | | TCTCGTACAACGAGCAGCTTGAGGCGGGAGACAACCCCTACCTCAAGTAC |
| | | | AACCACGCGGACGCCGAGTTTCAGGAGAAGCTCGCCGACGACACATCCTT |
| | | | CGGGGGAAACCTCGGAAAGGCAGTCTTTCAGGCCAAGAAAAGGGTTCTCG |
| | | | AACCTTTTGGCCTGGTTGAAGAGGGTGCTAAGACGGCCCCTACCGGAAAG |
| | | | CGGATAG |
| 77 | 20 | VP1_aa | MSFVDHPPDWLEEVGEGLREFLGLEAGPPKPKPNQQHQDQARGLVLPGYN |
| | | | YLGPGNGLDRGEPVNRADEVAREHDISYNEQLNAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 78 | 20 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTAATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 79 | 20 | MAAP_aa | MRETTPTSSTTTRTPSFRRSSPTTHPSGETSERQSFRPRKGFSNLLAWLK |
| | | | RVLRRPLPESG* |
| 80 | 20 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 81 | 21 | VP1_aa | MSFVDHPPDWLEDEVGEGLREWLKLEAGPPKPKPNEQHQDQARGLVLPGY |
| | | | NYLGPGNGLDRGEPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 82 | 21 | VP1_nt | ATGTCTTTTGTTGATCACCCTCCAGATTGGTTGGAAGATGAAGTTGGTGA |
| | | | AGGCCTTCGCGAGTGGTTGAAGCTGGAAGCGGGCCCACCGAAACCAAAAC |
| | | | CCAATGAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 83 | 21 | MAAP_aa | MSSIKIKPVVLCCLVITISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 84 | 21 | MAAP_nt | ATGAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 85 | 22 | VP1_aa | MSFVDHPPDWLEEVGDGLREFLGLEAGPPKPKPSQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 86 | 22 | VP1_nt | ATGTCTTTTGTTGATCACCCTCCAGATTGGTTGGAAGAAGTTGGTGATGG |
| | | | TCTGCGCGAGTTTTTGGGCCTTGAAGCGGGCCCACCGAAACCAAAACCCT |
| | | | CTCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 87 | 22 | MAAP_aa | MVCASFWALKRAHRNQNPLSSIKIKPVVLCCLVITISDPETGSIEESLST |
| | | | GQTRSRESTTSRTTSSLRRETTPTSSTTTRTPSFRRSSPTTHPSGETSER |
| | | | QSFRPRKGFSNLLAWLKRVLRRPLPESG* |
| 88 | 22 | MAAP_nt | ATGGTCTGCGCGAGTTTTTGGGCCTTGAAGCGGGCCCACCGAAACCAAAA |
| | | | CCCTCTCAGCAGCATCAAGATCAAGCCCGTGGTCTTGTGCTGCCTGGTTA |
| | | | TAACTATCTCGGACCCGGAAACGGGCTCGATCGAGGAGAGCCTGTCAACA |
| | | | GGGCAGACGAGGTCGCGCGAGAGCACGACATCTCGTACAACGAGCAGCTT |
| | | | GAGGCGGGAGACAACCCCTACCTCAAGTACAACCACGCGGACGCCGAGTT |
| | | | TCAGGAGAAGCTCGCCGACGACACATCCTTCGGGGGAAACCTCGGAAAGG |
| | | | CAGTCTTTCAGGCCAAGAAAAGGGTTCTCGAACCTTTTGGCCTGGTTGAA |
| | | | GAGGGTGCTAAGACGGCCCCTACCGGAAAGCGGATAG |
| 89 | 23 | VP1_aa | MSFVDHPPDWLHEVGEGLREFLGLHAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 90 | 23 | VP1_nt | ATGTCTTTTGTTGATCACCCTCCAGATTGGTTGCATGAAGTTGGTGAAGG |
| | | | GCTTCGCGAGTTTTTGGGCCTTCACGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 91 | 23 | MAAP_aa | MKLVKGFASFWAFTRAHRNQNPISSIKIKPVVLCCLVITISDPETGSIEE |
| | | | SLSTGQTRSRESTTSRTTSSLRRETTPTSSTTTRTPSFRRSSPTTHPSGE |
| | | | TSERQSFRPRKGFSNLLAWLKRVLRRPLPESG* |
| 92 | 23 | MAAP_nt | ATGAAGTTGGTGAAGGGCTTCGCGAGTTTTTGGGCCTTCACGCGGGCCCA |
| | | | CCGAAACCAAAACCCAATCAGCAGCATCAAGATCAAGCCCGTGGTCTTGT |
| | | | GCTGCCTGGTTATAACTATCTCGGACCCGGAAACGGGCTCGATCGAGGAG |
| | | | AGCCTGTCAACAGGGCAGACGAGGTCGCGCGAGAGCACGACATCTCGTAC |
| | | | AACGAGCAGCTTGAGGCGGGAGACAACCCCTACCTCAAGTACAACCACGC |
| | | | GGACGCCGAGTTTCAGGAGAAGCTCGCCGACGACACATCCTTCGGGGGAA |
| | | | ACCTCGGAAAGGCAGTCTTTCAGGCCAAGAAAAGGGTTCTCGAACCTTTT |
| | | | GGCCTGGTTGAAGAGGGTGCTAAGACGGCCCCTACCGGAAAGCGGATAG |
| 93 | 24 | VP1_aa | MSFVDHPPDWLEEVGEGLREFLGLEAGPPKPKPNQQHYDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 94 | 24 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATTATGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 95 | 24 | MAAP_aa | MIKPVVLCCLVITISDPETGSIEESLSTGQTRSRESTTSRTTSSLRRETT |
| | | | PTSSTTTRTPSFRRSSPTTHPSGETSERQSFRPRKGFSNLLAWLKRVLRR |
| | | | PLPESG* |
| 96 | 24 | MAAP_nt | ATGATCAAGCCCGTGGTCTTGTGCTGCCTGGTTATAACTATCTCGGACCC |
| | | | GGAAACGGGCTCGATCGAGGAGAGCCTGTCAACAGGGCAGACGAGGTCGC |
| | | | GCGAGAGCACGACATCTCGTACAACGAGCAGCTTGAGGCGGGAGACAACC |
| | | | CCTACCTCAAGTACAACCACGCGGACGCCGAGTTTCAGGAGAAGCTCGCC |
| | | | GACGACACATCCTTCGGGGGAAACCTCGGAAAGGCAGTCTTTCAGGCCAA |
| | | | GAAAAGGGTTCTCGAACCTTTTGGCCTGGTTGAAGAGGGTGCTAAGACGG |
| | | | CCCCTACCGGAAAGCGGATAG |
| 97 | 25 | VP1_aa | MSFVDHPPDWLEEVGEGLREFLGLEAGPPKPKPNQQHQDQARGLVLPGYN |
| | | | YLGPGNGLDRGEPVNEADAAAREHDISYNEQLDAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 98 | 25 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACGAAGC |
| | | | AGACGCCGCAGCGCGAGAGCACGACATCTCGTACAACGAGCAGCTTGATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 99 | 25 | MAAP_aa | MRETTPTSSTTTRTPSFRRSSPTTHPSGETSERQSFRPRKGFSNLLAWLK |
| | | | RVLRRPLPESG* |
| 100 | 25 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 101 | 26 | VP1_aa | MSFVDHPPDWLEEVGEGLREFLGLEAGPPKPKPNEQHKDQARGLVLPGYN |
| | | | YLGPFNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 102 | 26 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGAGCAGCATAAGGATCAAGCCCGTGGTCTTGTGCTGCCTGGTTATAAC |
| | | | TATCTCGGACCCTTCAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 103 | 26 | MAAP_aa | MSSIRIKPVVLCCLVITISDPSTGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 104 | 26 | MAAP_nt | ATGAGCAGCATAAGGATCAAGCCCGTGGTCTTGTGCTGCCTGGTTATAAC |
| | | | TATCTCGGACCCTTCAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 105 | 27 | VP1_aa | MSFVDHPPDWLEEVGEGLREFLGLEAGPPKPKPNQQHQDQARGLVLPGYN |
| | | | YLGPGNGLDRGEPVNEADAVALEHDISYNEQLDAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 106 | 27 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACGAAGC |
| | | | AGACGCGGTCGCGCTTGAGCACGACATCTCGTACAACGAGCAGCTTGATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 107 | 27 | MAAP_aa | MRETTPTSSTTTRTPSFRRSSPTTHPSGETSERQSFRPRKGFSNLLAWLK |
| | | | RVLRRPLPESG* |
| 108 | 27 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 109 | 28 | VP1_aa | MSFVDHPPDWLEEVGEGLREFLGLEAGPPKPKPNQQHQDNARGLVLPGYK |
| | | | YLGPGNGLDRGEPVNAADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 110 | 28 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATAATGCCCGTGGTCTTGTGCTGCCTGGTTATAAG |
| | | | TATCTCGGACCCGGAAACGGGCTCGATCGAGGAGAGCCTGTCAACGCTGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 111 | 28 | MAAP_aa | MPVVLCCLVISISDPETGSIEESLSTLQTRSRESTTSRTTSSLRRETTPT |
| | | | SSTTTRTPSFRRSSPTTHPSGETSERQSFRPRKGFSNLLAWLKRVLRRPL |
| | | | PESG* |
| 112 | 28 | MAAP_nt | ATGCCCGTGGTCTTGTGCTGCCTGGTTATAAGTATCTCGGACCCGGAAAC |
| | | | GGGCTCGATCGAGGAGAGCCTGTCAACGCTGCAGACGAGGTCGCGCGAGA |
| | | | GCACGACATCTCGTACAACGAGCAGCTTGAGGCGGGAGACAACCCCTACC |
| | | | TCAAGTACAACCACGCGGACGCCGAGTTTCAGGAGAAGCTCGCCGACGAC |
| | | | ACATCCTTCGGGGGAAACCTCGGAAAGGCAGTCTTTCAGGCCAAGAAAAG |
| | | | GGTTCTCGAACCTTTTGGCCTGGTTGAAGAGGGTGCTAAGACGGCCCCTA |
| | | | CCGGAAAGCGGATAG |
| 113 | 29 | VP1_aa | MSFVDHPPDWLEEVGEGLREFLGLEAGPPKPKPNQQHQDQARGLVLPGYN |
| | | | YLGPGNGLDRGEPVNRADEVAREHDISYYEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 114 | 29 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACTATGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 115 | 29 | MAAP_aa | MSSLRRETTPTSSTTTRTPSFRRSSPTTHPSGETSERQSFRPRKGFSNLL |
| | | | AWLKRVLRRPLPESG* |
| 116 | 29 | MAAP_nt | ATGAGCAGCTTGAGGCGGGAGACAACCCCTACCTCAAGTACAACCACGCG |
| | | | GACGCCGAGTTTCAGGAGAAGCTCGCCGACGACACATCCTTCGGGGGAAA |
| | | | CCTCGGAAAGGCAGTCTTTCAGGCCAAGAAAAGGGTTCTCGAACCTTTTG |
| | | | GCCTGGTTGAAGAGGGTGCTAAGACGGCCCCTACCGGAAAGCGGATAG |
| 117 | 30 | VP1_aa | MSFVDHPPDWLEEVGEGLREFLGLEAGPPKPKPNQQHQDQARGLVLPGYN |
| | | | YLGPGNGLDAGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 118 | 30 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATGCCGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 119 | 30 | MAAP_aa | MPESLSTGQTRSRESTTSRTTSSLRRETTPTSSTTTRTPSFRRSSPTTHP |
| | | | SGETSERQSFRPRKGFSNLLAWLKRVLRRPLPESG* |
| 120 | 30 | MAAP_nt | ATGCCGGAGAGCCTGTCAACAGGGCAGACGAGGTCGCGCGAGAGCACGAC |
| | | | ATCTCGTACAACGAGCAGCTTGAGGCGGGAGACAACCCCTACCTCAAGTA |
| | | | CAACCACGCGGACGCCGAGTTTCAGGAGAAGCTCGCCGACGACACATCCT |
| | | | TCGGGGGAAACCTCGGAAAGGCAGTCTTTCAGGCCAAGAAAAGGGTTCTC |
| | | | GAACCTTTTGGCCTGGTTGAAGAGGGTGCTAAGACGGCCCCTACCGGAAA |
| | | | GCGGATAG |
| 121 | 31 | VP1_aa | MSFVDHPPDWLEEVGEGLREFLGLEAGPPKPKPNQQHQDQARGLVLPGYV |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 122 | 31 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATGTT |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 123 | 31 | MAAP_aa | MFISDPETGSIEESLSTGQTRSRESTTSRTTSSLRRETTPTSSTTTRTPS |
| | | | FRRSSPTTHPSGETSERQSFRPRKGFSNLLAWLKRVLRRPLPESG* |
| 124 | 31 | MAAP_nt | ATGTTTATCTCGGACCCGGAAACGGGCTCGATCGAGGAGAGCCTGTCAAC |
| | | | AGGGCAGACGAGGTCGCGCGAGAGCACGACATCTCGTACAACGAGCAGCT |
| | | | TGAGGCGGGAGACAACCCCTACCTCAAGTACAACCACGCGGACGCCGAGT |
| | | | TTCAGGAGAAGCTCGCCGACGACACATCCTTCGGGGGAAACCTCGGAAAG |
| | | | GCAGTCTTTCAGGCCAAGAAAAGGGTTCTCGAACCTTTTGGCCTGGTTGA |
| | | | AGAGGGTGCTAAGACGGCCCCTACCGGAAAGCGGATAG |
| 125 | 32 | VP1_aa | MSFVDHPPDWLETLGEGLREFLKLEAGPPKPKPNERHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 126 | 32 | VP1_nt | ATGTCTTTTGTTGATCACCCTCCAGATTGGTTGGAAACCCTTGGTGAAGG |
| | | | CCTTCGCGAGTTTTTGAAACTTGAAGCGGGCCCACCGAAACCAAAACCCA |
| | | | ATGAACGGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 127 | 32 | MAAP_aa | MNGIKIKPVVLCCLVITISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 128 | 32 | MAAP_nt | ATGAACGGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 129 | 33 | VP1_aa | MSFVDHPPDWLEEVGEGLREFLGLEAGPPKPKPNQQHQDQARGLVLPGYN |
| | | | YLGPGNGLDRGEPVNEADEAAREHDISYNRQLDAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 130 | 33 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACGAGGC |
| | | | AGACGAGGCAGCGCGAGAGCACGACATCTCGTACAACCGCCAGCTTGATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 131 | 33 | MAAP_aa | MRETTPTSSTTTRTPSFRRSSPTTHPSGETSERQSFRPRKGFSNLLAWLK |
| | | | RVLRRPLPESG* |
| 132 | 33 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 133 | 34 | VP1_aa | MSFVDHPPDWLEEVGEGLREFLGLEAGPPKPKPNQQHQDQARGLVLPGYN |
| | | | YLGPGNGLDRGEPVNYADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 134 | 34 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACTATGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 135 | 34 | MAAP_aa | MQTRSRESTTSRTTSSLRRETTPTSSTTTRTPSFRRSSPTTHPSGETSER |
| | | | QSFRPRKGFSNLLAWLKRVLRRPLPESG* |
| 136 | 34 | MAAP_nt | ATGCAGACGAGGTCGCGCGAGAGCACGACATCTCGTACAACGAGCAGCTT |
| | | | GAGGCGGGAGACAACCCCTACCTCAAGTACAACCACGCGGACGCCGAGTT |
| | | | TCAGGAGAAGCTCGCCGACGACACATCCTTCGGGGGAAACCTCGGAAAGG |
| | | | CAGTCTTTCAGGCCAAGAAAAGGGTTCTCGAACCTTTTGGCCTGGTTGAA |
| | | | GAGGGTGCTAAGACGGCCCCTACCGGAAAGCGGATAG |
| 137 | 35 | VP1_aa | MSFVDHPPDWLEEVGEGLREFLGLEAGPPKPKPNQQHQDQARGLVLPGYN |
| | | | YLGPGNGLDRGEPVNRADEAAREHDKAYNEQLDAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 138 | 35 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGCGGCGCGAGAGCACGACAAGGCTTACAACGAGCAGCTTGATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 139 | 35 | MAAP_aa | MRETTPTSSTTTRTPSFRRSSPTTHPSGETSERQSFRPRKGFSNLLAWLK |
| | | | RVLRRPLPESG* |
| 140 | 35 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 141 | 36 | VP1_aa | MSFVDHPPDWLEEVGEGLREFLGLEAGPPKPKPNGQQHQDQARGLVLPGY |
| | | | NYLGPGNGLDRGEPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 142 | 36 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGGGCAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 143 | 36 | MAAP_aa | MGSSIKIKPVVLCCLVITISDPETGSIEESLSTGQTRSRESTTSRTTSSL |
| | | | RRETTPTSSTTTRTPSFRRSSPTTHPSGETSERQSFRPRKGFSNLLAWLK |
| | | | RVLRRPLPESG* |
| 144 | 36 | MAAP_nt | ATGGGCAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 145 | 37 | VP1_aa | MSFVDHPPDWLEEVGEGLREFLGLEAGPPKPKPNRVHDQSRGLVFPGYKY |
| | | | LGPGNGLDRGEPVNRADEVAREHDISYNEQLEAGDNPYLKYNHADAEFQE |
| | | | KLADDTSFGGNLGKAVFQAKKRVLEPFGLVEEGAKTAPTGKRIDDHFPKR |
| | | | KKARTEEDSKPSTSSDAEAGPSGSQQLQIPAQPASSLGADTMSAGGGGPL |
| | | | GDNNQGADGVGNASGDWHCDSTWMGDRVVTKSTRTWVLPSYNNHQYREIK |
| | | | SGSVDGSNANAYFGYSTPWGYFDFNRFHSHWSPRDWQRLINNYWGFRPRS |
| | | | LRVKIFNIQVKEVTVQDSTTTIANNLTSTVQVFTDDDYQLPYVVGNGTEG |
| | | | CLPAFPPQVFTLPQYGYATLNRDNTENPTERSSFFCLEYFPSKMLRTGNN |
| | | | FEFTYNFEEVPFHSSFAPSQNLFKLANPLVDQYLYRFVSTNNTGGVQFNK |
| | | | NLAGRYANTYKNWFPGPMGRTQGWNLGSGVNRASVSAFATTNRMELEGAS |
| | | | YQVPPQPNGMTNNLQGSNTYALENTMIFNSQPANPGTTATYLEGNMLITS |
| | | | ESETQPVNRVAYNVGGQMATNNQSSTTAPATGTYNLQEIVPGSVWMERDV |
| | | | YLQGPIWAKIPETGAHFHPSPAMGGFGLKHPPPMMLIKNTPVPGNITSFS |
| | | | DVPVSSFITQYSTGQVTVEMEWELKKENSKRWNPEIQYTNNYNDPQFVDF |
| | | | APDSTGEYRTTRPIGTRYLTRPL* |
| 146 | 37 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATAGGGTACATGATCAATCTCGTGGTCTTGTGTTTCCTGGTTATAAGTAT |
| | | | CTCGGACCCGGAAACGGGCTCGATCGAGGAGAGCCTGTCAACAGGGCAGA |
| | | | CGAGGTCGCGCGAGAGCACGACATCTCGTACAACGAGCAGCTTGAGGCGG |
| | | | GAGACAACCCCTACCTCAAGTACAACCACGCGGACGCCGAGTTTCAGGAG |
| | | | AAGCTCGCCGACGACACATCCTTCGGGGGAAACCTCGGAAAGGCAGTCTT |
| | | | TCAGGCCAAGAAAAGGGTTCTCGAACCTTTTGGCCTGGTTGAAGAGGGTG |
| | | | CTAAGACGGCCCCTACCGGAAAGCGGATAGACGACCACTTTCCAAAAAGA |
| | | | AAGAAGGCTCGGACCGAAGAGGACTCCAAGCCTTCCACCTCGTCAGACGC |
| | | | CGAAGCTGGACCCAGCGGATCCCAGCAGCTGCAAATCCCAGCCCAACCAG |
| | | | CCTCAAGTTTGGGAGCTGATACAATGTCTGCGGGAGGTGGCGGCCCATTG |
| | | | GGCGACAATAACCAAGGTGCCGATGGAGTGGGCAATGCCTCGGGAGATTG |
| | | | GCATTGCGATTCCACGTGGATGGGGGACAGAGTCGTCACCAAGTCCACCC |
| | | | GAACCTGGGTGCTGCCCAGCTACAACAACCACCAGTACCGAGAGATCAAA |
| | | | AGCGGCTCCGTCGACGGAAGCAACGCCAACGCCTACTTTGGATACAGCAC |
| | | | CCCCTGGGGGTACTTTGACTTTAACCGCTTCCACAGCCACTGGAGCCCCC |
| | | | GAGACTGGCAAAGACTCATCAACAACTACTGGGGCTTCAGACCCCGGTCC |
| | | | CTCAGAGTCAAAATCTTCAACATTCAAGTCAAAGAGGTCACGGTGCAGGA |
| | | | CTCCACCACCACCATCGCCAACAACCTCACCTCCACCGTCCAAGTGTTTA |
| | | | CGGACGACGACTACCAGCTGCCCTACGTCGTCGGCAACGGGACCGAGGGA |
| | | | TGCCTGCCGGCCTTCCCTCCGCAGGTCTTTACGCTGCCGCAGTACGGTTA |
| | | | CGCGACGCTGAACCGCGACAACACAGAAAATCCCACCGAGAGGAGCAGCT |
| | | | TCTTCTGCCTAGAGTACTTTCCCAGCAAGATGCTGAGAACGGGCAACAAC |
| | | | TTTGAGTTTACCTACAACTTTGAGGAGGTGCCCTTCCACTCCAGCTTCGC |
| | | | TCCCAGTCAGAACCTGTTCAAGCTGGCCAACCCGCTGGTGGACCAGTACT |
| | | | TGTACCGCTTCGTGAGCACAAATAACACTGGCGGAGTCCAGTTCAACAAG |
| | | | AACCTGGCCGGGAGATACGCCAACACCTACAAAAACTGGTTCCCGGGGCC |
| | | | CATGGGCCGAACCCAGGGCTGGAACCTGGGCTCCGGGGTCAACCGCGCCA |
| | | | GTGTCAGCGCCTTCGCCACGACCAATAGGATGGAGCTCGAGGGCGCGAGT |
| | | | TACCAGGTGCCCCCGCAGCCGAACGGCATGACCAACAACCTCCAGGGCAG |
| | | | CAACACCTATGCCCTGGAGAACACTATGATCTTCAACAGCCAGCCGGCGA |
| | | | ACCCGGGCACCACCGCCACGTACCTCGAGGGCAACATGCTCATCACCAGC |
| | | | GAGAGCGAGACACAGCCGGTGAACCGCGTGGCGTACAACGTCGGCGGGCA |
| | | | GATGGCCACCAACAACCAGAGCTCCACCACTGCCCCCGCGACCGGCACGT |
| | | | ACAACCTCCAGGAAATCGTGCCCGGCAGCGTGTGGATGGAGAGGGACGTG |
| | | | TACCTCCAAGGACCCATCTGGGCCAAGATCCCAGAGACAGGGGCGCACTT |
| | | | TCACCCCTCTCCGGCCATGGGCGGATTCGGACTCAAACACCCACCGCCCA |
| | | | TGATGCTCATCAAGAACACGCCTGTGCCCGGAAATATCACCAGCTTCTCG |
| | | | GACGTGCCCGTCAGCAGCTTCATCACCCAGTACAGCACCGGGCAGGTCAC |
| | | | CGTGGAGATGGAGTGGGAGCTCAAGAAGGAAAACTCCAAGAGGTGGAACC |
| | | | CAGAGATCCAGTACACAAACAACTACAACGACCCCCAGTTTGTGGACTTT |
| | | | GCCCCGGACAGCACCGGGGAATACAGAACCACCAGACCTATCGGAACCCG |
| | | | ATACCTTACCCGACCCCTTTAA |
| 147 | 37 | MAAP_aa | MINLVVLCFLVISISDPETGSIEESLSTGQTRSRESTTSRTTSSLRRETT |
| | | | PTSSTTTRTPSFRRSSPTTHPSGETSERQSFRPRKGFSNLLAWLKRVLRR |
| | | | PLPESG* |
| 148 | 37 | MAAP_nt | ATGATCAATCTCGTGGTCTTGTGTTTCCTGGTTATAAGTATCTCGGACCC |
| | | | GGAAACGGGCTCGATCGAGGAGAGCCTGTCAACAGGGCAGACGAGGTCGC |
| | | | GCGAGAGCACGACATCTCGTACAACGAGCAGCTTGAGGCGGGAGACAACC |
| | | | CCTACCTCAAGTACAACCACGCGGACGCCGAGTTTCAGGAGAAGCTCGCC |
| | | | GACGACACATCCTTCGGGGGAAACCTCGGAAAGGCAGTCTTTCAGGCCAA |
| | | | GAAAAGGGTTCTCGAACCTTTTGGCCTGGTTGAAGAGGGTGCTAAGACGG |
| | | | CCCCTACCGGAAAGCGGATAG |
| 149 | 38 | VP1_aa | MSFVDHPPDWLEEVGEGLREFLDGLEAGPPKPKPNQQHQDQARGLVLPGY |
| | | | NYLGPGNGLDRGEPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 150 | 38 | VP1_nt | ATGTCTTTTGTTGATCACCCTCCAGATTGGTTGGAAGAAGTTGGTGAAGG |
| | | | CCTTCGCGAGTTTTTGGATGGCCTTGAAGCGGGCCCACCGAAACCAAAAC |
| | | | CCAATCAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 151 | 38 | MAAP_aa | MALKRAHRNQNPISSIKIKPVVLCCLVITISDPETGSIEESLSTGQTRSR |
| | | | ESTTSRTTSSLRRETTPTSSTTTRTPSFRRSSPTTHPSGETSERQSFRPR |
| | | | KGFSNLLAWLKRVLRRPLPESG* |
| 152 | 38 | MAAP_nt | ATGGCCTTGAAGCGGGCCCACCGAAACCAAAACCCAATCAGCAGCATCAA |
| | | | GATCAAGCCCGTGGTCTTGTGCTGCCTGGTTATAACTATCTCGGACCCGG |
| | | | AAACGGGCTCGATCGAGGAGAGCCTGTCAACAGGGCAGACGAGGTCGCGC |
| | | | GAGAGCACGACATCTCGTACAACGAGCAGCTTGAGGCGGGAGACAACCCC |
| | | | TACCTCAAGTACAACCACGCGGACGCCGAGTTTCAGGAGAAGCTCGCCGA |
| | | | CGACACATCCTTCGGGGGAAACCTCGGAAAGGCAGTCTTTCAGGCCAAGA |
| | | | AAAGGGTTCTCGAACCTTTTGGCCTGGTTGAAGAGGGTGCTAAGACGGCC |
| | | | CCTACCGGAAAGCGGATAG |
| 153 | 39 | VP1_aa | MSFVDHPPDWLEEVGEGLREFLGLYAGPPKPDPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 154 | 39 | VP1_nt | ATGTCTTTTGTTGATCACCCTCCAGATTGGTTGGAAGAAGTTGGTGAAGG |
| | | | GCTTCGCGAGTTTTTGGGCCTTTATGCGGGCCCACCGAAACCAGACCCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 155 | 39 | MAAP_aa | MRAHRNQTPISSIKIKPVVLCCLVITISDPETGSIEESLSTGQTRSREST |
| | | | TSRTTSSLRRETTPTSSTTTRTPSFRRSSPTTHPSGETSERQSFRPRKGF |
| | | | SNLLAWLKRVLRRPLPESG* |
| 156 | 39 | MAAP_nt | ATGCGGGCCCACCGAAACCAGACCCCAATCAGCAGCATCAAGATCAAGCC |
| | | | CGTGGTCTTGTGCTGCCTGGTTATAACTATCTCGGACCCGGAAACGGGCT |
| | | | CGATCGAGGAGAGCCTGTCAACAGGGCAGACGAGGTCGCGCGAGAGCACG |
| | | | ACATCTCGTACAACGAGCAGCTTGAGGCGGGAGACAACCCCTACCTCAAG |
| | | | TACAACCACGCGGACGCCGAGTTTCAGGAGAAGCTCGCCGACGACACATC |
| | | | CTTCGGGGGAAACCTCGGAAAGGCAGTCTTTCAGGCCAAGAAAAGGGTTC |
| | | | TCGAACCTTTTGGCCTGGTTGAAGAGGGTGCTAAGACGGCCCCTACCGGA |
| | | | AAGCGGATAG |
| 157 | 40 | VP1_aa | MSFVDHPPDWLEEVGEGLREFLGLEAGPPKPKPNQQHQDQARGLVLPGYN |
| | | | YLGPGNGLDRGEPVNRADEVAREHDKAYDRQLDAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 158 | 40 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACAAAGCCTACGATCGGCAGCTTGATG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 159 | 40 | MAAP_aa | MRETTPTSSTTTRTPSFRRSSPTTHPSGETSERQSFRPRKGFSNLLAWLK |
| | | | RVLRRPLPESG* |
| 160 | 40 | MAAP_nt | ATGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAG |
| 161 | 41 | VP1_aa | MSFVDHPPDWLEEVGEGLREFLGLEAGPPKPKPNQQHQDQARGLVLPGYN |
| | | | YLGPGNGLDRGEPVNRADEVAREHDISYNEQLEAYGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 162 | 41 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGTATGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 163 | 41 | MAAP_aa | METTPTSSTTTRTPSFRRSSPTTHPSGETSERQSFRPRKGFSNLLAWLKR |
| | | | VLRRPLPESG* |
| 164 | 41 | MAAP_nt | ATGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 165 | 42 | VP1_aa | MSFVDHPPDWLEEVGEGLREFLGLEAGPPKPKPNQQHQDQARGLVLPGYV |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 166 | 42 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATGTC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 167 | 42 | MAAP_aa | MSISDPETGSIEESLSTGQTRSRESTTSRTTSSLRRETTPTSSTTTRTPS |
| | | | FRRSSPTTHPSGETSERQSFRPRKGFSNLLAWLKRVLRRPLPESG* |
| 168 | 42 | MAAP_nt | ATGTCTATCTCGGACCCGGAAACGGGCTCGATCGAGGAGAGCCTGTCAAC |
| | | | AGGGCAGACGAGGTCGCGCGAGAGCACGACATCTCGTACAACGAGCAGCT |
| | | | TGAGGCGGGAGACAACCCCTACCTCAAGTACAACCACGCGGACGCCGAGT |
| | | | TTCAGGAGAAGCTCGCCGACGACACATCCTTCGGGGGAAACCTCGGAAAG |
| | | | GCAGTCTTTCAGGCCAAGAAAAGGGTTCTCGAACCTTTTGGCCTGGTTGA |
| | | | AGAGGGTGCTAAGACGGCCCCTACCGGAAAGCGGATAG |
| 169 | 43 | VP1_aa | MSFVDHPPDWLEEVGEGLREFLGLHEAGPPKPKPNQQHQDQARGLVLPGY |
| | | | NYLGPGNGLDRGEPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 170 | 43 | VP1_nt | ATGTCTTTTGTTGATCACCCTCCAGATTGGTTGGAAGAAGTTGGTGAAGG |
| | | | ACTTCGCGAGTTTTTGGGCCTTCATGAAGCGGGCCCACCGAAACCAAAAC |
| | | | CCAATCAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 171 | 43 | MAAP_aa | MKRAHRNQNPISSIKIKPVVLCCLVITISDPETGSIEESLSTGQTRSRES |
| | | | TTSRTTSSLRRETTPTSSTTTRTPSFRRSSPTTHPSGETSERQSFRPRKG |
| | | | FSNLLAWLKRVLRRPLPESG* |
| 172 | 43 | MAAP_nt | ATGAAGCGGGCCCACCGAAACCAAAACCCAATCAGCAGCATCAAGATCAA |
| | | | GCCCGTGGTCTTGTGCTGCCTGGTTATAACTATCTCGGACCCGGAAACGG |
| | | | GCTCGATCGAGGAGAGCCTGTCAACAGGGCAGACGAGGTCGCGCGAGAGC |
| | | | ACGACATCTCGTACAACGAGCAGCTTGAGGCGGGAGACAACCCCTACCTC |
| | | | AAGTACAACCACGCGGACGCCGAGTTTCAGGAGAAGCTCGCCGACGACAC |
| | | | ATCCTTCGGGGGAAACCTCGGAAAGGCAGTCTTTCAGGCCAAGAAAAGGG |
| | | | TTCTCGAACCTTTTGGCCTGGTTGAAGAGGGTGCTAAGACGGCCCCTACC |
| | | | GGAAAGCGGATAG |
| 173 | 44 | VP1_aa | MSFVDHPPDWLEEVGEGLREFLGLEAGPPKPKPNEQHQDYARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 174 | 44 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGAGCAGCATCAAGATTACGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 175 | 44 | MAAP_aa | MSSIKITPVVLCCLVITISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 176 | 44 | MAAP_nt | ATGAGCAGCATCAAGATTACGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 177 | 45 | VP1_aa | MSFVDHPPDWLEEVGEGLREFLGLEAGPPKPKPNGQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 178 | 45 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGGACAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 179 | 45 | MAAP_aa | MDSIKIKPVVLCCLVITISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 180 | 45 | MAAP_nt | ATGGACAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 181 | 46 | VP1_aa | MSFVDHPPDWLEEVGEGLREFLGLEAGPPKPKPVQQHWDQARGLVLPGYE |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 182 | 46 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCG |
| | | | TCCAGCAGCATTGGGATCAAGCCCGTGGTCTTGTGCTGCCTGGTTATGAG |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 183 | 46 | MAAP_aa | MSISDPETGSIEESLSTGQTRSRESTTSRTTSSLRRETTPTSSTTTRTPS |
| | | | FRRSSPTTHPSGETSERQSFRPRKGFSNLLAWLKRVLRRPLPESG* |
| 184 | 46 | MAAP_nt | ATGAGTATCTCGGACCCGGAAACGGGCTCGATCGAGGAGAGCCTGTCAAC |
| | | | AGGGCAGACGAGGTCGCGCGAGAGCACGACATCTCGTACAACGAGCAGCT |
| | | | TGAGGCGGGAGACAACCCCTACCTCAAGTACAACCACGCGGACGCCGAGT |
| | | | TTCAGGAGAAGCTCGCCGACGACACATCCTTCGGGGGAAACCTCGGAAAG |
| | | | GCAGTCTTTCAGGCCAAGAAAAGGGTTCTCGAACCTTTTGGCCTGGTTGA |
| | | | AGAGGGTGCTAAGACGGCCCCTACCGGAAAGCGGATAG |
| 185 | 47 | VP1_aa | MSFVDHPPDWLEEVGEGLREFLGLEAGPPKPKPNQQISEHSPGSRGLVLP |
| | | | GYRYLGPGNGLDRGEPVNRADEVAREHDISYNEQLEAGDNPYLKYNHADA |
| | | | EFQEKLADDTSFGGNLGKAVFQAKKRVLEPFGLVEEGAKTAPTGKRIDDH |
| | | | FPKRKKARTEEDSKPSTSSDAEAGPSGSQQLQIPAQPASSLGADTMSAGG |
| | | | GGPLGDNNQGADGVGNASGDWHCDSTWMGDRVVTKSTRTWVLPSYNNHQY |
| | | | REIKSGSVDGSNANAYFGYSTPWGYFDFNRFHSHWSPRDWQRLINNYWGF |
| | | | RPRSLRVKIFNIQVKEVTVQDSTTTIANNLTSTVQVFTDDDYQLPYVVGN |
| | | | GTEGCLPAFPPQVFTLPQYGYATLNRDNTENPTERSSFFCLEYFPSKMLR |
| | | | TGNNFEFTYNFEEVPFHSSFAPSQNLFKLANPLVDQYLYRFVSTNNTGGV |
| | | | QFNKNLAGRYANTYKNWFPGPMGRTQGWNLGSGVNRASVSAFATTNRMEL |
| | | | EGASYQVPPQPNGMTNNLQGSNTYALENTMIFNSQPANPGTTATYLEGNM |
| | | | LITSESETQPVNRVAYNVGGQMATNNQSSTTAPATGTYNLQEIVPGSVWM |
| | | | ERDVYLQGPIWAKIPETGAHFHPSPAMGGFGLKHPPPMMLIKNTPVPGNI |
| | | | TSFSDVPVSSFITQYSTGQVTVEMEWELKKENSKRWNPEIQYTNNYNDPQ |
| | | | FVDFAPDSTGEYRTTRPIGTRYLTRPL* |
| 186 | 47 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGATCTCGGAACATAGTCCTGGCAGTCGTGGTCTTGTGCTGCCT |
| | | | GGTTATAGGTATCTCGGACCCGGAAACGGGCTCGATCGAGGAGAGCCTGT |
| | | | CAACAGGGCAGACGAGGTCGCGCGAGAGCACGACATCTCGTACAACGAGC |
| | | | AGCTTGAGGCGGGAGACAACCCCTACCTCAAGTACAACCACGCGGACGCC |
| | | | GAGTTTCAGGAGAAGCTCGCCGACGACACATCCTTCGGGGGAAACCTCGG |
| | | | AAAGGCAGTCTTTCAGGCCAAGAAAAGGGTTCTCGAACCTTTTGGCCTGG |
| | | | TTGAAGAGGGTGCTAAGACGGCCCCTACCGGAAAGCGGATAGACGACCAC |
| | | | TTTCCAAAAAGAAAGAAGGCTCGGACCGAAGAGGACTCCAAGCCTTCCAC |
| | | | CTCGTCAGACGCCGAAGCTGGACCCAGCGGATCCCAGCAGCTGCAAATCC |
| | | | CAGCCCAACCAGCCTCAAGTTTGGGAGCTGATACAATGTCTGCGGGAGGT |
| | | | GGCGGCCCATTGGGCGACAATAACCAAGGTGCCGATGGAGTGGGCAATGC |
| | | | CTCGGGAGATTGGCATTGCGATTCCACGTGGATGGGGGACAGAGTCGTCA |
| | | | CCAAGTCCACCCGAACCTGGGTGCTGCCCAGCTACAACAACCACCAGTAC |
| | | | CGAGAGATCAAAAGCGGCTCCGTCGACGGAAGCAACGCCAACGCCTACTT |
| | | | TGGATACAGCACCCCCTGGGGGTACTTTGACTTTAACCGCTTCCACAGCC |
| | | | ACTGGAGCCCCCGAGACTGGCAAAGACTCATCAACAACTACTGGGGCTTC |
| | | | AGACCCCGGTCCCTCAGAGTCAAAATCTTCAACATTCAAGTCAAAGAGGT |
| | | | CACGGTGCAGGACTCCACCACCACCATCGCCAACAACCTCACCTCCACCG |
| | | | TCCAAGTGTTTACGGACGACGACTACCAGCTGCCCTACGTCGTCGGCAAC |
| | | | GGGACCGAGGGATGCCTGCCGGCCTTCCCTCCGCAGGTCTTTACGCTGCC |
| | | | GCAGTACGGTTACGCGACGCTGAACCGCGACAACACAGAAAATCCCACCG |
| | | | AGAGGAGCAGCTTCTTCTGCCTAGAGTACTTTCCCAGCAAGATGCTGAGA |
| | | | ACGGGCAACAACTTTGAGTTTACCTACAACTTTGAGGAGGTGCCCTTCCA |
| | | | CTCCAGCTTCGCTCCCAGTCAGAACCTGTTCAAGCTGGCCAACCCGCTGG |
| | | | TGGACCAGTACTTGTACCGCTTCGTGAGCACAAATAACACTGGCGGAGTC |
| | | | CAGTTCAACAAGAACCTGGCCGGGAGATACGCCAACACCTACAAAAACTG |
| | | | GTTCCCGGGGCCCATGGGCCGAACCCAGGGCTGGAACCTGGGCTCCGGGG |
| | | | TCAACCGCGCCAGTGTCAGCGCCTTCGCCACGACCAATAGGATGGAGCTC |
| | | | GAGGGCGCGAGTTACCAGGTGCCCCCGCAGCCGAACGGCATGACCAACAA |
| | | | CCTCCAGGGCAGCAACACCTATGCCCTGGAGAACACTATGATCTTCAACA |
| | | | GCCAGCCGGCGAACCCGGGCACCACCGCCACGTACCTCGAGGGCAACATG |
| | | | CTCATCACCAGCGAGAGCGAGACACAGCCGGTGAACCGCGTGGCGTACAA |
| | | | CGTCGGCGGGCAGATGGCCACCAACAACCAGAGCTCCACCACTGCCCCCG |
| | | | CGACCGGCACGTACAACCTCCAGGAAATCGTGCCCGGCAGCGTGTGGATG |
| | | | GAGAGGGACGTGTACCTCCAAGGACCCATCTGGGCCAAGATCCCAGAGAC |
| | | | AGGGGCGCACTTTCACCCCTCTCCGGCCATGGGCGGATTCGGACTCAAAC |
| | | | ACCCACCGCCCATGATGCTCATCAAGAACACGCCTGTGCCCGGAAATATC |
| | | | ACCAGCTTCTCGGACGTGCCCGTCAGCAGCTTCATCACCCAGTACAGCAC |
| | | | CGGGCAGGTCACCGTGGAGATGGAGTGGGAGCTCAAGAAGGAAAACTCCA |
| | | | AGAGGTGGAACCCAGAGATCCAGTACACAAACAACTACAACGACCCCCAG |
| | | | TTTGTGGACTTTGCCCCGGACAGCACCGGGGAATACAGAACCACCAGACC |
| | | | TATCGGAACCCGATACCTTACCCGACCCCTTTAA |
| 187 | 47 | MAAP_aa | LAVVVLCCLVIGISDPETGSIEESLSTGQTRSRESTTSRTTSSLRRETTP |
| | | | TSSTTTRTPSFRRSSPTTHPSGETSERQSFRPRKGFSNLLAWLKRVLRRP |
| | | | LPESG* |
| 188 | 47 | MAAP_nt | CTGGCAGTCGTGGTCTTGTGCTGCCTGGTTATAGGTATCTCGGACCCGGA |
| | | | AACGGGCTCGATCGAGGAGAGCCTGTCAACAGGGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 189 | 48 | VP1_aa | MSFVDHPPDWLEEVGEGLREFLGLEAGPPKPKPNDQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 190 | 48 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGATCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 191 | 48 | MAAP_aa | MISIKIKPVVLCCLVITISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 192 | 48 | MAAP_nt | ATGATCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 193 | 49 | VP1_aa | MSFVDHPPDWLEEVGEGLREFLGLEAGPPKPKPNQQHQDQARGLVLPGYN |
| | | | YLGPGNGLDRGEPVNRADEVAREHDKSYDEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 194 | 49 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACAAGTCGTACGATGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 195 | 49 | MAAP_aa | MSSLRRETTPTSSTTTRTPSFRRSSPTTHPSGETSERQSFRPRKGFSNLL |
| | | | AWLKRVLRRPLPESG* |
| 196 | 49 | MAAP_nt | ATGAGCAGCTTGAGGCGGGAGACAACCCCTACCTCAAGTACAACCACGCG |
| | | | GACGCCGAGTTTCAGGAGAAGCTCGCCGACGACACATCCTTCGGGGGAAA |
| | | | CCTCGGAAAGGCAGTCTTTCAGGCCAAGAAAAGGGTTCTCGAACCTTTTG |
| | | | GCCTGGTTGAAGAGGGTGCTAAGACGGCCCCTACCGGAAAGCGGATAG |
| 197 | 50 | VP1_aa | MSFVDHPPDWLEEVGEGLREFHGLEAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 198 | 50 | VP1_nt | ATGTCTTTTGTTGATCACCCTCCAGATTGGTTGGAAGAAGTTGGTGAAGG |
| | | | CCTTCGCGAGTTTCATGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 199 | 50 | MAAP_aa | MALKRAHRNQNPISSIKIKPVVLCCLVITISDPETGSIEESLSTGQTRSR |
| | | | ESTTSRTTSSLRRETTPTSSTTTRTPSFRRSSPTTHPSGETSERQSFRPR |
| | | | KGFSNLLAWLKRVLRRPLPESG* |
| 200 | 50 | MAAP_nt | ATGGCCTTGAAGCGGGCCCACCGAAACCAAAACCCAATCAGCAGCATCAA |
| | | | GATCAAGCCCGTGGTCTTGTGCTGCCTGGTTATAACTATCTCGGACCCGG |
| | | | AAACGGGCTCGATCGAGGAGAGCCTGTCAACAGGGCAGACGAGGTCGCGC |
| | | | GAGAGCACGACATCTCGTACAACGAGCAGCTTGAGGCGGGAGACAACCCC |
| | | | TACCTCAAGTACAACCACGCGGACGCCGAGTTTCAGGAGAAGCTCGCCGA |
| | | | CGACACATCCTTCGGGGGAAACCTCGGAAAGGCAGTCTTTCAGGCCAAGA |
| | | | AAAGGGTTCTCGAACCTTTTGGCCTGGTTGAAGAGGGTGCTAAGACGGCC |
| | | | CCTACCGGAAAGCGGATAG |
| 201 | 51 | VP1_aa | MSFVDHPPDWLETLGEGLREFWGLKPGPPKPKPAEQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 202 | 51 | VP1_nt | ATGTCTTTTGTTGATCACCCTCCAGATTGGTTGGAAACGCTCGGTGAAGG |
| | | | TCTACGCGAGTTTTGGGGCCTTAAACCTGGCCCACCGAAACCAAAACCCG |
| | | | CTGAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 203 | 51 | MAAP_aa | LAHRNQNPLSSIKIKPVVLCCLVITISDPETGSIEESLSTGQTRSRESTT |
| | | | SRTTSSLRRETTPTSSTTTRTPSFRRSSPTTHPSGETSERQSFRPRKGFS |
| | | | NLLAWLKRVLRRPLPESG* |
| 204 | 51 | MAAP_nt | CTGGCCCACCGAAACCAAAACCCGCTGAGCAGCATCAAGATCAAGCCCGT |
| | | | GGTCTTGTGCTGCCTGGTTATAACTATCTCGGACCCGGAAACGGGCTCGA |
| | | | TCGAGGAGAGCCTGTCAACAGGGCAGACGAGGTCGCGCGAGAGCACGACA |
| | | | TCTCGTACAACGAGCAGCTTGAGGCGGGAGACAACCCCTACCTCAAGTAC |
| | | | AACCACGCGGACGCCGAGTTTCAGGAGAAGCTCGCCGACGACACATCCTT |
| | | | CGGGGGAAACCTCGGAAAGGCAGTCTTTCAGGCCAAGAAAAGGGTTCTCG |
| | | | AACCTTTTGGCCTGGTTGAAGAGGGTGCTAAGACGGCCCCTACCGGAAAG |
| | | | CGGATAG |
| 205 | 52 | VP1_aa | MSFVDHPPDWLEEVGEGLREFLGLEAGPPKPKPNQQHDDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 206 | 52 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATGACGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 207 | 52 | MAAP_aa | MTIKPVVLCCLVITISDPETGSIEESLSTGQTRSRESTTSRTTSSLRRET |
| | | | TPTSSTTTRTPSFRRSSPTTHPSGETSERQSFRPRKGFSNLLAWLKRVLR |
| | | | RPLPESG* |
| 208 | 52 | MAAP_nt | ATGACGATCAAGCCCGTGGTCTTGTGCTGCCTGGTTATAACTATCTCGGA |
| | | | CCCGGAAACGGGCTCGATCGAGGAGAGCCTGTCAACAGGGCAGACGAGGT |
| | | | CGCGCGAGAGCACGACATCTCGTACAACGAGCAGCTTGAGGCGGGAGACA |
| | | | ACCCCTACCTCAAGTACAACCACGCGGACGCCGAGTTTCAGGAGAAGCTC |
| | | | GCCGACGACACATCCTTCGGGGGAAACCTCGGAAAGGCAGTCTTTCAGGC |
| | | | CAAGAAAAGGGTTCTCGAACCTTTTGGCCTGGTTGAAGAGGGTGCTAAGA |
| | | | CGGCCCCTACCGGAAAGCGGATAG |
| 209 | 53 | VP1_aa | MSFVDGPPDWLEEVGEGLREFLGLEAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 210 | 53 | VP1_nt | ATGTCTTTTGTTGATGGACCTCCAGATTGGTTGGAAGAAGTTGGTGAAGG |
| | | | TCTGCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 211 | 53 | MAAP_aa | MDLQIGWKKLVKVCASFWALKRAHRNQNPISSIKIKPVVLCCLVITISDP |
| | | | ETGSIEESLSTGQTRSRESTTSRTTSSLRRETTPTSSTTTRTPSFRRSSP |
| | | | TTHPSGETSERQSFRPRKGFSNLLAWLKRVLRRPLPESG* |
| 212 | 53 | MAAP_nt | ATGGACCTCCAGATTGGTTGGAAGAAGTTGGTGAAGGTCTGCGCGAGTTT |
| | | | TTGGGCCTTGAAGCGGGCCCACCGAAACCAAAACCCAATCAGCAGCATCA |
| | | | AGATCAAGCCCGTGGTCTTGTGCTGCCTGGTTATAACTATCTCGGACCCG |
| | | | GAAACGGGCTCGATCGAGGAGAGCCTGTCAACAGGGCAGACGAGGTCGCG |
| | | | CGAGAGCACGACATCTCGTACAACGAGCAGCTTGAGGCGGGAGACAACCC |
| | | | CTACCTCAAGTACAACCACGCGGACGCCGAGTTTCAGGAGAAGCTCGCCG |
| | | | ACGACACATCCTTCGGGGGAAACCTCGGAAAGGCAGTCTTTCAGGCCAAG |
| | | | AAAAGGGTTCTCGAACCTTTTGGCCTGGTTGAAGAGGGTGCTAAGACGGC |
| | | | CCCTACCGGAAAGCGGATAG |
| 213 | 54 | VP1_aa | MSFVDHPPDWLEEVGEGLREFLGLEAGPPKPKPAQRHKDDSRGLVLPGYR |
| | | | YLGPFNGLDKGEPVNEADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 214 | 54 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCG |
| | | | CTCAGCGCCATAAGGATGATAGTCGTGGTCTTGTGCTGCCTGGTTATCGC |
| | | | TATCTCGGACCCTTCAACGGGCTCGATAAGGGAGAGCCTGTCAACGAGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 215 | 54 | MAAP_aa | MIVVVLCCLVIAISDPSTGSIRESLSTRQTRSRESTTSRTTSSLRRETTP |
| | | | TSSTTTRTPSFRRSSPTTHPSGETSERQSFRPRKGFSNLLAWLKRVLRRP |
| | | | LPESG* |
| 216 | 54 | MAAP_nt | ATGATAGTCGTGGTCTTGTGCTGCCTGGTTATCGCTATCTCGGACCCTTC |
| | | | AACGGGCTCGATAAGGGAGAGCCTGTCAACGAGGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 217 | 55 | VP1_aa | MSFVDHPPDWLEEVGEGLREFLGLEAGPPKPKPNQQHDQARGLVLPGYNY |
| | | | LGPGNGLDRGEPVNAADEVAREHDISYNEQLEAGDNPYLKYNHADAEFQE |
| | | | KLADDTSFGGNLGKAVFQAKKRVLEPFGLVEEGAKTAPTGKRIDDHFPKR |
| | | | KKARTEEDSKPSTSSDAEAGPSGSQQLQIPAQPASSLGADTMSAGGGGPL |
| | | | GDNNQGADGVGNASGDWHCDSTWMGDRVVTKSTRTWVLPSYNNHQYREIK |
| | | | SGSVDGSNANAYFGYSTPWGYFDFNRFHSHWSPRDWQRLINNYWGFRPRS |
| | | | LRVKIFNIQVKEVTVQDSTTTIANNLTSTVQVFTDDDYQLPYVVGNGTEG |
| | | | CLPAFPPQVFTLPQYGYATLNRDNTENPTERSSFFCLEYFPSKMLRTGNN |
| | | | FEFTYNFEEVPFHSSFAPSQNLFKLANPLVDQYLYRFVSTNNTGGVQFNK |
| | | | NLAGRYANTYKNWFPGPMGRTQGWNLGSGVNRASVSAFATTNRMELEGAS |
| | | | YQVPPQPNGMTNNLQGSNTYALENTMIFNSQPANPGTTATYLEGNMLITS |
| | | | ESETQPVNRVAYNVGGQMATNNQSSTTAPATGTYNLQEIVPGSVWMERDV |
| | | | YLQGPIWAKIPETGAHFHPSPAMGGFGLKHPPPMMLIKNTPVPGNITSFS |
| | | | DVPVSSFITQYSTGQVTVEMEWELKKENSKRWNPEIQYTNNYNDPQFVDF |
| | | | APDSTGEYRTTRPIGTRYLTRPL* |
| 218 | 55 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATGATCAAGCCCGTGGTCTTGTGCTGCCTGGTTATAACTAT |
| | | | CTCGGACCCGGAAACGGGCTCGATCGAGGAGAGCCTGTCAACGCCGCAGA |
| | | | CGAGGTCGCGCGAGAGCACGACATCTCGTACAACGAGCAGCTTGAGGCGG |
| | | | GAGACAACCCCTACCTCAAGTACAACCACGCGGACGCCGAGTTTCAGGAG |
| | | | AAGCTCGCCGACGACACATCCTTCGGGGGAAACCTCGGAAAGGCAGTCTT |
| | | | TCAGGCCAAGAAAAGGGTTCTCGAACCTTTTGGCCTGGTTGAAGAGGGTG |
| | | | CTAAGACGGCCCCTACCGGAAAGCGGATAGACGACCACTTTCCAAAAAGA |
| | | | AAGAAGGCTCGGACCGAAGAGGACTCCAAGCCTTCCACCTCGTCAGACGC |
| | | | CGAAGCTGGACCCAGCGGATCCCAGCAGCTGCAAATCCCAGCCCAACCAG |
| | | | CCTCAAGTTTGGGAGCTGATACAATGTCTGCGGGAGGTGGCGGCCCATTG |
| | | | GGCGACAATAACCAAGGTGCCGATGGAGTGGGCAATGCCTCGGGAGATTG |
| | | | GCATTGCGATTCCACGTGGATGGGGGACAGAGTCGTCACCAAGTCCACCC |
| | | | GAACCTGGGTGCTGCCCAGCTACAACAACCACCAGTACCGAGAGATCAAA |
| | | | AGCGGCTCCGTCGACGGAAGCAACGCCAACGCCTACTTTGGATACAGCAC |
| | | | CCCCTGGGGGTACTTTGACTTTAACCGCTTCCACAGCCACTGGAGCCCCC |
| | | | GAGACTGGCAAAGACTCATCAACAACTACTGGGGCTTCAGACCCCGGTCC |
| | | | CTCAGAGTCAAAATCTTCAACATTCAAGTCAAAGAGGTCACGGTGCAGGA |
| | | | CTCCACCACCACCATCGCCAACAACCTCACCTCCACCGTCCAAGTGTTTA |
| | | | CGGACGACGACTACCAGCTGCCCTACGTCGTCGGCAACGGGACCGAGGGA |
| | | | TGCCTGCCGGCCTTCCCTCCGCAGGTCTTTACGCTGCCGCAGTACGGTTA |
| | | | CGCGACGCTGAACCGCGACAACACAGAAAATCCCACCGAGAGGAGCAGCT |
| | | | TCTTCTGCCTAGAGTACTTTCCCAGCAAGATGCTGAGAACGGGCAACAAC |
| | | | TTTGAGTTTACCTACAACTTTGAGGAGGTGCCCTTCCACTCCAGCTTCGC |
| | | | TCCCAGTCAGAACCTGTTCAAGCTGGCCAACCCGCTGGTGGACCAGTACT |
| | | | TGTACCGCTTCGTGAGCACAAATAACACTGGCGGAGTCCAGTTCAACAAG |
| | | | AACCTGGCCGGGAGATACGCCAACACCTACAAAAACTGGTTCCCGGGGCC |
| | | | CATGGGCCGAACCCAGGGCTGGAACCTGGGCTCCGGGGTCAACCGCGCCA |
| | | | GTGTCAGCGCCTTCGCCACGACCAATAGGATGGAGCTCGAGGGCGCGAGT |
| | | | TACCAGGTGCCCCCGCAGCCGAACGGCATGACCAACAACCTCCAGGGCAG |
| | | | CAACACCTATGCCCTGGAGAACACTATGATCTTCAACAGCCAGCCGGCGA |
| | | | ACCCGGGCACCACCGCCACGTACCTCGAGGGCAACATGCTCATCACCAGC |
| | | | GAGAGCGAGACACAGCCGGTGAACCGCGTGGCGTACAACGTCGGCGGGCA |
| | | | GATGGCCACCAACAACCAGAGCTCCACCACTGCCCCCGCGACCGGCACGT |
| | | | ACAACCTCCAGGAAATCGTGCCCGGCAGCGTGTGGATGGAGAGGGACGTG |
| | | | TACCTCCAAGGACCCATCTGGGCCAAGATCCCAGAGACAGGGGCGCACTT |
| | | | TCACCCCTCTCCGGCCATGGGCGGATTCGGACTCAAACACCCACCGCCCA |
| | | | TGATGCTCATCAAGAACACGCCTGTGCCCGGAAATATCACCAGCTTCTCG |
| | | | GACGTGCCCGTCAGCAGCTTCATCACCCAGTACAGCACCGGGCAGGTCAC |
| | | | CGTGGAGATGGAGTGGGAGCTCAAGAAGGAAAACTCCAAGAGGTGGAACC |
| | | | CAGAGATCCAGTACACAAACAACTACAACGACCCCCAGTTTGTGGACTTT |
| | | | GCCCCGGACAGCACCGGGGAATACAGAACCACCAGACCTATCGGAACCCG |
| | | | ATACCTTACCCGACCCCTTTAA |
| 219 | 55 | MAAP_aa | MIKPVVLCCLVITISDPETGSIEESLSTPQTRSRESTTSRTTSSLRRETT |
| | | | PTSSTTTRTPSFRRSSPTTHPSGETSERQSFRPRKGFSNLLAWLKRVLRR |
| | | | PLPESG* |
| 220 | 55 | MAAP_nt | ATGATCAAGCCCGTGGTCTTGTGCTGCCTGGTTATAACTATCTCGGACCC |
| | | | GGAAACGGGCTCGATCGAGGAGAGCCTGTCAACGCCGCAGACGAGGTCGC |
| | | | GCGAGAGCACGACATCTCGTACAACGAGCAGCTTGAGGCGGGAGACAACC |
| | | | CCTACCTCAAGTACAACCACGCGGACGCCGAGTTTCAGGAGAAGCTCGCC |
| | | | GACGACACATCCTTCGGGGGAAACCTCGGAAAGGCAGTCTTTCAGGCCAA |
| | | | GAAAAGGGTTCTCGAACCTTTTGGCCTGGTTGAAGAGGGTGCTAAGACGG |
| | | | CCCCTACCGGAAAGCGGATAG |
| 221 | 56 | VP1_aa | MSFVDHPPDWLEEVGEGLREFLGLEAGPPKPKPNQQHVDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 222 | 56 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATGTGGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 223 | 56 | MAAP_aa | MWIKPVVLCCLVITISDPETGSIEESLSTGQTRSRESTTSRTTSSLRRET |
| | | | TPTSSTTTRTPSFRRSSPTTHPSGETSERQSFRPRKGFSNLLAWLKRVLR |
| | | | RPLPESG* |
| 224 | 56 | MAAP_nt | ATGTGGATCAAGCCCGTGGTCTTGTGCTGCCTGGTTATAACTATCTCGGA |
| | | | CCCGGAAACGGGCTCGATCGAGGAGAGCCTGTCAACAGGGCAGACGAGGT |
| | | | CGCGCGAGAGCACGACATCTCGTACAACGAGCAGCTTGAGGCGGGAGACA |
| | | | ACCCCTACCTCAAGTACAACCACGCGGACGCCGAGTTTCAGGAGAAGCTC |
| | | | GCCGACGACACATCCTTCGGGGGAAACCTCGGAAAGGCAGTCTTTCAGGC |
| | | | CAAGAAAAGGGTTCTCGAACCTTTTGGCCTGGTTGAAGAGGGTGCTAAGA |
| | | | CGGCCCCTACCGGAAAGCGGATAG |
| 225 | 57 | VP1_aa | MSFVDHPPDWLEEVGEGLREFLGLEAGPPKPKPNQQKQDARGLVLPGYKY |
| | | | LGPFNGLDRGEPVNRADEVAREHDISYNEQLEAGDNPYLKYNHADAEFQE |
| | | | KLADDTSFGGNLGKAVFQAKKRVLEPFGLVEEGAKTAPTGKRIDDHFPKR |
| | | | KKARTEEDSKPSTSSDAEAGPSGSQQLQIPAQPASSLGADTMSAGGGGPL |
| | | | GDNNQGADGVGNASGDWHCDSTWMGDRVVTKSTRTWVLPSYNNHQYREIK |
| | | | SGSVDGSNANAYFGYSTPWGYFDFNRFHSHWSPRDWQRLINNYWGFRPRS |
| | | | LRVKIFNIQVKEVTVQDSTTTIANNLTSTVQVFTDDDYQLPYVVGNGTEG |
| | | | CLPAFPPQVFTLPQYGYATLNRDNTENPTERSSFFCLEYFPSKMLRTGNN |
| | | | FEFTYNFEEVPFHSSFAPSQNLFKLANPLVDQYLYRFVSTNNTGGVQFNK |
| | | | NLAGRYANTYKNWFPGPMGRTQGWNLGSGVNRASVSAFATTNRMELEGAS |
| | | | YQVPPQPNGMTNNLQGSNTYALENTMIFNSQPANPGTTATYLEGNMLITS |
| | | | ESETQPVNRVAYNVGGQMATNNQSSTTAPATGTYNLQEIVPGSVWMERDV |
| | | | YLQGPIWAKIPETGAHFHPSPAMGGFGLKHPPPMMLIKNTPVPGNITSFS |
| | | | DVPVSSFITQYSTGQVTVEMEWELKKENSKRWNPEIQYTNNYNDPQFVDF |
| | | | APDSTGEYRTTRPIGTRYLTRPL* |
| 226 | 57 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGAAGCAAGATGCCCGTGGTCTTGTGCTGCCTGGTTATAAATAT |
| | | | CTCGGACCCTTCAACGGGCTCGATCGAGGAGAGCCTGTCAACAGGGCAGA |
| | | | CGAGGTCGCGCGAGAGCACGACATCTCGTACAACGAGCAGCTTGAGGCGG |
| | | | GAGACAACCCCTACCTCAAGTACAACCACGCGGACGCCGAGTTTCAGGAG |
| | | | AAGCTCGCCGACGACACATCCTTCGGGGGAAACCTCGGAAAGGCAGTCTT |
| | | | TCAGGCCAAGAAAAGGGTTCTCGAACCTTTTGGCCTGGTTGAAGAGGGTG |
| | | | CTAAGACGGCCCCTACCGGAAAGCGGATAGACGACCACTTTCCAAAAAGA |
| | | | AAGAAGGCTCGGACCGAAGAGGACTCCAAGCCTTCCACCTCGTCAGACGC |
| | | | CGAAGCTGGACCCAGCGGATCCCAGCAGCTGCAAATCCCAGCCCAACCAG |
| | | | CCTCAAGTTTGGGAGCTGATACAATGTCTGCGGGAGGTGGCGGCCCATTG |
| | | | GGCGACAATAACCAAGGTGCCGATGGAGTGGGCAATGCCTCGGGAGATTG |
| | | | GCATTGCGATTCCACGTGGATGGGGGACAGAGTCGTCACCAAGTCCACCC |
| | | | GAACCTGGGTGCTGCCCAGCTACAACAACCACCAGTACCGAGAGATCAAA |
| | | | AGCGGCTCCGTCGACGGAAGCAACGCCAACGCCTACTTTGGATACAGCAC |
| | | | CCCCTGGGGGTACTTTGACTTTAACCGCTTCCACAGCCACTGGAGCCCCC |
| | | | GAGACTGGCAAAGACTCATCAACAACTACTGGGGCTTCAGACCCCGGTCC |
| | | | CTCAGAGTCAAAATCTTCAACATTCAAGTCAAAGAGGTCACGGTGCAGGA |
| | | | CTCCACCACCACCATCGCCAACAACCTCACCTCCACCGTCCAAGTGTTTA |
| | | | CGGACGACGACTACCAGCTGCCCTACGTCGTCGGCAACGGGACCGAGGGA |
| | | | TGCCTGCCGGCCTTCCCTCCGCAGGTCTTTACGCTGCCGCAGTACGGTTA |
| | | | CGCGACGCTGAACCGCGACAACACAGAAAATCCCACCGAGAGGAGCAGCT |
| | | | TCTTCTGCCTAGAGTACTTTCCCAGCAAGATGCTGAGAACGGGCAACAAC |
| | | | TTTGAGTTTACCTACAACTTTGAGGAGGTGCCCTTCCACTCCAGCTTCGC |
| | | | TCCCAGTCAGAACCTGTTCAAGCTGGCCAACCCGCTGGTGGACCAGTACT |
| | | | TGTACCGCTTCGTGAGCACAAATAACACTGGCGGAGTCCAGTTCAACAAG |
| | | | AACCTGGCCGGGAGATACGCCAACACCTACAAAAACTGGTTCCCGGGGCC |
| | | | CATGGGCCGAACCCAGGGCTGGAACCTGGGCTCCGGGGTCAACCGCGCCA |
| | | | GTGTCAGCGCCTTCGCCACGACCAATAGGATGGAGCTCGAGGGCGCGAGT |
| | | | TACCAGGTGCCCCCGCAGCCGAACGGCATGACCAACAACCTCCAGGGCAG |
| | | | CAACACCTATGCCCTGGAGAACACTATGATCTTCAACAGCCAGCCGGCGA |
| | | | ACCCGGGCACCACCGCCACGTACCTCGAGGGCAACATGCTCATCACCAGC |
| | | | GAGAGCGAGACACAGCCGGTGAACCGCGTGGCGTACAACGTCGGCGGGCA |
| | | | GATGGCCACCAACAACCAGAGCTCCACCACTGCCCCCGCGACCGGCACGT |
| | | | ACAACCTCCAGGAAATCGTGCCCGGCAGCGTGTGGATGGAGAGGGACGTG |
| | | | TACCTCCAAGGACCCATCTGGGCCAAGATCCCAGAGACAGGGGCGCACTT |
| | | | TCACCCCTCTCCGGCCATGGGCGGATTCGGACTCAAACACCCACCGCCCA |
| | | | TGATGCTCATCAAGAACACGCCTGTGCCCGGAAATATCACCAGCTTCTCG |
| | | | GACGTGCCCGTCAGCAGCTTCATCACCCAGTACAGCACCGGGCAGGTCAC |
| | | | CGTGGAGATGGAGTGGGAGCTCAAGAAGGAAAACTCCAAGAGGTGGAACC |
| | | | CAGAGATCCAGTACACAAACAACTACAACGACCCCCAGTTTGTGGACTTT |
| | | | GCCCCGGACAGCACCGGGGAATACAGAACCACCAGACCTATCGGAACCCG |
| | | | ATACCTTACCCGACCCCTTTAA |
| 227 | 57 | MAAP_aa | MPVVLCCLVINISDPSTGSIEESLSTGQTRSRESTTSRTTSSLRRETTPT |
| | | | SSTTTRTPSFRRSSPTTHPSGETSERQSFRPRKGFSNLLAWLKRVLRRPL |
| | | | PESG* |
| 228 | 57 | MAAP_nt | ATGCCCGTGGTCTTGTGCTGCCTGGTTATAAATATCTCGGACCCTTCAAC |
| | | | GGGCTCGATCGAGGAGAGCCTGTCAACAGGGCAGACGAGGTCGCGCGAGA |
| | | | GCACGACATCTCGTACAACGAGCAGCTTGAGGCGGGAGACAACCCCTACC |
| | | | TCAAGTACAACCACGCGGACGCCGAGTTTCAGGAGAAGCTCGCCGACGAC |
| | | | ACATCCTTCGGGGGAAACCTCGGAAAGGCAGTCTTTCAGGCCAAGAAAAG |
| | | | GGTTCTCGAACCTTTTGGCCTGGTTGAAGAGGGTGCTAAGACGGCCCCTA |
| | | | CCGGAAAGCGGATAG |
| 229 | 58 | VP1_aa | MSFVDHPPDWLEEVGEGLREFLGLEAGPPKPKPNQQHDQDQARGLVLPGY |
| | | | NYLGPGNGLDRGEPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 230 | 58 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATGACCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 231 | 58 | MAAP_aa | MTKIKPVVLCCLVITISDPETGSIEESLSTGQTRSRESTTSRTTSSLRRE |
| | | | TTPTSSTTTRTPSFRRSSPTTHPSGETSERQSFRPRKGFSNLLAWLKRVL |
| | | | RRPLPESG* |
| 232 | 58 | MAAP_nt | ATGACCAAGATCAAGCCCGTGGTCTTGTGCTGCCTGGTTATAACTATCTC |
| | | | GGACCCGGAAACGGGCTCGATCGAGGAGAGCCTGTCAACAGGGCAGACGA |
| | | | GGTCGCGCGAGAGCACGACATCTCGTACAACGAGCAGCTTGAGGCGGGAG |
| | | | ACAACCCCTACCTCAAGTACAACCACGCGGACGCCGAGTTTCAGGAGAAG |
| | | | CTCGCCGACGACACATCCTTCGGGGGAAACCTCGGAAAGGCAGTCTTTCA |
| | | | GGCCAAGAAAAGGGTTCTCGAACCTTTTGGCCTGGTTGAAGAGGGTGCTA |
| | | | AGACGGCCCCTACCGGAAAGCGGATAG |
| 233 | 59 | VP1_aa | MSFVDHPPDWLEEVGEGLREFLGLEAGPPKPKPNEQHQDQARGLVLPGYK |
| | | | YLGPFNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 234 | 59 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGAACAGCATCAAGATCAAGCCCGTGGTCTTGTGCTGCCTGGTTATAAA |
| | | | TATCTCGGACCCTTCAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 235 | 59 | MAAP_aa | MNSIKIKPVVLCCLVINISDPSTGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 236 | 59 | MAAP_nt | ATGAACAGCATCAAGATCAAGCCCGTGGTCTTGTGCTGCCTGGTTATAAA |
| | | | TATCTCGGACCCTTCAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 237 | 60 | VP1_aa | MSFVDHPPDWLEEVGEGLREFLGLEAGPPKPKPNQQHQDQARGLVLPGYN |
| | | | YLGPGNGLDRGEPVNRADAVAREHDKAYDEQLKAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 238 | 60 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGCCGTCGCGCGAGAGCACGACAAAGCATACGATGAGCAGCTTAAAG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 239 | 60 | MAAP_aa | MSSLKRETTPTSSTTTRTPSFRRSSPTTHPSGETSERQSFRPRKGFSNLL |
| | | | AWLKRVLRRPLPESG* |
| 240 | 60 | MAAP_nt | ATGAGCAGCTTAAAGCGGGAGACAACCCCTACCTCAAGTACAACCACGCG |
| | | | GACGCCGAGTTTCAGGAGAAGCTCGCCGACGACACATCCTTCGGGGGAAA |
| | | | CCTCGGAAAGGCAGTCTTTCAGGCCAAGAAAAGGGTTCTCGAACCTTTTG |
| | | | GCCTGGTTGAAGAGGGTGCTAAGACGGCCCCTACCGGAAAGCGGATAG |
| 241 | 61 | VP1_aa | MSFVDHPPDWLEEVGEGLREFLGLEAGPPKPKPNQQHEDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 242 | 61 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATGAGGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 243 | 61 | MAAP_aa | MRIKPVVLCCLVITISDPETGSIEESLSTGQTRSRESTTSRTTSSLRRET |
| | | | TPTSSTTTRTPSFRRSSPTTHPSGETSERQSFRPRKGFSNLLAWLKRVLR |
| | | | RPLPESG* |
| 244 | 61 | MAAP_nt | ATGAGGATCAAGCCCGTGGTCTTGTGCTGCCTGGTTATAACTATCTCGGA |
| | | | CCCGGAAACGGGCTCGATCGAGGAGAGCCTGTCAACAGGGCAGACGAGGT |
| | | | CGCGCGAGAGCACGACATCTCGTACAACGAGCAGCTTGAGGCGGGAGACA |
| | | | ACCCCTACCTCAAGTACAACCACGCGGACGCCGAGTTTCAGGAGAAGCTC |
| | | | GCCGACGACACATCCTTCGGGGGAAACCTCGGAAAGGCAGTCTTTCAGGC |
| | | | CAAGAAAAGGGTTCTCGAACCTTTTGGCCTGGTTGAAGAGGGTGCTAAGA |
| | | | CGGCCCCTACCGGAAAGCGGATAG |
| 245 | 62 | VP1_aa | MSFVDHPPDWLEEVGEGLREFLGLEPGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 246 | 62 | VP1_nt | ATGTCTTTTGTTGATCACCCTCCAGATTGGTTGGAAGAAGTTGGTGAAGG |
| | | | GCTTCGCGAGTTTTTGGGCCTTGAACCTGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 247 | 62 | MAAP_aa | LAHRNQNPISSIKIKPVVLCCLVITISDPETGSIEESLSTGQTRSRESTT |
| | | | SRTTSSLRRETTPTSSTTTRTPSFRRSSPTTHPSGETSERQSFRPRKGFS |
| | | | NLLAWLKRVLRRPLPESG* |
| 248 | 62 | MAAP_nt | CTGGCCCACCGAAACCAAAACCCAATCAGCAGCATCAAGATCAAGCCCGT |
| | | | GGTCTTGTGCTGCCTGGTTATAACTATCTCGGACCCGGAAACGGGCTCGA |
| | | | TCGAGGAGAGCCTGTCAACAGGGCAGACGAGGTCGCGCGAGAGCACGACA |
| | | | TCTCGTACAACGAGCAGCTTGAGGCGGGAGACAACCCCTACCTCAAGTAC |
| | | | AACCACGCGGACGCCGAGTTTCAGGAGAAGCTCGCCGACGACACATCCTT |
| | | | CGGGGGAAACCTCGGAAAGGCAGTCTTTCAGGCCAAGAAAAGGGTTCTCG |
| | | | AACCTTTTGGCCTGGTTGAAGAGGGTGCTAAGACGGCCCCTACCGGAAAG |
| | | | CGGATAG |
| 249 | 63 | VP1_aa | MSFVDHPPDWLEEVGEGLREFLGLEAGPPKPKPNQQHQDQARGLVLPGYN |
| | | | YLGPGNGLDRGEPVNRADEVAREHDISYDEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 250 | 63 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACGATGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 251 | 63 | MAAP_aa | MSSLRRETTPTSSTTTRTPSFRRSSPTTHPSGETSERQSFRPRKGFSNLL |
| | | | AWLKRVLRRPLPESG* |
| 252 | 63 | MAAP_nt | ATGAGCAGCTTGAGGCGGGAGACAACCCCTACCTCAAGTACAACCACGCG |
| | | | GACGCCGAGTTTCAGGAGAAGCTCGCCGACGACACATCCTTCGGGGGAAA |
| | | | CCTCGGAAAGGCAGTCTTTCAGGCCAAGAAAAGGGTTCTCGAACCTTTTG |
| | | | GCCTGGTTGAAGAGGGTGCTAAGACGGCCCCTACCGGAAAGCGGATAG |
| 253 | 64 | VP1_aa | MSFVDHPPDWLEEVGEGLREFLGLEAGPPKPKPNQQHQHADQARGLVLPG |
| | | | YNYLGPFNGLDRGEPVNRADEVAREHDISYNEQLEAGDNPYLKYNHADAE |
| | | | FQEKLADDTSFGGNLGKAVFQAKKRVLEPFGLVEEGAKTAPTGKRIDDHF |
| | | | PKRKKARTEEDSKPSTSSDAEAGPSGSQQLQIPAQPASSLGADTMSAGGG |
| | | | GPLGDNNQGADGVGNASGDWHCDSTWMGDRVVTKSTRTWVLPSYNNHQYR |
| | | | EIKSGSVDGSNANAYFGYSTPWGYFDFNRFHSHWSPRDWQRLINNYWGFR |
| | | | PRSLRVKIFNIQVKEVTVQDSTTTIANNLTSTVQVFTDDDYQLPYVVGNG |
| | | | TEGCLPAFPPQVFTLPQYGYATLNRDNTENPTERSSFFCLEYFPSKMLRT |
| | | | GNNFEFTYNFEEVPFHSSFAPSQNLFKLANPLVDQYLYRFVSTNNTGGVQ |
| | | | FNKNLAGRYANTYKNWFPGPMGRTQGWNLGSGVNRASVSAFATTNRMELE |
| | | | GASYQVPPQPNGMTNNLQGSNTYALENTMIFNSQPANPGTTATYLEGNML |
| | | | ITSESETQPVNRVAYNVGGQMATNNQSSTTAPATGTYNLQEIVPGSVWME |
| | | | RDVYLQGPIWAKIPETGAHFHPSPAMGGFGLKHPPPMMLIKNTPVPGNIT |
| | | | SFSDVPVSSFITQYSTGQVTVEMEWELKKENSKRWNPEIQYTNNYNDPQF |
| | | | VDFAPDSTGEYRTTRPIGTRYLTRPL* |
| 254 | 64 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAACATGCCGATCAAGCCCGTGGTCTTGTGCTGCCTGGT |
| | | | TATAACTATCTCGGACCCTTCAACGGGCTCGATCGAGGAGAGCCTGTCAA |
| | | | CAGGGCAGACGAGGTCGCGCGAGAGCACGACATCTCGTACAACGAGCAGC |
| | | | TTGAGGCGGGAGACAACCCCTACCTCAAGTACAACCACGCGGACGCCGAG |
| | | | TTTCAGGAGAAGCTCGCCGACGACACATCCTTCGGGGGAAACCTCGGAAA |
| | | | GGCAGTCTTTCAGGCCAAGAAAAGGGTTCTCGAACCTTTTGGCCTGGTTG |
| | | | AAGAGGGTGCTAAGACGGCCCCTACCGGAAAGCGGATAGACGACCACTTT |
| | | | CCAAAAAGAAAGAAGGCTCGGACCGAAGAGGACTCCAAGCCTTCCACCTC |
| | | | GTCAGACGCCGAAGCTGGACCCAGCGGATCCCAGCAGCTGCAAATCCCAG |
| | | | CCCAACCAGCCTCAAGTTTGGGAGCTGATACAATGTCTGCGGGAGGTGGC |
| | | | GGCCCATTGGGCGACAATAACCAAGGTGCCGATGGAGTGGGCAATGCCTC |
| | | | GGGAGATTGGCATTGCGATTCCACGTGGATGGGGGACAGAGTCGTCACCA |
| | | | AGTCCACCCGAACCTGGGTGCTGCCCAGCTACAACAACCACCAGTACCGA |
| | | | GAGATCAAAAGCGGCTCCGTCGACGGAAGCAACGCCAACGCCTACTTTGG |
| | | | ATACAGCACCCCCTGGGGGTACTTTGACTTTAACCGCTTCCACAGCCACT |
| | | | GGAGCCCCCGAGACTGGCAAAGACTCATCAACAACTACTGGGGCTTCAGA |
| | | | CCCCGGTCCCTCAGAGTCAAAATCTTCAACATTCAAGTCAAAGAGGTCAC |
| | | | GGTGCAGGACTCCACCACCACCATCGCCAACAACCTCACCTCCACCGTCC |
| | | | AAGTGTTTACGGACGACGACTACCAGCTGCCCTACGTCGTCGGCAACGGG |
| | | | ACCGAGGGATGCCTGCCGGCCTTCCCTCCGCAGGTCTTTACGCTGCCGCA |
| | | | GTACGGTTACGCGACGCTGAACCGCGACAACACAGAAAATCCCACCGAGA |
| | | | GGAGCAGCTTCTTCTGCCTAGAGTACTTTCCCAGCAAGATGCTGAGAACG |
| | | | GGCAACAACTTTGAGTTTACCTACAACTTTGAGGAGGTGCCCTTCCACTC |
| | | | CAGCTTCGCTCCCAGTCAGAACCTGTTCAAGCTGGCCAACCCGCTGGTGG |
| | | | ACCAGTACTTGTACCGCTTCGTGAGCACAAATAACACTGGCGGAGTCCAG |
| | | | TTCAACAAGAACCTGGCCGGGAGATACGCCAACACCTACAAAAACTGGTT |
| | | | CCCGGGGCCCATGGGCCGAACCCAGGGCTGGAACCTGGGCTCCGGGGTCA |
| | | | ACCGCGCCAGTGTCAGCGCCTTCGCCACGACCAATAGGATGGAGCTCGAG |
| | | | GGCGCGAGTTACCAGGTGCCCCCGCAGCCGAACGGCATGACCAACAACCT |
| | | | CCAGGGCAGCAACACCTATGCCCTGGAGAACACTATGATCTTCAACAGCC |
| | | | AGCCGGCGAACCCGGGCACCACCGCCACGTACCTCGAGGGCAACATGCTC |
| | | | ATCACCAGCGAGAGCGAGACACAGCCGGTGAACCGCGTGGCGTACAACGT |
| | | | CGGCGGGCAGATGGCCACCAACAACCAGAGCTCCACCACTGCCCCCGCGA |
| | | | CCGGCACGTACAACCTCCAGGAAATCGTGCCCGGCAGCGTGTGGATGGAG |
| | | | AGGGACGTGTACCTCCAAGGACCCATCTGGGCCAAGATCCCAGAGACAGG |
| | | | GGCGCACTTTCACCCCTCTCCGGCCATGGGCGGATTCGGACTCAAACACC |
| | | | CACCGCCCATGATGCTCATCAAGAACACGCCTGTGCCCGGAAATATCACC |
| | | | AGCTTCTCGGACGTGCCCGTCAGCAGCTTCATCACCCAGTACAGCACCGG |
| | | | GCAGGTCACCGTGGAGATGGAGTGGGAGCTCAAGAAGGAAAACTCCAAGA |
| | | | GGTGGAACCCAGAGATCCAGTACACAAACAACTACAACGACCCCCAGTTT |
| | | | GTGGACTTTGCCCCGGACAGCACCGGGGAATACAGAACCACCAGACCTAT |
| | | | CGGAACCCGATACCTTACCCGACCCCTTTAA |
| 255 | 64 | MAAP_aa | MPIKPVVLCCLVITISDPSTGSIEESLSTGQTRSRESTTSRTTSSLRRET |
| | | | TPTSSTTTRTPSFRRSSPTTHPSGETSERQSFRPRKGFSNLLAWLKRVLR |
| | | | RPLPESG* |
| 256 | 64 | MAAP_nt | ATGCCGATCAAGCCCGTGGTCTTGTGCTGCCTGGTTATAACTATCTCGGA |
| | | | CCCTTCAACGGGCTCGATCGAGGAGAGCCTGTCAACAGGGCAGACGAGGT |
| | | | CGCGCGAGAGCACGACATCTCGTACAACGAGCAGCTTGAGGCGGGAGACA |
| | | | ACCCCTACCTCAAGTACAACCACGCGGACGCCGAGTTTCAGGAGAAGCTC |
| | | | GCCGACGACACATCCTTCGGGGGAAACCTCGGAAAGGCAGTCTTTCAGGC |
| | | | CAAGAAAAGGGTTCTCGAACCTTTTGGCCTGGTTGAAGAGGGTGCTAAGA |
| | | | CGGCCCCTACCGGAAAGCGGATAG |
| 257 | 65 | VP1_aa | MSFVDHPPDWLDEEVGEGLREFLGLEAGPPKPKPNQQHQDQARGLVLPGY |
| | | | NYLGPGNGLDRGEPVNRADEVAREHDISYNEQLEAGDNPYLKYNHADAEF |
| | | | QEKLADDTSFGGNLGKAVFQAKKRVLEPFGLVEEGAKTAPTGKRIDDHFP |
| | | | KRKKARTEEDSKPSTSSDAEAGPSGSQQLQIPAQPASSLGADTMSAGGGG |
| | | | PLGDNNQGADGVGNASGDWHCDSTWMGDRVVTKSTRTWVLPSYNNHQYRE |
| | | | IKSGSVDGSNANAYFGYSTPWGYFDFNRFHSHWSPRDWQRLINNYWGFRP |
| | | | RSLRVKIFNIQVKEVTVQDSTTTIANNLTSTVQVFTDDDYQLPYVVGNGT |
| | | | EGCLPAFPPQVFTLPQYGYATLNRDNTENPTERSSFFCLEYFPSKMLRTG |
| | | | NNFEFTYNFEEVPFHSSFAPSQNLFKLANPLVDQYLYRFVSTNNTGGVQF |
| | | | NKNLAGRYANTYKNWFPGPMGRTQGWNLGSGVNRASVSAFATTNRMELEG |
| | | | ASYQVPPQPNGMTNNLQGSNTYALENTMIFNSQPANPGTTATYLEGNMLI |
| | | | TSESETQPVNRVAYNVGGQMATNNQSSTTAPATGTYNLQEIVPGSVWMER |
| | | | DVYLQGPIWAKIPETGAHFHPSPAMGGFGLKHPPPMMLIKNTPVPGNITS |
| | | | FSDVPVSSFITQYSTGQVTVEMEWELKKENSKRWNPEIQYTNNYNDPQFV |
| | | | DFAPDSTGEYRTTRPIGTRYLTRPL* |
| 258 | 65 | VP1_nt | ATGTCTTTTGTTGATCACCCTCCAGATTGGTTGGATGAAGAAGTTGGTGA |
| | | | AGGACTTCGCGAGTTTTTGGGCCTTGAAGCGGGCCCACCGAAACCAAAAC |
| | | | CCAATCAGCAGCATCAAGATCAAGCCCGTGGTCTTGTGCTGCCTGGTTAT |
| | | | AACTATCTCGGACCCGGAAACGGGCTCGATCGAGGAGAGCCTGTCAACAG |
| | | | GGCAGACGAGGTCGCGCGAGAGCACGACATCTCGTACAACGAGCAGCTTG |
| | | | AGGCGGGAGACAACCCCTACCTCAAGTACAACCACGCGGACGCCGAGTTT |
| | | | CAGGAGAAGCTCGCCGACGACACATCCTTCGGGGGAAACCTCGGAAAGGC |
| | | | AGTCTTTCAGGCCAAGAAAAGGGTTCTCGAACCTTTTGGCCTGGTTGAAG |
| | | | AGGGTGCTAAGACGGCCCCTACCGGAAAGCGGATAGACGACCACTTTCCA |
| | | | AAAAGAAAGAAGGCTCGGACCGAAGAGGACTCCAAGCCTTCCACCTCGTC |
| | | | AGACGCCGAAGCTGGACCCAGCGGATCCCAGCAGCTGCAAATCCCAGCCC |
| | | | AACCAGCCTCAAGTTTGGGAGCTGATACAATGTCTGCGGGAGGTGGCGGC |
| | | | CCATTGGGCGACAATAACCAAGGTGCCGATGGAGTGGGCAATGCCTCGGG |
| | | | AGATTGGCATTGCGATTCCACGTGGATGGGGGACAGAGTCGTCACCAAGT |
| | | | CCACCCGAACCTGGGTGCTGCCCAGCTACAACAACCACCAGTACCGAGAG |
| | | | ATCAAAAGCGGCTCCGTCGACGGAAGCAACGCCAACGCCTACTTTGGATA |
| | | | CAGCACCCCCTGGGGGTACTTTGACTTTAACCGCTTCCACAGCCACTGGA |
| | | | GCCCCCGAGACTGGCAAAGACTCATCAACAACTACTGGGGCTTCAGACCC |
| | | | CGGTCCCTCAGAGTCAAAATCTTCAACATTCAAGTCAAAGAGGTCACGGT |
| | | | GCAGGACTCCACCACCACCATCGCCAACAACCTCACCTCCACCGTCCAAG |
| | | | TGTTTACGGACGACGACTACCAGCTGCCCTACGTCGTCGGCAACGGGACC |
| | | | GAGGGATGCCTGCCGGCCTTCCCTCCGCAGGTCTTTACGCTGCCGCAGTA |
| | | | CGGTTACGCGACGCTGAACCGCGACAACACAGAAAATCCCACCGAGAGGA |
| | | | GCAGCTTCTTCTGCCTAGAGTACTTTCCCAGCAAGATGCTGAGAACGGGC |
| | | | AACAACTTTGAGTTTACCTACAACTTTGAGGAGGTGCCCTTCCACTCCAG |
| | | | CTTCGCTCCCAGTCAGAACCTGTTCAAGCTGGCCAACCCGCTGGTGGACC |
| | | | AGTACTTGTACCGCTTCGTGAGCACAAATAACACTGGCGGAGTCCAGTTC |
| | | | AACAAGAACCTGGCCGGGAGATACGCCAACACCTACAAAAACTGGTTCCC |
| | | | GGGGCCCATGGGCCGAACCCAGGGCTGGAACCTGGGCTCCGGGGTCAACC |
| | | | GCGCCAGTGTCAGCGCCTTCGCCACGACCAATAGGATGGAGCTCGAGGGC |
| | | | GCGAGTTACCAGGTGCCCCCGCAGCCGAACGGCATGACCAACAACCTCCA |
| | | | GGGCAGCAACACCTATGCCCTGGAGAACACTATGATCTTCAACAGCCAGC |
| | | | CGGCGAACCCGGGCACCACCGCCACGTACCTCGAGGGCAACATGCTCATC |
| | | | ACCAGCGAGAGCGAGACACAGCCGGTGAACCGCGTGGCGTACAACGTCGG |
| | | | CGGGCAGATGGCCACCAACAACCAGAGCTCCACCACTGCCCCCGCGACCG |
| | | | GCACGTACAACCTCCAGGAAATCGTGCCCGGCAGCGTGTGGATGGAGAGG |
| | | | GACGTGTACCTCCAAGGACCCATCTGGGCCAAGATCCCAGAGACAGGGGC |
| | | | GCACTTTCACCCCTCTCCGGCCATGGGCGGATTCGGACTCAAACACCCAC |
| | | | CGCCCATGATGCTCATCAAGAACACGCCTGTGCCCGGAAATATCACCAGC |
| | | | TTCTCGGACGTGCCCGTCAGCAGCTTCATCACCCAGTACAGCACCGGGCA |
| | | | GGTCACCGTGGAGATGGAGTGGGAGCTCAAGAAGGAAAACTCCAAGAGGT |
| | | | GGAACCCAGAGATCCAGTACACAAACAACTACAACGACCCCCAGTTTGTG |
| | | | GACTTTGCCCCGGACAGCACCGGGGAATACAGAACCACCAGACCTATCGG |
| | | | AACCCGATACCTTACCCGACCCCTTTAA |
| 259 | 65 | MAAP_aa | MKKLVKDFASFWALKRAHRNQNPISSIKIKPVVLCCLVITISDPETGSIE |
| | | | ESLSTGQTRSRESTTSRTTSSLRRETTPTSSTTTRTPSFRRSSPTTHPSG |
| | | | ETSERQSFRPRKGFSNLLAWLKRVLRRPLPESG* |
| 260 | 65 | MAAP_nt | ATGAAGAAGTTGGTGAAGGACTTCGCGAGTTTTTGGGCCTTGAAGCGGGC |
| | | | CCACCGAAACCAAAACCCAATCAGCAGCATCAAGATCAAGCCCGTGGTCT |
| | | | TGTGCTGCCTGGTTATAACTATCTCGGACCCGGAAACGGGCTCGATCGAG |
| | | | GAGAGCCTGTCAACAGGGCAGACGAGGTCGCGCGAGAGCACGACATCTCG |
| | | | TACAACGAGCAGCTTGAGGCGGGAGACAACCCCTACCTCAAGTACAACCA |
| | | | CGCGGACGCCGAGTTTCAGGAGAAGCTCGCCGACGACACATCCTTCGGGG |
| | | | GAAACCTCGGAAAGGCAGTCTTTCAGGCCAAGAAAAGGGTTCTCGAACCT |
| | | | TTTGGCCTGGTTGAAGAGGGTGCTAAGACGGCCCCTACCGGAAAGCGGAT |
| | | | AG |
| 261 | 66 | VP1_aa | MSFVDHPPDWLEEVGEGLREFLGLEAGPPKPKPAEQHKDDARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 262 | 66 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCG |
| | | | CCGAGCAGCATAAGGATGACGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 263 | 66 | MAAP_aa | MTPVVLCCLVITISDPETGSIEESLSTGQTRSRESTTSRTTSSLRRETTP |
| | | | TSSTTTRTPSFRRSSPTTHPSGETSERQSFRPRKGFSNLLAWLKRVLRRP |
| | | | LPESG* |
| 264 | 66 | MAAP_nt | ATGACGCCCGTGGTCTTGTGCTGCCTGGTTATAACTATCTCGGACCCGGA |
| | | | AACGGGCTCGATCGAGGAGAGCCTGTCAACAGGGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 265 | 67 | VP1_aa | MSFVDHPPDWLEEVGEGLREFLGLEAGPPKPKPAERHKDDSRGLVLPGYR |
| | | | YLGPFNGLDKGEPVNEADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 266 | 67 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCG |
| | | | CTGAACGCCATAAGGATGACTCTCGTGGTCTTGTGCTGCCTGGTTATCGT |
| | | | TATCTCGGACCCTTCAACGGGCTCGATAAAGGAGAGCCTGTCAACGAGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 267 | 67 | MAAP_aa | MTLVVLCCLVIVISDPSTGSIKESLSTRQTRSRESTTSRTTSSLRRETTP |
| | | | TSSTTTRTPSFRRSSPTTHPSGETSERQSFRPRKGFSNLLAWLKRVLRRP |
| | | | LPESG* |
| 268 | 67 | MAAP_nt | ATGACTCTCGTGGTCTTGTGCTGCCTGGTTATCGTTATCTCGGACCCTTC |
| | | | AACGGGCTCGATAAAGGAGAGCCTGTCAACGAGGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 269 | 68 | VP1_aa | MSFVDHPPDWLEEVGEGLREFLGLEAGPPKPKPVQQISEKSPGARGLVLP |
| | | | GYNYLGPGNSLDRGEPVNEADEVAREHDISYNEQLEAGDNPYLKYNHADA |
| | | | EFQEKLADDTSFGGNLGKAVFQAKKRVLEPFGLVEEGAKTAPTGKRIDDH |
| | | | FPKRKKARTEEDSKPSTSSDAEAGPSGSQQLQIPAQPASSLGADTMSAGG |
| | | | GGPLGDNNQGADGVGNASGDWHCDSTWMGDRVVTKSTRTWVLPSYNNHQY |
| | | | REIKSGSVDGSNANAYFGYSTPWGYFDFNRFHSHWSPRDWQRLINNYWGF |
| | | | RPRSLRVKIFNIQVKEVTVQDSTTTIANNLTSTVQVFTDDDYQLPYVVGN |
| | | | GTEGCLPAFPPQVFTLPQYGYATLNRDNTENPTERSSFFCLEYFPSKMLR |
| | | | TGNNFEFTYNFEEVPFHSSFAPSQNLFKLANPLVDQYLYRFVSTNNTGGV |
| | | | QFNKNLAGRYANTYKNWFPGPMGRTQGWNLGSGVNRASVSAFATTNRMEL |
| | | | EGASYQVPPQPNGMTNNLQGSNTYALENTMIFNSQPANPGTTATYLEGNM |
| | | | LITSESETQPVNRVAYNVGGQMATNNQSSTTAPATGTYNLQEIVPGSVWM |
| | | | ERDVYLQGPIWAKIPETGAHFHPSPAMGGFGLKHPPPMMLIKNTPVPGNI |
| | | | TSFSDVPVSSFITQYSTGQVTVEMEWELKKENSKRWNPEIQYTNNYNDPQ |
| | | | FVDFAPDSTGEYRTTRPIGTRYLTRPL* |
| 270 | 68 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCG |
| | | | TCCAGCAAATATCTGAAAAAAGCCCTGGCGCCCGTGGTCTTGTGCTGCCT |
| | | | GGTTATAACTATCTCGGACCCGGAAACAGCCTCGATCGAGGAGAGCCTGT |
| | | | CAACGAAGCAGACGAGGTCGCGCGAGAGCACGACATCTCGTACAACGAGC |
| | | | AGCTTGAGGCGGGAGACAACCCCTACCTCAAGTACAACCACGCGGACGCC |
| | | | GAGTTTCAGGAGAAGCTCGCCGACGACACATCCTTCGGGGGAAACCTCGG |
| | | | AAAGGCAGTCTTTCAGGCCAAGAAAAGGGTTCTCGAACCTTTTGGCCTGG |
| | | | TTGAAGAGGGTGCTAAGACGGCCCCTACCGGAAAGCGGATAGACGACCAC |
| | | | TTTCCAAAAAGAAAGAAGGCTCGGACCGAAGAGGACTCCAAGCCTTCCAC |
| | | | CTCGTCAGACGCCGAAGCTGGACCCAGCGGATCCCAGCAGCTGCAAATCC |
| | | | CAGCCCAACCAGCCTCAAGTTTGGGAGCTGATACAATGTCTGCGGGAGGT |
| | | | GGCGGCCCATTGGGCGACAATAACCAAGGTGCCGATGGAGTGGGCAATGC |
| | | | CTCGGGAGATTGGCATTGCGATTCCACGTGGATGGGGGACAGAGTCGTCA |
| | | | CCAAGTCCACCCGAACCTGGGTGCTGCCCAGCTACAACAACCACCAGTAC |
| | | | CGAGAGATCAAAAGCGGCTCCGTCGACGGAAGCAACGCCAACGCCTACTT |
| | | | TGGATACAGCACCCCCTGGGGGTACTTTGACTTTAACCGCTTCCACAGCC |
| | | | ACTGGAGCCCCCGAGACTGGCAAAGACTCATCAACAACTACTGGGGCTTC |
| | | | AGACCCCGGTCCCTCAGAGTCAAAATCTTCAACATTCAAGTCAAAGAGGT |
| | | | CACGGTGCAGGACTCCACCACCACCATCGCCAACAACCTCACCTCCACCG |
| | | | TCCAAGTGTTTACGGACGACGACTACCAGCTGCCCTACGTCGTCGGCAAC |
| | | | GGGACCGAGGGATGCCTGCCGGCCTTCCCTCCGCAGGTCTTTACGCTGCC |
| | | | GCAGTACGGTTACGCGACGCTGAACCGCGACAACACAGAAAATCCCACCG |
| | | | AGAGGAGCAGCTTCTTCTGCCTAGAGTACTTTCCCAGCAAGATGCTGAGA |
| | | | ACGGGCAACAACTTTGAGTTTACCTACAACTTTGAGGAGGTGCCCTTCCA |
| | | | CTCCAGCTTCGCTCCCAGTCAGAACCTGTTCAAGCTGGCCAACCCGCTGG |
| | | | TGGACCAGTACTTGTACCGCTTCGTGAGCACAAATAACACTGGCGGAGTC |
| | | | CAGTTCAACAAGAACCTGGCCGGGAGATACGCCAACACCTACAAAAACTG |
| | | | GTTCCCGGGGCCCATGGGCCGAACCCAGGGCTGGAACCTGGGCTCCGGGG |
| | | | TCAACCGCGCCAGTGTCAGCGCCTTCGCCACGACCAATAGGATGGAGCTC |
| | | | GAGGGCGCGAGTTACCAGGTGCCCCCGCAGCCGAACGGCATGACCAACAA |
| | | | CCTCCAGGGCAGCAACACCTATGCCCTGGAGAACACTATGATCTTCAACA |
| | | | GCCAGCCGGCGAACCCGGGCACCACCGCCACGTACCTCGAGGGCAACATG |
| | | | CTCATCACCAGCGAGAGCGAGACACAGCCGGTGAACCGCGTGGCGTACAA |
| | | | CGTCGGCGGGCAGATGGCCACCAACAACCAGAGCTCCACCACTGCCCCCG |
| | | | CGACCGGCACGTACAACCTCCAGGAAATCGTGCCCGGCAGCGTGTGGATG |
| | | | GAGAGGGACGTGTACCTCCAAGGACCCATCTGGGCCAAGATCCCAGAGAC |
| | | | AGGGGCGCACTTTCACCCCTCTCCGGCCATGGGCGGATTCGGACTCAAAC |
| | | | ACCCACCGCCCATGATGCTCATCAAGAACACGCCTGTGCCCGGAAATATC |
| | | | ACCAGCTTCTCGGACGTGCCCGTCAGCAGCTTCATCACCCAGTACAGCAC |
| | | | CGGGCAGGTCACCGTGGAGATGGAGTGGGAGCTCAAGAAGGAAAACTCCA |
| | | | AGAGGTGGAACCCAGAGATCCAGTACACAAACAACTACAACGACCCCCAG |
| | | | TTTGTGGACTTTGCCCCGGACAGCACCGGGGAATACAGAACCACCAGACC |
| | | | TATCGGAACCCGATACCTTACCCGACCCCTTTAA |
| 271 | 68 | MAAP_aa | LKKALAPVVLCCLVITISDPETASIEESLSTKQTRSRESTTSRTTSSLRR |
| | | | ETTPTSSTTTRTPSFRRSSPTTHPSGETSERQSFRPRKGFSNLLAWLKRV |
| | | | LRRPLPESG* |
| 272 | 68 | MAAP_nt | CTGAAAAAAGCCCTGGCGCCCGTGGTCTTGTGCTGCCTGGTTATAACTAT |
| | | | CTCGGACCCGGAAACAGCCTCGATCGAGGAGAGCCTGTCAACGAAGCAGA |
| | | | CGAGGTCGCGCGAGAGCACGACATCTCGTACAACGAGCAGCTTGAGGCGG |
| | | | GAGACAACCCCTACCTCAAGTACAACCACGCGGACGCCGAGTTTCAGGAG |
| | | | AAGCTCGCCGACGACACATCCTTCGGGGGAAACCTCGGAAAGGCAGTCTT |
| | | | TCAGGCCAAGAAAAGGGTTCTCGAACCTTTTGGCCTGGTTGAAGAGGGTG |
| | | | CTAAGACGGCCCCTACCGGAAAGCGGATAG |
| 273 | 69 | VP1_aa | MSFVDHPPDWLEEVGEGLREFLGLEAGPPKPKPNDQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 274 | 69 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGACCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 275 | 69 | MAAP_aa | MTSIKIKPVVLCCLVITISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 276 | 69 | MAAP_nt | ATGACCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 277 | 70 | VP1_aa | MSFVDHPPDWLEEVGEGLREFLGLPAGPPKPKPNQQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 278 | 70 | VP1_nt | ATGTCTTTTGTTGATCACCCTCCAGATTGGTTGGAAGAAGTTGGTGAAGG |
| | | | CCTTCGCGAGTTTTTGGGCCTTCCTGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 279 | 70 | MAAP_aa | LRAHRNQNPISSIKIKPVVLCCLVITISDPETGSIEESLSTGQTRSREST |
| | | | TSRTTSSLRRETTPTSSTTTRTPSFRRSSPTTHPSGETSERQSFRPRKGF |
| | | | SNLLAWLKRVLRRPLPESG* |
| 280 | 70 | MAAP_nt | CTGCGGGCCCACCGAAACCAAAACCCAATCAGCAGCATCAAGATCAAGCC |
| | | | CGTGGTCTTGTGCTGCCTGGTTATAACTATCTCGGACCCGGAAACGGGCT |
| | | | CGATCGAGGAGAGCCTGTCAACAGGGCAGACGAGGTCGCGCGAGAGCACG |
| | | | ACATCTCGTACAACGAGCAGCTTGAGGCGGGAGACAACCCCTACCTCAAG |
| | | | TACAACCACGCGGACGCCGAGTTTCAGGAGAAGCTCGCCGACGACACATC |
| | | | CTTCGGGGGAAACCTCGGAAAGGCAGTCTTTCAGGCCAAGAAAAGGGTTC |
| | | | TCGAACCTTTTGGCCTGGTTGAAGAGGGTGCTAAGACGGCCCCTACCGGA |
| | | | AAGCGGATAG |
| 281 | 71 | VP1_aa | MSFVDHPPDWLEEVGEGLREFLGLEAGPPKPKPNQQHQDQARGLVLPGYV |
| | | | YLGPGNGLHRGVPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 282 | 71 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATGTG |
| | | | TATCTCGGACCCGGAAACGGGCTCCACCGAGGAGTTCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 283 | 71 | MAAP_aa | MCISDPETGSTEEFLSTGQTRSRESTTSRTTSSLRRETTPTSSTTTRTPS |
| | | | FRRSSPTTHPSGETSERQSFRPRKGFSNLLAWLKRVLRRPLPESG* |
| 284 | 71 | MAAP_nt | ATGTGTATCTCGGACCCGGAAACGGGCTCCACCGAGGAGTTCCTGTCAAC |
| | | | AGGGCAGACGAGGTCGCGCGAGAGCACGACATCTCGTACAACGAGCAGCT |
| | | | TGAGGCGGGAGACAACCCCTACCTCAAGTACAACCACGCGGACGCCGAGT |
| | | | TTCAGGAGAAGCTCGCCGACGACACATCCTTCGGGGGAAACCTCGGAAAG |
| | | | GCAGTCTTTCAGGCCAAGAAAAGGGTTCTCGAACCTTTTGGCCTGGTTGA |
| | | | AGAGGGTGCTAAGACGGCCCCTACCGGAAAGCGGATAG |
| 285 | 72 | VP1_aa | MSFVDHPPDWLEEVGEGLREFLGLEAGPPKPKPAEQHKDDARGLVLPGYN |
| | | | YLGPFNGLDKGEPVNEADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 286 | 72 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCG |
| | | | CCGAACAGCATAAGGATGACGCCCGTGGTCTTGTGCTGCCTGGTTATAAC |
| | | | TATCTCGGACCCTTCAACGGGCTCGATAAAGGAGAGCCTGTCAACGAAGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 287 | 72 | MAAP_aa | MTPVVLCCLVITISDPSTGSIKESLSTKQTRSRESTTSRTTSSLRRETTP |
| | | | TSSTTTRTPSFRRSSPTTHPSGETSERQSFRPRKGFSNLLAWLKRVLRRP |
| | | | LPESG* |
| 288 | 72 | MAAP_nt | ATGACGCCCGTGGTCTTGTGCTGCCTGGTTATAACTATCTCGGACCCTTC |
| | | | AACGGGCTCGATAAAGGAGAGCCTGTCAACGAAGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 289 | 73 | VP1_aa | MSFVDHPPDWLEEVGEGLREFLGLEAGPPKPKPAEQHKDDARGLVLPGYK |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 290 | 73 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCG |
| | | | CCGAGCAGCATAAAGATGATGCCCGTGGTCTTGTGCTGCCTGGTTATAAG |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 291 | 73 | MAAP_aa | MMPVVLCCLVISISDPETGSIEESLSTGQTRSRESTTSRTTSSLRRETTP |
| | | | TSSTTTRTPSFRRSSPTTHPSGETSERQSFRPRKGFSNLLAWLKRVLRRP |
| | | | LPESG* |
| 292 | 73 | MAAP_nt | ATGATGCCCGTGGTCTTGTGCTGCCTGGTTATAAGTATCTCGGACCCGGA |
| | | | AACGGGCTCGATCGAGGAGAGCCTGTCAACAGGGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 293 | 74 | VP1_aa | MSFVDHPPDWLEEVGEGLREFLGLEAGPPKPKPNQQHQDDARGLVLPGYN |
| | | | YLGPFNGLDRGEPVNAADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 294 | 74 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATGATGCCCGTGGTCTTGTGCTGCCTGGTTATAAC |
| | | | TATCTCGGACCCTTTAACGGGCTCGATCGAGGAGAGCCTGTCAACGCCGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 295 | 74 | MAAP_aa | MMPVVLCCLVITISDPLIGSIEESLSTPQTRSRESTTSRTTSSLRRETTP |
| | | | TSSTTTRTPSFRRSSPTTHPSGETSERQSFRPRKGFSNLLAWLKRVLRRP |
| | | | LPESG* |
| 296 | 74 | MAAP_nt | ATGATGCCCGTGGTCTTGTGCTGCCTGGTTATAACTATCTCGGACCCTTT |
| | | | AACGGGCTCGATCGAGGAGAGCCTGTCAACGCCGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 297 | 75 | VP1_aa | MSFVDHPPDWLEEVGEGLREFLGLEAGPPKPKPNQQKQDDARGLVLPGYK |
| | | | YLGPFNGLDRGEPVNEADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 298 | 75 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGAAGCAAGATGATGCCCGTGGTCTTGTGCTGCCTGGTTATAAG |
| | | | TATCTCGGACCCTTCAACGGGCTCGATCGAGGAGAGCCTGTCAACGAGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 299 | 75 | MAAP_aa | MMPVVLCCLVISISDPSTGSIEESLSTRQTRSRESTTSRTTSSLRRETTP |
| | | | TSSTTTRTPSFRRSSPTTHPSGETSERQSFRPRKGFSNLLAWLKRVLRRP |
| | | | LPESG* |
| 300 | 75 | MAAP_nt | ATGATGCCCGTGGTCTTGTGCTGCCTGGTTATAAGTATCTCGGACCCTTC |
| | | | AACGGGCTCGATCGAGGAGAGCCTGTCAACGAGGCAGACGAGGTCGCGCG |
| | | | AGAGCACGACATCTCGTACAACGAGCAGCTTGAGGCGGGAGACAACCCCT |
| | | | ACCTCAAGTACAACCACGCGGACGCCGAGTTTCAGGAGAAGCTCGCCGAC |
| | | | GACACATCCTTCGGGGGAAACCTCGGAAAGGCAGTCTTTCAGGCCAAGAA |
| | | | AAGGGTTCTCGAACCTTTTGGCCTGGTTGAAGAGGGTGCTAAGACGGCCC |
| | | | CTACCGGAAAGCGGATAG |
| 301 | 76 | VP1_aa | MSFVDHPPDWLEEVGEGLREFLGLEAGPPKPKPNQQIQHADQARGLVLPG |
| | | | YNYLGPFNGLDRGEPVNAADEVAREHDISYNEQLEAGDNPYLKYNHADAE |
| | | | FQEKLADDTSFGGNLGKAVFQAKKRVLEPFGLVEEGAKTAPTGKRIDDHF |
| | | | PKRKKARTEEDSKPSTSSDAEAGPSGSQQLQIPAQPASSLGADTMSAGGG |
| | | | GPLGDNNQGADGVGNASGDWHCDSTWMGDRVVTKSTRTWVLPSYNNHQYR |
| | | | EIKSGSVDGSNANAYFGYSTPWGYFDFNRFHSHWSPRDWQRLINNYWGFR |
| | | | PRSLRVKIFNIQVKEVTVQDSTTTIANNLTSTVQVFTDDDYQLPYVVGNG |
| | | | TEGCLPAFPPQVFTLPQYGYATLNRDNTENPTERSSFFCLEYFPSKMLRT |
| | | | GNNFEFTYNFEEVPFHSSFAPSQNLFKLANPLVDQYLYRFVSTNNTGGVQ |
| | | | FNKNLAGRYANTYKNWFPGPMGRTQGWNLGSGVNRASVSAFATTNRMELE |
| | | | GASYQVPPQPNGMTNNLQGSNTYALENTMIFNSQPANPGTTATYLEGNML |
| | | | ITSESETQPVNRVAYNVGGQMATNNQSSTTAPATGTYNLQEIVPGSVWME |
| | | | RDVYLQGPIWAKIPETGAHFHPSPAMGGFGLKHPPPMMLIKNTPVPGNIT |
| | | | SFSDVPVSSFITQYSTGQVTVEMEWELKKENSKRWNPEIQYTNNYNDPQF |
| | | | VDFAPDSTGEYRTTRPIGTRYLTRPL* |
| 302 | 76 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGATCCAACATGCGGACCAAGCCCGTGGTCTTGTGCTGCCTGGT |
| | | | TATAACTATCTCGGACCCTTCAACGGGCTCGATCGAGGAGAGCCTGTCAA |
| | | | CGCGGCAGACGAGGTCGCGCGAGAGCACGACATCTCGTACAACGAGCAGC |
| | | | TTGAGGCGGGAGACAACCCCTACCTCAAGTACAACCACGCGGACGCCGAG |
| | | | TTTCAGGAGAAGCTCGCCGACGACACATCCTTCGGGGGAAACCTCGGAAA |
| | | | GGCAGTCTTTCAGGCCAAGAAAAGGGTTCTCGAACCTTTTGGCCTGGTTG |
| | | | AAGAGGGTGCTAAGACGGCCCCTACCGGAAAGCGGATAGACGACCACTTT |
| | | | CCAAAAAGAAAGAAGGCTCGGACCGAAGAGGACTCCAAGCCTTCCACCTC |
| | | | GTCAGACGCCGAAGCTGGACCCAGCGGATCCCAGCAGCTGCAAATCCCAG |
| | | | CCCAACCAGCCTCAAGTTTGGGAGCTGATACAATGTCTGCGGGAGGTGGC |
| | | | GGCCCATTGGGCGACAATAACCAAGGTGCCGATGGAGTGGGCAATGCCTC |
| | | | GGGAGATTGGCATTGCGATTCCACGTGGATGGGGGACAGAGTCGTCACCA |
| | | | AGTCCACCCGAACCTGGGTGCTGCCCAGCTACAACAACCACCAGTACCGA |
| | | | GAGATCAAAAGCGGCTCCGTCGACGGAAGCAACGCCAACGCCTACTTTGG |
| | | | ATACAGCACCCCCTGGGGGTACTTTGACTTTAACCGCTTCCACAGCCACT |
| | | | GGAGCCCCCGAGACTGGCAAAGACTCATCAACAACTACTGGGGCTTCAGA |
| | | | CCCCGGTCCCTCAGAGTCAAAATCTTCAACATTCAAGTCAAAGAGGTCAC |
| | | | GGTGCAGGACTCCACCACCACCATCGCCAACAACCTCACCTCCACCGTCC |
| | | | AAGTGTTTACGGACGACGACTACCAGCTGCCCTACGTCGTCGGCAACGGG |
| | | | ACCGAGGGATGCCTGCCGGCCTTCCCTCCGCAGGTCTTTACGCTGCCGCA |
| | | | GTACGGTTACGCGACGCTGAACCGCGACAACACAGAAAATCCCACCGAGA |
| | | | GGAGCAGCTTCTTCTGCCTAGAGTACTTTCCCAGCAAGATGCTGAGAACG |
| | | | GGCAACAACTTTGAGTTTACCTACAACTTTGAGGAGGTGCCCTTCCACTC |
| | | | CAGCTTCGCTCCCAGTCAGAACCTGTTCAAGCTGGCCAACCCGCTGGTGG |
| | | | ACCAGTACTTGTACCGCTTCGTGAGCACAAATAACACTGGCGGAGTCCAG |
| | | | TTCAACAAGAACCTGGCCGGGAGATACGCCAACACCTACAAAAACTGGTT |
| | | | CCCGGGGCCCATGGGCCGAACCCAGGGCTGGAACCTGGGCTCCGGGGTCA |
| | | | ACCGCGCCAGTGTCAGCGCCTTCGCCACGACCAATAGGATGGAGCTCGAG |
| | | | GGCGCGAGTTACCAGGTGCCCCCGCAGCCGAACGGCATGACCAACAACCT |
| | | | CCAGGGCAGCAACACCTATGCCCTGGAGAACACTATGATCTTCAACAGCC |
| | | | AGCCGGCGAACCCGGGCACCACCGCCACGTACCTCGAGGGCAACATGCTC |
| | | | ATCACCAGCGAGAGCGAGACACAGCCGGTGAACCGCGTGGCGTACAACGT |
| | | | CGGCGGGCAGATGGCCACCAACAACCAGAGCTCCACCACTGCCCCCGCGA |
| | | | CCGGCACGTACAACCTCCAGGAAATCGTGCCCGGCAGCGTGTGGATGGAG |
| | | | AGGGACGTGTACCTCCAAGGACCCATCTGGGCCAAGATCCCAGAGACAGG |
| | | | GGCGCACTTTCACCCCTCTCCGGCCATGGGCGGATTCGGACTCAAACACC |
| | | | CACCGCCCATGATGCTCATCAAGAACACGCCTGTGCCCGGAAATATCACC |
| | | | AGCTTCTCGGACGTGCCCGTCAGCAGCTTCATCACCCAGTACAGCACCGG |
| | | | GCAGGTCACCGTGGAGATGGAGTGGGAGCTCAAGAAGGAAAACTCCAAGA |
| | | | GGTGGAACCCAGAGATCCAGTACACAAACAACTACAACGACCCCCAGTTT |
| | | | GTGGACTTTGCCCCGGACAGCACCGGGGAATACAGAACCACCAGACCTAT |
| | | | CGGAACCCGATACCTTACCCGACCCCTTTAA |
| 303 | 76 | MAAP_aa | MRTKPVVLCCLVITISDPSTGSIEESLSTRQTRSRESTTSRTTSSLRRET |
| | | | TPTSSTTTRTPSFRRSSPTTHPSGETSERQSFRPRKGFSNLLAWLKRVLR |
| | | | RPLPESG* |
| 304 | 76 | MAAP_nt | ATGCGGACCAAGCCCGTGGTCTTGTGCTGCCTGGTTATAACTATCTCGGA |
| | | | CCCTTCAACGGGCTCGATCGAGGAGAGCCTGTCAACGCGGCAGACGAGGT |
| | | | CGCGCGAGAGCACGACATCTCGTACAACGAGCAGCTTGAGGCGGGAGACA |
| | | | ACCCCTACCTCAAGTACAACCACGCGGACGCCGAGTTTCAGGAGAAGCTC |
| | | | GCCGACGACACATCCTTCGGGGGAAACCTCGGAAAGGCAGTCTTTCAGGC |
| | | | CAAGAAAAGGGTTCTCGAACCTTTTGGCCTGGTTGAAGAGGGTGCTAAGA |
| | | | CGGCCCCTACCGGAAAGCGGATAG |
| 305 | 77 | VP1_aa | MSFVDHPPDWLEEVGEGLREFLGLEAGPPKPKPNERHKDQARGLVLPGYK |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 306 | 77 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGAGCGCCATAAGGATCAAGCCCGTGGTCTTGTGCTGCCTGGTTATAAG |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 307 | 77 | MAAP_aa | MSAIRIKPVVLCCLVISISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 308 | 77 | MAAP_nt | ATGAGCGCCATAAGGATCAAGCCCGTGGTCTTGTGCTGCCTGGTTATAAG |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 309 | 78 | VP1_aa | MSFVDHPPDWLEEVGEGLREFLGLEAGPPKPKPNQQHQDQARGLVLPGYN |
| | | | YLGPGNGLDRGEPVNRADAVAREHDISYDEQLKAGDNPYLRYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 310 | 78 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGCGGTCGCGCGAGAGCACGACATCTCGTACGATGAGCAGCTTAAGG |
| | | | CGGGAGACAACCCCTACCTCAGATACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 311 | 78 | MAAP_aa | MSSLRRETTPTSDTTTRTPSFRRSSPTTHPSGETSERQSFRPRKGFSNLL |
| | | | AWLKRVLRRPLPESG* |
| 312 | 78 | MAAP_nt | ATGAGCAGCTTAAGGCGGGAGACAACCCCTACCTCAGATACAACCACGCG |
| | | | GACGCCGAGTTTCAGGAGAAGCTCGCCGACGACACATCCTTCGGGGGAAA |
| | | | CCTCGGAAAGGCAGTCTTTCAGGCCAAGAAAAGGGTTCTCGAACCTTTTG |
| | | | GCCTGGTTGAAGAGGGTGCTAAGACGGCCCCTACCGGAAAGCGGATAG |
| 313 | 79 | VP1_aa | MSFVDHPPDWLEEVGEGLREFLGLEAGPPKPKPNAQHQDQARGLVLPGYN |
| | | | YLGPGNGLDRGEPVNRADEVAREHDISYNEQLEAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 314 | 79 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATGCACAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 315 | 79 | MAAP_aa | MHSIKIKPVVLCCLVITISDPETGSIEESLSTGQTRSRESTTSRTTSSLR |
| | | | RETTPTSSTTTRTPSFRRSSPTTHPSGETSERQSFRPRKGFSNLLAWLKR |
| | | | VLRRPLPESG* |
| 316 | 79 | MAAP_nt | ATGCACAGCATCAAGATCAAGCCCGTGGTCTTGTGCTGCCTGGTTATAAC |
| | | | TATCTCGGACCCGGAAACGGGCTCGATCGAGGAGAGCCTGTCAACAGGGC |
| | | | AGACGAGGTCGCGCGAGAGCACGACATCTCGTACAACGAGCAGCTTGAGG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAG |
| 317 | 80 | VP1_aa | MSFVDHPPDWLEEVGEGLREFLGLEAGPPKPKPNQQHQDQARGLVLPGYN |
| | | | YLGPGNGLDRGEPVNAADEVAREHDIAYDEQLKAGDNPYLKYNHADAEFQ |
| | | | EKLADDTSFGGNLGKAVFQAKKRVLEPFGLVEEGAKTAPTGKRIDDHFPK |
| | | | RKKARTEEDSKPSTSSDAEAGPSGSQQLQIPAQPASSLGADTMSAGGGGP |
| | | | LGDNNQGADGVGNASGDWHCDSTWMGDRVVTKSTRTWVLPSYNNHQYREI |
| | | | KSGSVDGSNANAYFGYSTPWGYFDFNRFHSHWSPRDWQRLINNYWGFRPR |
| | | | SLRVKIFNIQVKEVTVQDSTTTIANNLTSTVQVFTDDDYQLPYVVGNGTE |
| | | | GCLPAFPPQVFTLPQYGYATLNRDNTENPTERSSFFCLEYFPSKMLRTGN |
| | | | NFEFTYNFEEVPFHSSFAPSQNLFKLANPLVDQYLYRFVSTNNTGGVQFN |
| | | | KNLAGRYANTYKNWFPGPMGRTQGWNLGSGVNRASVSAFATTNRMELEGA |
| | | | SYQVPPQPNGMTNNLQGSNTYALENTMIFNSQPANPGTTATYLEGNMLIT |
| | | | SESETQPVNRVAYNVGGQMATNNQSSTTAPATGTYNLQEIVPGSVWMERD |
| | | | VYLQGPIWAKIPETGAHFHPSPAMGGFGLKHPPPMMLIKNTPVPGNITSF |
| | | | SDVPVSSFITQYSTGQVTVEMEWELKKENSKRWNPEIQYTNNYNDPQFVD |
| | | | FAPDSTGEYRTTRPIGTRYLTRPL* |
| 318 | 80 | VP1_nt | ATGTCTTTTGTTGATCACCCTCCAGATTGGTTGGAAGAAGTTGGTGAAGG |
| | | | TCTTCGCGAGTTTTTGGGCCTTGAAGCGGGCCCACCGAAACCAAAACCCA |
| | | | ATCAGCAGCATCAAGATCAAGCCCGTGGTCTTGTGCTGCCTGGTTATAAC |
| | | | TATCTCGGACCCGGAAACGGGCTCGATCGAGGAGAGCCTGTCAACGCTGC |
| | | | AGACGAGGTCGCGCGAGAGCACGACATCGCGTACGATGAGCAGCTTAAAG |
| | | | CGGGAGACAACCCCTACCTCAAGTACAACCACGCGGACGCCGAGTTTCAG |
| | | | GAGAAGCTCGCCGACGACACATCCTTCGGGGGAAACCTCGGAAAGGCAGT |
| | | | CTTTCAGGCCAAGAAAAGGGTTCTCGAACCTTTTGGCCTGGTTGAAGAGG |
| | | | GTGCTAAGACGGCCCCTACCGGAAAGCGGATAGACGACCACTTTCCAAAA |
| | | | AGAAAGAAGGCTCGGACCGAAGAGGACTCCAAGCCTTCCACCTCGTCAGA |
| | | | CGCCGAAGCTGGACCCAGCGGATCCCAGCAGCTGCAAATCCCAGCCCAAC |
| | | | CAGCCTCAAGTTTGGGAGCTGATACAATGTCTGCGGGAGGTGGCGGCCCA |
| | | | TTGGGCGACAATAACCAAGGTGCCGATGGAGTGGGCAATGCCTCGGGAGA |
| | | | TTGGCATTGCGATTCCACGTGGATGGGGGACAGAGTCGTCACCAAGTCCA |
| | | | CCCGAACCTGGGTGCTGCCCAGCTACAACAACCACCAGTACCGAGAGATC |
| | | | AAAAGCGGCTCCGTCGACGGAAGCAACGCCAACGCCTACTTTGGATACAG |
| | | | CACCCCCTGGGGGTACTTTGACTTTAACCGCTTCCACAGCCACTGGAGCC |
| | | | CCCGAGACTGGCAAAGACTCATCAACAACTACTGGGGCTTCAGACCCCGG |
| | | | TCCCTCAGAGTCAAAATCTTCAACATTCAAGTCAAAGAGGTCACGGTGCA |
| | | | GGACTCCACCACCACCATCGCCAACAACCTCACCTCCACCGTCCAAGTGT |
| | | | TTACGGACGACGACTACCAGCTGCCCTACGTCGTCGGCAACGGGACCGAG |
| | | | GGATGCCTGCCGGCCTTCCCTCCGCAGGTCTTTACGCTGCCGCAGTACGG |
| | | | TTACGCGACGCTGAACCGCGACAACACAGAAAATCCCACCGAGAGGAGCA |
| | | | GCTTCTTCTGCCTAGAGTACTTTCCCAGCAAGATGCTGAGAACGGGCAAC |
| | | | AACTTTGAGTTTACCTACAACTTTGAGGAGGTGCCCTTCCACTCCAGCTT |
| | | | CGCTCCCAGTCAGAACCTGTTCAAGCTGGCCAACCCGCTGGTGGACCAGT |
| | | | ACTTGTACCGCTTCGTGAGCACAAATAACACTGGCGGAGTCCAGTTCAAC |
| | | | AAGAACCTGGCCGGGAGATACGCCAACACCTACAAAAACTGGTTCCCGGG |
| | | | GCCCATGGGCCGAACCCAGGGCTGGAACCTGGGCTCCGGGGTCAACCGCG |
| | | | CCAGTGTCAGCGCCTTCGCCACGACCAATAGGATGGAGCTCGAGGGCGCG |
| | | | AGTTACCAGGTGCCCCCGCAGCCGAACGGCATGACCAACAACCTCCAGGG |
| | | | CAGCAACACCTATGCCCTGGAGAACACTATGATCTTCAACAGCCAGCCGG |
| | | | CGAACCCGGGCACCACCGCCACGTACCTCGAGGGCAACATGCTCATCACC |
| | | | AGCGAGAGCGAGACACAGCCGGTGAACCGCGTGGCGTACAACGTCGGCGG |
| | | | GCAGATGGCCACCAACAACCAGAGCTCCACCACTGCCCCCGCGACCGGCA |
| | | | CGTACAACCTCCAGGAAATCGTGCCCGGCAGCGTGTGGATGGAGAGGGAC |
| | | | GTGTACCTCCAAGGACCCATCTGGGCCAAGATCCCAGAGACAGGGGCGCA |
| | | | CTTTCACCCCTCTCCGGCCATGGGCGGATTCGGACTCAAACACCCACCGC |
| | | | CCATGATGCTCATCAAGAACACGCCTGTGCCCGGAAATATCACCAGCTTC |
| | | | TCGGACGTGCCCGTCAGCAGCTTCATCACCCAGTACAGCACCGGGCAGGT |
| | | | CACCGTGGAGATGGAGTGGGAGCTCAAGAAGGAAAACTCCAAGAGGTGGA |
| | | | ACCCAGAGATCCAGTACACAAACAACTACAACGACCCCCAGTTTGTGGAC |
| | | | TTTGCCCCGGACAGCACCGGGGAATACAGAACCACCAGACCTATCGGAAC |
| | | | CCGATACCTTACCCGACCCCTTTAA |
| 319 | 80 | MAAP_aa | MSSLKRETTPTSSTTTRTPSFRRSSPTTHPSGETSERQSFRPRKGFSNLL |
| | | | AWLKRVLRRPLPESG* |
| 320 | 80 | MAAP_nt | ATGAGCAGCTTAAAGCGGGAGACAACCCCTACCTCAAGTACAACCACGCG |
| | | | GACGCCGAGTTTCAGGAGAAGCTCGCCGACGACACATCCTTCGGGGGAAA |
| | | | CCTCGGAAAGGCAGTCTTTCAGGCCAAGAAAAGGGTTCTCGAACCTTTTG |
| | | | GCCTGGTTGAAGAGGGTGCTAAGACGGCCCCTACCGGAAAGCGGATAG |

Exemplary reference, e.g., wildtype, MAAP encoding sequences, MAAP polypeptide sequences, Cap (e.g., VP1, VP2, and VP3) polypeptide or nucleic acid sequences, and Rep polypeptide or nucleic acid sequences are provided in Table 3.

TABLE 3

| Name | Description | Amino acid sequence | Nucleotide sequence |
|---|---|---|---|
| AAV5 | The full | MSFVDHPPDWLEEVGEGLREFLGLEAG | ATGTCTTTTGTTGATCACCCTCCAGATT |
| VP1 | wild type | PPKPKPNQQHQDQARGLVLPGYNYLGP | GGTTGGAAGAAGTTGGTGAAGGTCTTCG |
| | sequence of | GNGLDRGEPVNRADEVAREHDISYNEQ | CGAGTTTTTGGGCCTTGAAGCGGGCCCA |
| | AAV5 VP1 | LEAGDNPYLKYNHADAEFQEKLADDTS | CCGAAACCAAAACCCAATCAGCAGCATC |
| | | FGGNLGKAVFQAKKRVLEPFGLVEEGA | AAGATCAAGCCCGTGGTCTTGTGCTGCC |
| | | KTAPTGKRIDDHFPKRKKARTEEDSKP | TGGTTATAACTATCTCGGACCCGGAAAC |
| | | STSSDAEAGPSGSQQLQIPAQPASSLG | GGGCTCGATCGAGGAGAGCCTGTCAACA |
| | | ADTMSAGGGGPLGDNNQGADGVGNASG | GGGCAGACGAGGTCGCGCGAGAGCACGA |
| | | DWHCDSTWMGDRVVTKSTRTWVLPSYN | CATCTCGTACAACGAGCAGCTTGAGGCG |
| | | NHQYREIKSGSVDGSNANAYFGYSTPW | GGAGACAACCCCTACCTCAAGTACAACC |
| | | GYFDFNRFHSHWSPRDWQRLINNYWGF | ACGCGGACGCCGAGTTTCAGGAGAAGCT |
| | | RPRSLRVKIFNIQVKEVTVQDSTTTIA | CGCCGACGACACATCCTTCGGGGGAAAC |
| | | NNLTSTVQVFTDDDYQLPYVVGNGTEG | CTCGGAAAGGCAGTCTTTCAGGCCAAGA |
| | | CLPAFPPQVFTLPQYGYATLNRDNTEN | AAAGGGTTCTCGAACCTTTTGGCCTGGT |
| | | PTERSSFFCLEYFPSKMLRTGNNFEFT | TGAAGAGGGTGCTAAGACGGCCCCTACC |
| | | YNFEEVPFHSSFAPSQNLFKLANPLVD | GGAAAGCGGATAGACGACCACTTTCCAA |
| | | QYLYRFVSTNNTGGVQFNKNLAGRYAN | AAAGAAAGAAGGCTCGGACCGAAGAGGA |
| | | TYKNWFPGPMGRTQGWNLGSGVNRASV | CTCCAAGCCTTCCACCTCGTCAGACGCC |
| | | SAFATTNRMELEGASYQVPPQPNGMTN | GAAGCTGGACCCAGCGGATCCCAGCAGC |
| | | NLQGSNTYALENTMIFNSQPANPGTTA | TGCAAATCCCAGCCCAACCAGCCTCAAG |
| | | TYLEGNMLITSESETQPVNRVAYNVGG | TTTGGGAGCTGATACAATGTCTGCGGGA |
| | | QMATNNQSSTTAPATGTYNLQEIVPGS | GGTGGCGGCCCATTGGGCGACAATAACC |
| | | VWMERDVYLQGPIWAKIPETGAHFHPS | AAGGTGCCGATGGAGTGGGCAATGCCTC |
| | | PAMGGFGLKHPPPMMLIKNTPVPGNIT | GGGAGATTGGCATTGCGATTCCACGTGG |
| | | SFSDVPVSSFITQYSTGQVTVEMEWEL | ATGGGGGACAGAGTCGTCACCAAGTCCA |
| | | KKENSKRWNPEIQYTNNYNDPQFVDFA | CCCGAACCTGGGTGCTGCCCAGCTACAA |
| | | PDSTGEYRTTRPIGTRYLTRPL* | CAACCACCAGTACCGAGAGATCAAAAGC |
| | | (SEQ ID NO: 321) | GGCTCCGTCGACGGAAGCAACGCCAACG |
| | | | CCTACTTTGGATACAGCACCCCCTGGGG |
| | | | GTACTTTGACTTTAACCGCTTCCACAGC |
| | | | CACTGGAGCCCCCGAGACTGGCAAAGAC |
| | | | TCATCAACAACTACTGGGGCTTCAGACC |
| | | | CCGGTCCCTCAGAGTCAAAATCTTCAAC |
| | | | ATTCAAGTCAAAGAGGTCACGGTGCAGG |
| | | | ACTCCACCACCACCATCGCCAACAACCT |
| | | | CACCTCCACCGTCCAAGTGTTTACGGAC |
| | | | GACGACTACCAGCTGCCCTACGTCGTCG |
| | | | GCAACGGGACCGAGGGATGCCTGCCGGC |
| | | | CTTCCCTCCGCAGGTCTTTACGCTGCCG |
| | | | CAGTACGGTTACGCGACGCTGAACCGCG |
| | | | ACAACACAGAAAATCCCACCGAGAGGAG |
| | | | CAGCTTCTTCTGCCTAGAGTACTTTCCC |
| | | | AGCAAGATGCTGAGAACGGGCAACAACT |
| | | | TTGAGTTTACCTACAACTTTGAGGAGGT |
| | | | GCCCTTCCACTCCAGCTTCGCTCCCAGT |
| | | | CAGAACCTGTTCAAGCTGGCCAACCCGC |
| | | | TGGTGGACCAGTACTTGTACCGCTTCGT |
| | | | GAGCACAAATAACACTGGCGGAGTCCAG |
| | | | TTCAACAAGAACCTGGCCGGGAGATACG |
| | | | CCAACACCTACAAAAACTGGTTCCCGGG |
| | | | GCCCATGGGCCGAACCCAGGGCTGGAAC |
| | | | CTGGGCTCCGGGGTCAACCGCGCCAGTG |
| | | | TCAGCGCCTTCGCCACGACCAATAGGAT |
| | | | GGAGCTCGAGGGCGCGAGTTACCAGGTG |
| | | | CCCCCGCAGCCGAACGGCATGACCAACA |
| | | | ACCTCCAGGGCAGCAACACCTATGCCCT |
| | | | GGAGAACACTATGATCTTCAACAGCCAG |
| | | | CCGGCGAACCCGGGCACCACCGCCACGT |
| | | | ACCTCGAGGGCAACATGCTCATCACCAG |
| | | | CGAGAGCGAGACACAGCCGGTGAACCGC |
| | | | GTGGCGTACAACGTCGGCGGGCAGATGG |
| | | | CCACCAACAACCAGAGCTCCACCACTGC |
| | | | CCCCGCGACCGGCACGTACAACCTCCAG |
| | | | GAAATCGTGCCCGGCAGCGTGTGGATGG |
| | | | AGAGGGACGTGTACCTCCAAGGACCCAT |
| | | | CTGGGCCAAGATCCCAGAGACAGGGGCG |
| | | | CACTTTCACCCCTCTCCGGCCATGGGCG |
| | | | GATTCGGACTCAAACACCCACCGCCCAT |
| | | | GATGCTCATCAAGAACACGCCTGTGCCC |
| | | | GGAAATATCACCAGCTTCTCGGACGTGC |
| | | | CCGTCAGCAGCTTCATCACCCAGTACAG |
| | | | CACCGGGCAGGTCACCGTGGAGATGGAG |
| | | | TGGGAGCTCAAGAAGGAAAACTCCAAGA |
| | | | GGTGGAACCCAGAGATCCAGTACACAAA |
| | | | CAACTACAACGACCCCCAGTTTGTGGAC |
| | | | TTTGCCCCGGACAGCACCGGGGAATACA |
| | | | GAACCACCAGACCTATCGGAACCCGATA |
| | | | CCTTACCCGACCCCTTTAA (SEQ TD |
| | | | NO: 327) |
| AAV5 | The full | TAPTGKRIDDHFPKRKKARTEEDSKPS | ACGGCCCCTACCGGAAAGCGGATAGACG |
| VP2 | wild type | TSSDAEAGPSGSQQLQIPAQPASSLGA | ACCACTTTCCAAAAAGAAAGAAGGCTCG |
| | sequence of | DTMSAGGGGPLGDNNQGADGVGNASGD | GACCGAAGAGGACTCCAAGCCTTCCACC |
| | AAV5 VP2 | WHCDSTWMGDRVVTKSTRTWVLPSYNN | TCGTCAGACGCCGAAGCTGGACCCAGCG |
| | | HQYREIKSGSVDGSNANAYFGYSTPWG | GATCCCAGCAGCTGCAAATCCCAGCCCA |
| | | YFDFNRFHSHWSPRDWQRLINNYWGFR | ACCAGCCTCAAGTTTGGGAGCTGATACA |
| | | PRSLRVKIFNIQVKEVTVQDSTTTIAN | ATGTCTGCGGGAGGTGGCGGCCCATTGG |
| | | NLTSTVQVFTDDDYQLPYVVGNGTEGC | GCGACAATAACCAAGGTGCCGATGGAGT |
| | | LPAFPPQVFTLPQYGYATLNRDNTENP | GGGCAATGCCTCGGGAGATTGGCATTGC |
| | | TERSSFFCLEYFPSKMLRTGNNFEFTY | GATTCCACGTGGATGGGGGACAGAGTCG |
| | | NFEEVPFHSSFAPSQNLFKLANPLVDQ | TCACCAAGTCCACCCGAACCTGGGTGCT |
| | | YLYRFVSTNNTGGVQFNKNLAGRYANT | GCCCAGCTACAACAACCACCAGTACCGA |
| | | YKNWFPGPMGRTQGWNLGSGVNRASVS | GAGATCAAAAGCGGCTCCGTCGACGGAA |
| | | AFATTNRMELEGASYQVPPQPNGMTNN | GCAACGCCAACGCCTACTTTGGATACAG |
| | | LQGSNTYALENTMIFNSQPANPGTTAT | CACCCCCTGGGGGTACTTTGACTTTAAC |
| | | YLEGNMLITSESETQPVNRVAYNVGGQ | CGCTTCCACAGCCACTGGAGCCCCCGAG |
| | | MATNNQSSTTAPATGTYNLQEIVPGSV | ACTGGCAAAGACTCATCAACAACTACTG |
| | | WMERDVYLQGPIWAKIPETGAHFHPSP | GGGCTTCAGACCCCGGTCCCTCAGAGTC |
| | | AMGGFGLKHPPPMMLIKNTPVPGNITS | AAAATCTTCAACATTCAAGTCAAAGAGG |
| | | FSDVPVSSFITQYSTGQVTVEMEWELK | TCACGGTGCAGGACTCCACCACCACCAT |
| | | KENSKRWNPEIQYTNNYNDPQFVDFAP | CGCCAACAACCTCACCTCCACCGTCCAA |
| | | DSTGEYRTTRPIGTRYLTRPL* (SEQ | GTGTTTACGGACGACGACTACCAGCTGC |
| | | ID NO: 322) | CCTACGTCGTCGGCAACGGGACCGAGGG |
| | | | ATGCCTGCCGGCCTTCCCTCCGCAGGTC |
| | | | TTTACGCTGCCGCAGTACGGTTACGCGA |
| | | | CGCTGAACCGCGACAACACAGAAAATCC |
| | | | CACCGAGAGGAGCAGCTTCTTCTGCCTA |
| | | | GAGTACTTTCCCAGCAAGATGCTGAGAA |
| | | | CGGGCAACAACTTTGAGTTTACCTACAA |
| | | | CTTTGAGGAGGTGCCCTTCCACTCCAGC |
| | | | TTCGCTCCCAGTCAGAACCTGTTCAAGC |
| | | | TGGCCAACCCGCTGGTGGACCAGTACTT |
| | | | GTACCGCTTCGTGAGCACAAATAACACT |
| | | | GGCGGAGTCCAGTTCAACAAGAACCTGG |
| | | | CCGGGAGATACGCCAACACCTACAAAAA |
| | | | CTGGTTCCCGGGGCCCATGGGCCGAACC |
| | | | CAGGGCTGGAACCTGGGCTCCGGGGTCA |
| | | | ACCGCGCCAGTGTCAGCGCCTTCGCCAC |
| | | | GACCAATAGGATGGAGCTCGAGGGCGCG |
| | | | AGTTACCAGGTGCCCCCGCAGCCGAACG |
| | | | GCATGACCAACAACCTCCAGGGCAGCAA |
| | | | CACCTATGCCCTGGAGAACACTATGATC |
| | | | TTCAACAGCCAGCCGGCGAACCCGGGCA |
| | | | CCACCGCCACGTACCTCGAGGGCAACAT |
| | | | GCTCATCACCAGCGAGAGCGAGACACAG |
| | | | CCGGTGAACCGCGTGGCGTACAACGTCG |
| | | | GCGGGCAGATGGCCACCAACAACCAGAG |
| | | | CTCCACCACTGCCCCCGCGACCGGCACG |
| | | | TACAACCTCCAGGAAATCGTGCCCGGCA |
| | | | GCGTGTGGATGGAGAGGGACGTGTACCT |
| | | | CCAAGGACCCATCTGGGCCAAGATCCCA |
| | | | GAGACAGGGGCGCACTTTCACCCCTCTC |
| | | | CGGCCATGGGCGGATTCGGACTCAAACA |
| | | | CCCACCGCCCATGATGCTCATCAAGAAC |
| | | | ACGCCTGTGCCCGGAAATATCACCAGCT |
| | | | TCTCGGACGTGCCCGTCAGCAGCTTCAT |
| | | | CACCCAGTACAGCACCGGGCAGGTCACC |
| | | | GTGGAGATGGAGTGGGAGCTCAAGAAGG |
| | | | AAAACTCCAAGAGGTGGAACCCAGAGAT |
| | | | CCAGTACACAAACAACTACAACGACCCC |
| | | | CAGTTTGTGGACTTTGCCCCGGACAGCA |
| | | | CCGGGGAATACAGAACCACCAGACCTAT |
| | | | CGGAACCCGATACCTTACCCGACCCCTT |
| | | | TAA (SEQ ID NO: 328) |
| AAV5 | The wild | MSAGGGGPLGDNNQGADGVGNASGDWH | ATGTCTGCGGGAGGTGGCGGCCCATTGG |
| VP3 | type | CDSTWMGDRVVTKSTRTWVLPSYNNHQ | GCGACAATAACCAAGGTGCCGATGGAGT |
| | sequence of | YREIKSGSVDGSNANAYFGYSTPWGYF | GGGCAATGCCTCGGGAGATTGGCATTGC |
| | AAV5 VP3 | DENRFHSHWSPRDWQRLINNYWGFRPR | GATTCCACGTGGATGGGGGACAGAGTCG |
| | | SLRVKIFNIQVKEVTVQDSTTTIANNL | TCACCAAGTCCACCCGAACCTGGGTGCT |
| | | TSTVQVFTDDDYQLPYVVGNGTEGCLP | GCCCAGCTACAACAACCACCAGTACCGA |
| | | AFPPQVFTLPQYGYATLNRDNTENPTE | GAGATCAAAAGCGGCTCCGTCGACGGAA |
| | | RSSFFCLEYFPSKMLRTGNNFEFTYNF | GCAACGCCAACGCCTACTTTGGATACAG |
| | | EEVPFHSSFAPSQNLFKLANPLVDQYL | CACCCCCTGGGGGTACTTTGACTTTAAC |
| | | YRFVSTNNTGGVQFNKNLAGRYANTYK | CGCTTCCACAGCCACTGGAGCCCCCGAG |
| | | NWFPGPMGRTQGWNLGSGVNRASVSAF | ACTGGCAAAGACTCATCAACAACTACTG |
| | | ATTNRMELEGASYQVPPQPNGMTNNLQ | GGGCTTCAGACCCCGGTCCCTCAGAGTC |
| | | GSNTYALENTMIFNSQPANPGTTATYL | AAAATCTTCAACATTCAAGTCAAAGAGG |
| | | EGNMLITSESETQPVNRVAYNVGGQMA | TCACGGTGCAGGACTCCACCACCACCAT |
| | | TNNQSSTTAPATGTYNLQEIVPGSVWM | CGCCAACAACCTCACCTCCACCGTCCAA |
| | | ERDVYLQGPIWAKIPETGAHFHPSPAM | GTGTTTACGGACGACGACTACCAGCTGC |
| | | GGFGLKHPPPMMLIKNTPVPGNITSFS | CCTACGTCGTCGGCAACGGGACCGAGGG |
| | | DVPVSSFITQYSTGQVTVEMEWELKKE | ATGCCTGCCGGCCTTCCCTCCGCAGGTC |
| | | NSKRWNPEIQYTNNYNDPQFVDFAPDS | TTTACGCTGCCGCAGTACGGTTACGCGA |
| | | TGEYRTTRPIGTRYLTRPL* (SEQ | CGCTGAACCGCGACAACACAGAAAATCC |
| | | ID NO: 323) | CACCGAGAGGAGCAGCTTCTTCTGCCTA |
| | | | GAGTACTTTCCCAGCAAGATGCTGAGAA |
| | | | CGGGCAACAACTTTGAGTTTACCTACAA |
| | | | CTTTGAGGAGGTGCCCTTCCACTCCAGC |
| | | | TTCGCTCCCAGTCAGAACCTGTTCAAGC |
| | | | TGGCCAACCCGCTGGTGGACCAGTACTT |
| | | | GTACCGCTTCGTGAGCACAAATAACACT |
| | | | GGCGGAGTCCAGTTCAACAAGAACCTGG |
| | | | CCGGGAGATACGCCAACACCTACAAAAA |
| | | | CTGGTTCCCGGGGCCCATGGGCCGAACC |
| | | | CAGGGCTGGAACCTGGGCTCCGGGGTCA |
| | | | ACCGCGCCAGTGTCAGCGCCTTCGCCAC |
| | | | GACCAATAGGATGGAGCTCGAGGGCGCG |
| | | | AGTTACCAGGTGCCCCCGCAGCCGAACG |
| | | | GCATGACCAACAACCTCCAGGGCAGCAA |
| | | | CACCTATGCCCTGGAGAACACTATGATC |
| | | | TTCAACAGCCAGCCGGCGAACCCGGGCA |
| | | | CCACCGCCACGTACCTCGAGGGCAACAT |
| | | | GCTCATCACCAGCGAGAGCGAGACACAG |
| | | | CCGGTGAACCGCGTGGCGTACAACGTCG |
| | | | GCGGGCAGATGGCCACCAACAACCAGAG |
| | | | CTCCACCACTGCCCCCGCGACCGGCACG |
| | | | TACAACCTCCAGGAAATCGTGCCCGGCA |
| | | | GCGTGTGGATGGAGAGGGACGTGTACCT |
| | | | CCAAGGACCCATCTGGGCCAAGATCCCA |
| | | | GAGACAGGGGCGCACTTTCACCCCTCTC |
| | | | CGGCCATGGGCGGATTCGGACTCAAACA |
| | | | CCCACCGCCCATGATGCTCATCAAGAAC |
| | | | ACGCCTGTGCCCGGAAATATCACCAGCT |
| | | | TCTCGGACGTGCCCGTCAGCAGCTTCAT |
| | | | CACCCAGTACAGCACCGGGCAGGTCACC |
| | | | GTGGAGATGGAGTGGGAGCTCAAGAAGG |
| | | | AAAACTCCAAGAGGTGGAACCCAGAGAT |
| | | | CCAGTACACAAACAACTACAACGACCCC |
| | | | CAGTTTGTGGACTTTGCCCCGGACAGCA |
| | | | CCGGGGAATACAGAACCACCAGACCTAT |
| | | | CGGAACCCGATACCTTACCCGACCCCTT |
| | | | TAA (SEQ ID NO: 329) |
| AAV5 | This is the | RAHRNQNPISSIKIKPVVLCCLVITIS | CGGGCCCACCGAAACCAAAACCCAATCA |
| MAAP | full | DPETGSIEESLSTGQTRSRESTTSRTT | GCAGCATCAAGATCAAGCCCGTGGTCTT |
| (AAV2 | subsequence | SSLRRETTPTSSTTTRIPSFRRSSPTT | GTGCTGCCTGGTTATAACTATCTCGGAC |
| coordin | of AAV5 | HPSGETSERQSFRPRKGFSNLLAWLKR | CCGGAAACGGGCTCGATCGAGGAGAGCC |
| ates) | which | VLRRPLPESG* (SEQ ID NO: | TGTCAACAGGGCAGACGAGGTCGCGCGA |
| | corresponds | 325) | GAGCACGACATCTCGTACAACGAGCAGC |
| | to the | | TTGAGGCGGGAGACAACCCCTACCTCAA |
| | location of | | GTACAACCACGCGGACGCCGAGTTTCAG |
| | MAAP in | | GAGAAGCTCGCCGACGACACATCCTTCG |
| | AAV2. | | GGGGAAACCTCGGAAAGGCAGTCTTTCA |
| | Note that | | GGCCAAGAAAAGGGTTCTCGAACCTTTT |
| | this | | GGCCTGGTTGAAGAGGGTGCTAAGACGG |
| | sequence | | CCCCTACCGGAAAGCGGATAG (SEQ |
| | does not | | ID NO: 331) |
| | begin with a | | |
| | CTG. | | |
| AAV5 | The | LDPADPSSCKSQPNQPQVWELIQCLRE | CTGGACCCAGCGGATCCCAGCAGCTGCA |
| AAP | sequence of | VAAHWATITKVPMEWAMPREIGIAIPR | AATCCCAGCCCAACCAGCCTCAAGTTTG |
| | AAP in | GWGTESSPSPPEPGCCPATTTTSTERS | GGAGCTGATACAATGTCTGCGGGAGGTG |
| | AAV5 | KAAPSTEATPTPTLDTAPPGGTLTLTA | GCGGCCCATTGGGCGACAATAACCAAGG |
| | | STATGAPETGKDSSTTTGASDPGPSES | TGCCGATGGAGTGGGCAATGCCTCGGGA |
| | | KSSTFKSKRSRCRTPPPPSPTTSPPPS | GATTGGCATTGCGATTCCACGTGGATGG |
| | | KCLRTTTTSCPTSSATGPRDACRPSLR | GGGACAGAGTCGTCACCAAGTCCACCCG |
| | | RSLRCRSTVTRR* (SEQ ID NO: | AACCTGGGTGCTGCCCAGCTACAACAAC |
| | | 326) | CACCAGTACCGAGAGATCAAAAGCGGCT |
| | | | CCGTCGACGGAAGCAACGCCAACGCCTA |
| | | | CTTTGGATACAGCACCCCCTGGGGGTAC |
| | | | TTTGACTTTAACCGCTTCCACAGCCACT |
| | | | GGAGCCCCCGAGACTGGCAAAGACTCAT |
| | | | CAACAACTACTGGGGCTTCAGACCCCGG |
| | | | TCCCTCAGAGTCAAAATCTTCAACATTC |
| | | | AAGTCAAAGAGGTCACGGTGCAGGACTC |
| | | | CACCACCACCATCGCCAACAACCTCACC |
| | | | TCCACCGTCCAAGTGTTTACGGACGACG |
| | | | ACTACCAGCTGCCCTACGTCGTCGGCAA |
| | | | CGGGACCGAGGGATGCCTGCCGGCCTTC |
| | | | CCTCCGCAGGTCTTTACGCTGCCGCAGT |
| | | | ACGGTTACGCGACGCTGA (SEQ TD |
| | | | NO: 332) |
| AAV2 | The longer | MPGFYEIVIKVPSDLDEHLPGISDSFV | ATGCCGGGGTTTTACGAGATTGTGATTA |
| Rep 68 | Rep | NWVAEKEWELPPDSDMDLNLIEQAPLT | AGGTCCCCAGCGACCTTGACGAGCATCT |
| | transcript | VAEKLQRDFLTEWRRVSKAPEALFFVQ | GCCCGGCATTTCTGACAGCTTTGTGAAC |
| | without | FEKGESYFHMHVLVETTGVKSMVLGRF | TGGGTGGCCGAGAAGGAATGGGAGTTGC |
| | splicing | LSQIREKLIQRIYRGIEPTLPNWFAVT | CGCCAGATTCTGACATGGATCTGAATCT |
| | | KTRNGAGGGNKVVDECYIPNYLLPKTQ | GATTGAGCAGGCACCCCTGACCGTGGCC |
| | | PELQWAWTNMEQYLSACLNLTERKRLV | GAGAAGCTGCAGCGCGACTTTCTGACGG |
| | | AQHLTHVSQTQEQNKENQNPNSDAPVI | AATGGCGCCGTGTGAGTAAGGCCCCGGA |
| | | RSKTSARYMELVGWLVDKGITSEKQWI | GGCCCTTTTCTTTGTGCAATTTGAGAAG |
| | | QEDQASYISFNAASNSRSQIKAALDNA | GGAGAGAGCTACTTCCACATGCACGTGC |
| | | GKIMSLTKTAPDYLVGQQPVEDISSNR | TCGTGGAAACCACCGGGGTGAAATCCAT |
| | | IYKILELNGYDPQYAASVFLGWATKKF | GGTTTTGGGACGTTTCCTGAGTCAGATT |
| | | GKRNTIWLFGPATTGKTNIAEAIAHTV | CGCGAAAAACTGATTCAGAGAATTTACC |
| | | PFYGCVNWTNENFPFNDCVDKMVIWWE | GCGGGATCGAGCCGACTTTGCCAAACTG |
| | | EGKMTAKVVESAKAILGGSKVRVDQKC | GTTCGCGGTCACAAAGACCAGAAATGGC |
| | | KSSAQIDPTPVIVTSNTNMCAVIDGNS | GCCGGAGGCGGGAACAAGGTGGTGGATG |
| | | TTFEHQQPLQDRMFKFELTRRLDHDFG | AGTGCTACATCCCCAATTACTTGCTCCC |
| | | KVTKQEVKDFFRWAKDHVVEVEHEFYV | CAAAACCCAGCCTGAGCTCCAGTGGGCG |
| | | KKGGAKKRPAPSDADISEPKRVRESVA | TGGACTAATATGGAACAGTATTTAAGCG |
| | | QPSTSDAEASINYADRLARGHSL* | CCTGTTTGAATCTCACGGAGCGTAAACG |
| | | (SEQ ID NO: 333) | GTTGGTGGCGCAGCATCTGACGCACGTG |
| | | | TCGCAGACGCAGGAGCAGAACAAAGAGA |
| | | | ATCAGAATCCCAATTCTGATGCGCCGGT |
| | | | GATCAGATCAAAAACTTCAGCCAGGTAC |
| | | | ATGGAGCTGGTCGGGTGGCTCGTGGACA |
| | | | AGGGGATTACCTCGGAGAAGCAGTGGAT |
| | | | CCAGGAGGACCAGGCCTCATACATCTCC |
| | | | TTCAATGCGGCCTCCAACTCGCGGTCCC |
| | | | AAATCAAGGCTGCCTTGGACAATGCGGG |
| | | | AAAGATTATGAGCCTGACTAAAACCGCC |
| | | | CCCGACTACCTGGTGGGCCAGCAGCCCG |
| | | | TGGAGGACATTTCCAGCAATCGGATTTA |
| | | | TAAAATTTTGGAACTAAACGGGTACGAT |
| | | | CCCCAATATGCGGCTTCCGTCTTTCTGG |
| | | | GATGGGCCACGAAAAAGTTCGGCAAGAG |
| | | | GAACACCATCTGGCTGTTTGGGCCTGCA |
| | | | ACTACCGGGAAGACCAACATCGCGGAGG |
| | | | CCATAGCCCACACTGTGCCCTTCTACGG |
| | | | GTGCGTAAACTGGACCAATGAGAACTTT |
| | | | CCCTTCAACGACTGTGTCGACAAGATGG |
| | | | TGATCTGGTGGGAGGAGGGGAAGATGAC |
| | | | CGCCAAGGTCGTGGAGTCGGCCAAAGCC |
| | | | ATTCTCGGAGGAAGCAAGGTGCGCGTGG |
| | | | ACCAGAAATGCAAGTCCTCGGCCCAGAT |
| | | | AGACCCGACTCCCGTGATCGTCACCTCC |
| | | | AACACCAACATGTGCGCCGTGATTGACG |
| | | | GGAACTCAACGACCTTCGAACACCAGCA |
| | | | GCCGTTGCAAGACCGGATGTTCAAATTT |
| | | | GAACTCACCCGCCGTCTGGATCATGACT |
| | | | TTGGGAAGGTCACCAAGCAGGAAGTCAA |
| | | | AGACTTTTTCCGGTGGGCAAAGGATCAC |
| | | | GTGGTTGAGGTGGAGCATGAATTCTACG |
| | | | TCAAAAAGGGTGGAGCCAAGAAAAGACC |
| | | | CGCCCCCAGTGACGCAGATATAAGTGAG |
| | | | CCCAAACGGGTGCGCGAGTCAGTTGCGC |
| | | | AGCCATCGACGTCAGACGCGGAAGCTTC |
| | | | GATCAACTACGCAGACAGGTACCAAAAC |
| | | | AAATGTTCTCGTCACGTGGGCATGAATC |
| | | | TGATGCTGTTTCCCTGCAGACAATGCGA |
| | | | GAGAATGAATCAGAATTCAAATATCTGC |
| | | | TTCACTCACGGACAGAAAGACTGTTTAG |
| | | | AGTGCTTTCCCGTGTCAGAATCTCAACC |
| | | | CGTTTCTGTCGTCAAAAAGGCGTATCAG |
| | | | AAACTGTGCTACATTCATCATATCATGG |
| | | | GAAAGGTGCCAGACGCTTGCACTGCCTG |
| | | | CGATCTGGTCAATGTGGATTTGGATGAC |
| | | | TGCATCTTTGAACAATAAATGATTTAAA |
| | | | TCAGGTATGGCTGCCGATGGTTATCTTC |
| | | | CAGATTGGCTCGAGGACACTCTCTCTGA |
| | | | (SEQ ID NO: 337) |
| AAV2 | The longer | MPGFYEIVIKVPSDLDEHLPGISDSFV | ATGCCGGGGTTTTACGAGATTGTGATTA |
| Rep 78 | Rep | NWVAEKEWELPPDSDMDLNLIEQAPLT | AGGTCCCCAGCGACCTTGACGAGCATCT |
| | transcript | VAEKLQRDFLTEWRRVSKAPEALFFVQ | GCCCGGCATTTCTGACAGCTTTGTGAAC |
| | with | FEKGESYFHMHVLVETTGVKSMVLGRF | TGGGTGGCCGAGAAGGAATGGGAGTTGC |
| | splicing | LSQIREKLIQRIYRGIEPTLPNWFAVT | CGCCAGATTCTGACATGGATCTGAATCT |
| | | KTRNGAGGGNKVVDECYIPNYLLPKTQ | GATTGAGCAGGCACCCCTGACCGTGGCC |
| | | PELQWAWTNMEQYLSACLNLTERKRLV | GAGAAGCTGCAGCGCGACTTTCTGACGG |
| | | AQHLTHVSQTQEQNKENQNPNSDAPVI | AATGGCGCCGTGTGAGTAAGGCCCCGGA |
| | | RSKTSARYMELVGWLVDKGITSEKQWI | GGCCCTTTTCTTTGTGCAATTTGAGAAG |
| | | QEDQASYISFNAASNSRSQIKAALDNA | GGAGAGAGCTACTTCCACATGCACGTGC |
| | | GKIMSLTKTAPDYLVGQQPVEDISSNR | TCGTGGAAACCACCGGGGTGAAATCCAT |
| | | IYKILELNGYDPQYAASVFLGWATKKF | GGTTTTGGGACGTTTCCTGAGTCAGATT |
| | | GKRNTIWLFGPATTGKTNIAEAIAHTV | CGCGAAAAACTGATTCAGAGAATTTACC |
| | | PFYGCVNWTNENFPFNDCVDKMVIWWE | GCGGGATCGAGCCGACTTTGCCAAACTG |
| | | EGKMTAKVVESAKAILGGSKVRVDQKC | GTTCGCGGTCACAAAGACCAGAAATGGC |
| | | KSSAQIDPTPVIVTSNTNMCAVIDGNS | GCCGGAGGCGGGAACAAGGTGGTGGATG |
| | | TTFEHQQPLQDRMFKFELTRRLDHDFG | AGTGCTACATCCCCAATTACTTGCTCCC |
| | | KVTKQEVKDFFRWAKDHVVEVEHEFYV | CAAAACCCAGCCTGAGCTCCAGTGGGCG |
| | | KKGGAKKRPAPSDADISEPKRVRESVA | TGGACTAATATGGAACAGTATTTAAGCG |
| | | QPSTSDAEASINYADRYQNKCSRHVGM | CCTGTTTGAATCTCACGGAGCGTAAACG |
| | | NLMLFPCRQCERMNQNSNICFTHGQKD | GTTGGTGGCGCAGCATCTGACGCACGTG |
| | | CLECFPVSESQPVSVVKKAYQKLCYIH | TCGCAGACGCAGGAGCAGAACAAAGAGA |
| | | HIMGKVPDACTACDLVNVDLDDCIFEQ | ATCAGAATCCCAATTCTGATGCGCCGGT |
| | | * (SEQ ID NO: 334) | GATCAGATCAAAAACTTCAGCCAGGTAC |
| | | | ATGGAGCTGGTCGGGTGGCTCGTGGACA |
| | | | AGGGGATTACCTCGGAGAAGCAGTGGAT |
| | | | CCAGGAGGACCAGGCCTCATACATCTCC |
| | | | TTCAATGCGGCCTCCAACTCGCGGTCCC |
| | | | AAATCAAGGCTGCCTTGGACAATGCGGG |
| | | | AAAGATTATGAGCCTGACTAAAACCGCC |
| | | | CCCGACTACCTGGTGGGCCAGCAGCCCG |
| | | | TGGAGGACATTTCCAGCAATCGGATTTA |
| | | | TAAAATTTTGGAACTAAACGGGTACGAT |
| | | | CCCCAATATGCGGCTTCCGTCTTTCTGG |
| | | | GATGGGCCACGAAAAAGTTCGGCAAGAG |
| | | | GAACACCATCTGGCTGTTTGGGCCTGCA |
| | | | ACTACCGGGAAGACCAACATCGCGGAGG |
| | | | CCATAGCCCACACTGTGCCCTTCTACGG |
| | | | GTGCGTAAACTGGACCAATGAGAACTTT |
| | | | CCCTTCAACGACTGTGTCGACAAGATGG |
| | | | TGATCTGGTGGGAGGAGGGGAAGATGAC |
| | | | CGCCAAGGTCGTGGAGTCGGCCAAAGCC |
| | | | ATTCTCGGAGGAAGCAAGGTGCGCGTGG |
| | | | ACCAGAAATGCAAGTCCTCGGCCCAGAT |
| | | | AGACCCGACTCCCGTGATCGTCACCTCC |
| | | | AACACCAACATGTGCGCCGTGATTGACG |
| | | | GGAACTCAACGACCTTCGAACACCAGCA |
| | | | GCCGTTGCAAGACCGGATGTTCAAATTT |
| | | | GAACTCACCCGCCGTCTGGATCATGACT |
| | | | TTGGGAAGGTCACCAAGCAGGAAGTCAA |
| | | | AGACTTTTTCCGGTGGGCAAAGGATCAC |
| | | | GTGGTTGAGGTGGAGCATGAATTCTACG |
| | | | TCAAAAAGGGTGGAGCCAAGAAAAGACC |
| | | | CGCCCCCAGTGACGCAGATATAAGTGAG |
| | | | CCCAAACGGGTGCGCGAGTCAGTTGCGC |
| | | | AGCCATCGACGTCAGACGCGGAAGCTTC |
| | | | GATCAACTACGCAGACAGGTACCAAAAC |
| | | | AAATGTTCTCGTCACGTGGGCATGAATC |
| | | | TGATGCTGTTTCCCTGCAGACAATGCGA |
| | | | GAGAATGAATCAGAATTCAAATATCTGC |
| | | | TTCACTCACGGACAGAAAGACTGTTTAG |
| | | | AGTGCTTTCCCGTGTCAGAATCTCAACC |
| | | | CGTTTCTGTCGTCAAAAAGGCGTATCAG |
| | | | AAACTGTGCTACATTCATCATATCATGG |
| | | | GAAAGGTGCCAGACGCTTGCACTGCCTG |
| | | | CGATCTGGTCAATGTGGATTTGGATGAC |
| | | | TGCATCTTTGAACAATAA (SEQ TD |
| | | | NO: 338) |
| AAV2 | The shorter | MELVGWLVDKGITSEKQWIQEDQASYI | ATGGAGCTGGTCGGGTGGCTCGTGGACA |
| Rep 52 | Rep | SFNAASNSRSQIKAALDNAGKIMSLTK | AGGGGATTACCTCGGAGAAGCAGTGGAT |
| | transcript | TAPDYLVGQQPVEDISSNRIYKILELN | CCAGGAGGACCAGGCCTCATACATCTCC |
| | without | GYDPQYAASVFLGWATKKFGKRNTIWL | TTCAATGCGGCCTCCAACTCGCGGTCCC |
| | splicing | FGPATTGKTNIAEAIAHTVPFYGCVNW | AAATCAAGGCTGCCTTGGACAATGCGGG |
| | | TNENFPFNDCVDKMVIWWEEGKMTAKV | AAAGATTATGAGCCTGACTAAAACCGCC |
| | | VESAKAILGGSKVRVDQKCKSSAQIDP | CCCGACTACCTGGTGGGCCAGCAGCCCG |
| | | TPVIVTSNTNMCAVIDGNSTTFEHQQP | TGGAGGACATTTCCAGCAATCGGATTTA |
| | | LQDRMFKFELTRRLDHDFGKVTKQEVK | TAAAATTTTGGAACTAAACGGGTACGAT |
| | | DFFRWAKDHVVEVEHEFYVKKGGAKKR | CCCCAATATGCGGCTTCCGTCTTTCTGG |
| | | PAPSDADISEPKRVRESVAQPSTSDAE | GATGGGCCACGAAAAAGTTCGGCAAGAG |
| | | ASINYADRYQNKCSRHVGMNLMLFPCR | GAACACCATCTGGCTGTTTGGGCCTGCA |
| | | QCERMNQNSNICFTHGQKDCLECFPVS | ACTACCGGGAAGACCAACATCGCGGAGG |
| | | ESQPVSVVKKAYQKLCYIHHIMGKVPD | CCATAGCCCACACTGTGCCCTTCTACGG |
| | | ACTACDLVNVDLDDCIFEQ* (SEQ | GTGCGTAAACTGGACCAATGAGAACTTT |
| | | ID NO: 335) | CCCTTCAACGACTGTGTCGACAAGATGG |
| | | | TGATCTGGTGGGAGGAGGGGAAGATGAC |
| | | | CGCCAAGGTCGTGGAGTCGGCCAAAGCC |
| | | | ATTCTCGGAGGAAGCAAGGTGCGCGTGG |
| | | | ACCAGAAATGCAAGTCCTCGGCCCAGAT |
| | | | AGACCCGACTCCCGTGATCGTCACCTCC |
| | | | AACACCAACATGTGCGCCGTGATTGACG |
| | | | GGAACTCAACGACCTTCGAACACCAGCA |
| | | | GCCGTTGCAAGACCGGATGTTCAAATTT |
| | | | GAACTCACCCGCCGTCTGGATCATGACT |
| | | | TTGGGAAGGTCACCAAGCAGGAAGTCAA |
| | | | AGACTTTTTCCGGTGGGCAAAGGATCAC |
| | | | GTGGTTGAGGTGGAGCATGAATTCTACG |
| | | | TCAAAAAGGGTGGAGCCAAGAAAAGACC |
| | | | CGCCCCCAGTGACGCAGATATAAGTGAG |
| | | | CCCAAACGGGTGCGCGAGTCAGTTGCGC |
| | | | AGCCATCGACGTCAGACGCGGAAGCTTC |
| | | | GATCAACTACGCAGACAGGTACCAAAAC |
| | | | AAATGTTCTCGTCACGTGGGCATGAATC |
| | | | TGATGCTGTTTCCCTGCAGACAATGCGA |
| | | | GAGAATGAATCAGAATTCAAATATCTGC |
| | | | TTCACTCACGGACAGAAAGACTGTTTAG |
| | | | AGTGCTTTCCCGTGTCAGAATCTCAACC |
| | | | CGTTTCTGTCGTCAAAAAGGCGTATCAG |
| | | | AAACTGTGCTACATTCATCATATCATGG |
| | | | GAAAGGTGCCAGACGCTTGCACTGCCTG |
| | | | CGATCTGGTCAATGTGGATTTGGATGAC |
| | | | TGCATCTTTGAACAATAA (SEQ TD |
| | | | NO: 339) |
| AAV2 | The shorter | MELVGWLVDKGITSEKQWIQEDQASYI | ATGGAGCTGGTCGGGTGGCTCGTGGACA |
| Rep 40 | Rep | SFNAASNSRSQIKAALDNAGKIMSLTK | AGGGGATTACCTCGGAGAAGCAGTGGAT |
| | transcript | TAPDYLVGQQPVEDISSNRIYKILELN | CCAGGAGGACCAGGCCTCATACATCTCC |
| | with | GYDPQYAASVFLGWATKKFGKRNTIWL | TTCAATGCGGCCTCCAACTCGCGGTCCC |
| | splicing | FGPATTGKTNIAEAIAHTVPFYGCVNW | AAATCAAGGCTGCCTTGGACAATGCGGG |
| | | TNENFPFNDCVDKMVIWWEEGKMTAKV | AAAGATTATGAGCCTGACTAAAACCGCC |
| | | VESAKAILGGSKVRVDQKCKSSAQIDP | CCCGACTACCTGGTGGGCCAGCAGCCCG |
| | | TPVIVTSNTNMCAVIDGNSTTFEHQQP | TGGAGGACATTTCCAGCAATCGGATTTA |
| | | LQDRMFKFELTRRLDHDFGKVTKQEVK | TAAAATTTTGGAACTAAACGGGTACGAT |
| | | DFFRWAKDHVVEVEHEFYVKKGGAKKR | CCCCAATATGCGGCTTCCGTCTTTCTGG |
| | | PAPSDADISEPKRVRESVAQPSTSDAE | GATGGGCCACGAAAAAGTTCGGCAAGAG |
| | | ASINYADRLARGHSL* (SEQ ID | GAACACCATCTGGCTGTTTGGGCCTGCA |
| | | NO: 336) | ACTACCGGGAAGACCAACATCGCGGAGG |
| | | | CCATAGCCCACACTGTGCCCTTCTACGG |
| | | | GTGCGTAAACTGGACCAATGAGAACTTT |
| | | | CCCTTCAACGACTGTGTCGACAAGATGG |
| | | | TGATCTGGTGGGAGGAGGGGAAGATGAC |
| | | | CGCCAAGGTCGTGGAGTCGGCCAAAGCC |
| | | | ATTCTCGGAGGAAGCAAGGTGCGCGTGG |
| | | | ACCAGAAATGCAAGTCCTCGGCCCAGAT |
| | | | AGACCCGACTCCCGTGATCGTCACCTCC |
| | | | AACACCAACATGTGCGCCGTGATTGACG |
| | | | GGAACTCAACGACCTTCGAACACCAGCA |
| | | | GCCGTTGCAAGACCGGATGTTCAAATTT |
| | | | GAACTCACCCGCCGTCTGGATCATGACT |
| | | | TTGGGAAGGTCACCAAGCAGGAAGTCAA |
| | | | AGACTTTTTCCGGTGGGCAAAGGATCAC |
| | | | GTGGTTGAGGTGGAGCATGAATTCTACG |
| | | | TCAAAAAGGGTGGAGCCAAGAAAAGACC |
| | | | CGCCCCCAGTGACGCAGATATAAGTGAG |
| | | | CCCAAACGGGTGCGCGAGTCAGTTGCGC |
| | | | AGCCATCGACGTCAGACGCGGAAGCTTC |
| | | | GATCAACTACGCAGACAGGTACCAAAAC |
| | | | AAATGTTCTCGTCACGTGGGCATGAATC |
| | | | TGATGCTGTTTCCCTGCAGACAATGCGA |
| | | | GAGAATGAATCAGAATTCAAATATCTGC |
| | | | TTCACTCACGGACAGAAAGACTGTTTAG |
| | | | AGTGCTTTCCCGTGTCAGAATCTCAACC |
| | | | CGTTTCTGTCGTCAAAAAGGCGTATCAG |
| | | | AAACTGTGCTACATTCATCATATCATGG |
| | | | GAAAGGTGCCAGACGCTTGCACTGCCTG |
| | | | CGATCTGGTCAATGTGGATTTGGATGAC |
| | | | TGCATCTTTGAACAATAAATGATTTAAA |
| | | | TCAGGTATGGCTGCCGATGGTTATCTTC |
| | | | CAGATTGGCTCGAGGACACTCTCTCTGA |
| | | | (SEQ ID NO: 340) |

Additional exemplary AAV2 wildtype sequences are provided in Table 4.

Table 4

| Name | Description | Amino acid sequence | Nucleotide sequence |
|---|---|---|---|
| AAV2 | The full | MAADGYLPDWLEDTLSEGIRQWWKLKP | ATGGCTGCCGATGGTTATCTTCCAGATT |
| VP1 | wild type | GPPPPKPAERHKDDSRGLVLPGYKYLG | GGCTCGAGGACACTCTCTCTGAAGGAAT |
| | sequence of | PFNGLDKGEPVNEADAAALEHDKAYDR | AAGACAGTGGTGGAAGCTCAAACCTGGC |
| | AAV2 VP1 | QLDSGDNPYLKYNHADAEFQERLKEDT | CCACCACCACCAAAGCCCGCAGAGCGGC |
| | | SFGGNLGRAVFQAKKRVLEPLGLVEEP | ATAAGGACGACAGCAGGGGTCTTGTGCT |
| | | VKTAPGKKRPVEHSPVEPDSSSGTGKA | TCCTGGGTACAAGTACCTCGGACCCTTC |
| | | GQQPARKRLNFGQTGDADSVPDPQPLG | AACGGACTCGACAAGGGAGAGCCGGTCA |
| | | QPPAAPSGLGTNTMATGSGAPMADNNE | ACGAGGCAGACGCCGCGGCCCTCGAGCA |
| | | GADGVGNSSGNWHCDSTWMGDRVITTS | CGACAAAGCCTACGACCGGCAGCTCGAC |
| | | TRTWALPTYNNHLYKQISSQSGASNDN | AGCGGAGACAACCCGTACCTCAAGTACA |
| | | HYFGYSTPWGYFDFNRFHCHFSPRDWQ | ACCACGCCGACGCGGAGTTTCAGGAGCG |
| | | RLINNNWGFRPKRLNFKLFNIQVKEVT | CCTTAAAGAAGATACGTCTTTTGGGGGC |
| | | QNDGTTTIANNLTSTVQVFTDSEYQLP | AACCTCGGACGAGCAGTCTTCCAGGCGA |
| | | YVLGSAHQGCLPPFPADVFMVPQYGYL | AAAAGAGGGTTCTTGAACCTCTGGGCCT |
| | | TLNNGSQAVGRSSFYCLEYFPSQMLRT | GGTTGAGGAACCTGTTAAGACGGCTCCG |
| | | GNNFTFSYTFEDVPFHSSYAHSQSLDR | GGAAAAAAGAGGCCGGTAGAGCACTCTC |
| | | LMNPLIDQYLYYLSRINTPSGTTTQSR | CTGTGGAGCCAGACTCCTCCTCGGGAAC |
| | | LQFSQAGASDIRDQSRNWLPGPCYRQQ | CGGAAAGGCGGGCCAGCAGCCTGCAAGA |
| | | RVSKTSADNNNSEYSWTGATKYHLNGR | AAAAGATTGAATTTTGGTCAGACTGGAG |
| | | DSLVNPGPAMASHKDDEEKFFPQSGVL | ACGCAGACTCAGTACCTGACCCCCAGCC |
| | | IFGKQGSEKTNVDIEKVMITDEEEIRT | TCTCGGACAGCCACCAGCAGCCCCCTCT |
| | | TNPVATEQYGSVSTNLQRGNRQAATAD | GGTCTGGGAACTAATACGATGGCTACAG |
| | | VNTQGVLPGMVWQDRDVYLQGPIWAKI | GCAGTGGCGCACCAATGGCAGACAATAA |
| | | PHTDGHFHPSPLMGGFGLKHPPPQILI | CGAGGGCGCCGACGGAGTGGGTAATTCC |
| | | KNTPVPANPSTTFSAAKFASFITQYST | TCGGGAAATTGGCATTGCGATTCCACAT |
| | | GQVSVEIEWELQKENSKRWNPEIQYTS | GGATGGGCGACAGAGTCATCACCACCAG |
| | | NYNKSVNVDFTVDINGVYSEPRPIGTR | CACCCGAACCTGGGCCCTGCCCACCTAC |
| | | YLTRNL* (SEQ ID NO: 341) | AACAACCACCTCTACAAACAAATTTCCA |
| | | | GCCAATCAGGAGCCTCGAACGACAATCA |
| | | | CTACTTTGGCTACAGCACCCCTTGGGGG |
| | | | TATTTTGACTTCAACAGATTCCACTGCC |
| | | | ACTTTTCACCACGTGACTGGCAAAGACT |
| | | | CATCAACAACAACTGGGGATTCCGACCC |
| | | | AAGAGACTCAACTTCAAGCTCTTTAACA |
| | | | TTCAAGTCAAAGAGGTCACGCAGAATGA |
| | | | CGGTACGACGACGATTGCCAATAACCTT |
| | | | ACCAGCACGGTTCAGGTGTTTACTGACT |
| | | | CGGAGTACCAGCTCCCGTACGTCCTCGG |
| | | | CTCGGCGCATCAAGGATGCCTCCCGCCG |
| | | | TTCCCAGCAGACGTCTTCATGGTGCCAC |
| | | | AGTATGGATACCTCACCCTGAACAACGG |
| | | | GAGTCAGGCAGTAGGACGCTCTTCATTT |
| | | | TACTGCCTGGAGTACTTTCCTTCTCAGA |
| | | | TGCTGCGTACCGGAAACAACTTTACCTT |
| | | | CAGCTACACTTTTGAGGACGTTCCTTTC |
| | | | CACAGCAGCTACGCTCACAGCCAGAGTC |
| | | | TGGACCGTCTCATGAATCCTCTCATCGA |
| | | | CCAGTACCTGTATTACTTGAGCAGAACA |
| | | | AACACTCCAAGTGGAACCACCACGCAGT |
| | | | CAAGGCTTCAGTTTTCTCAGGCCGGAGC |
| | | | GAGTGACATTCGGGACCAGTCTAGGAAC |
| | | | TGGCTTCCTGGACCCTGTTACCGCCAGC |
| | | | AGCGAGTATCAAAGACATCTGCGGATAA |
| | | | CAACAACAGTGAATACTCGTGGACTGGA |
| | | | GCTACCAAGTACCACCTCAATGGCAGAG |
| | | | ACTCTCTGGTGAATCCGGGCCCGGCCAT |
| | | | GGCAAGCCACAAGGACGATGAAGAAAAG |
| | | | TTTTTTCCTCAGAGCGGGGTTCTCATCT |
| | | | TTGGGAAGCAAGGCTCAGAGAAAACAAA |
| | | | TGTGGACATTGAAAAGGTCATGATTACA |
| | | | GACGAAGAGGAAATCAGGACAACCAATC |
| | | | CCGTGGCTACGGAGCAGTATGGTTCTGT |
| | | | ATCTACCAACCTCCAGAGAGGCAACAGA |
| | | | CAAGCAGCTACCGCAGATGTCAACACAC |
| | | | AAGGCGTTCTTCCAGGCATGGTCTGGCA |
| | | | GGACAGAGATGTGTACCTTCAGGGGCCC |
| | | | ATCTGGGCAAAGATTCCACACACGGACG |
| | | | GACATTTTCACCCCTCTCCCCTCATGGG |
| | | | TGGATTCGGACTTAAACACCCTCCTCCA |
| | | | CAGATTCTCATCAAGAACACCCCGGTAC |
| | | | CTGCGAATCCTTCGACCACCTTCAGTGC |
| | | | GGCAAAGTTTGCTTCCTTCATCACACAG |
| | | | TACTCCACGGGACAGGTCAGCGTGGAGA |
| | | | TCGAGTGGGAGCTGCAGAAGGAAAACAG |
| | | | CAAACGCTGGAATCCCGAAATTCAGTAC |
| | | | ACTTCCAACTACAACAAGTCTGTTAATG |
| | | | TGGACTTTACTGTGGACACTAATGGCGT |
| | | | GTATTCAGAGCCTCGCCCCATTGGCACC |
| | | | AGATACCTGACTCGTAATCTGTAA |
| | | | (SEQ ID NO: 346) |
| AAV2 | The full | TAPGKKRPVEHSPVEPDSSSGTGKAGQ | ACGGCTCCGGGAAAAAAGAGGCCGGTAG |
| VP2 | wild type | QPARKRLNFGQTGDADSVPDPQPLGQP | AGCACTCTCCTGTGGAGCCAGACTCCTC |
| | sequence of | PAAPSGLGTNTMATGSGAPMADNNEGA | CTCGGGAACCGGAAAGGCGGGCCAGCAG |
| | AAV2 VP2 | DGVGNSSGNWHCDSTWMGDRVITTSTR | CCTGCAAGAAAAAGATTGAATTTTGGTC |
| | | TWALPTYNNHLYKQISSQSGASNDNHY | AGACTGGAGACGCAGACTCAGTACCTGA |
| | | FGYSTPWGYFDFNRFHCHFSPRDWQRL | CCCCCAGCCTCTCGGACAGCCACCAGCA |
| | | INNNWGFRPKRLNFKLFNIQVKEVTQN | GCCCCCTCTGGTCTGGGAACTAATACGA |
| | | DGTTTIANNLTSTVQVFTDSEYQLPYV | TGGCTACAGGCAGTGGCGCACCAATGGC |
| | | LGSAHQGCLPPFPADVFMVPQYGYLTL | AGACAATAACGAGGGCGCCGACGGAGTG |
| | | NNGSQAVGRSSFYCLEYFPSQMLRTGN | GGTAATTCCTCGGGAAATTGGCATTGCG |
| | | NFTFSYTFEDVPFHSSYAHSQSLDRLM | ATTCCACATGGATGGGCGACAGAGTCAT |
| | | NPLIDQYLYYLSRINTPSGTTTQSRLQ | CACCACCAGCACCCGAACCTGGGCCCTG |
| | | FSQAGASDIRDQSRNWLPGPCYRQQRV | CCCACCTACAACAACCACCTCTACAAAC |
| | | SKTSADNNNSEYSWTGATKYHLNGRDS | AAATTTCCAGCCAATCAGGAGCCTCGAA |
| | | LVNPGPAMASHKDDEEKFFPQSGVLIF | CGACAATCACTACTTTGGCTACAGCACC |
| | | GKQGSEKTNVDIEKVMITDEEEIRTTN | CCTTGGGGGTATTTTGACTTCAACAGAT |
| | | PVATEQYGSVSTNLQRGNRQAATADVN | TCCACTGCCACTTTTCACCACGTGACTG |
| | | TQGVLPGMVWQDRDVYLQGPIWAKIPH | GCAAAGACTCATCAACAACAACTGGGGA |
| | | TDGHFHPSPLMGGFGLKHPPPQILIKN | TTCCGACCCAAGAGACTCAACTTCAAGC |
| | | TPVPANPSTTFSAAKFASFITQYSTGQ | TCTTTAACATTCAAGTCAAAGAGGTCAC |
| | | VSVEIEWELQKENSKRWNPEIQYTSNY | GCAGAATGACGGTACGACGACGATTGCC |
| | | NKSVNVDFTVDINGVYSEPRPIGTRYL | AATAACCTTACCAGCACGGTTCAGGTGT |
| | | TRNL* (SEQ ID NO: 342) | TTACTGACTCGGAGTACCAGCTCCCGTA |
| | | | CGTCCTCGGCTCGGCGCATCAAGGATGC |
| | | | CTCCCGCCGTTCCCAGCAGACGTCTTCA |
| | | | TGGTGCCACAGTATGGATACCTCACCCT |
| | | | GAACAACGGGAGTCAGGCAGTAGGACGC |
| | | | TCTTCATTTTACTGCCTGGAGTACTTTC |
| | | | CTTCTCAGATGCTGCGTACCGGAAACAA |
| | | | CTTTACCTTCAGCTACACTTTTGAGGAC |
| | | | GTTCCTTTCCACAGCAGCTACGCTCACA |
| | | | GCCAGAGTCTGGACCGTCTCATGAATCC |
| | | | TCTCATCGACCAGTACCTGTATTACTTG |
| | | | AGCAGAACAAACACTCCAAGTGGAACCA |
| | | | CCACGCAGTCAAGGCTTCAGTTTTCTCA |
| | | | GGCCGGAGCGAGTGACATTCGGGACCAG |
| | | | TCTAGGAACTGGCTTCCTGGACCCTGTT |
| | | | ACCGCCAGCAGCGAGTATCAAAGACATC |
| | | | TGCGGATAACAACAACAGTGAATACTCG |
| | | | TGGACTGGAGCTACCAAGTACCACCTCA |
| | | | ATGGCAGAGACTCTCTGGTGAATCCGGG |
| | | | CCCGGCCATGGCAAGCCACAAGGACGAT |
| | | | GAAGAAAAGTTTTTTCCTCAGAGCGGGG |
| | | | TTCTCATCTTTGGGAAGCAAGGCTCAGA |
| | | | GAAAACAAATGTGGACATTGAAAAGGTC |
| | | | ATGATTACAGACGAAGAGGAAATCAGGA |
| | | | CAACCAATCCCGTGGCTACGGAGCAGTA |
| | | | TGGTTCTGTATCTACCAACCTCCAGAGA |
| | | | GGCAACAGACAAGCAGCTACCGCAGATG |
| | | | TCAACACACAAGGCGTTCTTCCAGGCAT |
| | | | GGTCTGGCAGGACAGAGATGTGTACCTT |
| | | | CAGGGGCCCATCTGGGCAAAGATTCCAC |
| | | | ACACGGACGGACATTTTCACCCCTCTCC |
| | | | CCTCATGGGTGGATTCGGACTTAAACAC |
| | | | CCTCCTCCACAGATTCTCATCAAGAACA |
| | | | CCCCGGTACCTGCGAATCCTTCGACCAC |
| | | | CTTCAGTGCGGCAAAGTTTGCTTCCTTC |
| | | | ATCACACAGTACTCCACGGGACAGGTCA |
| | | | GCGTGGAGATCGAGTGGGAGCTGCAGAA |
| | | | GGAAAACAGCAAACGCTGGAATCCCGAA |
| | | | ATTCAGTACACTTCCAACTACAACAAGT |
| | | | CTGTTAATGTGGACTTTACTGTGGACAC |
| | | | TAATGGCGTGTATTCAGAGCCTCGCCCC |
| | | | ATTGGCACCAGATACCTGACTCGTAATC |
| | | | TGTAA (SEQ ID NO: 347) |
| AAV2 | The wild | MATGSGAPMADNNEGADGVGNSSGNWH | ATGGCTACAGGCAGTGGCGCACCAATGG |
| VP3 | type | CDSTWMGDRVITTSTRIWALPTYNNHL | CAGACAATAACGAGGGCGCCGACGGAGT |
| | sequence of | YKQISSQSGASNDNHYFGYSTPWGYFD | GGGTAATTCCTCGGGAAATTGGCATTGC |
| | AAV2 VP3 | FNRFHCHFSPRDWQRLINNNWGFRPKR | GATTCCACATGGATGGGCGACAGAGTCA |
| | | LNFKLFNIQVKEVTQNDGTTTIANNLT | TCACCACCAGCACCCGAACCTGGGCCCT |
| | | STVQVFTDSEYQLPYVLGSAHQGCLPP | GCCCACCTACAACAACCACCTCTACAAA |
| | | FPADVFMVPQYGYLTLNNGSQAVGRSS | CAAATTTCCAGCCAATCAGGAGCCTCGA |
| | | FYCLEYFPSQMLRTGNNFTFSYTFEDV | ACGACAATCACTACTTTGGCTACAGCAC |
| | | PFHSSYAHSQSLDRLMNPLIDQYLYYL | CCCTTGGGGGTATTTTGACTTCAACAGA |
| | | SRINTPSGTTTQSRLQFSQAGASDIRD | TTCCACTGCCACTTTTCACCACGTGACT |
| | | QSRNWLPGPCYRQQRVSKTSADNNNSE | GGCAAAGACTCATCAACAACAACTGGGG |
| | | YSWTGATKYHLNGRDSLVNPGPAMASH | ATTCCGACCCAAGAGACTCAACTTCAAG |
| | | KDDEEKFFPQSGVLIFGKQGSEKTNVD | CTCTTTAACATTCAAGTCAAAGAGGTCA |
| | | IEKVMITDEEEIRTTNPVATEQYGSVS | CGCAGAATGACGGTACGACGACGATTGC |
| | | TNLQRGNRQAATADVNTQGVLPGMVWQ | CAATAACCTTACCAGCACGGTTCAGGTG |
| | | DRDVYLQGPIWAKIPHTDGHFHPSPLM | TTTACTGACTCGGAGTACCAGCTCCCGT |
| | | GGFGLKHPPPQILIKNTPVPANPSTTF | ACGTCCTCGGCTCGGCGCATCAAGGATG |
| | | SAAKFASFITQYSTGQVSVEIEWELQK | CCTCCCGCCGTTCCCAGCAGACGTCTTC |
| | | ENSKRWNPEIQYTSNYNKSVNVDFTVD | ATGGTGCCACAGTATGGATACCTCACCC |
| | | TNGVYSEPRPIGTRYLTRNL* (SEQ | TGAACAACGGGAGTCAGGCAGTAGGACG |
| | | ID NO: 343) | CTCTTCATTTTACTGCCTGGAGTACTTT |
| | | | CCTTCTCAGATGCTGCGTACCGGAAACA |
| | | | ACTTTACCTTCAGCTACACTTTTGAGGA |
| | | | CGTTCCTTTCCACAGCAGCTACGCTCAC |
| | | | AGCCAGAGTCTGGACCGTCTCATGAATC |
| | | | CTCTCATCGACCAGTACCTGTATTACTT |
| | | | GAGCAGAACAAACACTCCAAGTGGAACC |
| | | | ACCACGCAGTCAAGGCTTCAGTTTTCTC |
| | | | AGGCCGGAGCGAGTGACATTCGGGACCA |
| | | | GTCTAGGAACTGGCTTCCTGGACCCTGT |
| | | | TACCGCCAGCAGCGAGTATCAAAGACAT |
| | | | CTGCGGATAACAACAACAGTGAATACTC |
| | | | GTGGACTGGAGCTACCAAGTACCACCTC |
| | | | AATGGCAGAGACTCTCTGGTGAATCCGG |
| | | | GCCCGGCCATGGCAAGCCACAAGGACGA |
| | | | TGAAGAAAAGTTTTTTCCTCAGAGCGGG |
| | | | GTTCTCATCTTTGGGAAGCAAGGCTCAG |
| | | | AGAAAACAAATGTGGACATTGAAAAGGT |
| | | | CATGATTACAGACGAAGAGGAAATCAGG |
| | | | ACAACCAATCCCGTGGCTACGGAGCAGT |
| | | | ATGGTTCTGTATCTACCAACCTCCAGAG |
| | | | AGGCAACAGACAAGCAGCTACCGCAGAT |
| | | | GTCAACACACAAGGCGTTCTTCCAGGCA |
| | | | TGGTCTGGCAGGACAGAGATGTGTACCT |
| | | | TCAGGGGCCCATCTGGGCAAAGATTCCA |
| | | | CACACGGACGGACATTTTCACCCCTCTC |
| | | | CCCTCATGGGTGGATTCGGACTTAAACA |
| | | | CCCTCCTCCACAGATTCTCATCAAGAAC |
| | | | ACCCCGGTACCTGCGAATCCTTCGACCA |
| | | | CCTTCAGTGCGGCAAAGTTTGCTTCCTT |
| | | | CATCACACAGTACTCCACGGGACAGGTC |
| | | | AGCGTGGAGATCGAGTGGGAGCTGCAGA |
| | | | AGGAAAACAGCAAACGCTGGAATCCCGA |
| | | | AATTCAGTACACTTCCAACTACAACAAG |
| | | | TCTGTTAATGTGGACTTTACTGTGGACA |
| | | | CTAATGGCGTGTATTCAGAGCCTCGCCC |
| | | | CATTGGCACCAGATACCTGACTCGTAAT |
| | | | CTGTAA (SEQ ID NO: 348) |
| AAV2 | The | LAHHHQSPQSGIRTTAGVLCFLGTSTS | CTGGCCCACCACCACCAAAGCCCGCAGA |
| MAAP | sequence of | DPSTDSTRESRSTRQTPRPSSTTKPTT | GCGGCATAAGGACGACAGCAGGGGTCTT |
| | MAAP in | GSSTAETTRISSTTTPTRSFRSALKKI | GTGCTTCCTGGGTACAAGTACCTCGGAC |
| | AAV2 for | RLLGATSDEQSSRRKRGFLNLWAWLRN | CCTTCAACGGACTCGACAAGGGAGAGCC |
| | reference | LLRRLREKRGR* (SEQ ID NO: | GGTCAACGAGGCAGACGCCGCGGCCCTC |
| | | 344) | GAGCACGACAAAGCCTACGACCGGCAGC |
| | | | TCGACAGCGGAGACAACCCGTACCTCAA |
| | | | GTACAACCACGCCGACGCGGAGTTTCAG |
| | | | GAGCGCCTTAAAGAAGATACGTCTTTTG |
| | | | GGGGCAACCTCGGACGAGCAGTCTTCCA |
| | | | GGCGAAAAAGAGGGTTCTTGAACCTCTG |
| | | | GGCCTGGTTGAGGAACCTGTTAAGACGG |
| | | | CTCCGGGAAAAAAGAGGCCGGTAG |
| | | | (SEQ ID NO: 349) |
| AAV2 | The | LETQTQYLTPSLSDSHQQPPLVWELIR | CTGGAGACGCAGACTCAGTACCTGACCC |
| AAP | sequence of | WLQAVAHQWQTITRAPTEWVIPREIGI | CCAGCCTCTCGGACAGCCACCAGCAGCC |
| | AAP in | AIPHGWATESSPPAPEPGPCPPTTTTS | CCCTCTGGTCTGGGAACTAATACGATGG |
| | AAV2 for | TNKFPANQEPRTTITTLATAPLGGILT | CTACAGGCAGTGGCGCACCAATGGCAGA |
| | reference | STDSTATFHHVTGKDSSTTTGDSDPRD | CAATAACGAGGGCGCCGACGGAGTGGGT |
| | | STSSSLTFKSKRSRRMTVRRRLPITLP | AATTCCTCGGGAAATTGGCATTGCGATT |
| | | ARFRCLLTRSTSSRTSSARRIKDASRR | CCACATGGATGGGCGACAGAGTCATCAC |
| | | SQQTSSWCHSMDTSP* (SEQ ID | CACCAGCACCCGAACCTGGGCCCTGCCC |
| | | NO: 345) | ACCTACAACAACCACCTCTACAAACAAA |
| | | | TTTCCAGCCAATCAGGAGCCTCGAACGA |
| | | | CAATCACTACTTTGGCTACAGCACCCCT |
| | | | TGGGGGTATTTTGACTTCAACAGATTCC |
| | | | ACTGCCACTTTTCACCACGTGACTGGCA |
| | | | AAGACTCATCAACAACAACTGGGGATTC |
| | | | CGACCCAAGAGACTCAACTTCAAGCTCT |
| | | | TTAACATTCAAGTCAAAGAGGTCACGCA |
| | | | GAATGACGGTACGACGACGATTGCCAAT |
| | | | AACCTTACCAGCACGGTTCAGGTGTTTA |
| | | | CTGACTCGGAGTACCAGCTCCCGTACGT |
| | | | CCTCGGCTCGGCGCATCAAGGATGCCTC |
| | | | CCGCCGTTCCCAGCAGACGTCTTCATGG |
| | | | TGCCACAGTATGGATACCTCACCCTGA |
| | | | (SEQ ID NO: 350) |

In some embodiments, a nucleic acid of the disclosure (e.g., comprising an ORF encoding MAAP comprising an exogenous start codon, or comprising a payload, e.g., a transgene) comprises conventional control elements or sequences which are operably linked to the ORF encoding MAAP or to the payload, e.g., transgene, in a manner which permits transcription, translation and/or expression in a cell transfected with the nucleic acid (e.g., a plasmid vector comprising said nucleic acid) or infected with a virus comprising said nucleic acid. As used herein, “operably linked” sequences include both expression control sequences that are contiguous with the gene of interest and expression control sequences that act in trans or at a distance to control the gene of interest.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and may be utilized with the compositions and methods disclosed herein.

In some embodiments, the native promoter for the transgene may be used. Without wishing to be bound by theory, the native promoter may mimic native expression of the transgene, or provide temporal, developmental, or tissue-specific expression, or expression in response to specific transcriptional stimuli. In some embodiment, the transgene may be operably linked to other native expression control elements, such as enhancer elements, polyadenylation sites or Kozak consensus sequences, e.g., to mimic the native expression.

In some embodiments, the transgene is operably linked to a tissue-specific promoter.

In some embodiments, a vector, e.g., a plasmid, carrying a transgene may also include a selectable marker or a reporter gene. Such selectable reporters or marker genes can be used to signal the presence of the vector, e.g., plasmid, in bacterial cells. Other components of the vector, e.g., plasmid, may include an origin of replication. Selection of these and other promoters and vector elements are conventional and many such sequences are available [see, e.g., Sambrook et al, and references cited therein].

### MAAP Polypeptides

The disclosure is directed, in part, to a MAAP polypeptide encoded by a nucleic acid described herein (e.g., a nucleic acid comprising an ORF encoding a functional MAAP polypeptide comprising an exogenous start codon), and to a MAAP polypeptide comprising a mutation corresponding to the presence of an exogenous start codon in the nucleic acid encoding said MAAP polypeptide.

In some embodiments, the exogenous start codon is an ATG. In some embodiments, a MAAP polypeptide comprises an amino acid corresponding to the exogenous start codon (e.g., the first amino acid of the MAAP polypeptide). In some embodiments, the amino acid is a methionine.

In some embodiments, the exogenous start codon is a CTG. In some embodiments, a MAAP polypeptide comprises an amino acid corresponding to the exogenous start codon (e.g., the first amino acid of the MAAP polypeptide). In some embodiments, the amino acid is a leucine.

In some embodiments, a MAAP polypeptide (e.g., encoded by an ORF of a nucleic acid described herein) is a functional MAAP polypeptide. In some embodiments, the presence of the MAAP polypeptide in a cell, cell-free system, or translation system improves (e.g., increases) a production characteristic of the cell, cell-free system, or translation system, dependoparvovirus particle produced by the cell, cell-free system, or translation system, and/or a method of making the dependoparvovirus particle using the cell, cell-free system, or translation system.

In some embodiments, a MAAP polypeptide is an isolated or purified polypeptide (e.g., isolated or purified from a cell, other biological component, or contaminant). In some embodiments, a MAAP polypeptide is present in a dependoparvovirus particle, e.g., described herein. In some embodiments, a MAAP polypeptide is present in a cell, cell-free system, or translation system, e.g., described herein.

In some embodiments, the MAAP polypeptide is a dependoparvovirus B (e.g., AAV5) MAAP polypeptide. In some embodiments, the MAAP polypeptide is a functional MAAP polypeptide. MAAP polypeptides may comprise one or more structural regions. In some embodiments, a MAAP polypeptide comprises one, two, three, four, five, or all of: an N-terminal disordered region; a short hydrophobic region comprising a beta-strand; a T/S rich disordered region; a region devoid of predicted secondary structure; a disordered region; or a C-terminal amphipathic region comprising an alpha-helix. In some embodiments, a MAAP polypeptide comprises, from most N-terminal to most C-terminal, one, two, three, four, five, or all of the following domains: an N-terminal disordered region; a short hydrophobic region comprising a beta-strand; a T/S rich disordered region; a region devoid of predicted secondary structure; a disordered region; or a C-terminal amphipathic region comprising an alpha-helix. In some embodiments, a MAAP polypeptide comprises, from most N-terminal to most C-terminal: an N-terminal disordered region; a short hydrophobic region comprising a beta-strand; a T/S rich disordered region; a region devoid of predicted secondary structure; a disordered region; and a C-terminal amphipathic region comprising an alpha-helix.

In some embodiments, the N-terminal disordered region is capable of binding to a polypeptide. In some embodiments, the short hydrophobic region comprising a beta-strand is capable of binding to a polypeptide. In some embodiments, the T/S rich disordered region is enriched in charged amino acids. In some embodiments, the region devoid of predicted secondary structure is capable of binding to a polypeptide. In some embodiments, the disordered region is capable of forming an alpha helix. In some embodiments, the C-terminal amphipathic region comprising an alpha-helix is capable of binding a membrane.

In some embodiments, a MAAP polypeptide comprises a full length MAAP, e.g., the MAAP polypeptide is not missing a region or amino acids present in a reference MAAP (e.g., a naturally occurring MAAP) or a region or amino acids corresponding to those positions of a reference MAAP. In some embodiments, a MAAP polypeptide comprises a truncation and/or deletion relative to a reference MAAP, e.g., is missing a region or amino acids present in a reference MAAP (e.g., a naturally occurring MAAP) or a region or amino acids corresponding to those positions of a reference MAAP. In some embodiments, a MAAP polypeptide comprises at least 80, 85, 90, 95, 100, 105, 110, 115, or 116 amino acids (e.g., a full length MAAP) and optionally no more than 120, 119, 118, 117, 116, 115, 110, 105, or 100 amino acids.

In some embodiments, the MAAP polypeptide comprises an alteration relative to a reference sequence. In some embodiments, the reference sequence is a naturally occurring dependoparvovirus B MAAP, e.g., a naturally occurring AAV5 MAAP. In some embodiments, the reference sequence is a mutant, artificial, or synthetic MAAP known in the art. In some embodiments, the reference sequence comprises a wildtype sequence, e.g., SEQ ID NO: 325, or a sequence with at least 90, 91, 92, 93, 94, 95, 96, 97, 98, or 99% sequence identity with a wildtype sequence, e.g., SEQ ID NO: 325. In some embodiments, the reference sequences comprises a subset (e.g., a truncation) of a wildtype sequence, e.g., wherein the position of the exogenous start codon results in a truncated MAAP polypeptide relative to the putative ORF encoding MAAP. In some embodiments, the alteration comprises substitution, deletion, or insertion of one or more amino acids, or a combination of a substitution, deletion, or insertion. In some embodiments, a MAAP polypeptide comprises an alteration specified by the CIGAR string of column 8 of Table 1 relative to AAV5 MAAP, or at a corresponding position in another dependoparvovirus MAAP, e.g., resulting from the presence of an exogenous start codon in the nucleic acid sequence encoding the MAAP polypeptide. In some embodiments, a MAAP polypeptide comprises one or more additional amino acids at the N- and/or C-termini, e.g., relative to a reference MAAP polypeptide.

In some embodiments, the MAAP polypeptide comprises an amino acid sequence that has at least 80, 85, 90, 91, 92, 93, 94, 95, 96, 97, 98, 99, or 100% identity to the amino acid sequence of a wildtype dependoparvovirus B MAAP polypeptide (e.g., SEQ ID NO: 325). In some embodiments, the MAAP polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a wildtype dependoparvovirus B MAAP polypeptide (e.g., SEQ ID NO: 325). In some embodiments, the MAAP polypeptide is an AAV5 MAAP polypeptide. In some embodiments, the MAAP polypeptide comprises an amino acid sequence that has at least 80, 85, 90, 91, 92, 93, 94, 95, 96, 97, 98, 99, or 100% identity to the amino acid sequence of a wildtype AAV5 MAAP polypeptide (e.g., SEQ ID NO: 325). In some embodiments, the MAAP polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a wildtype AAV5 MAAP polypeptide (e.g., SEQ ID NO: 325). In some embodiments, the MAAP polypeptide differs by 1-30, 5-30, 10-30, 15-30, 20-30, 25-30, 1-25, 5-25, 10-25, 15-25, 20-25, 1-20, 5-20, 10-20, 15-20, 1-15, 5-15, 10-15, 1-10, 5-10, or 1-5 amino acids from the amino acid sequence of a wildtype AAV5 MAAP polypeptide (e.g., SEQ ID NO: 325).

In some embodiments, the MAAP polypeptide comprises an amino acid sequence that has at least 80, 85, 90, 91, 92, 93, 94, 95, 96, 97, 98, 99, or 100% identity to an amino acid sequence of Table 2, e.g., SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319. In some embodiments, the MAAP polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from an amino acid sequence of Table 2, e.g., SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319.

In some embodiments, the MAAP polypeptide is a wildtype MAAP polypeptide (e.g., a wildtype dependoparvovirus B, e.g., an AAV5, MAAP polypeptide) at all other positions besides those affected by the exogenous start codon. In some embodiments, the MAAP polypeptide is a wildtype MAAP polypeptide (e.g., a wildtype dependoparvovirus B, e.g., an AAV5, MAAP polypeptide) at all other positions besides those affected by the exogenous start codon and a position that is altered (relative to a wildtype sequence, e.g., SEQ ID NO: 325) in any of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319. In some embodiments, a plurality of the positions altered in any of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319 relative to relative to a wildtype sequence, e.g., SEQ ID NO: 325, is altered in the amino acid sequence of the MAAP polypeptide. For example, the MAAP polypeptide may be a wildtype MAAP polypeptide (e.g., a wildtype dependoparvovirus B, e.g., an AAV5, MAAP polypeptide) at all other positions besides those affected by the exogenous start codon and the positions altered (relative to a wildtype sequence, e.g., SEQ ID NO: 325) in any two, three, four, five, six, seven, eight, nine, or ten of of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319.

In some embodiments, the MAAP polypeptide further comprises an additional alteration (e.g., a substitution, insertion, or deletion) relative to a wildtype sequence (e.g., SEQ ID NO: 325) in addition to any amino acid change resulting from the presence of the exogenous start codon in the ORF encoding MAAP and any alteration present relative to a wildtype sequence in SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319. In some embodiments, the additional alteration improves a production characteristic of a dependoparvovirus particle or method of making the same. In some embodiments, the additional alteration improves or alters another characteristic of a dependoparvovirus particle, e.g., tropism.

### Other Polypeptides and Nucleic Acids

The disclosure is further directed, in part, to a nucleic acid comprising a sequence encoding an ORF for a functional MAAP polypeptide comprising an exogenous start codon that further comprises a sequence encoding one or more dependoparvovirus genes. In some embodiments, the nucleic acid comprising a sequence encoding an ORF for a functional MAAP polypeptide further comprises a dependoparvovirus gene. In some embodiments, the nucleic acid comprising a sequence encoding an ORF for a functional MAAP polypeptide further comprises a plurality of dependoparvovirus genes. In some embodiments, the nucleic acid, e.g., the plurality of dependoparvovirus genes, is sufficient to direct production of functional dependoparvovirus particles in a cell, e.g., a human cell, cell-free system, or other translation system (e.g., all of the genes in a dependoparvovirus genome). In some embodiments, the nucleic acid comprises one or more helper sequences.

In some embodiments, the one or more dependoparvovirus genes are of the same species (e.g., dependoparvovirus B) and/or serotype (e.g., AAV5) as the ORF encoding MAAP polypeptide. In some embodiments, the one or more dependoparvovirus genes have at least 80, 85, 90, 91, 92, 93, 94, 95, 96, 97, 98, 99, or 100% identity to the amino acid sequence of a corresponding dependoparvovirus gene of the same species (e.g., dependoparvovirus B) and/or serotype (e.g., AAV5) as the ORF encoding MAAP polypeptide, or differ by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from a corresponding dependoparvovirus gene of the same species (e.g., dependoparvovirus B) and/or serotype (e.g., AAV5) as the ORF encoding MAAP polypeptide. In some embodiments, the one or more dependoparvovirus genes are of a different species (e.g., dependoparvovirus A) and/or serotype as the ORF encoding MAAP polypeptide. In some embodiments, the one or more dependoparvovirus genes are of AAV2 or AAV9. In some embodiments, the one or more dependoparvovirus genes have at least 80, 85, 90, 91, 92, 93, 94, 95, 96, 97, 98, 99, or 100% identity to the amino acid sequence of a corresponding dependoparvovirus gene of dependoparvovirus A and/or AAV2 or AAV9, or differ by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from a corresponding dependoparvovirus gene of dependoparvovirus A and/or AAV2 or AAV9.

In some embodiments, the nucleic acid comprising a sequence encoding an ORF for a functional MAAP polypeptide comprising an exogenous start codon further comprises a Cap gene (e.g., a sequence encoding a Cap polypeptide) or a functional variant or portion thereof. In some embodiments, the Cap polypeptide has at least 80, 85, 90, 91, 92, 93, 94, 95, 96, 97, 98, 99, or 100% identity to the amino acid sequence of a wildtype dependoparvovirus A or B Cap polypeptide (e.g., an AAV2, AAV5, or AAV9 Cap polypeptide), e.g., SEQ ID NO: 321, or differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from a wildtype dependoparvovirus A or B Cap polypeptide (e.g., an AAV2, AAV5, or AAV9 Cap polypeptide), e.g., SEQ ID NO: 321.

In some embodiments, the nucleic acid comprising a sequence encoding an ORF for a functional MAAP polypeptide comprising an exogenous start codon further comprises a Rep gene (e.g., a sequence encoding a Rep polypeptide) or a functional variant or portion thereof. In some embodiments, the Rep polypeptide has at least 80, 85, 90, 91, 92, 93, 94, 95, 96, 97, 98, 99, or 100% identity to the amino acid sequence of a wildtype dependoparvovirus A or B Rep polypeptide (e.g., an AAV2, AAV5, or AAV9 Rep polypeptide), e.g., any of SEQ ID NOs: 333-336, or differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from a wildtype dependoparvovirus A or B Rep polypeptide (e.g., an AAV2, AAV5, or AAV9 Rep polypeptide), e.g., any of SEQ ID NOs: 333-336.

In some embodiments, the nucleic acid comprising a sequence encoding an ORF for a functional MAAP polypeptide comprising an exogenous start codon further comprises a sequence encoding a VP1 polypeptide or a functional variant or portion thereof. In some embodiments, the VP1 polypeptide has at least 80, 85, 90, 91, 92, 93, 94, 95, 96, 97, 98, 99, or 100% identity to the amino acid sequence of a wildtype dependoparvovirus A or B VP1 polypeptide (e.g., an AAV2, AAV5, or AAV9 VP1 polypeptide), e.g., SEQ ID NO: 321, or differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from a wildtype dependoparvovirus A or B VP1 polypeptide (e.g., an AAV2, AAV5, or AAV9 VP1 polypeptide), e.g., SEQ ID NO: 321.

In some embodiments, the nucleic acid comprising a sequence encoding an ORF for a functional MAAP polypeptide comprising an exogenous start codon further comprises a sequence encoding VP2 polypeptide or a functional variant or portion thereof. In some embodiments, the VP2 polypeptide has at least 80, 85, 90, 91, 92, 93, 94, 95, 96, 97, 98, 99, or 100% identity to the amino acid sequence of a wildtype dependoparvovirus A or B VP2 polypeptide (e.g., an AAV2, AAV5, or AAV9 VP2 polypeptide), e.g., SEQ ID NO: 322, or differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from a wildtype dependoparvovirus A or B VP2 polypeptide (e.g., an AAV2, AAV5, or AAV9 VP2 polypeptide), e.g., SEQ ID NO: 322.

In some embodiments, the nucleic acid comprising a sequence encoding an ORF for a functional MAAP polypeptide comprising an exogenous start codon further comprises a sequence encoding VP3 polypeptide or a functional variant or portion thereof. In some embodiments, the VP3 polypeptide has at least 80, 85, 90, 91, 92, 93, 94, 95, 96, 97, 98, 99, or 100% identity to the amino acid sequence of a wildtype dependoparvovirus A or B VP3 polypeptide (e.g., an AAV2, AAV5, or AAV9 VP3 polypeptide), e.g., SEQ ID NO: 323, or differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from a wildtype dependoparvovirus A or B VP3 polypeptide (e.g., an AAV2, AAV5, or AAV9 VP3 polypeptide), e.g., SEQ ID NO: 323.

Given that dependoparvovirus genomes may comprise multiple genes wherein a plurality of the genes overlap one another, a nucleic acid comprising a sequence encoding an ORF for a functional MAAP polypeptide comprising an exogenous start codon may inherently comprise a portion of sequence encoding another dependoparvovirus gene. Accordingly, when the disclosure recites that such a nucleic acid further comprises a dependoparvovirus gene, it is meant that the nucleic acid comprises all or an additional portion of said dependoparvovirus gene. For example, when the disclosure recites that the nucleic acid further comprises a sequence encoding a VP1 polypeptide, said sequence encoding a VP1 polypeptide would be in addition to any sequence encoding a VP1 polypeptide inherently present in a sequence encoding an ORF for a MAAP polypeptide. In some embodiments of such an example, further comprising a sequence encoding a VP1 polypeptide means the nucleic acid comprises a single sequence that encodes a full length VP1 polypeptide, e.g., that partially overlaps the ORF encoding MAAP. In other embodiments of such an example, further comprising a sequence encoding a VP1 polypeptide means the nucleic acid comprises a VP1 polypeptide encoding sequence (or a functional variant or portion thereof) that does not overlap the ORF encoding MAAP, e.g., in addition to any VP1 encoding sequence inherently present in the ORF encoding MAAP.

VP1 Nucleic Acids and Polypeptides

The disclosure is further directed, in part, to a nucleic acid comprising a sequence encoding a dependoparvovirus (e.g., dependoparvovirus B, e.g., an AAV5) VP1 polypeptide, as well as to a VP1 polypeptide encoded by the same. In some embodiments, such nucleic acids further comprise a sequence encoding an ORF for a functional MAAP polypeptide comprising an exogenous start codon. In some embodiments, such nucleic acids comprise a Cap gene, or a portion of a Cap gene encoding VP1. Without wishing to be bound by theory, some naturally occurring dependoparvovirus genomes comprise multiple genes wherein a plurality of the genes overlap one another, with the overlapping genes each positioned in a different ORF (e.g., +0, +1, or +2). For example, the sequence encoding VP1 polypeptide of the Cap gene can overlap (e.g., partially overlap) with the sequence encoding a MAAP polypeptide. Accordingly, a change to the sequence comprising an ORF encoding one gene may affect the ORF of another gene as well. The disclosure is accordingly directed, in part, to a nucleic acid encoding a Cap, e.g., VP1, polypeptide comprising a mutation corresponding to an exogenous start codon in an ORF encoding a MAAP polypeptide, as well as to a Cap, e.g., VP1, polypeptide encoded by the same. In some embodiments, the Cap, e.g., VP1, polypeptide is a functional Cap, e.g., VP1, polypeptide. In some embodiments, the polypeptide produced from the Cap, e.g., VP1, encoding sequence is capable of assembling into a dependoparvovirus capsid, e.g., a dependoparvovirus capsid capable of infecting a target cell.

In some embodiments, a nucleic acid comprises a sequence encoding an ORF for a functional MAAP polypeptide comprising an exogenous start codon, wherein the exogenous start codon results in a silent mutation in the nucleic acid sequence encoding another dependoparvovirus gene present in the nucleic acid. In some embodiments, a nucleic acid comprises a sequence encoding an ORF for a functional MAAP polypeptide comprising an exogenous start codon, wherein the exogenous start codon results in a change in the amino acid sequence of another dependoparvovirus gene present in the nucleic acid.

In some embodiments, the exogenous start codon results in an amino acid change in a Cap polypeptide encoded by a sequence of the nucleic acid. In some embodiments, the amino acid change is a conservative mutation. In some embodiments, the amino acid change is not a conservative mutation. The term “conservative” mutation refers to a mutation (e.g., substitution) of an amino acid residue to another amino acid residue, including naturally occurring and non-naturally occurring amino acids, such that there is little or no effect on the polarity or charge of the amino acid residue at that position. For example, a conservative mutation results from the replacement of a non-polar residue in a polypeptide with any other non-polar residue. In some embodiments, any native residue in the polypeptide may also be substituted with alanine, according to the methods of “alanine scanning mutagenesis”. Naturally occurring amino acids are characterized based on their side chains as follows: acidic: glutamic acid, aspartic acid; basic: arginine, lysine, histidine; non-polar: phenylalanine, tryptophan, cysteine, glycine, alanine, valine, proline, methionine, leucine, norleucine, isoleucine; and uncharged polar: glutamine, asparagine, serine, threonine, tyrosine. In some embodiments, the exogenous start codon results in an amino acid change in a VP1 polypeptide encoded by a sequence of the nucleic acid. In some embodiments, the amino acid change is a conservative mutation. In some embodiments, the amino acid change is not a conservative mutation.

In some embodiments, the Cap polypeptide, e.g., VP1 polypeptide, comprises an amino acid sequence: provided in Table 2; that has at least 80, 85, 90, 91, 92, 93, 94, 95, 96, 97, 98, 99, or 100% identity to an amino acid sequence provided in Table 2; or that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from an amino acid sequence provided in Table 2. In some embodiments, the Cap polypeptide, e.g., VP1 polypeptide, comprises an alteration (e.g., a substitution) relative to a wildtype VP1 polypeptide sequence (e.g., an AAV2 or AAV5 wildtype VP1 polypeptide, e.g., SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317). In some embodiments, the alteration is an alteration at position specified by a CIGAR string of column 7 of Table 1. In some embodiments, the Cap, e.g., VP1, polypeptide is a wildtype Cap, e.g., VP1, polypeptide (e.g., a wildtype dependoparvovirus B, e.g., an AAV5, Cap, e.g., VP1, polypeptide) at all other positions besides those affected by the exogenous start codon. In some embodiments, the Cap, e.g., VP1, polypeptide is a wildtype Cap, e.g., VP1, polypeptide (e.g., a wildtype dependoparvovirus B, e.g., an AAV5, Cap, e.g., VP1, polypeptide) at all other positions besides those affected by the exogenous start codon and a position that is altered (relative to a wildtype sequence, e.g., SEQ ID NO: 321) in any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317. In some embodiments, a plurality of the positions altered in any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317 relative to relative to a wildtype sequence, e.g., SEQ ID NO: 321, is altered in the amino acid sequence of the VP1 polypeptide. For example, the VP1 polypeptide may be a wildtype VP1 polypeptide (e.g., a wildtype dependoparvovirus B, e.g., an AAV5, VP1 polypeptide) at all other positions besides those affected by the exogenous start codon and the positions altered (relative to a wildtype sequence, e.g., SEQ ID NO: 321) in any two, three, four, five, six, seven, eight, nine, or ten of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317.

In some embodiments, the Cap polypeptide, e.g., VP1 polypeptide, further comprises an additional alteration (e.g., a substitution, insertion, or deletion) relative to a wildtype sequence (e.g., SEQ ID NO: 321) in addition to any amino acid change resulting from the presence of the exogenous start codon in the ORF encoding MAAP and any alteration present relative to a wildtype sequence in SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317. In some embodiments, the additional alteration improves a production characteristic of a dependoparvovirus particle or method of making the same. In some embodiments, the additional alteration improves or alters another characteristic of a dependoparvovirus particle, e.g., tropism.

In some embodiments, the exogenous start codon does not result in an amino acid change in a VP1 polypeptide encoded by a sequence of the nucleic acid.

### Dependoparvovirus Particles

The disclosure is directed, in part, to a dependoparvovirus particle (e.g., a functional dependoparvovirus particle) comprising a nucleic acid or polypeptide described herein or produced by a method described herein.

Dependoparvovirus is a single-stranded DNA parvovirus that grows only in cells in which certain functions are provided, e.g., by a co-infecting helper virus. Several species of dependoparvovirus are known, including dependoparvovirus A and dependoparvovirus B, which include serotypes known in the art as adeno-associated viruses (AAV). At least thirteen serotypes of AAV that have been characterized. General information and reviews of AAV can be found in, for example, Carter, Handbook of Parvoviruses, Vol. 1, pp. 169-228 (1989), and Berns, Virology, pp. 1743-1764, Raven Press, (New York, 1990). AAV serotypes, and to a degree, dependoparvovirus species, are significantly interrelated structurally and functionally. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins. In addition, heteroduplex analysis reveals extensive cross-hybridization between serotypes along the length of the genome, further suggesting interrelatedness. Dependoparvoviruses genomes also comprise self-annealing segments at the termini that correspond to “inverted terminal repeat sequences” (ITRs).

The genomic organization of naturally occurring dependoparvoviruses, e.g., AAV serotypes, is very similar. For example, the genome of AAV is a linear, single-stranded DNA molecule that is approximately 5,000 nucleotides (nt) in length or less. Inverted terminal repeats (ITRs) flank the unique coding nucleotide sequences for the non-structural replication (Rep) proteins and the structural capsid (Cap) proteins. Three different viral particle (VP) proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins: Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a dependoparvovirus particle of the disclosure comprises a nucleic acid comprising an ORF encoding MAAP polypeptide comprising an exogenous start codon. In some embodiments, a dependoparvovirus particle of the disclosure does not comprise a nucleic acid comprising an ORF encoding MAAP polypeptide comprising an exogenous start codon, e.g., the particle was made by a cell, cell-free system, or other translation system comprising the nucleic acid comprising an ORF encoding MAAP polypeptide comprising an exogenous start codon. In some embodiments, a dependoparvovirus particle of the disclosure comprises a VP1 polypeptide, wherein the VP1 polypeptide comprises an amino acid change (e.g., relative to a wildtype VP1 amino acid sequence or a reference sequence) corresponding to an exogenous start codon in an ORF encoding a MAAP polypeptide. In some embodiments, a dependoparvovirus particle is produced by a method of making a dependoparvovirus particle described herein.

A dependoparvovirus particle of the disclosure may be a dependoparvovirus A particle. In some embodiments, the dependoparvovirus A particle comprises a nucleic acid comprising a complete dependoparvovirus genome wherein each gene is derived from a dependoparvovirus A gene (e.g., has at least 80, 85, 90, 91, 92, 93, 94, 95, 96, 97, 98, 99, or 100% identity to a naturally occurring dependoparvovirus A sequence). In some embodiments, the dependoparvovirus A particle comprises a nucleic acid comprising a complete dependoparvovirus genome wherein 1, 2, 3, 4, 5, 6, or more protein encoding sequences (e.g., VP1, VP2, VP3, MAAP, AAP, Rep, or X) is derived from a naturally occurring dependoparvovirus A gene (e.g., has at least 80, 85, 90, 91, 92, 93, 94, 95, 96, 97, 98, 99, or 100% identity to a naturally occurring dependoparvovirus A sequence). In some embodiments, the dependoparvovirus A particle comprises a nucleic acid comprising a complete dependoparvovirus genome wherein 1, 2, 3, or more protein encoding sequences (e.g., VP1, VP2, VP3, MAAP, AAP, Rep, or X) is not derived from a naturally occurring dependoparvovirus A gene (e.g., is derived from a different dependoparvovirus species' gene).

A dependoparvovirus particle of the disclosure may be a dependoparvovirus B particle. In some embodiments, the dependoparvovirus B particle comprises a nucleic acid comprising a complete dependoparvovirus genome wherein each gene is derived from a dependoparvovirus B gene (e.g., has at least 80, 85, 90, 91, 92, 93, 94, 95, 96, 97, 98, 99, or 100% identity to a naturally occurring dependoparvovirus B sequence). In some embodiments, the dependoparvovirus B particle comprises a nucleic acid comprising a complete dependoparvovirus genome wherein 1, 2, 3, 4, 5, 6, or more protein encoding sequences (e.g., VP1, VP2, VP3, MAAP, AAP, Rep, or X) is derived from a naturally occurring dependoparvovirus B gene (e.g., has at least 80, 85, 90, 91, 92, 93, 94, 95, 96, 97, 98, 99, or 100% identity to a naturally occurring dependoparvovirus B sequence). In some embodiments, the dependoparvovirus B particle comprises a nucleic acid comprising a complete dependoparvovirus genome wherein 1, 2, 3, or more protein encoding sequences (e.g., VP1, VP2, VP3, MAAP, AAP, Rep, or X) is not derived from a naturally occurring dependoparvovirus B gene (e.g., is derived from a different dependoparvovirus species' gene).

A dependoparvovirus particle of the disclosure may be an AAV5 particle. In some embodiments, the AAV5 particle comprises a nucleic acid comprising a complete dependoparvovirus, e.g., AAV, genome wherein each gene is derived from an AAV5 gene (e.g., has at least 80, 85, 90, 91, 92, 93, 94, 95, 96, 97, 98, 99, or 100% identity to a naturally occurring AAV5 sequence). In some embodiments, the AAV5 particle comprises a nucleic acid comprising a complete dependoparvovirus, e.g., AAV, genome wherein 1, 2, 3, 4, 5, 6, or more protein encoding sequences (e.g., VP1, VP2, VP3, MAAP, AAP, Rep, or X) is derived from a naturally occurring AAV5 gene (e.g., has at least 80, 85, 90, 91, 92, 93, 94, 95, 96, 97, 98, 99, or 100% identity to a naturally occurring AAV5 sequence). In some embodiments, the AAV5 particle comprises a nucleic acid comprising a complete dependoparvovirus, e.g., AAV, genome wherein 1, 2, 3, or more protein encoding sequences (e.g., VP1, VP2, VP3, MAAP, AAP, Rep, or X) is not derived from a naturally occurring AAV5 gene (e.g., is derived from a different dependoparvovirus species' or serotype's gene).

In some embodiments, a dependoparvovirus particle of the disclosure may be of a serotype other than AAV5. As used herein, ‘other than AAV5’ refers to a serotype of any dependoparvovirus species that is not dependoparvovirus B AAV5. Examples of serotypes other than AAV5 include, but are not limited to: AAV1, AAV2, AAV3a, AAV3b, AAV4, AAV6, AAV7, AAV8, AAV9, AAV10, AAV11, AAV12, AAVrh8, AAVrh10, AAVrh12, AAVrh32.33, AAVrh74, AAV-I-587, AAV-588NGR, AAV-MO7A, AAV-MO7T, AAV-MecA, AAV-MecB, rRGD587, AAV-C4, AAV-D10, AAV-SIG, AAV-MTP, AAV-QPE, AAV-VNT, AAV-CNH, AAV-CAP, AAV-EYH, AAV 587MTP, AAV-r3.45, AAV2-LSS, AAV2-PFG, AAV2-PPS, AAV2-TLH, AAV2-GMN, AAV2-7m8, AAV-Kera1, AAV-Kera2, AAV-Kera3, AAV-588Myc, AAV2-Z34C, AAV2.N587_R588insBAP, AAV2 Ald13, DMD4, DMD6, A588-RGD4C, A588-RGD4CGLS, AAV-VTAGRAP, AAV-APVTRPA, AAV-DLSNLTR, AAV-NQVGSWS, AAV-EARVRPP, AAV-NSVSLYT, AAV-LS1, AAV-LS2, AAV-LS3, AAV-LS4, AAV-RGDLGLS, AAV-RGDMSRE, AAV-ESGLSQS, AAV-EYRDSSG, AAV-DLGSARA, AAV-NDVRSAN, AAV-GPQGKNS, AAV-NSSRDLG, AAV-NDVRAVS, AAV-NDVRSAN, AAV-NDVRAVS, AAV-PRSTSDP, AAV-DIIRA, AAV-SYENV, AAV-PENSV, AAV-LSLAS, AAV-NDVWN, AAV-NRTYS, rAAV2-ESGHGYF, AAV-GQHPRPG, AAV-PSVSPRP, AAV2-VNSTRLP, AAV-GQHPR, AAV-LSPVR, AAV-MSSDP, AAV-GARPS, AAV-GNEVL, AAV-KMRPG, AAV 588MTP, rRGD453ko, AAV-MNVRGDL, AAV-ENVRGDL, A520/N584 (RGD), A584-RGD4C, A584-RGD4CALS, AAV-ΔIV-NGR, AAV-PTP, BAP-AAV1, BAP-AAV1, AAV1-RGD, AAV1-RGD/BAP (90/10) (mosaic capsid), Tet1c-AAV1 (mosaic capsid), AAV1.9-3-SKAGRSP, BAP-AAV3, BAP-AAV4, BAP-AAV4, AAV5-7m8, AAV6-RGD, AAV6-RGD-Y705-731F+T492V, AAV6-RGD-Y705-731F+T492V+K531E, AAV2/8-BP2, AAV8-PRSTSDP, AAV8-ESGLSOS, AAV8-VNSTRLP, AAV8-ASSLNIA, AAV8-PSVSPRP, AAV8-GQHPRPG, AAV8-SEGLKNL, AAV8-7m8, AAV-SLRSPPS, AAV-RGDLRVS, AAV9-NDVRAVS, AAV9-PRSTSDP, AAV9-ESGLSOS, AAV-PHP.B, AAV-PHP.A, AAV9-7m8, or AAV9P1. In some embodiments, a serotype other than AAV5 includes a serotype described in Table 4 of Büning, H, and Srivastava, A. Mol Ther Methods Clin Dev. 2019 Jan. 26; 12:248-265. doi: 10.1016/j.omtm.2019.01.008. eCollection 2019 Mar. 15, which is hereby incorporated by reference.

A dependoparvovirus particle of the disclosure may be an AAV9 particle. In some embodiments, the AAV9 particle comprises a nucleic acid comprising a complete dependoparvovirus, e.g., AAV, genome wherein each gene is derived from an AAV9 gene (e.g., has at least 80, 85, 90, 91, 92, 93, 94, 95, 96, 97, 98, 99, or 100% identity to a naturally occurring AAV9 sequence). In some embodiments, the AAV9 particle comprises a nucleic acid comprising a complete dependoparvovirus, e.g., AAV, genome wherein 1, 2, 3, 4, 5, 6, or more protein encoding sequences (e.g., VP1, VP2, VP3, MAAP, AAP, Rep, or X) is derived from a naturally occurring AAV9 gene (e.g., has at least 80, 85, 90, 91, 92, 93, 94, 95, 96, 97, 98, 99, or 100% identity to a naturally occurring AAV9 sequence). In some embodiments, the AAV9 particle comprises a nucleic acid comprising a complete dependoparvovirus, e.g., AAV, genome wherein 1, 2, 3, or more protein encoding sequences (e.g., VP1, VP2, VP3, MAAP, AAP, Rep, or X) is not derived from a naturally occurring AAV9 gene (e.g., is derived from a different dependoparvovirus species' or serotype's gene).

A dependoparvovirus particle of the disclosure may be an AAV2 particle. In some embodiments, the AAV2 particle comprises a nucleic acid comprising a complete dependoparvovirus, e.g., AAV, genome wherein each gene is derived from an AAV2 gene (e.g., has at least 80, 85, 90, 91, 92, 93, 94, 95, 96, 97, 98, 99, or 100% identity to a naturally occurring AAV2 sequence). In some embodiments, the AAV2 particle comprises a nucleic acid comprising a complete dependoparvovirus, e.g., AAV, genome wherein 1, 2, 3, 4, 5, 6, or more protein encoding sequences (e.g., VP1, VP2, VP3, MAAP, AAP, Rep, or X) is derived from a naturally occurring AAV2 gene (e.g., has at least 80, 85, 90, 91, 92, 93, 94, 95, 96, 97, 98, 99, or 100% identity to a naturally occurring AAV2 sequence). In some embodiments, the AAV2 particle comprises a nucleic acid comprising a complete dependoparvovirus, e.g., AAV, genome wherein 1, 2, 3, or more protein encoding sequences (e.g., VP1, VP2, VP3, MAAP, AAP, Rep, or X) is not derived from a naturally occurring AAV2 gene (e.g., is derived from a different dependoparvovirus species' or serotype's gene).

A dependoparvovirus particle of the disclosure may comprise a nucleic acid comprising an ORF encoding MAAP polypeptide comprising an exogenous start codon described herein. In some embodiments, the dependoparvovirus particle comprises a nucleic acid comprising a dependoparvovirus genome, and the nucleic acid comprising the dependoparvovirus genome also comprises the ORF encoding MAAP polypeptide comprising an exogenous start codon. In some embodiments, the dependoparvovirus particle comprises a first nucleic acid comprising an ORF encoding MAAP polypeptide comprising an exogenous start codon, and a second nucleic acid comprising one or more components of a dependoparvovirus genome (e.g., the rest of the dependoparvovirus genome), wherein if a MAAP encoding sequence is present in said genome it does not comprise an exogenous start codon.

A dependoparvovirus particle of the disclosure may not comprise a nucleic acid comprising an ORF encoding MAAP polypeptide comprising an exogenous start codon described herein. In some embodiments, a cell, cell free system, or translation system described herein and used to make a dependoparvovirus particle comprises a nucleic acid comprising an ORF encoding MAAP polypeptide comprising an exogenous start codon, but the nucleic acid is not packaged into the dependoparvovirus particle. In some embodiments, the cell, cell free system, or translation system comprises a first nucleic acid comprising a dependoparvovirus genome (e.g., sufficient to promote production of the components of a dependoparvovirus particle) and a second nucleic acid comprising an ORF encoding MAAP polypeptide comprising an exogenous start codon, wherein the first nucleic acid or copies thereof are packaged into a dependoparvovirus particle but the second nucleic acid is not. The first and second nucleic acids may be integrated into the genome of a host cell, disposed on non-genomic nucleic acid (e.g., a vector, e.g., a plasmid), or a combination of both (e.g., the first nucleic acid is disposed on a non-genomic nucleic acid and the second nucleic acid is integrated into the genome of a host cell).

A dependoparvovirus particle of the disclosure may further comprise a payload. In some embodiments, a dependoparvovirus particle can be used to deliver a payload to a target cell, e.g., in a subject, e.g., a human subject. In some embodiments, delivery of the payload treats a disease or condition in a subject. In some embodiments, delivery of the payload modifies the target cell, e.g., modifies expression of one or more genes in the target cell. In some embodiments, the payload is a therapeutic product, e.g., a product described herein. In some embodiments, the payload is selected from any of: a nucleic acid (e.g., DNA or RNA, e.g., mRNA, siRNA, iRNA, miRNA, piRNA, gRNA, or a sequence encoding the same), a polypeptide, a lipid, or a small molecule (e.g., a drug product). In some embodiments, the payload is a nucleic acid and the payload integrates into a target cell genome. In some embodiments, the payload comprises a sequence encoding a polypeptide product, e.g., a therapeutic polypeptide.

In some embodiments, a dependoparvovirus particle comprises a dependoparvovirus capsid and a nucleic acid (e.g., comprising a dependoparvovirus genome). In some embodiments, the dependoparvovirus capsid comprises one or more polypeptide products of the Cap gene. In some embodiments, the dependoparvovirus capsid comprises a VP1 polypeptide. In some embodiments, the dependoparvovirus capsid comprises a VP2 polypeptide. In some embodiments, the dependoparvovirus capsid comprises a VP3 polypeptide. In some embodiments, the dependoparvovirus capsid comprises a VP1 polypeptide and a VP2 polypeptide. In some embodiments, the dependoparvovirus capsid comprises a VP1 polypeptide, a VP2 polypeptide, and a VP3 polypeptide. In some embodiments, the dependoparvovirus capsid does not comprise a VP3 polypeptide.

Without wishing to be bound by theory, it is thought that a method of making a dependoparvovirus particle described herein may produce a dependoparvovirus particle comprising a dependoparvovirus capsid wherein the ratio of VP1 polypeptide to VP2 polypeptide (and optionally to VP3 polypeptide) is altered relative to a dependoparvovirus particle produced by a method or cell, cell free system, or translation system not utilizing a nucleic acid comprising an ORF encoding MAAP polypeptide comprising an exogenous start codon. It is thought that the presence of a mutant MAAP polypeptide, e.g., a MAAP polypeptide comprising a mutation corresponding to the presence of an exogenous start codon in the ORF encoding the MAAP polypeptide, may alter the ratio of VP1, VP2, and optionally VP3 polypeptide present in a dependoparvovirus capsid produced a cell, cell-free system, or translation system. Thus this alteration to the ratio of VP1 polypeptide to VP2 polypeptide (and optionally to VP3 polypeptide) is thought to occur in a mutant MAAP polypeptide dependent fashion.

In some embodiments, the ratio of VP1 polypeptide to VP2 polypeptide in a dependoparvovirus capsid of a dependoparvovirus particle described herein is greater than the ratio in a reference particle, wherein the production of the reference particle was mediated by a wild type MAAP polypeptide (e.g., a MAAP polypeptide encoded by an ORF not comprising an exogenous start codon). In some embodiments, the ratio of VP1 polypeptide to VP2 polypeptide in a dependoparvovirus capsid of a dependoparvovirus particle described herein is at least 10, 20, 30, 40, 50, 60, 70, 80, 90, or 100% greater than the ratio in a reference particle, wherein the production of the reference particle was mediated by a wild type MAAP polypeptide (e.g., a MAAP polypeptide encoded by an ORF not comprising an exogenous start codon). In some embodiments, the ratio of VP1 polypeptide to VP3 polypeptide in a dependoparvovirus capsid of a dependoparvovirus particle described herein is greater than the ratio in a reference particle, wherein the production of the reference particle was mediated by a wild type MAAP polypeptide (e.g., a MAAP polypeptide encoded by an ORF not comprising an exogenous start codon). In some embodiments, the ratio of VP1 polypeptide to VP3 polypeptide in a dependoparvovirus capsid of a dependoparvovirus particle described herein is at least 10, 20, 30, 40, 50, 60, 70, 80, 90, or 100% greater than the ratio in a reference particle, wherein the production of the reference particle was mediated by a wild type MAAP polypeptide (e.g., a MAAP polypeptide encoded by an ORF not comprising an exogenous start codon).

In some embodiments, the ratio of VP1 polypeptide to VP2 polypeptide in a dependoparvovirus capsid of a dependoparvovirus particle described herein is greater than 1.1:1, 1.2:1, 1.3:1, 1.4:1, 1.5:1, 1.6:1, 1.7:1, 1.8:1, 1.9:1, 2:1, 2.1:1, 2.2:1, or 2.3:1 (and optionally no more than 2.3:1). In some embodiments, the ratio of VP1 polypeptide to VP2 polypeptide in a dependoparvovirus capsid of a dependoparvovirus particle described herein is between 1.2:1 and 2:1, 1.4:1 and 2:1, 1.6:1 and 2:1, 1.8:1 and 2:1, 1.2:1 and 1.8:1, 1.4:1 and 1.8:1, 1.6:1 and 1.8:1, 1.2:1 and 1.6:1, 1.4:1 and 1.6:1, or 1.2:1 and 1.4:1. In some embodiments, the ratio of VP1 polypeptide to VP2 polypeptide in a dependoparvovirus capsid of a dependoparvovirus particle described herein is about 1.2:1, 1.5:1, or 2:1, e.g., 1.2:1, 1.5:1, or 2:1.

In some embodiments, the ratio of VP1 polypeptide to VP3 polypeptide in a dependoparvovirus capsid of a dependoparvovirus particle described herein is greater than 1.1:1, 1.2:1, 1.3:1, 1.4:1, 1.5:1, 1.6:1, 1.7:1, 1.8:1, 1.9:1, 2:1, 2.1:1, 2.2:1, or 2.3:1 (and optionally no more than 2.3:1). In some embodiments, the ratio of VP1 polypeptide to VP3 polypeptide in a dependoparvovirus capsid of a dependoparvovirus particle described herein is between 1.2:1 and 2:1, 1.4:1 and 2:1, 1.6:1 and 2:1, 1.8:1 and 2:1, 1.2:1 and 1.8:1, 1.4:1 and 1.8:1, 1.6:1 and 1.8:1, 1.2:1 and 1.6:1, 1.4:1 and 1.6:1, or 1.2:1 and 1.4:1. In some embodiments, the ratio of VP1 polypeptide to VP3 polypeptide in a dependoparvovirus capsid of a dependoparvovirus particle described herein is about 1.2:1, 1.5:1, or 2:1, e.g., 1.2:1, 1.5:1, or 2:1.

In some embodiments, the ratio of VP1 polypeptide:VP2 polypeptide:VP3 polypeptide in a dependoparvovirus capsid of a dependoparvovirus particle described herein is 1:1:X, wherein X is less than 10, 9, 8, 7, 6, 5, 4, 3, 2, or 1 (e.g., less than 8). In some embodiments, VP3 polypeptide is not present in the dependoparvovirus capsid of a dependoparvovirus particle described herein.

In some embodiments, the VP3 polypeptide encoding sequence used by a cell, cell-free system, or translation system to make a dependoparvovirus particle described herein does not comprise a mutation that decreases or abrogates the expression of the VP3 polypeptide (e.g., relative to a reference dependoparvovirus VP3 encoding sequence). In some embodiments, the ratio of VP1, VP2, and VP3 polypeptides is altered in a mutant MAAP polypeptide dependent fashion or dependent upon the exogenous start codon in the ORF encoding MAAP (e.g., and not by a mutation to the VP3 polypeptide encoding sequence itself).

In some embodiments, the VP2 polypeptide encoding sequence used by a cell, cell-free system, or translation system to make a dependoparvovirus particle described herein does not comprise a mutation that decreases or abrogates the expression of the VP2 polypeptide (e.g., relative to a reference dependoparvovirus VP2 encoding sequence). In some embodiments, the ratio of VP1, VP2, and optionally VP3 polypeptides is altered in a mutant MAAP polypeptide dependent fashion or dependent upon the exogenous start codon in the ORF encoding MAAP (e.g., and not by a mutation to the VP2 polypeptide encoding sequence itself).

### Production Characteristics

The disclosure is directed, in part, to nucleic acids, polypeptides, cells, cell free systems, translation systems, viral particles, and methods associated with improved production of dependoparvovirus particles and based upon use of a nucleic acid comprising an ORF encoding MAAP polypeptide comprising an exogenous start codon. In some embodiments, use of a nucleic acid comprising an ORF encoding MAAP polypeptide comprising an exogenous start codon improves a production characteristic of a cell, cell-free system, or other translation system comprising said nucleic acid, a dependoparvovirus particle produced by said cell or system, and/or a method of making a dependoparvovirus utilizing or producing the same (e.g., relative to an otherwise similar cell, system, particle or method not utilizing the nucleic acid).

Production characteristics include, but are not limited to: the amount of a dependoparvovirus polypeptide or particle produced intracellularly, the amount of correctly folded dependoparvovirus polypeptide, the amount of correctly assembled dependoparvovirus capsid, the amount of correctly packaged dependoparvovirus particle, the amount of dependoparvovirus particle secreted from the cell, the overall amount of dependoparvovirus particle produced, or any preceding characteristic relative to a unit of time or resource expended, or any preceding characteristic relative to an otherwise similar cell (e.g., comprising an ORF encoding MAAP not comprising the exogenous start codon).

In some embodiments, a cell, cell-free system, or other translation system, comprising the nucleic acid, or a method of making a dependoparvovirus particle utilizing the same, produces dependoparvovirus particles intracellularly at a level of at least 50, 55, 60, 65, 70, 75, 80, 85, 90, 95, 100, 110, 120, 130, 140, 150, 175, 200, 250, 300, 350, 400, 450, 500, or 1000% the level of an otherwise similar cell, cell-free system, or other translation system (or method utilizing the same) comprising an otherwise similar nucleic acid that does not comprise the exogenous start codon.

In some embodiments, a cell, cell-free system, or other translation system, comprising the nucleic acid, or a method of making a dependoparvovirus particle utilizing the same, produces dependoparvovirus polypeptides (e.g., Cap, Rep, VP1, VP2, or VP3) at a level of at least 50, 55, 60, 65, 70, 75, 80, 85, 90, 95, 100, 110, 120, 130, 140, 150, 175, 200, 250, 300, 350, 400, 450, 500, or 1000% the level of an otherwise similar cell, cell-free system, or other translation system (or method utilizing the same) comprising an otherwise similar nucleic acid that does not comprise the exogenous start codon.

In some embodiments, a cell, cell-free system, or other translation system, comprising the nucleic acid, or a method of making a dependoparvovirus particle utilizing the same, produces correctly folded dependoparvovirus polypeptides (e.g., Cap, Rep, VP1, VP2, or VP3) at a level of at least 50, 55, 60, 65, 70, 75, 80, 85, 90, 95, 100, 110, 120, 130, 140, 150, 175, 200, 250, 300, 350, 400, 450, 500, or 1000% the level of an otherwise similar cell, cell-free system, or other translation system (or method utilizing the same) comprising an otherwise similar nucleic acid that does not comprise the exogenous start codon. In some embodiments, correctly folded means a native, wildtype or wildtype-like conformation, e.g., a stable and/or functional conformation.

In some embodiments, a cell, cell-free system, or other translation system, comprising the nucleic acid, or a method of making a dependoparvovirus particle utilizing the same, produces correctly assembled dependoparvovirus capsids at a level of at least 50, 55, 60, 65, 70, 75, 80, 85, 90, 95, 100, 110, 120, 130, 140, 150, 175, 200, 250, 300, 350, 400, 450, 500, or 1000% the level of an otherwise similar cell, cell-free system, or other translation system (or method utilizing the same) comprising an otherwise similar nucleic acid that does not comprise the exogenous start codon. In some embodiments, correctly assembled means that the capsid assumes a stable structure and/or is functional (e.g., competent for packaging, secretion, and/or infection).

In some embodiments, a cell, cell-free system, or other translation system, comprising the nucleic acid, or a method of making a dependoparvovirus particle utilizing the same, produces correctly packaged dependoparvovirus particles at a level of at least 50, 55, 60, 65, 70, 75, 80, 85, 90, 95, 100, 110, 120, 130, 140, 150, 175, 200, 250, 300, 350, 400, 450, 500, or 1000% the level of an otherwise similar cell, cell-free system, or other translation system (or method utilizing the same) comprising an otherwise similar nucleic acid that does not comprise the exogenous start codon. In some embodiments, correctly packaged means the dependoparvovirus particle comprises a nucleic acid (e.g., comprising a dependoparvovirus genome and/or a payload), has a stable structure and/or is functional (e.g., competent secretion, and/or infection).

In some embodiments, a cell, cell-free system, or other translation system, comprising the nucleic acid, or a method of making a dependoparvovirus particle utilizing the same, secretes dependoparvovirus particles at a level of at least 50, 55, 60, 65, 70, 75, 80, 85, 90, 95, 100, 110, 120, 130, 140, 150, 175, 200, 250, 300, 350, 400, 450, 500, or 1000% the level of an otherwise similar cell, cell-free system, or other translation system (or method utilizing the same) comprising an otherwise similar nucleic acid that does not comprise the exogenous start codon.

In some embodiments, a cell, cell-free system, or other translation system, comprising the nucleic acid, or a method of making a dependoparvovirus particle utilizing the same, produces dependoparvovirus particles (e.g., functional dependoparvovirus particles) at a level of at least 50, 55, 60, 65, 70, 75, 80, 85, 90, 95, 100, 110, 120, 130, 140, 150, 175, 200, 250, 300, 350, 400, 450, 500, or 1000% the level of an otherwise similar cell, cell-free system, or other translation system (or method utilizing the same) comprising an otherwise similar nucleic acid that does not comprise the exogenous start codon.

Dependoparvovirus variants (e.g., comprising an exogenous start codon in an ORF encoding MAAP) can be characterized by their production efficiency in a cell, cell free system, or other translation system. Production efficiency, as used herein, refers to the abundance of a packaged dependoparvovirus particle, e.g., in a purified viral library. In some embodiments, the production efficiency is given relative to the abundance of a variant in a plasmid library. In some embodiments, abundance is determined by measuring the abundance of packaged dependoparvovirus genomes or of packaged payloads, e.g., by sequencing. In some embodiments, the log (e.g., log 2) of the production efficiency is calculated as the log (e.g., log 2) of the ratio of the production efficiency of a dependoparvovirus particle variant comprising an alteration (e.g., an exogenous start codon in an ORF encoding MAAP) to the production efficiency of an otherwise similar dependoparvovirus particle not comprising the alteration (e.g., wildtype AAV5). In some embodiments, a cell, cell-free system, or other translation system, comprising the nucleic acid, or a method of making a dependoparvovirus particle utilizing the same, has a log 2(production efficiency) value, e.g., log 2(production efficiency relative to AAV5) value, that indicates an increase in production efficiency relative to an otherwise similar cell, cell-free system, or other translation system (or method utilizing the same) comprising an otherwise similar nucleic acid that does not comprise the exogenous start codon. In some embodiments, the log 2(production efficiency) value, e.g., log 2(production efficiency relative to AAV5) value, is at least 1.5, 1.6, 1.7, 1.8, 1.9, 2, 2.5, 3, 3.5, 4, 4.5, 5, 5.5, 6, 6.5, 7, 7.5, 8, 8.5, or 9.

In some embodiments, the level change is relative to a unit time (e.g., minutes, hours, days, weeks, production cycles, cell divisions, or culture media turnovers) expended. In some embodiments, the level change is relative to a unit of resource expended (e.g., media consumed, nutrients consumed, cells utilized, energy expended (e.g., to operate a bioreactor), or reagent consumed).

In some embodiments, changes (e.g., improvements) in a production characteristic are dependent upon the dependoparvovirus clade, species, or serotype of the ORF encoding MAAP. The disclosure is based, in part, on the discovery that some naturally occurring ORFs encoding MAAP comprise a non-canonical start codon or no discernable start codon proximal to the beginning of the MAAP encoding sequence. Without wishing to be bound by theory, it is thought that introducing an exogenous start codon, e.g., that is stronger than and/or replaces a weaker non-canonical start codon that might be present proximal to the beginning of the MAAP encoding sequence, increases MAAP expression and improves one or more production characteristics of a cell, cell-free system, other translation system, or a method for making a dependoparvovirus particle. Without wishing to be bound by theory, the expression of a dependoparvovirus ORF encoding MAAP which already comprises a strong (e.g., canonical) endogenous start codon proximal to the start of the MAAP encoding sequence may not increase, e.g., substantially increase, from introduction of an exogenous start codon. However, this in no way limits the type of dependoparvovirus particles which may benefit from application of the improved production characteristics associated with a nucleic acid comprising an ORF encoding a MAAP polypeptide comprising an exogenous start codon. As described herein, a cell, cell-free system, or other translation system may comprise a nucleic acid comprising an ORF encoding a MAAP polypeptide comprising an exogenous start codon and be used to make a dependoparvovirus particle that does not comprise said nucleic acid.

In some embodiments, a nucleic acid comprises an ORF encoding a MAAP polypeptide comprising an exogenous start codon, wherein the ORF encoding the MAAP polypeptide comprises a non-canonical, e.g., weak, start codon or no discernable start codon proximal to the beginning of the MAAP polypeptide encoding sequence. In some embodiments, a weak start codon is a start codon that promotes translation initiation less strongly than an ATG positioned similarly in an otherwise similar sequence. In some embodiments, a weak start codon is a start codon that promotes translation initiation less strongly than a CTG positioned similarly in an otherwise similar sequence.

### Methods of Making Compositions Described Herein

The disclosure is directed, in part, to a method of making a dependoparvovirus particle, e.g., a dependoparvovirus particle described herein. In some embodiments, a method of making dependoparvovirus particle comprises providing a cell, cell-free system, or other translation system, comprising a nucleic acid described herein (e.g., a nucleic acid comprising an ORF encoding a functional MAAP polypeptide comprising an exogenous start codon); and cultivating the cell, cell-free system, or other translation system under conditions suitable for the production of the dependoparvovirus particle, thereby making the dependoparvovirus particle.

The disclosure is based, in part, on the discovery that a method of making a dependoparvovirus particle utilizing a cell, cell-free system, or other translation system comprising a nucleic acid comprising an ORF encoding a functional MAAP polypeptide comprising an exogenous start codon may have one or more improved production characteristics relative to an otherwise similar method utilizing an otherwise similar a cell, cell-free system, or other translation system that lacks the ORF encoding a functional MAAP polypeptide comprising an exogenous start codon. In some embodiments, a method of making a dependoparvovirus particle described herein exhibit an improvement in a production characteristic described herein.

In some embodiments, providing a cell comprising a nucleic acid described herein comprises introducing the nucleic acid to the cell, e.g., transfecting or transforming the cell with the nucleic acid. The nucleic acids of the disclosure may be situated as a part of any genetic element (vector) which may be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon. Such a vector may be delivered by any suitable method, including transfection, liposome delivery, electroporation, membrane fusion techniques, viral infection, high velocity DNA-coated pellets, and protoplast fusion. A person of skill in the art possesses the knowledge and skill in nucleic acid manipulation to construct any embodiment of this invention and said skills include genetic engineering, recombinant engineering, and synthetic techniques. See, e.g., Sambrook et al, Molecular Cloning: A Laboratory Manual, Cold Spring Harbor Press, Cold Spring Harbor, NY.

In some embodiments, a vector of the disclosure comprises sequences encoding a dependoparvovirus capsid or a fragment thereof. In some embodiments, a vectors of the disclosure comprises sequences encoding a dependoparvovirus rep protein or a fragment thereof. In some embodiments, such vectors may contain both dependoparvovirus cap and rep proteins. In vectors in which both AAV rep and cap are provided, the dependoparvovirus rep and dependoparvovirus cap sequences may both be of the same dependoparvovirus species or serotype origin. Alternatively, the present invention provides vectors in which the rep sequences are from a dependoparvovirus species or serotype which differs from that which is providing the cap sequences. In some embodiments, the rep and cap sequences are expressed from separate sources (e.g., separate vectors, or a host cell genome and a vector). In some embodiments, the rep sequences are fused in frame to cap sequences of a different dependoparvovirus species or serotype to form a chimeric dependoparvovirus vector. In some embodiments, the vectors of the invention further contain a payload, e.g., a minigene comprising a selected transgene, e.g., flanked by dependoparvovirus 5′ ITR and dependoparvovirus 3′ ITR.

The vectors described herein, e.g., a plasmid, are useful for a variety of purposes, but are particularly well suited for use in production of recombinant dependoparvovirus particles comprising dependoparvovirus sequences or a fragment thereof, and in some embodiments, a payload.

In one aspect, the disclosure provides a method of making a dependoparvovirus particle (e.g., a dependoparvovirus B particle, e.g., an AAV5 particle), or a portion thereof. In some embodiments, the method comprises culturing a host cell which contains a nucleic acid sequence encoding a dependoparvovirus capsid protein, or fragment thereof, as defined herein; a functional rep gene; a payload, e.g., a minigene comprising dependoparvovirus inverted terminal repeats (ITRs) and a transgene; and sufficient helper functions to promote packaging of the payload, e.g., minigene, into the dependoparvovirus capsid. The components necessary to be cultured in the host cell to package a payload, e.g., minigene, in a dependoparvovirus capsid may be provided to the host cell in trans. In some embodiments, any one or more of the required components (e.g., payload (e.g., minigene), rep sequences, cap sequences, and/or helper functions) may be provided by a host cell which has been engineered to stably comprise one or more of the required components using methods known to those of skill in the art. In some embodiments, a host cell which has been engineered to stably comprise the required component(s) comprises it under the control of an inducible promoter. In some embodiments, the required component may be under the control of a constitutive promoter. Examples of suitable inducible and constitutive promoters are provided herein and further examples are known to those of skill in the art. In some embodiments, a selected host cell which has been engineered to stably comprise one or more components may comprise a component under the control of a constitutive promoter and another component under the control of one or more inducible promoters. For example, a host cell which has been engineered to stably comprise the required components may be generated from 293 cells (e.g., which comprise helper functions under the control of a constitutive promoter), which comprises the rep and/or cap proteins under the control of one or more inducible promoters.

The payload (e.g., minigene), rep sequences, cap sequences, and helper functions required for producing a dependoparvovirus particle of the disclosure may be delivered to the packaging host cell in the form of any genetic element which transfers the sequences carried thereon (e.g., in a vector or combination of vectors). The genetic element may be delivered by any suitable method, including those described herein. Methods used to construct genetic elements, vectors, and other nucleic acids of the disclosure are known to those with skill and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, J. Virol, 70:520-532 (1993) and U.S. Pat. No. 5,478,745. Unless otherwise specified, the dependoparvovirus ITRs, and other selected dependoparvovirus components described herein, may be readily selected from among any dependoparvovirus species and serotypes, e.g., AAV1, AAV2, AAV3, AAV4, AAV5, AAV6, AAV7, AAV9. ITRs or other dependoparvovirus components may be readily isolated using techniques available to those of skill in the art from a dependoparvovirus species or serotype. Dependoparvovirus species and serotypes may be isolated or obtained from academic, commercial, or public sources (e.g., the American Type Culture Collection, Manassas, VA). In some embodiments, the dependoparvovirus sequences may be obtained through synthetic or other suitable means by reference to published sequences such as are available in the literature or in databases such as, e.g., GenBank or PubMed.

The dependoparvovirus particles comprising nucleic acids (e.g., including a payload) of the disclosure may be produced using any invertebrate cell type which allows for production of dependoparvovirus or biologic products and which can be maintained in culture. In some embodiments, an insect cell may be used in production of the compositions described herein or in the methods of making a dependoparvovirus particle described herein. For example, an insect cell line used can be from Spodoptera frugiperda, such as Sf9, SF21, SF900+, drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. In some embodiments, the insect cells are susceptible to baculovirus infection, including High Five, Sf9, Se301, SeIZD2109, SeUCR1, SP900+, Sf21, BTI-TN-5B1-4, MG-1, Tn368, HzAml, BM-N, Ha2302, Hz2E5 and Ao38.

In another aspect, the methods of the disclosure can be carried out with any mammalian cell type which allows for replication of dependoparvovirus or production of biologic products, and which can be maintained in culture. In some embodiments, the mammalian cells used can be HEK293, HeLa, CHO, NS0, SP2/0, PER.C6, Vero, RD, BHK, HT 1080, A549, Cos-7, ARPE-19 or MRC-5 cells.

Methods of expressing proteins (e.g., recombinant or heterologous proteins, e.g., dependoparvovirus polypeptides) in insect cells are well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); and Samulski et al., U.S. Pat. No. 6,204,059. In some embodiments, a nucleic acid construct encoding dependoparvovirus polypeptides (e.g., a dependoparvovirus genome) in insect cells is an insect cell-compatible vector. An “insect cell-compatible vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cell's genome or remain present extra-chromosomally. The vector may be present permanently or transiently, e.g., as an episomal vector. Vectors may be introduced by any means known in the art. Such means include but are not limited to chemical treatment of the cells, electroporation, or infection. In some embodiments, the vector is a baculovirus, a viral vector, or a plasmid.

In some embodiments, a nucleic acid sequence encoding an dependoparvovirus polypeptide is operably linked to regulatory expression control sequences for expression in a specific cell type, such as Sf9 or HEK cells. Techniques known to one skilled in the art for expressing foreign genes in insect host cells or mammalian host cells can be used with the compositions and methods of the disclosure. Methods for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. Promoters suitable for transcription of a nucleotide sequence encoding a dependoparvovirus polypeptide include the polyhedron, p10, p35 or IE-1 promoters and further promoters described in the above references are also contemplated.

In some embodiments, providing a cell comprising a nucleic acid described herein comprises acquiring a cell comprising the nucleic acid.

Methods of cultivating cells, cell-free systems, and other translation systems are known to those of skill in the art. In some embodiments, cultivating a cell comprises providing the cell with suitable media and incubating the cell and media for a time suitable to achieve viral particle production.

In some embodiments, a method of making a dependoparvovirus particle further comprises a purification step comprising isolating the dependoparvovirus particle from one or more other components (e.g., from a cell or media component).

In some embodiments, production of the dependoparvovirus particle comprises one or more (e.g., all) of: expression of dependoparvovirus polypeptides, assembly of a dependoparvovirus capsid, expression (e.g., duplication) of a dependoparvovirus genome, and packaging of the dependoparvovirus genome into the dependoparvovirus capsid to produce a dependoparvovirus particle. In some embodiments, production of the dependoparvovirus particle further comprises secretion of the dependoparvovirus particle.

In some embodiments, and as described elsewhere herein, the nucleic acid comprising an ORF encoding a functional MAAP polypeptide comprising an exogenous start codon is disposed in a dependoparvovirus genome. In some embodiments, the nucleic acid comprising an ORF encoding a functional MAAP polypeptide comprising an exogenous start codon is packaged into a dependoparvovirus particle along with the dependoparvovirus genome as part of a method of making a dependoparvovirus particle described herein. In other embodiments, the nucleic acid comprising an ORF encoding a functional MAAP polypeptide comprising an exogenous start codon is not packaged into a dependoparvovirus particle made by a method described herein.

In some embodiments, a method of making a dependoparvovirus particle described herein produces a dependoparvovirus particle comprising a payload (e.g., a payload described herein). In some embodiments, the payload comprises a second nucleic acid (e.g., in addition to the dependoparvovirus genome), and production of the dependoparvovirus particle comprises packaging the second nucleic acid into the dependoparvovirus particle. In some embodiments, a cell, cell-free system, or other translation system for use in a method of making a dependoparvovirus particle comprises the second nucleic acid. In some embodiments, the second nucleic acid comprises an exogenous sequence (e.g., exogenous to the dependoparvovirus, the cell, or to a target cell or subject who will be administered the dependoparvovirus particle). In some embodiments, the exogenous sequence encodes an exogenous polypeptide. In some embodiments, the exogenous sequence encodes a therapeutic product.

The disclosure is based, in part, on the discovery that a method of making a dependoparvovirus particle utilizing a cell, cell-free system, or other translation system comprising a nucleic acid comprising an ORF encoding a functional MAAP polypeptide comprising an exogenous start codon may have one or more improved production characteristics relative to an otherwise similar method utilizing an otherwise similar a cell, cell-free system, or other translation system that lacks the ORF encoding a functional MAAP polypeptide comprising an exogenous start codon. In some embodiments, a method of making a dependoparvovirus particle described herein exhibit an improvement in a production characteristic described herein.

In some embodiments, a nucleic acid or polypeptide described herein is produced by a method known to one of skill in the art. The nucleic acids, polypeptides, and fragments thereof of the disclosure may be produced by any suitable means, including recombinant production, chemical synthesis, or other synthetic means. Such production methods are within the knowledge of those of skill in the art and are not a limitation of the present invention.

### Applications

The disclosure is directed, in part, to compositions comprising a nucleic acid, polypeptide, or particles described herein. The disclosure is further directed, in part, to methods utilizing a composition, nucleic acid, polypeptide, or particles described herein. As will be apparent based on the disclosure, nucleic acids, polypeptides, particles, and methods disclosed herein have a variety of utilities.

The disclosure is directed, in part, to a vector comprising a nucleic acid described herein, e.g., a nucleic acid comprising an ORF encoding a functional MAAP polypeptide comprising an exogenous start codon. Many types of vectors are known to those of skill in the art. In some embodiments, a vector comprises a plasmid. In some embodiments, the vector is an isolated vector, e.g., removed from a cell or other biological components.

The disclosure is directed, in part to a cell, cell-free system, or other translation system, comprising a nucleic acid or vector described herein, e.g., a nucleic acid or vector comprising an ORF encoding a functional MAAP polypeptide comprising an exogenous start codon. In some embodiments, the cell, cell-free system, or other translation system is capable of producing dependoparvovirus particles. In some embodiments, the cell, cell-free system, or other translation system comprises a nucleic acid comprising a dependoparvovirus genome or components of a dependoparvovirus genome sufficient to promote production of dependoparvovirus particles. In some embodiments, the cell, cell-free system, or other translation system has one or more improved production characteristics, e.g., by virtue of the ORF encoding a functional MAAP polypeptide comprising an exogenous start codon. In some embodiments, cell, cell-free system, or other translation system comprises a dependoparvovirus capsid and/or dependoparvovirus particle (e.g., as described herein).

In some embodiments, the cell, cell-free system, or other translation system further comprises one or more non-dependoparvovirus nucleic acid sequences that promote dependoparvovirus particle production and/or secretion. Said sequences are referred to herein as helper sequences. In some embodiments, a helper sequence comprises one or more genes from another virus, e.g., an adenovirus or herpes virus. In some embodiments, the presence of a helper sequence is necessary for production and/or secretion of a dependoparvovirus particle. In some embodiments, a cell, cell-free system, or other translation system comprises a vector, e.g., plasmid, comprising one or more helper sequences.

In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a helper sequence, and wherein the second nucleic acid comprises a payload. In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a payload, and wherein the second nucleic acid comprises a helper sequence. In some embodiments, a cell, cell-free system, or other translation system comprises a first nucleic acid and a second nucleic acid, wherein the first nucleic acid comprises a helper sequence and a payload, and wherein the second nucleic acid comprises a sequences encoding one or more dependoparvovirus genes (e.g., a Cap gene, a Rep gene, or a complete dependoparvovirus genome). In some embodiments, a cell, cell-free system, or other translation system comprises a first nucleic acid, a second nucleic acid, and a third nucleic acid, wherein the first nucleic acid comprises a sequences encoding one or more dependoparvovirus genes (e.g., a Cap gene, a Rep gene, or a complete dependoparvovirus genome), the second nucleic acid comprises a helper sequence, and the third nucleic acid comprises a payload. In some embodiments, the nucleic acid comprising an ORF encoding MAAP polypeptide comprising an exogenous start codon is part of the sequences encoding one or more dependoparvovirus genes (e.g., a Cap gene, a Rep gene, or a complete dependoparvovirus genome). In some embodiments, the nucleic acid comprising an ORF encoding MAAP polypeptide comprising an exogenous start codon is present as a separate sequence from the sequences encoding one or more dependoparvovirus genes (e.g., a Cap gene, a Rep gene, or a complete dependoparvovirus genome).

In some embodiments, the first nucleic acid, second nucleic acid, and optionally third nucleic acid are situated in separate molecules, e.g., separate vectors or a vector and genomic DNA. In some embodiments, one, two, or all of the first nucleic acid, second nucleic acid, and optionally third nucleic acid are integrated (e.g., stably integrated) into the genome of a cell.

A cell of the disclosure may be generated by transfecting a suitable cell with a nucleic acid described herein. In some embodiments, a method of making a dependoparvovirus particle or improving a method of making a dependoparvovirus particle comprises providing a cell described herein. In some embodiments, providing a cell comprises transfecting a suitable cell with one or more nucleic acids described herein.

Many types and kinds of cells suitable for use with the nucleic acids and vectors described herein are known in the art. In some embodiments, the cell is a human cell. In some embodiments, the cell is an immortalized cell or a cell from a cell line known in the art. In some embodiments, the cell is an HEK293 cell.

Methods of Delivering a Payload

The disclosure is directed, in part, to a method of delivering a payload to a cell, e.g., a cell in a subject or in a sample. In some embodiments, a method of delivering a payload to a cell comprises contacting the cell with a dependoparvovirus particle (e.g., described herein) comprising the payload. In some embodiments, the dependoparvovirus particle is a dependoparvovirus particle described herein and comprises a payload described herein.

In some embodiments, the payload comprises a transgene. In some embodiments, the transgene is a nucleic acid sequence heterologous to the vector sequences flanking the transgene which encodes a polypeptide, RNA (e.g., a miRNA or siRNA) or other product of interest. The nucleic acid of the transgene may be operatively linked to a regulatory component in a manner sufficient to promote transgene transcription, translation, and/or expression in a host cell.

A transgene may be any polypeptide or RNA encoding sequence and the transgene selected will depend upon the use envisioned. In some embodiments, a transgene comprises a reporter sequence, which upon expression produces a detectable signal. Such reporter sequences include, without limitation, DNA sequences encoding colorimetric reporters (e.g., β-lactamase, β-galactosidase (LacZ), alkaline phosphatase), cell division reporters (e.g., thymidine kinase), fluorescent or luminescence reporters (e.g., green fluorescent protein (GFP) or luciferase), resistance conveying sequences (e.g., chloramphenicol acetyltransferase (CAT)), or membrane bound proteins including to which high affinity antibodies directed thereto exist or can be produced by conventional means, e.g., comprising an antigen tag, e.g., hemagglutinin or Myc.

In some embodiments, a reporter sequence operably linked with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. In some embodiments, the transgene encodes a product which is useful in biology and medicine, such as RNA, proteins, peptides, enzymes, dominant negative mutants. In some embodiments, the RNA comprises a tRNA, ribosomal RNA, dsRNA, catalytic RNAs, small hairpin RNA, siRNA, trans-splicing RNA, and antisense RNAs. In some embodiments, the RNA inhibits or abolishes expression of a targeted nucleic acid sequence in a treated subject (e.g., a human or animal subject).

In some embodiments, the transgene may be used to correct or ameliorate gene deficiencies. In some embodiments, gene deficiencies include deficiencies in which normal genes are expressed at less than normal levels or deficiencies in which the functional gene product is not expressed. In some embodiments, the transgene encodes a therapeutic protein or polypeptide which is expressed in a host cell. In some embodiments, a dependoparvovirus particle may comprise or deliver multiple transgenes, e.g., to correct or ameliorate a gene defect caused by a multi-subunit protein. In some embodiments, a different transgene (e.g., each situated/delivered in a different dependoparvovirus particle, or in a single dependoparvovirus particle) may be used to encode each subunit of a protein, or to encode different peptides or proteins, e.g., when the size of the DNA encoding the protein subunit is large, e.g., for immunoglobulin, platelet-derived growth factor, or dystrophin protein. In some embodiments, different subunits of a protein may be encoded by the same transgene, e.g., a single transgene encoding each of the subunits with the DNA for each subunit separated by an internal ribozyme entry site (IRES). In some embodiments, the DNA may be separated by sequences encoding a 2A peptide, which self-cleaves in a post-translational event. See, e.g., Donnelly et al, J. Gen. Virol., 78(Pt 1):13-21 (January 1997); Furler, et al, Gene Ther., 8(11):864-873 (June 2001); Klump et al., Gene Ther 8(10):811-817 (May 2001).

The transgene may encode any biologically active product or other product, e.g., a product desirable for study. Suitable transgenes may be readily selected by persons of skill in the art.

In some embodiments, the transgene is a heterologous protein. In some embodiments, heterologous protein is a therapeutic protein. Exemplary therapeutic proteins include, but are not limited to, colony stimulating factors (CSF); blood factors, such as β-globin, hemoglobin, tissue plasminogen activator, and coagulation factors; interleukins; soluble receptors, such as soluble TNF-α. receptors, soluble VEGF receptors, soluble interleukin receptors (e.g., soluble IL-1 receptors and soluble type II IL-1 receptors), or ligand-binding fragments of a soluble receptor; growth factors, such as keratinocyte growth factor (KGF), stem cell factor (SCF), or fibroblast growth factor (FGF, such as basic FGF and acidic FGF); enzymes; chemokines; enzyme activators, such as tissue plasminogen activator; angiogenic agents, such as vascular endothelial growth factors, glioma-derived growth factor, angiogenin, or angiogenin-2; anti-angiogenic agents, such as a soluble VEGF receptor; a protein vaccine; neuroactive peptides, such as nerve growth factor (NGF) or oxytocin; thrombolytic agents; tissue factors; macrophage activating factors; tissue inhibitors of metalloproteinases; or IL-1 receptor antagonists

The disclosure is further directed, in part, to a method of delivering a payload to a subject, e.g., an animal or human subject. In some embodiments, a method of delivering a payload to a subject comprises administering to the subject a dependoparvovirus particle (e.g., described herein) comprising the payload, e.g., in a quantity and for a time sufficient to deliver the payload. In some embodiments, the dependoparvovirus particle is a dependoparvovirus particle described herein and comprises a payload described herein.

Methods of Improving a Dependoparvovirus Production Process

The disclosure is directed, in part, to a method of improving a dependoparvovirus particle production process (e.g., a method of making a dependoparvovirus particle). In some embodiments, the method of improving a dependoparvovirus particle production process comprises contacting a cell, cell-free system, or translation system with a nucleic acid comprising an ORF encoding a functional MAAP polypeptide comprising an exogenous start codon, thereby improving the dependoparvovirus particle production process. In some embodiments, introducing a nucleic acid comprising an ORF encoding a functional MAAP polypeptide comprising an exogenous start codon into a cell, cell-free system, or translation system used to make dependoparvovirus particles improves one or more production characteristics (e.g., a production characteristic described herein) of the cell, cell-free system, or translation system, or method of making a dependoparvovirus particle utilizing the same.

Methods of Treatment

The disclosure is directed, in part, to a method of treating a disease or condition in a subject, e.g., an animal or human subject. In some embodiments, a method of treating a disease or condition in a subject comprises administering to the subject a dependoparvovirus particle described herein, e.g., comprising a payload described herein. In some embodiments, the dependoparvovirus particle comprising a payload described herein is administered in an amount and/or time effective to treat the disease or condition. In some embodiments, the payload is a therapeutic product. In some embodiments, the payload is a nucleic acid, e.g., encoding an exogenous polypeptide.

The dependoparvovirus particles described herein or produced by the methods described herein can be used to express one or more therapeutic proteins to treat various diseases or disorders. In some embodiments, the disease or disorder is a cancer, e.g., a cancer such as carcinoma, sarcoma, leukemia, lymphoma; or an autoimmune disease, e.g., multiple sclerosis. Non-limiting examples of carcinomas include esophageal carcinoma; bronchogenic carcinoma; colon carcinoma; colorectal carcinoma; gastric carcinoma; hepatocellular carcinoma; basal cell carcinoma, squamous cell carcinoma (various tissues); bladder carcinoma, including transitional cell carcinoma; lung carcinoma, including small cell carcinoma and non-small cell carcinoma of the lung; adrenocortical carcinoma; sweat gland carcinoma; sebaceous gland carcinoma; thyroid carcinoma; pancreatic carcinoma; breast carcinoma; ovarian carcinoma; prostate carcinoma; adenocarcinoma; papillary carcinoma; papillary adenocarcinoma; cystadenocarcinoma; medullary carcinoma; renal cell carcinoma; uterine carcinoma; testicular carcinoma; osteogenic carcinoma; ductal carcinoma in situ or bile duct carcinoma; choriocarcinoma; seminoma; embryonal carcinoma; Wilm's tumor; cervical carcinoma; epithelial carcinoma; and nasopharyngeal carcinoma. Non-limiting examples of sarcomas include fibrosarcoma, myxosarcoma, liposarcoma, angiosarcoma, endotheliosarcoma, lymphangiosarcoma, chondrosarcoma, chordoma, osteogenic sarcoma, osteosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include ependymoma, pinealoma, hemangioblastoma, acoustic neuroma, oligodendroglioma, glioma, astrocytoma, medulloblastoma, craniopharyngioma, menangioma, melanoma, neuroblastoma, and retinoblastoma. Non-limiting examples of leukemias include chronic myeloproliferative syndromes; T-cell CLL prolymphocytic leukemia, acute myelogenous leukemias; chronic lymphocytic leukemias, including B-cell CLL, hairy cell leukemia; and acute lymphoblastic leukemias. Examples of lymphomas include, but are not limited to, B-cell lymphomas, such as Burkitt's lymphoma; and Hodgkin's lymphoma. In some embodiments, the disease or disorder is a genetic disorder. In some embodiments, the genetic disorder is sickle cell anemia, Glycogen storage diseases (GSD, e.g., GSD types I, II, III, IV, V, VI, VII, VIII, IX, X, XI, XII, XIII, and XIV), cystic fibrosis, lysosomal acid lipase (LAL) deficiency 1, Tay-Sachs disease, Phenylketonuria, Mucopolysaccharidoses, Galactosemia, muscular dystrophy (e.g., Duchenne muscular dystrophy), hemophilia such as hemophilia A (classic hemophilia) or hemophilia B (Christmas Disease), Wilson's disease, Fabry Disease, Gaucher Disease hereditary angioedema (HAE), and alpha 1 antitrypsin deficiency.

In some embodiments, administration of a dependoparvovirus particle comprising a payload (e.g., a transgene) to a subject induces expression of the payload (e.g., transgene) in a subject. The amount of a payload, e.g., transgene, e.g., heterologous protein, e.g., therapeutic polypeptide, expressed in a subject (e.g., the serum of the subject) can vary. For example, in some embodiments the payload, e.g., protein or RNA product of a transgene, can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload, e.g., protein or RNA product of a transgene, is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values.

Sequences disclosed herein may be described in terms of percent identity. A person of skill will understand that such characteristics involve alignment of two or more sequences. Alignments may be performed using any of a variety of publicly or commercially available Multiple Sequence Alignment Programs, such as “Clustal W”, accessible via the Internet. As another example, nucleic acid sequences may be compared using FASTA, a program in GCG Version 6.1. FASTA provides alignments and percent sequence identity of the regions of the best overlap between the query and search sequences. For instance, percent identity between nucleic acid sequences may be determined using FASTA with its default parameters as provided in GCG Version 6.1, herein incorporated by reference. Similar programs are available for amino acid sequences, e.g., the “Clustal X” program. Additional sequence alignment tools that may be used are provided by (protein sequence alignment; (Error! Hyperlink reference not valid. and (nucleic acid alignment; http://www“dot”ebi“dot”ac“dot”uk/Tools/psa/emboss_needle/nucleotide“dot”html)). Generally, any of these programs may be used at default settings, although one of skill in the art can alter these settings as needed. Alternatively, one of skill in the art can utilize another algorithm or computer program which provides at least the level of identity or alignment as that provided by the referenced algorithms and programs. Sequences disclosed herein may further be described in terms of edit distance. The minimum number of sequence edits (i.e., additions, substitutions, or deletions of a single base or nucleotide) which change one sequence into another sequence is the edit distance between the two sequences. In some embodiments, the distance between two sequences is calculated as the Levenshtein distance.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 21, 2020. When one gene or protein references a plurality of sequence accession numbers, all of the sequence variants are encompassed.

The invention is further illustrated by the following examples. The examples are provided for illustrative purposes only and are not to be construed as limiting the scope or content of the invention in any way.

### EXAMPLES

### Example 1: Introduction of ATG into AAV5 MAAP Encoding Sequence Improves Viral Particle Packaging

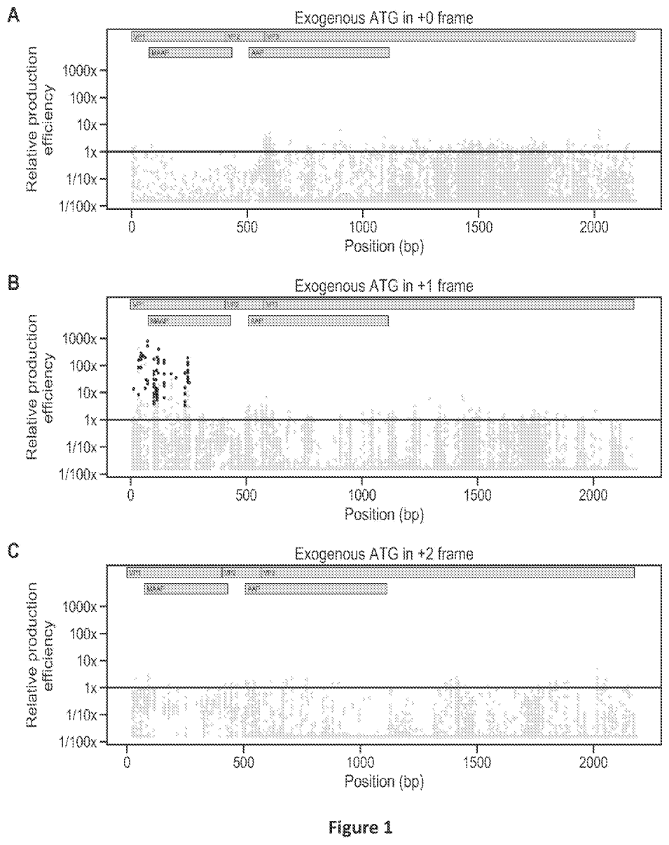

This example describes how introduction of an ATG start codon in the ORF for AAV5 MAAP improved one or more production characteristics, e.g., production of a resulting dependoparvovirus particle. A library of mutant dependoparvovirus B (e.g., AAV5) sequences were generated and tested for changes in one or more production characteristics. Introduction of new +1 frame ATGs proximal to the start of the MAAP encoding sequence resulted in an apparent “superpackager” phenotype characterized by an increased production efficiency. These new +1 ATGs clustered around the start of MAAP, both upstream and downstream. Introduction of new ATGs in other regions or in other frames did not significantly improve production. FIG. 1 shows the production rate for new AAV5 variants that introduce new ATGs.

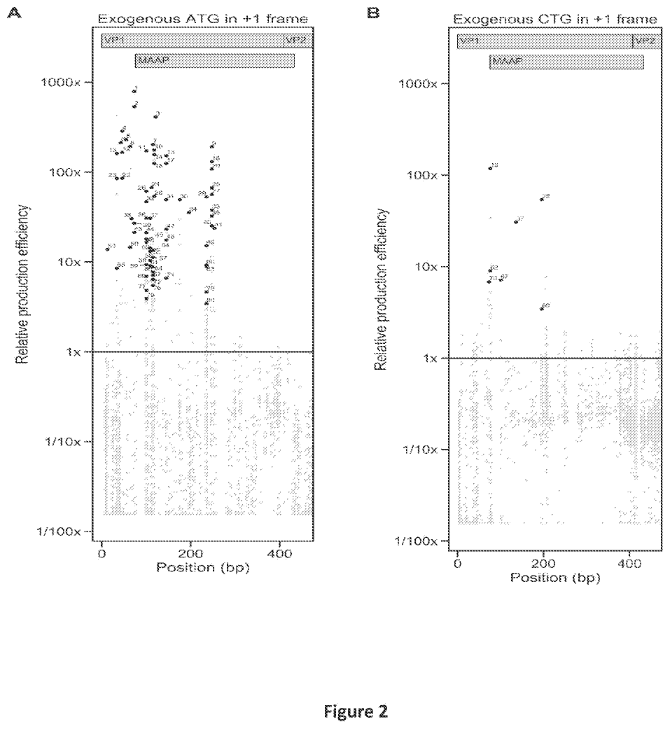

The superpackager phenotype resulted from +1 ATG in or near MAAP, and in particular in the region surrounding the putative beginning of the MAAP encoding sequence (see FIG. 2, graph A for a magnified view of said region). ATGs in other reading frames (the +0 VP1 reading frame or the +2 frame) did not produce superpackager phenotypes. The results show that introduction of new +1 frame exogenous start codons (ATGs) proximal to the start of the putative MAAP encoding sequence resulted in a significant increase in packaging and production efficiency of viral particles.

### Example 2: Introduction of CTG into AAV5 MAAP Encoding Sequence Improves Viral Particle Packaging

This example describes how introduction of a CTG start codon in the ORF for AAV5 MAAP improved one or more production characteristics, e.g., production of a resulting dependoparvovirus particle. The library generated in Example 1 was queried for the effect of CTG introduction in the +1 frame in and around the MAAP encoding sequence of AAV5. Several +1 CTGs improve production of dependoparvovirus particles (FIG. 2). Some CTGs that improved production were located at a position corresponding to the start position of MAAP in other dependoparvovirus serotypes.

The results show that introduction of new +1 frame exogenous start codons (CTGs) proximal to the start of the MAAP encoding sequence resulted in an increase in production efficiency of viral particles.

1. A nucleic acid comprising a sequence encoding an ORF for a functional dependoparvovirus B (e.g., an adeno-associated dependoparvovirus (AAV), e.g. AAV5) MAAP polypeptide, which ORF comprises an exogenous start codon.

2. The nucleic acid of claim 1, wherein a cell, cell-free system, or other translation system, comprising the nucleic acid packages, secretes, and/or produces a dependoparvovirus (e.g., dependoparvovirus A or B, e.g., an adeno-associated dependoparvovirus (AAV), e.g. AAV5 a serotype other than AAV5) particle at a level of at least 50% or more than that of a cell, cell-free system, or other translation system, comprising an otherwise similar nucleic acid that does not comprise the exogenous start codon.

3. The nucleic acid of either of embodiments 1 or 2, wherein the sequence comprises a change or mutation at a position between or including nucleotides 14 to 250 of a VP1 encoding sequence (e.g., a sequence encoding AAV5 VP1, e.g., SEQ ID NO: 327) that creates an exogenous start codon at the position.

4. The nucleic acid of any of claims 1-3, wherein the sequence comprises a change or mutation at any of the positions listed in columns 4 or 5 of Table 1, or at a site one or two nucleotides downstream of said position, that creates an exogenous start codon at the position.

5. The nucleic acid of either of claim 3 or 4, wherein the change or mutation is relative to a reference sequence comprising a wildtype sequence, e.g., SEQ ID NO: 331, or a sequence with at least 90 or 95% sequence identity with a wildtype sequence, e.g., SEQ ID NO: 331.

6. The nucleic acid of any of the above claims, wherein the functional dependoparvovirus B (e.g., AAV5) MAAP polypeptide ORF:
(a) mediates detectable translation initiation in a cell, e.g., a human cell, cell-free system, or other translation system, or
(b) if present in a cell, cell-free system, or other translation system, otherwise competent for producing dependoparvovirus particles, allows for the production of dependoparvovirus particles.

7. The nucleic acid of any of the above claims, wherein the MAAP polypeptide has at least 90% sequence identity to SEQ ID NO: 325.

8. The nucleic acid of any of the above claims, wherein the MAAP polypeptide, except for the amino acid specified by the exogenous start codon, differs from the sequence of SEQ ID NO: 325, by no more than 10 amino acid residues.

9. The nucleic acid of any of the above claims, wherein the exogenous start codon is an ATG or CTG.

10. The nucleic acid of any of the above claims wherein the MAAP polypeptide comprises at least 80, 85, 90, 95, 100, 105, 110, 115, or 116 amino acids (e.g., a full length MAAP polypeptide) and optionally no more than 120, 119, 118, 117, 116, 115, 110, 105, or 100 amino acids.

11. The nucleic acid of any of the above claims, wherein the ORF encoding MAAP comprises a nucleic acid sequence with at least 80, 85, 90, 91, 92, 93, 94, 95, 96, 97, 98, 99, or 100% identity to any of SEQ ID NOs: 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, or 320.

12. The nucleic acid of any of the above claims, wherein the MAAP polypeptide is an AAV5 MAAP polypeptide.

13. The nucleic acid of any of the above claims, further comprising a sequence encoding a dependoparvovirus (e.g., dependoparvovirus B, e.g., an AAV5) VP1 polypeptide.

14. The nucleic acid of claim 13, wherein the VP1 polypeptide has at least 90% sequence identity to SEQ ID NO: 321.

15. The nucleic acid of either claim 13 or 14, wherein the VP1 polypeptide, except for the amino acid specified by the exogenous start codon, differs from the sequence of SEQ ID NO: 321, by no more than 10 amino acid residues.

16. The nucleic acid of any of claims 13-45, wherein the sequence encoding the VP1 polypeptide comprises a nucleic acid sequence with at least 80, 85, 90, 91, 92, 93, 94, 95, 96, 97, 98, 99, or 100% identity to any of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, or 318.

17. The nucleic acid of any of the above claims, further comprising a sequence encoding a dependoparvovirus (e.g., dependoparvovirus B, e.g., AAV5 or a serotype other than AAV5) Cap polypeptide.

18. The nucleic acid of claim 17, wherein the Cap polypeptide has at least 90% sequence identity to SEQ ID NO: 321.

19. The nucleic acid of either of claim 17 or 18, wherein the Cap polypeptide, except for the amino acid specified by the exogenous start codon, differs from the sequence of SEQ ID NO: 321, by no more than 10 amino acid residues.

20. The nucleic acid of any of the above claims, further comprising a sequence encoding a dependoparvovirus (e.g., dependoparvovirus A or B) Rep polypeptide, e.g, encoding an AAV2 or AAV5 Rep polypeptide.

21. The nucleic acid of claim 20, wherein the Rep polypeptide has at least 90% sequence identity to any of SEQ ID NOs: 333-336.

22. The nucleic acid of either of claim 20 or 21, wherein the Rep polypeptide differs from the sequence of any of SEQ ID NOs: 333-336, by no more than 10 amino acid residues.

23. The nucleic acid of any of claims 13-22, wherein one or more or all of the VP1, Cap, or Rep polypeptides is, respectively, an AAV5 VP1, Cap, or Rep polypeptide.

24. The nucleic acid of any of the above claims, further comprising an AAV Cap gene that comprises a sequence encoding VP3, VP2, VP1, AAP, Rep, or X gene that does not naturally occur in an AAV5 genome.

25. The nucleic acid of any of claims 13-24, wherein the polypeptide sequence encoded by the dependoparvovirus Cap gene, e.g., the VP1 polypeptide sequence, comprises a mutation (e.g., a substitution) corresponding to the exogenous start codon in the MAAP polypeptide ORF.

26. The nucleic acid of any of claims 13-24, wherein the polypeptide sequence encoded by the dependoparvovirus Cap gene, e.g., the VP1 polypeptide sequence, does not comprises a mutation (e.g., a substitution) corresponding to the exogenous start codon in the MAAP polypeptide ORF.

27. The nucleic acid of any of claims 13-25, wherein the polypeptide sequence encoded by the dependoparvovirus Cap gene, e.g., the VP1 polypeptide sequence, comprises a mutation corresponding to a difference between any of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 265, 269, 273, 277, 281, 285, 289, 293, 297, 301, 305, 309, 313, or 317, and a wildtype VP1 polypeptide sequence, e.g., SEQ ID NO: 321.

28. The nucleic acid of any of claims 17-27, wherein the polypeptide produced from the Cap gene is functional, e.g., capable of assembling into a dependoparvovirus capsid, capable of packaging dependoparvovirus DNA into a dependoparvovirus capsid, or the dependoparvovirus capsid assembled from the polypeptide produced from the Cap gene is capable of infecting a target cell.

29. The nucleic acid of any of the above claims, wherein a cell, cell-free system, or other translation system, comprising the nucleic acid secretes functional dependoparvovirus particle at a level of at least 60, 70, 80, 90, 100, 110, 120, 130, 140, 150, 160, 170, 180, 190, 200, 250, 300, 350, 400, 450, 500, 600, 700, 800, 900, or 1000% that of a cell, cell-free system, or other translation system, comprising an otherwise similar nucleic acid that does not comprise the exogenous start codon.

30. The nucleic acid of any of the above claims, wherein a cell, cell-free system, or other translation system, comprising the nucleic acid secretes more functional dependoparvovirus particle than a cell, cell-free system, or other translation system, comprising an otherwise similar nucleic acid that does not comprise the exogenous start codon.

31. A dependoparvovirus particle comprising the nucleic acid of any of the above claims.

32. The dependoparvovirus particle of claim 31, wherein the dependoparvovirus particle is an adeno-associated dependoparvovirus (AAV) particle.

33. A vector, e.g., a plasmid, comprising the nucleic acid of any of claims 1-30.

34. A cell, cell-free system, or other translation system, comprising the nucleic acid, vector, or particle of any of claims 1-33.

35. A dependoparvovirus B (e.g., AAV5) MAAP polypeptide, an amino acid of which corresponds to an exogenous start codon.

36. A dependoparvovirus B (e.g., AAV5) MAAP polypeptide encoded by a nucleic acid of any of claims 1-30.

37. The dependoparvovirus B (e.g., AAV5) MAAP polypeptide of either of claim 35 or 36, wherein a cell, cell-free system, or other translation system, comprising the MAAP polypeptide packages, secretes, and/or produces dependoparvovirus (e.g., dependoparvovirus A or B, e.g., an adeno-associated dependoparvovirus (AAV), e.g. AAV5 or a serotype other than AAV5) particle at a level of at least 50% or more than that of a cell, cell-free system, or other translation system, comprising an otherwise similar MAAP polypeptide that does not comprise the amino acid corresponding to the exogenous start codon.

38. The dependoparvovirus B (e.g., AAV5) MAAP polypeptide of any of claims 35-37, wherein the amino acid corresponding to the exogenous start codon comprises a methionine or a leucine.

39. The dependoparvovirus B (e.g., AAV5) MAAP polypeptide of any of claims 35-38, wherein the MAAP polypeptide comprises an amino acid sequence with at least 80, 85, 90, 91, 92, 93, 94, 95, 96, 97, 98, 99, or 100% identity to any of SEQ ID NOs: 3, 7, 11, 15, 19, 23, 27, 31, 35, 39, 43, 47, 51, 55, 59, 63, 67, 71, 75, 79, 83, 87, 91, 95, 99, 103, 107, 111, 115, 119, 123, 127, 131, 135, 139, 143, 147, 151, 155, 159, 163, 167, 171, 175, 179, 183, 187, 191, 195, 199, 203, 207, 211, 215, 219, 223, 227, 231, 235, 239, 243, 247, 251, 255, 259, 263, 267, 271, 275, 279, 283, 287, 291, 295, 299, 303, 307, 311, 315, or 319.

40. A nucleic acid comprising a sequence encoding a VP1 polypeptide, wherein the VP1 encoding sequence comprises a change or mutation corresponding to or arising from the presence of sequence encoding an exogenous start codon in the MAAP polypeptide encoding sequence.

41. The nucleic acid of claim 40, wherein the sequence encoding the VP1 polypeptide comprises a nucleic acid sequence with at least 80, 85, 90, 91, 92, 93, 94, 95, 96, 97, 98, 99, or 100% identity to any of SEQ ID NOs: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158, 162, 166, 170, 174, 178, 182, 186, 190, 194, 198, 202, 206, 210, 214, 218, 222, 226, 230, 234, 238, 242, 246, 250, 254, 258, 262, 266, 270, 274, 278, 282, 286, 290, 294, 298, 302, 306, 310, 314, or 318.

42. The nucleic acid of either of claim 40 or 41, wherein the MAAP polypeptide is a dependoparvovirus B (e.g., AAV5) MAAP polypeptide.

43. The nucleic acid of any of claims 40-42, wherein the VP1 polypeptide is a dependoparvovirus (e.g., dependoparvovirus A or B, e.g., AAV5 or a serotype other than AAV5) VP1 polypeptide.

44. A VP1 polypeptide comprising (a) any one of SEQ ID NOs: 1, 5, 9, 13, 17, 21, 25, 29, 33, 37, 41, 45, 49, 53, 57, 61, 65, 69, 73, 77, 81, 85, 89, 93, 97, 101, 105, 109, 113, 117, 121, 125, 129, 133, 137, 141, 145, 149, 153, 157, 161, 165, 169, 173, 177, 181, 185, 189, 193, 197, 201, 205, 209, 213, 217, 221, 225, 229, 233, 237, 241, 245, 249, 253, 257, 261, 165, 269, 273, 277, 281, 285, 289, 293, 297, 301, 305, 309, 313, or 317; or (b) a sequence encoded by the nucleic acid of any of claims 38-41.

45. A dependoparvovirus (e.g., dependoparvovirus A or B, e.g., AAV5 or a serotype other than AAV5) particle comprising the nucleic acid or VP1 polypeptide of any of claims 40-44.

46. A vector, e.g., a plasmid, comprising the nucleic acid of any of claims 40-45.

47. A cell, cell-free system, or other translation system, comprising the nucleic acid, vector, VP1 polypeptide, or particle of any of claims 40-46.

48. A method of making a dependoparvovirus (e.g., dependoparvovirus A or B, e.g., an adeno-associated dependoparvovirus (AAV), e.g. AAV5 or a serotype other than AAV5) particle, comprising:
providing a cell, cell-free system, or other translation system, comprising:
a nucleic acid of any of claims 1-29; and
cultivating the cell, cell-free system, or other translation system, under conditions suitable for the production of the dependoparvovirus particle,
thereby making the dependoparvovirus particle.

49. The method of claim 48, wherein the cell, cell-free system, or other translation system comprises a second nucleic acid molecule and said second nucleic acid molecule is packaged in the dependoparvovirus particle.

50. The method of claim 49, wherein the second nucleic acid comprises an exogenous sequence, e.g., encoding an exogenous polypeptide, e.g., a therapeutic product.

51. The method of either of claim 49 or 50, wherein a nucleic acid of any of claims 1-30 mediates the production of a dependoparvovirus particle which does not include said nucleic acid of any of claims 1-30.

52. The method of any of claims 48-51, wherein the dependoparvovirus particle is an adeno-associated dependoparvovirus (AAV) particle, e.g., an AAV5 particle or a particle of a serotype other than AAV5.

53. A method of delivering a payload (e.g., a nucleic acid) to a cell comprising contacting the cell with a dependoparvovirus particle comprising the payload, wherein the dependoparvovirus particle is:
a dependoparvovirus particle of any of claim 31, 32, or 45,
a dependoparvovirus particle made by a method of any of claims 48-52, or
a dependoparvovirus particle comprising a nucleic acid or polypeptide of any of claim 1-30, 33, 35-44, or 46.

54. A method of delivering a payload (e.g., a nucleic acid) to a subject comprising administering to the subject a dependoparvovirus particle comprising the payload, wherein the dependoparvovirus particle is:
a dependoparvovirus particle of any of claim 31, 32, or 45,
a dependoparvovirus particle made by a method of any of claims 48-52, or
a dependoparvovirus particle comprising a nucleic acid or polypeptide of any of claim 1-30, 33, 35-44, or 46.

55. The method of either of claim 53 or 54, wherein the particle delivers the payload to a preselected target cell, organ, tissue, or region.

56. A method of treating a disease or condition in a subject, comprising administering to the subject a dependoparvovirus particle in an amount effective to treat the disease or condition, wherein the dependoparvovirus particle is:
a dependoparvovirus particle of any of claim 31, 32, or 45,
a dependoparvovirus particle made by a method of any of claims 48-52, or
a dependoparvovirus particle comprising a nucleic acid or polypeptide of any of claim 1-30, 33, 35-44, or 46.
